(19) United States
(12) Patent Application Publication
(10) Pub. No.: US 2023/0131832 A1
(43) Pub. Date: Apr. 27, 2023

(54) ORGANIC LIGHT EMITTING DIODE AND ORGANIC LIGHT EMITTING DEVICE INCLUDING THE SAME

(71) Applicant: LG DISPLAY CO., LTD., Seoul (KR)

(72) Inventors: Tae-Ryang HONG, Paju-si (KR); Dong-Young KIM, Paju-si (KR)

(73) Assignee: LG DISPLAY CO., LTD., Seoul (KR)

(21) Appl. No.: 17/843,378

(22) Filed: Jun. 17, 2022

(30) Foreign Application Priority Data
Sep. 16, 2021 (KR) 10-2021-0123976

(51) Int. Cl. C07D 495/04; H01L 51/50; C07D 491/048; C07D 487/04; C07C 15/38; C07D 209/82; C07D 405/10; C07D 409/10; C07D 405/14; C07D 409/14; H01L 51/00

(52) U.S. Cl. CPC C07D 495/04; H01L 51/5016; C07D 491/048; C07D 487/04; C07C 15/38; C07D 209/82; C07D 405/10; C07D 409/10; C07D 405/14; C07D 409/14; H01L 51/0054; H01L 51/0064; H01L 51/0067; H01L 51/0071; H01L 51/0072; H01L 51/0073; H01L 51/0074; H01L 2251/552

(57) ABSTRACT

An organic light emitting diode (OLED) includes at least one emitting material layer (EML) disposed between two electrodes and including a first compound of a pyrimidine-based organic compound substituted with at least one electron-withdrawing group and a second compound of an organic compound having a tetracene-based core. The OLED can be included in an organic light emitting device. The first compound and the second compound can be the same emitting material layer or adjacently disposed emitting material layers. The OLED can lower its driving voltage and improve its luminous efficiency utilizing the advantages of the first and second compounds by adjusting energy levels between the first and second compounds.

### CROSS-REFERENCE TO RELATED APPLICATIONS

This application claims the benefit under 35 U.S.C. § 119(a) to Korean Patent Application No. 10-2021-0123976, filed in the Republic of Korea on Sep. 16, 2021, the entire contents of which are incorporated herein by reference into the present application.

### BACKGROUND

### Technical Field

The present disclosure relates to an organic light emitting diode, and more specifically, to an organic light emitting diode having excellent luminous properties and an organic light emitting device having the diode.

### Discussion of the Related Art

As display devices have become larger, there exists a need for a flat display device with lower spacing occupation. Among the flat display devices, a display device using an organic light emitting diode (OLED) has come into the spotlight.

The OLED can be formed as a thin film having a thickness less than 2000 Å and can be implement unidirectional or bidirectional images as electrode configurations. Also, the OLED can be formed on a flexible transparent substrate such as a plastic substrate so that OLED can implement a flexible or foldable display with ease. In addition, the OLED has advantages over LCD (liquid crystal display device), for example, the OLED can be driven at a lower voltage of 10 V or less and has very high color purity.

In the OLED, when electrical charges are injected into an emitting material layer between an electron injection electrode (i.e., cathode) and a hole injection electrode (i.e., anode), electrical charges are recombined to form excitons, and then emit light as the recombined excitons are shifted to a stable ground state.

Fluorescent materials of the related art have shown low luminous efficiency because only the singlet excitons are involved in the luminescence process thereof. The phosphorescent materials in which triplet excitons as well as the singlet excitons are involved in the luminescence process have relatively high luminous efficiency compared to the fluorescent material. However, the metal complex as the representative phosphorescent material has too short luminous lifespan to be applicable into commercial devices.

### SUMMARY OF THE DISCLOSURE

Accordingly, embodiments of the present disclosure are directed to an OLED and an organic light emitting device including the OLED that substantially obviates one or more of the problems due to the limitations and disadvantages of the related art.

An aspect of the present disclosure is to provide an OLED that can improve luminous efficiency, color purity and luminous lifespan and an organic light emitting device including the diode.

Additional features and aspects will be set forth in the description that follows, and in part will be apparent from the description, or can be learned by practice of the inventive concepts provided herein. Other features and aspects of the inventive concepts can be realized and attained by the structure particularly pointed out in the written description, or derivable therefrom, and the claims hereof as well as the appended drawings.

To achieve these and other aspects of the inventive concepts, as embodied and broadly described, an organic light emitting diode includes: a first electrode; a second electrode facing the first electrode; and an emissive layer disposed between the first and second electrodes and including at least one emitting material layer, wherein the at least one emitting material layer includes a first compound and a second compound, and wherein the first compound has the following structure of Formula 1 and the second compound has the following structure of Formula 5:

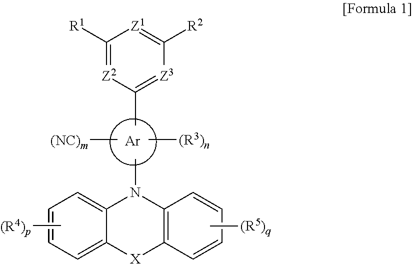

wherein, in Formula 1,

each of R¹ and R² is independently an unsubstituted or substituted C₆-C₃₀ aromatic group or an unsubstituted or substituted C₃-C₃₀ hetero aromatic group;

each of R³ to R⁵ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when n is an integer of 2 or more, each R³ is identical to or different from each other, when p is an integer of 2 or more, each R⁴ is identical to or different from each other, and when q is an integer of 2 or more, each R⁵ is identical to or different from each other;

optionally,

two adjacent elements to which R³ is attached when n is an integer of 2 or more, two adjacent elements to which R⁴ is attached when p is an integer of 2 or more, and/or two adjacent elements to which R⁵ is attached when q is an integer of 2 or more form an unsubstituted or substituted C₆-C₂₀ aromatic ring or an unsubstituted or substituted C₃-C₂₀ hetero aromatic ring;

X is a single bond, CR⁶R⁷, NR⁶, O or S, wherein each of R⁶ and R⁷ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group;

two of Z¹ to Z³ is N and other of Z¹ to Z³ is CR⁸, wherein R⁸ is —CN;

Ar is unsubstituted or substituted C₆-C₃₀ arylene or an unsubstituted or substituted C₃-C₃₀ hetero arylene;

m is an integer of 1 to 4;

n is an integer of 0 to 10; and

each of p and q is independently an integer of 0 to 4,

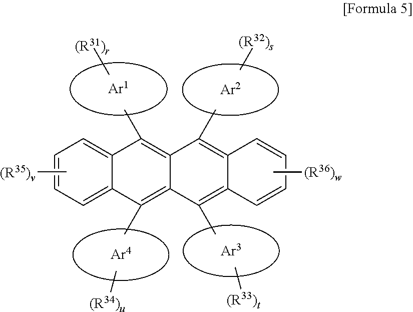

wherein, in Formula 5,

each of R³¹ to R³⁶ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when r is an integer of 2 or more, each R³¹ is identical to or different from each other, when s is an integer of 2 or more, each R³² is identical to or different from each other, when t is an integer of 2 or more, each R³³ is identical to or different from each other, when u is an integer of 2 or more, each R³⁴ is identical to or different from each other, when v is an integer of 2 or more, each R³⁵ is identical to or different from each other, when w is an integer of 2 or more, each R³⁶ is identical to or different from each other;

each of Ar¹ to Ar⁴ is independently an unsubstituted or substituted C₆-C₃₀ aromatic group or an unsubstituted or substituted C₃-C₃₀ hetero aromatic group;

each of r, s, t and u is independently an integer of 0 to 10; and

each of v and w is an integer of 0 to 4.

As an example, a Lowest Unoccupied Molecular Orbital (LUMO) energy level of the first compound and a LUMO energy level of the second compound can satisfy the following relationship in Equation (1):

LUMO^{FD}≥LUMO^{DF} (1)

wherein LUMO^{FD} is a LUMO energy level of the second compound and LUMO^{DF} is a LUMO energy level of the first compound.

Alternatively, a Highest Occupied Molecular Orbital (HOMO) energy level of the first compound and a HOMO energy level of the second compound can satisfy the following relationship in Equation (2):

HOMO^{FD}≥HOMO^{DF} (2)

wherein HOMO^{FD} is a HOMO energy level of the second compound and HOMO^{DF} is a HOMO energy level of the first compound.

As an example, the first compound can have an energy bandgap between a HOMO energy level and a LUMO energy level satisfying the following relationship in Equation (3):

2.0 eV≤Eg^{DF}≤3.0 eV (3)

wherein Eg^{DF} is an energy bandgap between a HOMO energy level and a LUMO energy level of the first compound.

In one exemplary aspect, the at least one emitting material layer can have a single-layered emitting material layer. The single-layered emitting material layer further includes a third compound.

Alternatively, the at least one emitting material layer includes a first emitting material layer disposed between the first and second electrodes and a second emitting material layer disposed between the first electrode and the first emitting material layer or between the first emitting material layer and the second electrode, and wherein the first emitting material layer includes the first compound and the second emitting material layer includes the second compound. The first emitting material layer can further include a third compound and the second emitting material layer can further include a fourth compound.

For example, the third compound and/or the fourth compound can have the following structure of Formula 8:

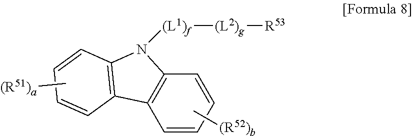

wherein, in Formula 8,

each of R⁵¹ and R⁵² is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when a is an integer of 2 or more, each R⁵¹ is identical to or different from each other, and when b is an integer of 2 or more, each R⁵² is identical to or different from each other;

R₅₃ is unsubstituted or substituted carbazolyl, unsubstituted or substituted dibenzofuranyl or unsubstituted or substituted dibenzothiophenyl;

each of L¹ and L² is independently unsubstituted or substituted C₆-C₃₀ arylene or unsubstituted or substituted C3-C30 hetero arylene; and

each of f and g is independently 0 or 1.

As an example, an excited triplet exciton energy level of the third compound and/or the fourth compound can be higher than an excited triplet exciton energy level of the first compound and the exited triplet exciton energy level of the first compound can be higher than an excited triplet exciton energy level of the second compound. Also, an excited singlet exciton energy level of the third compound and/or the fourth compound can be higher than an excited singlet exciton energy level of the first compound and the excited singlet exciton energy level of the first compound can be higher than an excited singlet exciton energy level of the second compound.

Optionally, when the at least one emitting material layer includes the first and second emitting material layers, the at least one emitting material layer can further includes a third emitting material layer disposed oppositely to the second emitting material layer with respect to the first emitting material layer.

In one exemplary aspect, the emissive layer can include a first emitting part disposed between the first and second electrodes, a second emitting part disposed between the first emitting part and the second electrode and a charge generation layer disposed between the first and second emitting parts, and wherein at least one of the first emitting part and the second emitting part can include the at least one emitting material layer.

In another aspect, an organic light emitting device, such as an organic light emitting display device or an organic light emitting luminescent device comprises a substrate and the OLED disposed over the substrate, as described above.

It is to be understood that both the foregoing general description and the following detailed description are exemplary and explanatory and are intended to provide further explanation of the inventive concepts as claimed.

### BRIEF DESCRIPTION OF THE DRAWINGS

The accompanying drawings, which are included to provide a further understanding of the disclosure, are incorporated in and constitute a part of this application, illustrate embodiments of the disclosure and together with the description serve to explain principles of the disclosure.

FIG. 1 is a schematic circuit diagram of an organic light emitting display device in accordance with the preset disclosure.

FIG. 2 is a schematic cross-sectional view illustrating an organic light emitting display device in accordance with an exemplary aspect of the present disclosure.

FIG. 3 is a schematic cross-sectional view illustrating an organic light emitting diode (OLED) in accordance with an exemplary aspect of the present disclosure.

FIG. 4 is a schematic diagram illustrating a state in which electrons are trapped in the second compound and emission zone in an EML is not formed uniformly when the LUMO energy levels among the first and second compounds are not properly adjusted.

FIG. 5 is a schematic diagram illustrating a state in which the LUMO energy levels among the first and second compounds are adjusted, holes and electrons are injected into an EML in balance, and therefore, emission zone is formed uniformly in the EML in accordance with an exemplary aspect of the present disclosure.

FIG. 6 is a schematic diagram illustrating luminous mechanism by singlet and triplet energy levels among luminous material in an EML in accordance with an exemplary aspect of the present disclosure.

FIG. 7 is a schematic cross-sectional view illustrating an OLED in accordance with another exemplary aspect of the present disclosure.

FIG. 8 is a schematic diagram illustrating a state in which the LUMO energy levels among the first and second compounds are adjusted, and therefore, electrons are not trapped in the second compound in accordance with another exemplary aspect of the present disclosure.

FIG. 9 is a schematic diagram illustrating luminous mechanism by singlet and triplet energy levels among luminous materials in EMLs in accordance with another exemplary aspect of the present disclosure.

FIG. 10 is a schematic cross-sectional view illustrating an OLED in accordance with still another exemplary aspect of the present disclosure.

FIG. 11 is a schematic diagram illustrating a state in which the LUMO energy levels among the first, second and fifth compounds are adjusted, and therefore, electrons are not trapped in the second and fifth compounds in accordance with still another exemplary aspect of the present disclosure.

FIG. 12 is a schematic diagram illustrating luminous mechanism by singlet and triplet energy levels among luminous materials in EMLs in accordance with still another exemplary aspect of the present disclosure.

FIG. 13 is a schematic cross-sectional view illustrating an OLED in accordance with still another exemplary aspect of the present disclosure.

FIG. 14 is a schematic cross-sectional view illustrating an organic light emitting display device in accordance with another exemplary aspect of the present disclosure.

FIG. 15 is a schematic cross-sectional view illustrating an OLED in accordance with still another exemplary aspect of the present disclosure.

FIG. 16 is a schematic cross-sectional view illustrating an organic light emitting display device in accordance with still another exemplary aspect of the present disclosure.

FIG. 17 is a schematic cross-sectional view illustrating an OLED in accordance with still another exemplary aspect of the present disclosure.

FIG. 18 is a schematic cross-sectional view illustrating an OLED in accordance with still another exemplary aspect of the present disclosure.

FIG. 19 is a schematic diagram illustrating an EML which includes four areas for estimating an emission area in accordance with Examples of the present disclosure.

FIG. 20 is a graph illustrating measurement results of photo-luminescence (PL) spectra in OLEDs fabricated in Examples in accordance with the present disclosure.

FIG. 21 is a graph illustrating measurement results of PL spectra in OLEDs fabricated in Comparative Examples.

FIG. 22 is a graph illustrating emission peak intensities in each emission area divided in EML of OLEDs fabricated in Examples in accordance with the present disclosure and Comparative Examples.

### DETAILED DESCRIPTION OF THE EMBODIMENTS

Reference and discussions will now be made below in detail to aspects, embodiments and examples of the disclosure, some examples of which are illustrated in the accompanying drawings.

The present disclosure relates to an organic light emitting diode (OLED) into which a first compound and a second compound having adjusted energy levels are applied in an identical EML or adjacently disposed EMLs and an organic light emitting device having the OLED. The OLED can be applied into an organic light emitting device such as an organic light emitting display device and an organic light emitting luminescent device. As an example, a display device applying the OLED will be described. All the components of an OLED or an organic light emitting device according to all embodiments of the present disclosure are operatively coupled and configured.

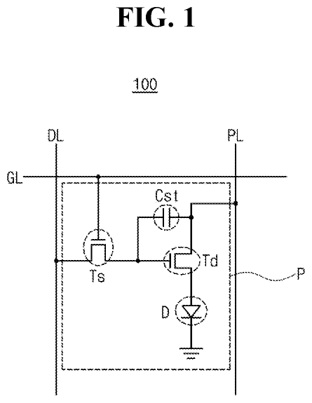

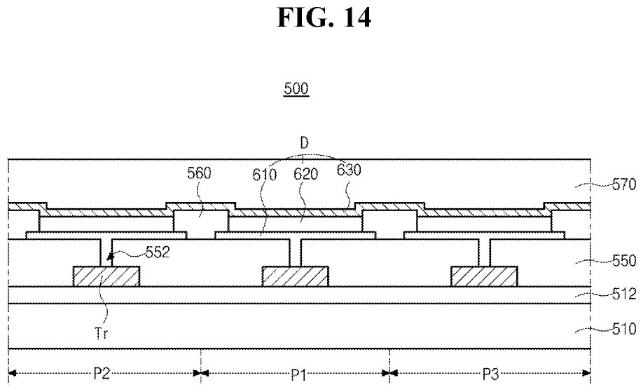

FIG. 1 is a schematic circuit diagram of an organic light emitting display device in accordance with the present disclosure. As illustrated in FIG. 1, a gate line GL, a data line DL and power line PL, each of which cross each other to define a pixel region P, in an organic light emitting display device 100. A switching thin film transistor Ts, a driving thin film transistor Td, a storage capacitor Cst and an organic light emitting diode D are formed within the pixel region P. The pixel region P can include a first pixel region P1, a second pixel region P2 and a third pixel region P3 (FIG. 14).

The switching thin film transistor Ts is connected to the gate line GL and the data line DL, and the driving thin film transistor Td and the storage capacitor Cst are connected between the switching thin film transistor Ts and the power line PL. The organic light emitting diode D is connected to the driving thin film transistor Td. When the switching thin film transistor Ts is turned on by a gate signal applied into the gate line GL, a data signal applied into the data line DL is applied into a gate electrode of the driving thin film transistor Td and one electrode of the storage capacitor Cst through the switching thin film transistor Ts.

The driving thin film transistor Td is turned on by the data signal applied into the gate electrode so that currents proportional to the data signal are supplied from the power line PL to the organic light emitting diode D through the driving thin film transistor Td. And then, the organic light emitting diode D emits light with a luminance proportional to the currents flowing through the driving thin film transistor Td. In this case, the storage capacitor Cst is charged with voltages proportional to the data signal so that the voltage of the gate electrode in the driving thin film transistor Td is kept constant during one frame. Therefore, the organic light emitting display device 100 can display a desired image.

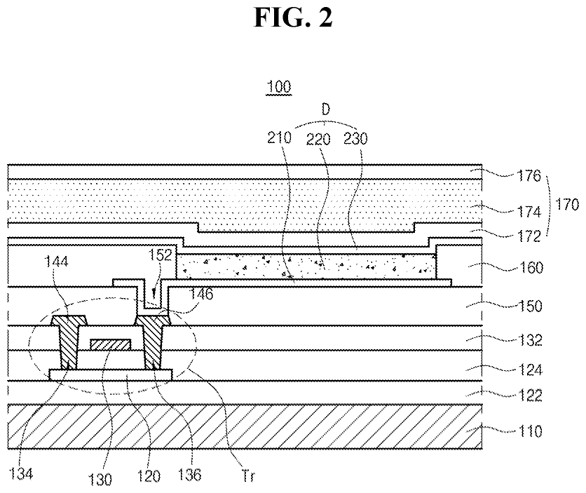

FIG. 2 is a schematic cross-sectional view of an organic light emitting display device 100 in accordance with an exemplary aspect of the present disclosure. All components of the organic light emitting device in accordance with all aspects of the present disclosure are operatively coupled and configured. As illustrated in FIG. 2, the organic light emitting display device 100 includes a substrate 110, a thin-film transistor Tr on the substrate 110, and an organic light emitting diode (OLED) D over the substrate 110 and connected to the thin film transistor Tr.

The substrate 110 can include, but is not limited to, glass, thin flexible material and/or polymer plastics. For example, the flexible material can include, but is not limited to, polyimide (PI), polyethersulfone (PES), polyethylenenaphthalate (PEN), polyethylene terephthalate (PET), polycarbonate (PC) and combination thereof. The substrate 110, over which the thin film transistor Tr and the OLED D are arranged, form an array substrate.

A buffer layer 122 can be disposed over the substrate 110, and the thin film transistor Tr is disposed over the buffer layer 122. The buffer layer 122 can be omitted.

A semiconductor layer 120 is disposed over the buffer layer 122. In one exemplary aspect, the semiconductor layer 120 can include, but is not limited to, oxide semiconductor materials. In this case, a light-shield pattern can be disposed under the semiconductor layer 120, and the light-shield pattern can prevent light from being incident toward the semiconductor layer 120, and thereby, preventing the semiconductor layer 120 from being deteriorated by the light. Alternatively, the semiconductor layer 120 can include, but is not limited to, polycrystalline silicon. In this case, opposite edges of the semiconductor layer 120 can be doped with impurities.

A gate insulating layer 124 made of an insulating material is disposed on the semiconductor layer 120. The gate insulating layer 124 can include, but is not limited to, an inorganic insulating material such as silicon oxide (SiOₓ) or silicon nitride (SiNₓ).

A gate electrode 130 made of a conductive material such as metal is disposed over the gate insulating layer 124 so as to correspond to a center of the semiconductor layer 120. While the gate insulating layer 124 is disposed over a whole area of the substrate 110 in FIG. 2, the gate insulating layer 124 can be patterned identically as the gate electrode 130.

An interlayer insulating layer 132 made of an insulating material is disposed on the gate electrode 130 with covering over an entire surface of the substrate 110. The interlayer insulating layer 132 can include, but is not limited to, an inorganic insulating material such as silicon oxide (SiOₓ) or silicon nitride (SiNₓ), or an organic insulating material such as benzocyclobutene or photo-acryl.

The interlayer insulating layer 132 has first and second semiconductor layer contact holes 134 and 136 that expose both sides of the semiconductor layer 120. The first and second semiconductor layer contact holes 134 and 136 are disposed over opposite sides of the gate electrode 130 with spacing apart from the gate electrode 130. The first and second semiconductor layer contact holes 134 and 136 are formed within the gate insulating layer 124 in FIG. 2. Alternatively, the first and second semiconductor layer contact holes 134 and 136 are formed only within the interlayer insulating layer 132 when the gate insulating layer 124 is patterned identically as the gate electrode 130.

A source electrode 144 and a drain electrode 146, which are made of conductive material such as a metal, are disposed on the interlayer insulating layer 132. The source electrode 144 and the drain electrode 146 are spaced apart from each other with respect to the gate electrode 130, and contact both sides of the semiconductor layer 120 through the first and second semiconductor layer contact holes 134 and 136, respectively.

The semiconductor layer 120, the gate electrode 130, the source electrode 144 and the drain electrode 146 constitute the thin film transistor Tr, which acts as a driving element. The thin film transistor Tr in FIG. 2 has a coplanar structure in which the gate electrode 130, the source electrode 144 and the drain electrode 146 are disposed over the semiconductor layer 120. Alternatively, the thin film transistor Tr can have an inverted staggered structure in which a gate electrode is disposed under a semiconductor layer and a source and drain electrodes are disposed over the semiconductor layer. In this case, the semiconductor layer can include amorphous silicon.

The gate line GL and the data line DL, which cross each other to define the pixel region P, and the switching element Ts, which is connected to the gate line GL and the data line DL, can be further formed in the pixel region P of FIG. 1. The switching element Ts is connected to the thin film transistor Tr, which is a driving element. Besides, the power line PL is spaced apart in parallel from the gate line GL or the data line DL, and the thin film transistor Tr can further include a storage capacitor Cst configured to constantly keep voltage of the gate electrode 130 for one frame.

In addition, the organic light emitting display device 100 can include a color filter layer that includes dyes or pigments for transmitting specific wavelength light of light emitted from the OLED D. For example, the color filter layer can transmit light of specific wavelength such as red (R), green (G) and/or blue (B). Each of red, green, and blue color filter patterns can be disposed separately in each pixel region P. In this case, the organic light emitting display device 100 can implement full-color through the color filter layer.

For example, when the organic light emitting display device 100 is a bottom-emission type, the color filter layer can be disposed on the interlayer insulating layer 132 with corresponding to the OLED D. Alternatively, when the organic light emitting display device 100 is a top-emission type, the color filter layer can be disposed over the OLED D, for example, a second electrode 230.

A passivation layer 150 is disposed on the source and drain electrodes 144 and 146 over the whole substrate 110. The passivation layer 150 has a flat top surface and a drain contact hole 152 that exposes the drain electrode 146 of the thin film transistor Tr. While the drain contact hole 152 is disposed on the second semiconductor layer contact hole 136, it can be spaced apart from the second semiconductor layer contact hole 136.

The OLED D includes a first electrode 210 that is disposed on the passivation layer 150 and connected to the drain electrode 146 of the thin film transistor Tr. The OLED D further includes an emissive layer 220 and a second electrode 230 each of which is disposed sequentially on the first electrode 210.

The first electrode 210 is disposed in each pixel region. The first electrode 210 can be an anode and include a conductive material having a relatively high work function value. For example, the first electrode 210 can include, but is not limited to, a transparent conductive material such as indium tin oxide (ITO), indium zinc oxide (IZO), indium tin zinc oxide (ITZO), tin oxide (SnO), zinc oxide (ZnO), cerium doped indium oxide (ICO), aluminum doped zinc oxide (Al:ZnO, AZO), and the like.

In one exemplary aspect, when the organic light emitting display device 100 is a bottom-emission type, the first electrode 210 can have a single-layered structure of a transparent conductive material. Alternatively, when the organic light emitting display device 100 is a top-emission type, a reflective electrode or a reflective layer can be disposed under the first electrode 210. For example, the reflective electrode or the reflective layer can include, but is not limited to, silver (Ag) or aluminum-palladium-copper (APC) alloy. In the OLED D of the top-emission type, the first electrode 210 can have a triple-layered structure of ITO/Ag/ITO or ITO/APC/ITO. In addition, a bank layer 160 is disposed on the passivation layer 150 in order to cover edges of the first electrode 210. The bank layer 160 exposes a center of the first electrode 210 corresponding to the pixel region P.

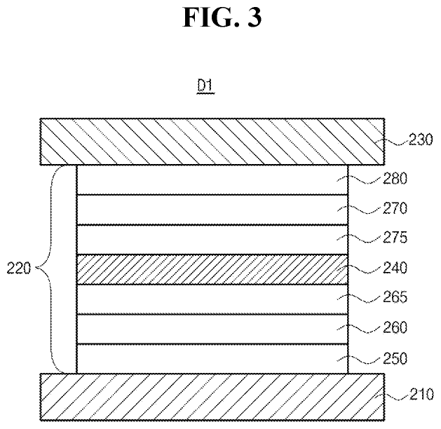

The emissive layer 220 is disposed on the first electrode 210. In one exemplary aspect, the emissive layer 220 can have a single-layered structure of an emitting material layer (EML). Alternatively, the emissive layer 220 can have a multiple-layered structure of a hole injection layer (HIL), a hole transport layer (HTL), an electron blocking layer (EBL), an EML, a hole blocking layer (HBL), an electron transport layer (ETL) and/or an electron injection layer (EIL) (see, FIGS. 3, 7, 10 and 13). In one aspect, the emissive layer 220 can have one emitting part. Alternatively, the emissive layer 220 can have multiple emitting parts to form a tandem structure.

The second electrode 230 is disposed over the substrate 110 above which the emissive layer 220 is disposed. The second electrode 230 can be disposed over a whole display area and can include a conductive material with a relatively low work function value compared to the first electrode 210. The second electrode 230 can be a cathode. For example, the second electrode 230 can include, but is not limited to, aluminum (Al), magnesium (Mg), calcium (Ca), silver (Ag), alloy thereof or combination thereof such as aluminum-magnesium alloy (Al—Mg). When the organic light emitting display device 100 is a top-emission type, the second electrode 230 is thin so as to have light-transmissive (semi-transmissive) property.

In addition, an encapsulation film 170 can be disposed over the second electrode 230 in order to prevent outer moisture from penetrating into the OLED D. The encapsulation film 170 can have, but is not limited to, a laminated structure of a first inorganic insulating film 172, an organic insulating film 174 and a second inorganic insulating film 176.

Moreover, the organic light emitting display device 100 can have a polarizer in order to decrease external light reflection. For example, the polarizer can be a circular polarizer. When the organic light emitting display device 100 is a bottom-emission type, the polarizer can be disposed under the substrate 110. Alternatively, when the organic light emitting display device 100 is a top-emission type, the polarizer can be disposed over the encapsulation film 170. In addition, a cover window can be attached to the encapsulation film 170 or the polarizer. In this case, the substrate 110 and the cover window can have a flexible property, thus the organic light emitting display device 100 can be a flexible display device.

Now, we will describe the OLED in more detail. FIG. 3 is a schematic cross-sectional view illustrating an OLED in accordance with an exemplary aspect of the present disclosure. As illustrated in FIG. 3, the OLED D1 comprises first and second electrodes 210 and 230 facing each other, and an emissive layer 220 having single emitting part disposed between the first and second electrodes 210 and 230. The organic light emitting display device 100 includes a red pixel region, a green pixel region and a blue pixel region, and the OLED D1 can be disposed in the green pixel region.

The emissive layer 220 includes an EML 240 disposed between the first and second electrodes 210 and 230. Also, the emissive layer 220 can include at least one of an HTL 260 disposed between the first electrode 210 and the EML 240 and an ETL 270 disposed between the second electrode 230 and the EML 240. In addition, the emissive layer 220 can further include at least one of an HIL 250 disposed between the first electrode 210 and the HTL 260 and an EIL 280 disposed between the second electrode 230 and the ETL 270. Alternatively, the emissive layer 220 can further include an EBL 265 disposed between the HTL 260 and the EML 240 and/or an HBL 275 disposed between the EML 240 and the ETL 270.

The first electrode 210 can be an anode that provides holes into the EML 240. The first electrode 210 can include, but is not limited to, a conductive material having a relatively high work function value, for example, a transparent conductive oxide (TCO). In an exemplary aspect, the first electrode 210 can include, but is not limited to, ITO, IZO, ITZO, SnO, ZnO, ICO, AZO, and the like.

The second electrode 230 can be a cathode that provides electrons into the EML 240. The second electrode 230 can include, but is not limited to, a conductive material having a relatively low work function values, i.e., a highly reflective material such as Al, Mg, Ca, Ag, alloy thereof, combination thereof, and the like.

The EML 240 can include a first compound (Compound 1) DF, a second compound (Compound 2) FD and, optionally a third compound (Compound 3) H. For example, the first compound DF can be delayed fluorescent material, the second compound FD can be fluorescent material, and the third compound H can be host.

When holes and electrons meet each other to form excitons in the EML 240, singlet exciton with a paired spin state and triplet exciton with an unpaired spin state are generated in a ratio of 1:3 by spin arrangement. Since the conventional fluorescent materials can utilize only the singlet excitons, they exhibit low luminous efficiency. The phosphorescent materials can utilize the triplet excitons as well as the singlet excitons, while they show too short luminous lifespan to be applicable to commercial devices.

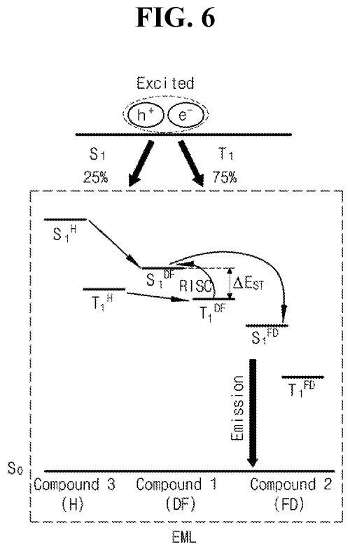

The first compound DF can be delayed fluorescent material having thermally activated delayed fluorescence (TADF) properties that can solve the problems accompanied by the conventional art fluorescent and/or phosphorescent materials. The delayed fluorescent material has very narrow energy level bandgap ΔE_{ST} between a singlet energy level S₁^{DF} and a triplet energy level T₁^{DF}(FIG. 6). Accordingly, the excitons of singlet energy level S₁^{DF} as well as the excitons of triplet energy level T₁^{DF} in the first compound DF of the delayed fluorescent material can be transferred to an intermediate energy level state, i.e. ICT (intramolecular charge transfer) state (S₁^{DF}→ICT←T₁^{DF}), and then the intermediate state excitons can be shifted to a ground state (ICT→S₀^{DF}).

The delayed fluorescent material must has an energy level bandgap ΔE_{ST} (FIG. 6) equal to or less than about 0.3 eV, for example, from about 0.05 to about 0.3 eV, between the singlet energy level S₁^{DF} and the triplet energy level T₁^{DF} so that exciton energy in both the singlet energy level S₁^{DF} and the triplet energy level T₁^{DF} can be transferred to the ICT state. The material having little energy level bandgap ΔE_{ST} between the singlet energy level S₁^{DF} and the triplet energy level T₁^{DF} can exhibit common fluorescence with Inter system Crossing (ISC) in which the excitons of singlet energy level S₁^{DF} can be shifted to its ground state S₀^{DF}, as well as delayed fluorescence with Reverse Inter System Crossing (RISC) in which the excitons of triplet energy level T₁^{DF} can be converted upwardly to the excitons of singlet energy level S₁^{DF}, and then the exciton of singlet energy level S₁^{DF} transferred from the triplet energy level T₁^{DF} can be transferred to the ground state S₀^{DF}.

The first compound DF can be delayed fluorescent material having an electron acceptor moiety consisting of a pyrimidine ring and an electron donor moiety of a fused hetero aromatic ring with at least one nitrogen atom as a nuclear atom. The first compound DF of the delayed fluorescent material can have the following structure of Formula 1:

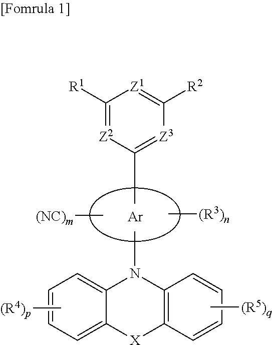

wherein, in Formula 1,

each of R¹ and R² is independently an unsubstituted or substituted C₆-C₃₀ aromatic group or an unsubstituted or substituted C₃-C₃₀ hetero aromatic group;

each of R³ to R⁵ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when n is an integer of 2 or more, each R³ is identical to or different from each other, when p is an integer of 2 or more, each R⁴ is identical to or different from each other, and when q is an integer of 2 or more, each R⁵ is identical to or different from each other;

optionally,

two adjacent elements to which R³ is attached when n is an integer of 2 or more, two adjacent elements to which R⁴ is attached when p is an integer of 2 or more, and/or two adjacent elements to which R⁵ is attached when q is an integer of 2 or more form an unsubstituted or substituted C₆-C₂₀ aromatic ring or an unsubstituted or substituted C₃-C₂₀ hetero aromatic ring;

X is a single bond, CR⁶R⁷, NR⁶, O or S, wherein each of R⁶ and R⁷ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group;

two of Z¹ to Z³ is N and other of Z¹ to Z³ is CR⁸, wherein R⁸ is —CN;

Ar is unsubstituted or substituted C₆-C₃₀ arylene or an unsubstituted or substituted C₃-C₃₀ hetero arylene;

m is an integer of 1 to 4;

n is an integer of 0 to 10; and

each of p and q is independently an integer of 0 to 4.

As used herein, substituent in the term “substituted” includes, but is not limited to, deuterium, tritium, unsubstituted or deuterium or halogen-substituted C₁-C₂₀ alkyl, unsubstituted or deuterium or halogen-substituted C₁-C₂₀ alkoxy, halogen, cyano, —CF₃, a hydroxyl group, a carboxylic group, a carbonyl group, an amino group, a C₁-C₁₀ alkyl amino group, a C₆-C₃₀ aryl amino group, a C₃-C₃₀ hetero aryl amino group, a C₆-C₃₀ aryl group, a C₃-C₃₀ hetero aryl group, a nitro group, a hydrazyl group, a sulfonate group, a C₁-C₂₀ alkyl silyl group, a C₆-C₃₀ aryl silyl group and a C₃-C₃₀ hetero aryl silyl group.

For example, each of the C₆-C₃₀ aromatic group, the C₃-C₃₀ hetero aromatic group, the C₆-C₂₀ aromatic ring, the C₃-C₃₀ hetero aromatic ring, the C₆-C₃₀ arylene and the C₃-C₃₀ hetero arylene constituting R¹ to R⁷ and Ar in Formula 1 can be independently unsubstituted or substituted with at least one of deuterium, tritium, C₁-C₂₀ alkyl, C₆-C₃₀ aryl and/or C₃-C₃₀ hetero aryl.

As used herein, the term “hetero” in such as “a hetero aromatic group”, “hetero aryl”, “hetero aryl alkyl”, “hetero aryl oxy”, “hetero aryl amino” and “hetero arylene group” means that at least one carbon atom, for example 1-5 carbons atoms, constituting an aromatic group or ring is substituted with at least one hetero atom selected from the group consisting of N, O, S, P and combination thereof.

As used herein, the term “aromatic” or “aryl” is well known in the art. The term includes monocyclic rings linked covalently or fused-ring polycyclic groups. An aromatic group or aryl can be unsubstituted or substituted. As an example, the C₆-C₃₀aromatic group, which can constitute R¹ to R⁷ in Formula 1, can include independently, but is not limited to, C₆-C₃₀ aryl, C₇-C₃₀ aryl alkyl, C₆-C₃₀ aryl oxy and C₆-C₃₀ aryl amino. As an example, the C₆-C₃₀ aryl group, which can constitute R¹ to R⁷ in Formula 1, can include independently, but is not limited to, a non-fused or fused aryl group such as phenyl, biphenyl, terphenyl, naphthyl, anthracenyl, pentalenyl, indenyl, indeno-indenyl, heptalenyl, biphenylenyl, indacenyl, phenalenyl, phenanthrenyl, benzo-phenanthrenyl, dibenzo-phenanthrenyl, azulenyl, pyrenyl, fluoranthenyl, triphenylenyl, chrysenyl, tetraphenylenyl, tetracenyl, pleiadenyl, picenyl, pentaphenylenyl, pentacenyl, fluorenyl, indeno-fluorenyl and spiro-fluorenyl.

As used herein, the term “hetero aromatic” or “hetero aryl” refers to a heterocycles including hetero atoms selected from N, O and S in a ring where the ring system is an aromatic ring. The term includes monocyclic rings linked covalently or fused-ring polycyclic groups. A hetero aromatic group can be unsubstituted or substituted. As an example, the C₃-C₃₀ hetero aromatic group, which can be constitute R¹ to R⁷ in Formula 1, can include independently, but is not limited to, C₃-C₃₀ hetero aryl, C₄-C₃₀ hetero aryl alkyl, C₃-C₃₀ hetero aryl oxy and C₃-C₃₀ hetero aryl amino.

As an example, the C₃-C₃₀ hetero aryl group, which can constitute R¹ to R⁷ in Formula 1, can include independently, but is not limited to, an unfused or fused hetero aryl group such as pyrrolyl, pyridinyl, pyrimidinyl, pyrazinyl, pyridazinyl, triazinyl, tetrazinyl, imidazolyl, pyrazolyl, indolyl, iso-indolyl, indazolyl, indolizinyl, pyrrolizinyl, carbazolyl, benzo-carbazolyl, dibenzo-carbazolyl, indolo-carbazolyl, indeno-carbazolyl, benzo-furo-carbazolyl, benzo-thieno-carbazolyl, carbolinyl, quinolinyl, iso-quinolinyl, phthalazinyl, quinoxalinyl, cinnolinyl, quinazolinyl, quinolizinyl, purinyl, benzo-quinolinyl, benzo-iso-quinolinyl, benzo-quinazolinyl, benzo-quinoxalinyl, acridinyl, phenazinyl, phenoxazinyl, phenothiazinyl, phenanthrolinyl, perimidinyl, phenanthridinyl, pteridinyl, naphthyridinyl, furanyl, pyranyl, oxazinyl, oxazolyl, oxadiazolyl, triazolyl, dioxanyl, benzo-furanyl, dibenzo-furanyl, thiopyranyl, xanthenyl, chromenyl, iso-chromenyl, thiazinyl, thiophenyl, benzo-thiophenyl, dibenzo-thiophenyl, difuro-pyrazinyl, benzofuro-dibenzo-furanyl, benzothieno-benzo-thiophenyl, benzothieno-dibenzo-thiophenyl, benzothieno-benzo-furanyl, benzothieno-dibenzo-furanyl, N-substituted spiro-fluorenyl, spiro-fluoreno-acridinyl and spiro-fluoreno-xanthenyl.

For example, the fused hetero aromatic ring, i.e. the ring including X, of the nuclear donor moiety in Formula 1 can include a fused hetero aromatic ring including 1 or 2 nitrogen atoms as a nuclear atom. As an example, such a fused hetero aromatic ring can include, but is not limited to, a carbazolyl moiety, an acridinyl moiety, an acridonyl moiety, a phenazinyl moiety, a phenoxazinyl moiety or a phenothiazinyl moiety.

In addition, the C₆-C₂₀ aromatic ring and the C₃-C₂₀ hetero aromatic ring formed by two adjacent elements to which R³ is attached, two adjacent elements to which R⁴ is attached and/or two adjacent elements to which R⁵ is attached can include, but is not limited to, a benzene ring, a naphthalene ring, an indene ring, a pyridine ring, an indole ring, a furan ring, a benzo-furan ring, a dibenzo-furan ring, a thiophene ring, a benzo-thiophene ring, a dibenzo-thiophene ring and/or combination thereof. For example, the C₆-C₂₀ aromatic ring and the C₃-C₂₀ hetero aromatic ring formed by two adjacent elements to which R³ is attached, two adjacent elements to which R⁴ is attached and/or two adjacent elements to which R⁵ is attached can include a benzo-thiophene ring, a benzo-furan ring, an indole ring, an indene ring, a dibenzo-thiophene ring and a dibenzo-furan ring, each of which is independently unsubstituted or substituted with at least one of deuterium, tritium, C₁-C₂₀ alkyl, C₆-C₃₀ aryl and/or C₃-C₃₀ hetero aryl.

For example, when each of R¹ to R⁷ is independently the C₆-C₃₀ aromatic group or the C₃-C₃₀ hetero aromatic group, or two adjacent groups among R³ to R⁵ form the C₆-C₃₀ aromatic ring or the C₃-C₂₀ hetero aromatic ring, each of the aromatic group, the hetero aromatic group, the aromatic ring and the hetero aromatic ring can be independently unsubstituted or substituted with at least one group selected from C₁-C₁₀ alkyl (ex. C₁-C₅ alkyl such as tert-butyl), C₆-C₃₀ aryl (ex. C₆-C₁₅ aryl such as phenyl) and/or C₃-C₃₀ hetero aryl (ex. C₃-C₁₅ hetero aryl such as pyridyl).

Each of the C₆-C₃₀ arylene and the C₃-C₃₀ hetero arylene of Ar in Formula 1 can include a divalent aromatic and hetero aromatic bridging group corresponding to each of the C₆-C₃₀ aryl and the C₃-C₃₀ hetero aryl which can be R¹ to R⁷, respectively.

In one exemplary aspect, each of R¹ and R² in Formula 1 can be phenyl, Z² or Z³ in Formula 1 can be CR⁸, Ar in Formula 1 can be phenylene, and the C₆-C₃₀ aromatic group and the C₃-C₃₀ hetero aromatic group constituting each of R³ to R⁵ in Formula 1 can be the C₆-C₃₀ aryl and the C₃-C₃₀ hetero aryl, respectively. For example, such an organic compound can have the following structure of Formula 2:

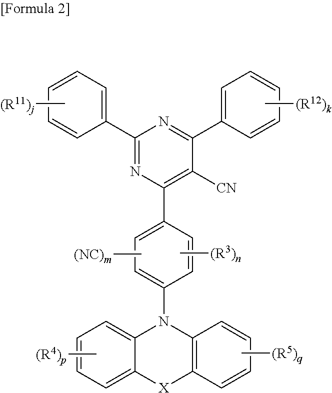

wherein, in Formula 2,

each of R³, R⁴, R⁵, R¹¹ and R¹² is independently hydrogen, protium, deuterium, tritium, unsubstituted or substituted C₁-C₂₀ alkyl, unsubstituted or substituted C₆-C₃₀ aryl or unsubstituted or substituted C₃-C₃₀ hetero aryl, when j is an integer of 2 or more, each R¹¹ is identical to or different from each other, when k is an integer of 2 or more, each R¹² is identical to or different from each other, when n is an integer of 2 or more, each R³ is identical to or different from each other, when p is an integer of 2 or more, each R⁴ is identical to or different from each other, and when q is an integer of 2 or more, each R⁵ is identical to or different from each other;

optionally,

two adjacent elements to which R¹¹ is attached when j is an integer of 2 or more, two adjacent elements to which R² is attached when k is an integer of 2 or more, two adjacent elements to which R³ is attached when n is an integer of 2 or more, two adjacent elements to which R⁴ is attached when p is an integer of 2 or more, two adjacent elements to which R⁵ is attached when q is an integer of 2 or more form an unsubstituted or substituted C₆-C₂₀ aromatic ring or an unsubstituted or substituted C₃-C₂₀ hetero aromatic ring;

X is a single bond, CR⁶R⁷, NR⁶, O or S, wherein each of R⁶ and R⁷ is independently hydrogen, protium, deuterium, tritium, unsubstituted or substituted C₁-C₂₀ alkyl, unsubstituted or substituted C₆-C₃₀ aryl or unsubstituted or substituted C₃-C₃₀ hetero aryl;

each of j and k is independently an integer of 0 to 5;

m is an integer of 1 to 4;

n is an integer of 0 to 3; and

each of p and q is independently an integer of 0 to 4.

For example, each of the C₆-C₃₀ aryl, the C₃-C₃₀ hetero aryl, the C₆-C₃₀ aromatic ring and the C₃-C₃₀ hetero aromatic ring constituting or formed by R³ to R⁷, R¹¹ and R¹² in Formula 2 can be independently unsubstituted or substituted with at least one of deuterium, tritium, C₁-C₂₀ alkyl, C₆-C₃₀ aryl and/or C₃-C₃₀ hetero aryl.

In still another exemplary aspect, each of R¹ and R² in Formula 1 can be phenyl, Z² or Z³ in Formula 1 can be CR⁸, Ar in Formula 1 can be phenylene, the C₆-C₃₀ aromatic group and the C₃-C₃₀ hetero aromatic group constituting each of R³ to R⁵ in formula 1 can be the C₆-C₃₀ aryl and the C₃-C₃₀ hetero aryl, X in Formula 1 can be a single bond, two adjacent elements to which R⁴ is attached or two adjacent elements to which R⁵ is attached in Formula 1 can form the C₆-C₂₀ aromatic ring or the C₃-C₃0 hetero aromatic ring, respectively. For example, such an organic compound can have the following structure of Formula 3:

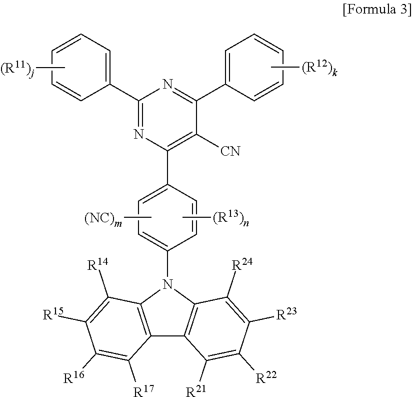

wherein, in Formula 3,

each of R¹¹ to R¹³ is independently hydrogen, protium, deuterium, tritium, unsubstituted or substituted C₁-C₂₀ alkyl, unsubstituted or substituted C₆-C₃₀ aryl or unsubstituted or substituted C₃-C₃₀ hetero aryl, when j is an integer of 2 or more, each R¹¹ is identical to or different from each other, when k is an integer of 2 or more, each R¹² is identical to or different from each other, and when n is an integer of 2 or more, each R³ is identical to or different from each other;

each of R¹⁴ to R¹⁷ is independently hydrogen, protium deuterium, tritium, unsubstituted or substituted C₁-C₂₀ alkyl, unsubstituted or substituted C₆-C₃₀ aryl or unsubstituted or substituted C₃-C₃₀ hetero aryl;

each of R²¹ to R²⁴ is independently hydrogen, protium deuterium, tritium, unsubstituted or substituted C₁-C₂₀ alkyl, unsubstituted or substituted C₆-C₃₀ aryl or unsubstituted or substituted C₃-C₃₀ hetero aryl, or two adjacent groups among R²¹ to R²⁴ form an unsubstituted or substituted C₆-C₂₀ aromatic ring or an unsubstituted or substituted C₃-C₂₀ hetero aromatic ring, wherein at least two adjacent groups among R²¹ to R²⁴ form an unsubstituted or substituted C₆-C₂₀ aromatic ring or an unsubstituted or substituted C₃-C₂₀ hetero aromatic ring;

m is an integer of 1 or 2; and

n is an integer of 0 to 3.

For example, each of the C₆-C₃₀ aryl, the C₃-C₃₀ hetero aryl, the C₆-C₃₀ aromatic ring and the C₃-C₃₀ hetero aromatic ring constituting or formed by R¹¹ to R¹⁷, R²¹ to R²⁴ in Formula 3 can be independently unsubstituted or substituted with at least one of deuterium, tritium, C₁-C₂₀ alkyl, C₆-C₃₀ aryl and/or C₃-C₃₀ hetero aryl.

More particularly, the first compound DF can be selected from, but is not limited to, the following compounds of Formula 4:

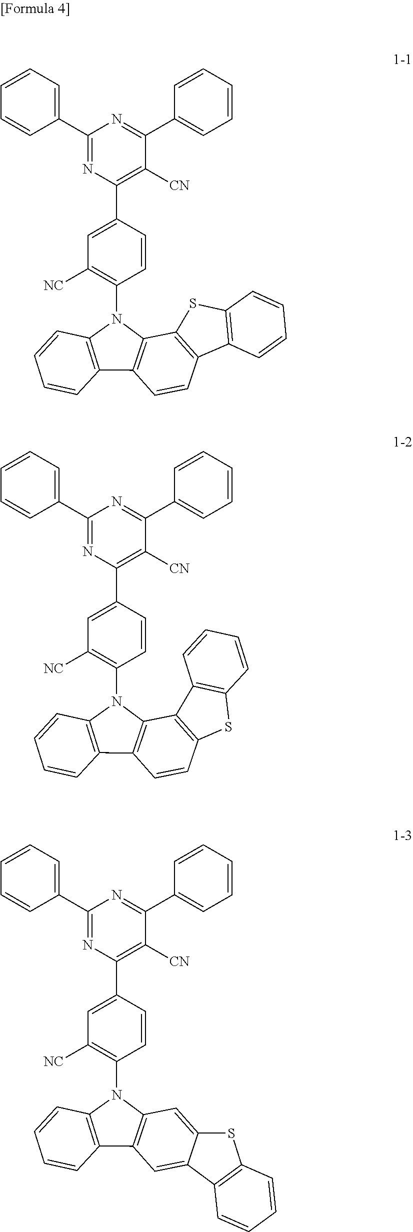 [SMILES: N#Cc1cc(-c2nc(-c3ccccc3)nc(-c3ccccc3)c2C#N)ccc1-n1c2ccccc2c2ccc3c4ccccc4sc3c21 ; N#Cc1cc(-c2nc(-c3ccccc3)nc(-c3ccccc3)c2C#N)ccc1-n1c2ccccc2c2ccc3sc4ccccc4c3c21 ; N#Cc1cc(-c2nc(-c3ccccc3)nc(-c3ccccc3)c2C#N)ccc1-n1c2ccccc2c2cc3c(cc21)sc1ccccc13]

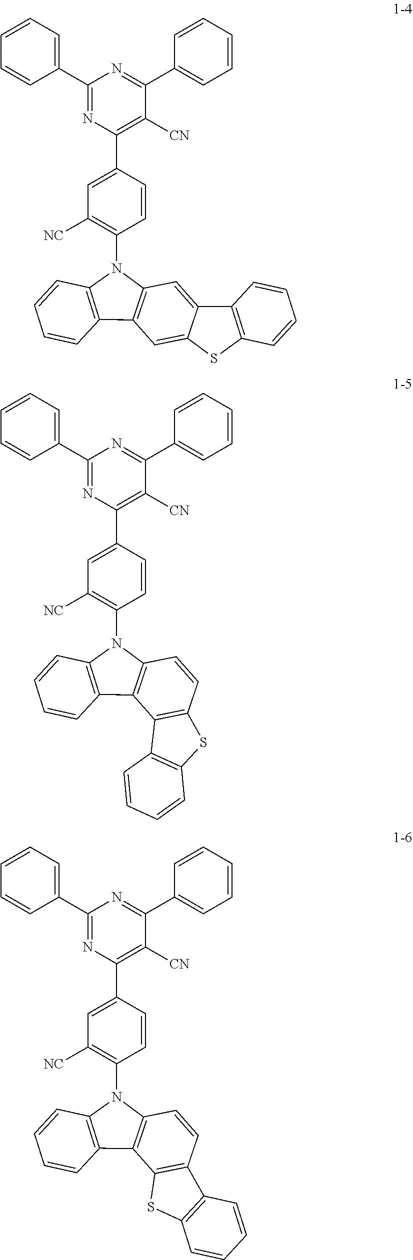

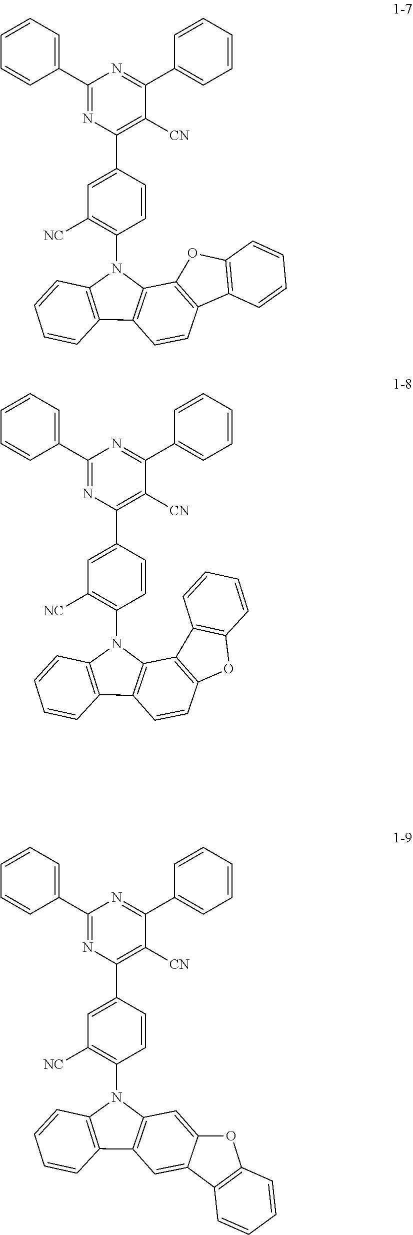 [SMILES: N#Cc1cc(-c2nc(-c3ccccc3)nc(-c3ccccc3)c2C#N)ccc1-n1c2ccccc2c2ccc3c4ccccc4oc3c21 ; N#Cc1cc(-c2nc(-c3ccccc3)nc(-c3ccccc3)c2C#N)ccc1-n1c2ccccc2c2ccc3oc4ccccc4c3c21 ; N#Cc1cc(-c2nc(-c3ccccc3)nc(-c3ccccc3)c2C#N)ccc1-n1c2ccccc2c2cc3c(cc21)oc1ccccc13]

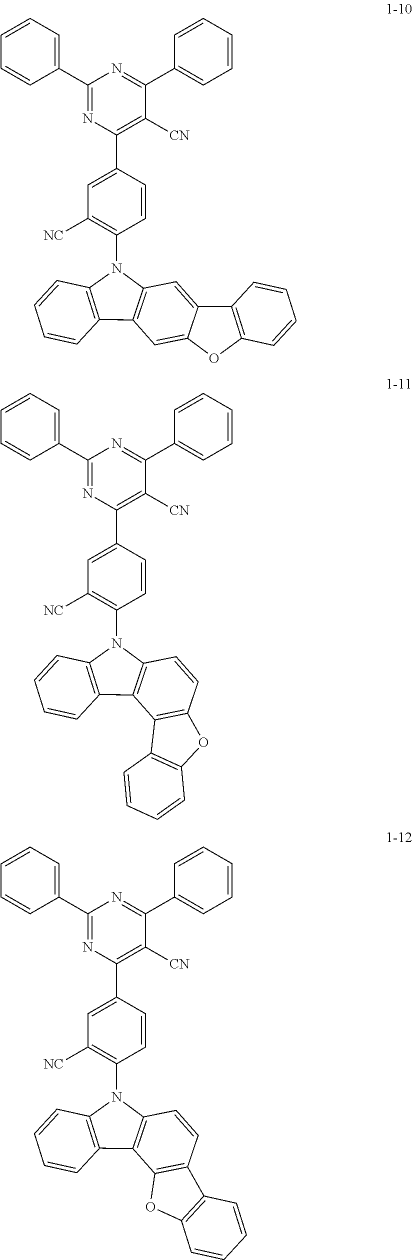

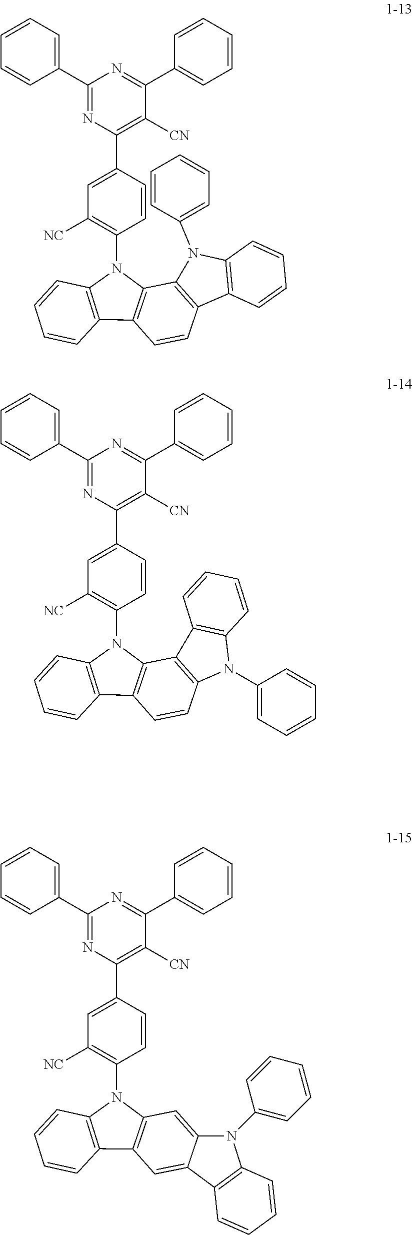 [SMILES: N#Cc1cc(-c2nc(-c3ccccc3)nc(-c3ccccc3)c2C#N)ccc1-n1c2ccccc2c2ccc3c4ccccc4n(-c4ccccc4)c3c21 ; N#Cc1cc(-c2nc(-c3ccccc3)nc(-c3ccccc3)c2C#N)ccc1-n1c2ccccc2c2ccc3c(c4ccccc4n3-c3ccccc3)c21 ; N#Cc1cc(-c2nc(-c3ccccc3)nc(-c3ccccc3)c2C#N)ccc1-n1c2ccccc2c2cc3c4ccccc4n(-c4ccccc4)c3cc21]

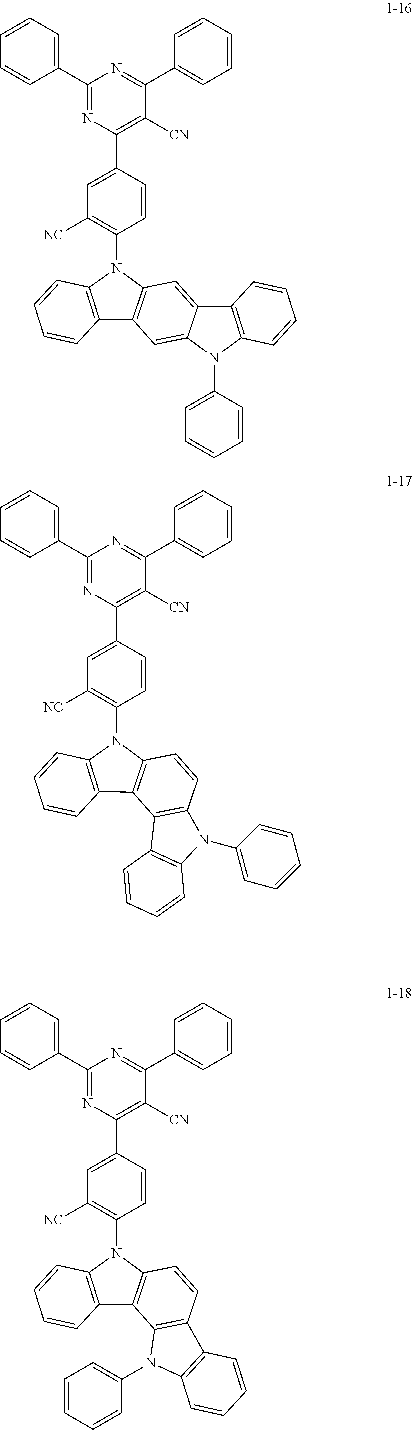

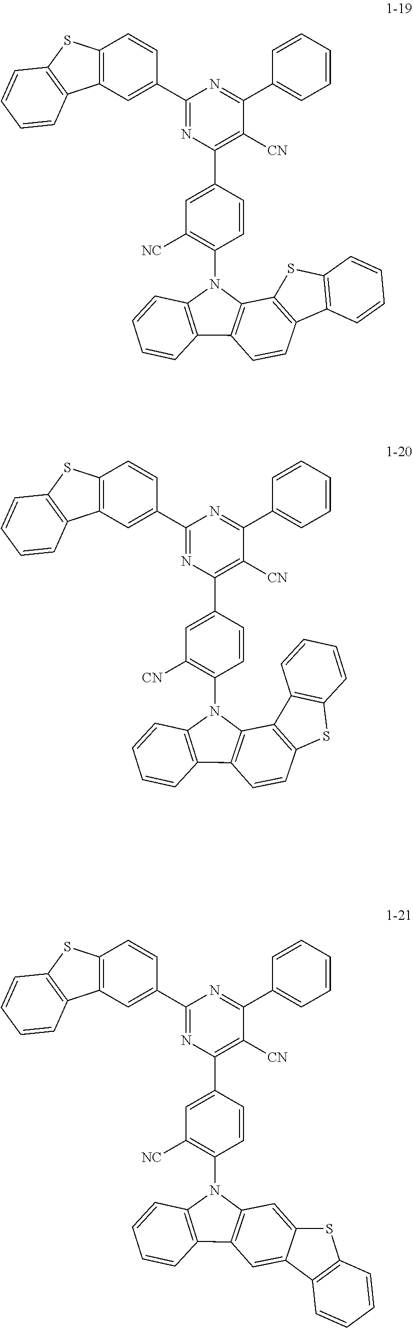

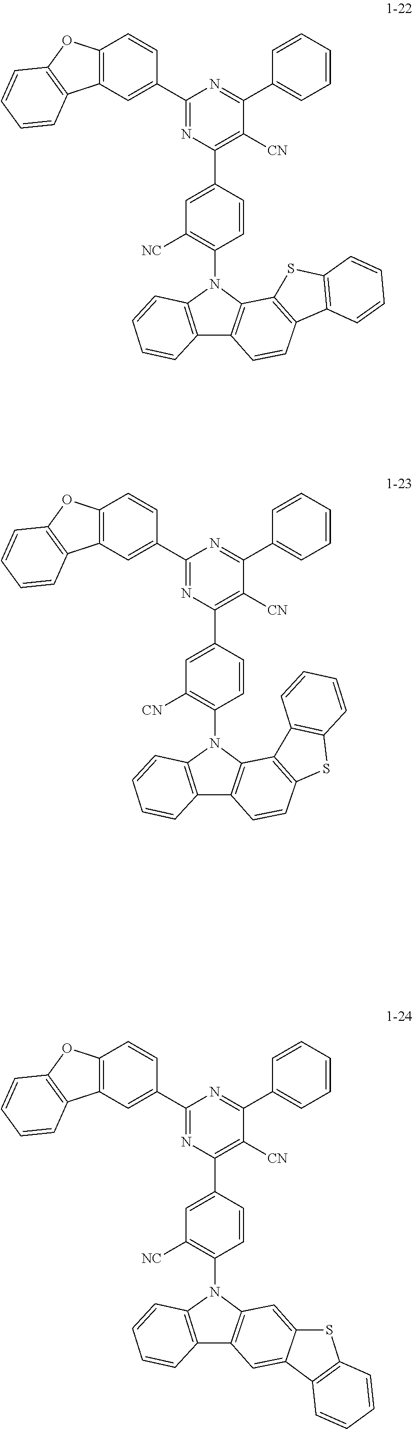

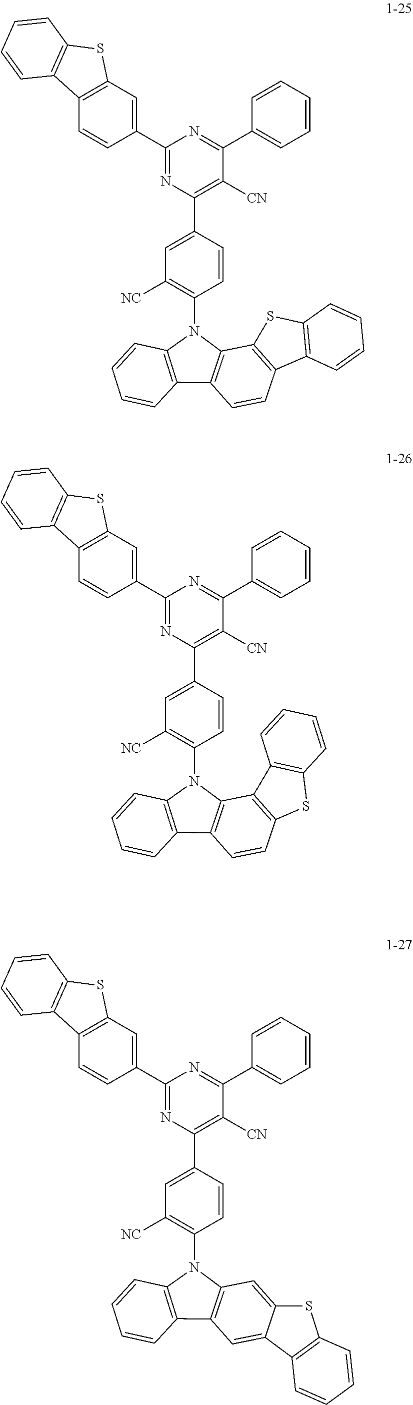 [SMILES: N#Cc1cc(-c2nc(-c3ccc4c(c3)sc3ccccc34)nc(-c3ccccc3)c2C#N)ccc1-n1c2ccccc2c2ccc3c4ccccc4sc3c21 ; N#Cc1cc(-c2nc(-c3ccc4c(c3)sc3ccccc34)nc(-c3ccccc3)c2C#N)ccc1-n1c2ccccc2c2ccc3sc4ccccc4c3c21 ; N#Cc1cc(-c2nc(-c3ccc4c(c3)sc3ccccc34)nc(-c3ccccc3)c2C#N)ccc1-n1c2ccccc2c2cc3c(cc21)sc1ccccc13]

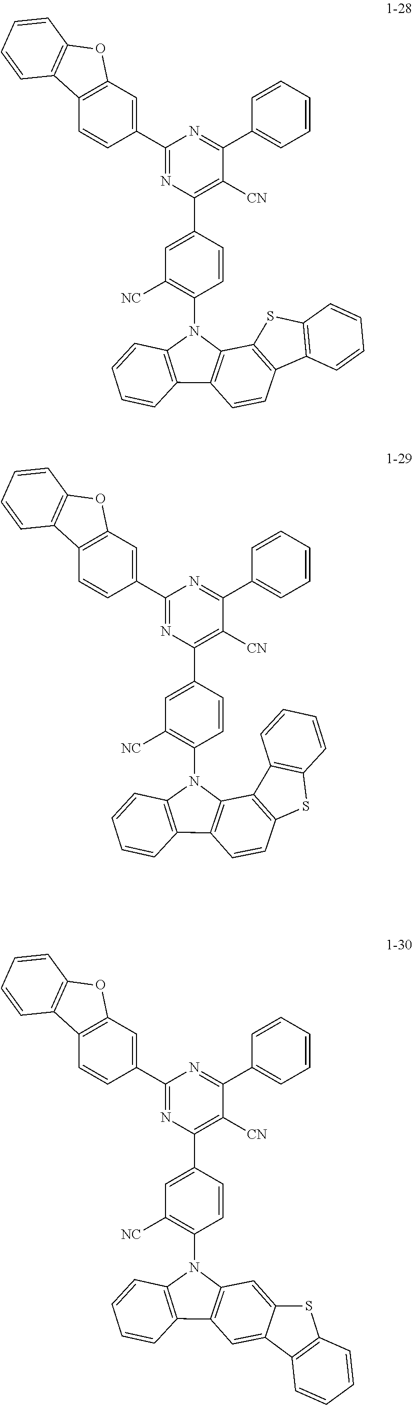 [SMILES: N#Cc1cc(-c2nc(-c3ccc4c(c3)oc3ccccc34)nc(-c3ccccc3)c2C#N)ccc1-n1c2ccccc2c2ccc3c4ccccc4sc3c21 ; N#Cc1cc(-c2nc(-c3ccc4c(c3)oc3ccccc34)nc(-c3ccccc3)c2C#N)ccc1-n1c2ccccc2c2ccc3sc4ccccc4c3c21 ; N#Cc1cc(-c2nc(-c3ccc4c(c3)oc3ccccc34)nc(-c3ccccc3)c2C#N)ccc1-n1c2ccccc2c2cc3c(cc21)sc1ccccc13]

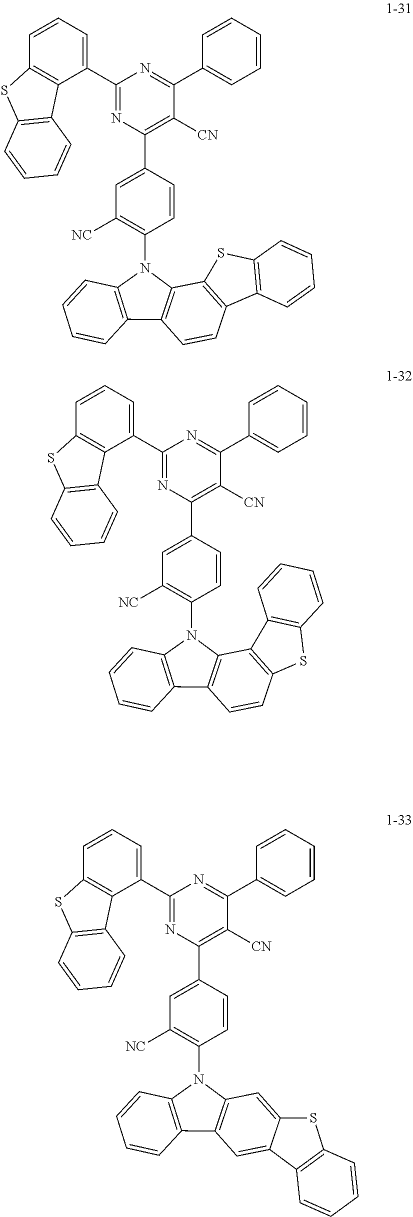

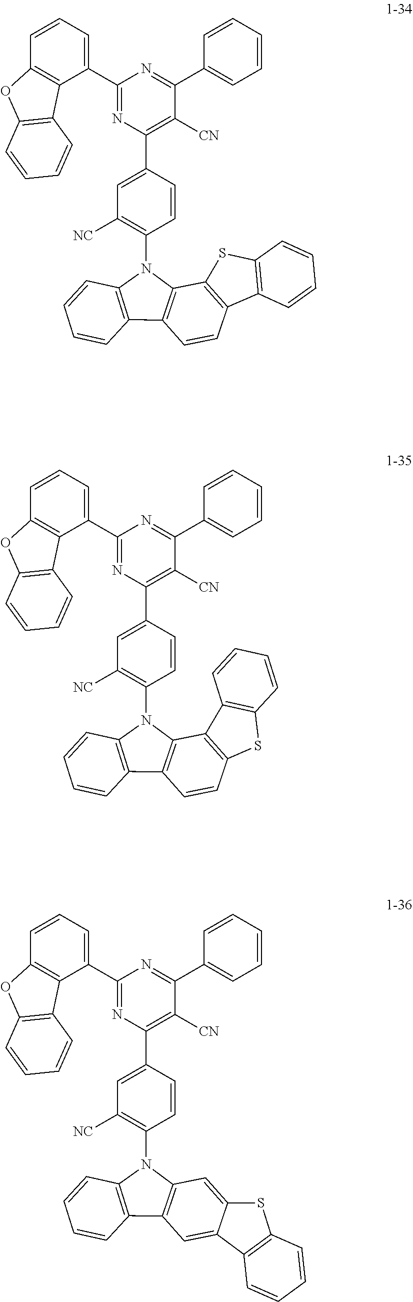 [SMILES: N#Cc1cc(-c2nc(-c3cccc4oc5ccccc5c34)nc(-c3ccccc3)c2C#N)ccc1-n1c2ccccc2c2ccc3c4ccccc4sc3c21 ; N#Cc1cc(-c2nc(-c3cccc4oc5ccccc5c34)nc(-c3ccccc3)c2C#N)ccc1-n1c2ccccc2c2ccc3sc4ccccc4c3c21 ; N#Cc1cc(-c2nc(-c3cccc4oc5ccccc5c34)nc(-c3ccccc3)c2C#N)ccc1-n1c2ccccc2c2cc3c(cc21)sc1ccccc13]

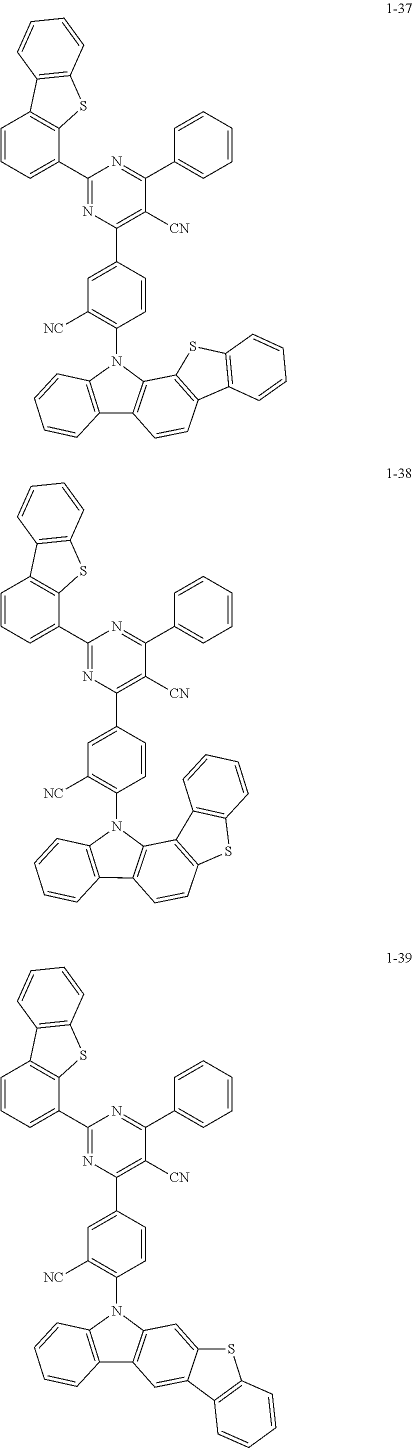 [SMILES: N#Cc1cc(-c2nc(-c3cccc4c3sc3ccccc34)nc(-c3ccccc3)c2C#N)ccc1-n1c2ccccc2c2ccc3c4ccccc4sc3c21 ; N#Cc1cc(-c2nc(-c3cccc4c3sc3ccccc34)nc(-c3ccccc3)c2C#N)ccc1-n1c2ccccc2c2ccc3sc4ccccc4c3c21 ; N#Cc1cc(-c2nc(-c3cccc4c3sc3ccccc34)nc(-c3ccccc3)c2C#N)ccc1-n1c2ccccc2c2cc3c(cc21)sc1ccccc13]

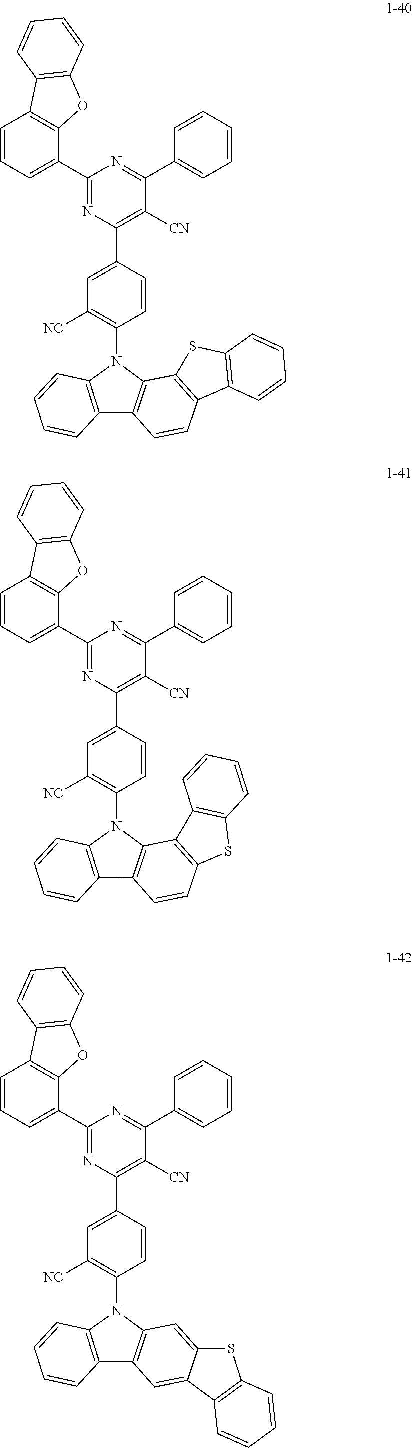 [SMILES: N#Cc1cc(-c2nc(-c3cccc4c3oc3ccccc34)nc(-c3ccccc3)c2C#N)ccc1-n1c2ccccc2c2ccc3c4ccccc4sc3c21 ; N#Cc1cc(-c2nc(-c3cccc4c3oc3ccccc34)nc(-c3ccccc3)c2C#N)ccc1-n1c2ccccc2c2ccc3sc4ccccc4c3c21 ; N#Cc1cc(-c2nc(-c3cccc4c3oc3ccccc34)nc(-c3ccccc3)c2C#N)ccc1-n1c2ccccc2c2cc3c(cc21)sc1ccccc13]

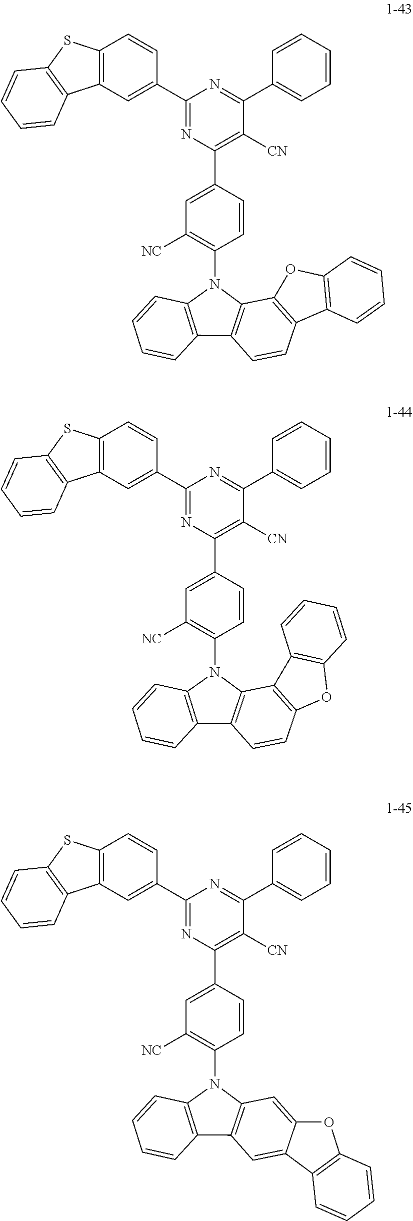 [SMILES: N#Cc1cc(-c2nc(-c3ccc4sc5ccccc5c4c3)nc(-c3ccccc3)c2C#N)ccc1-n1c2ccccc2c2ccc3c4ccccc4oc3c21 ; N#Cc1cc(-c2nc(-c3ccc4sc5ccccc5c4c3)nc(-c3ccccc3)c2C#N)ccc1-n1c2ccccc2c2ccc3oc4ccccc4c3c21 ; N#Cc1cc(-c2nc(-c3ccc4sc5ccccc5c4c3)nc(-c3ccccc3)c2C#N)ccc1-n1c2ccccc2c2cc3c(cc21)oc1ccccc13]

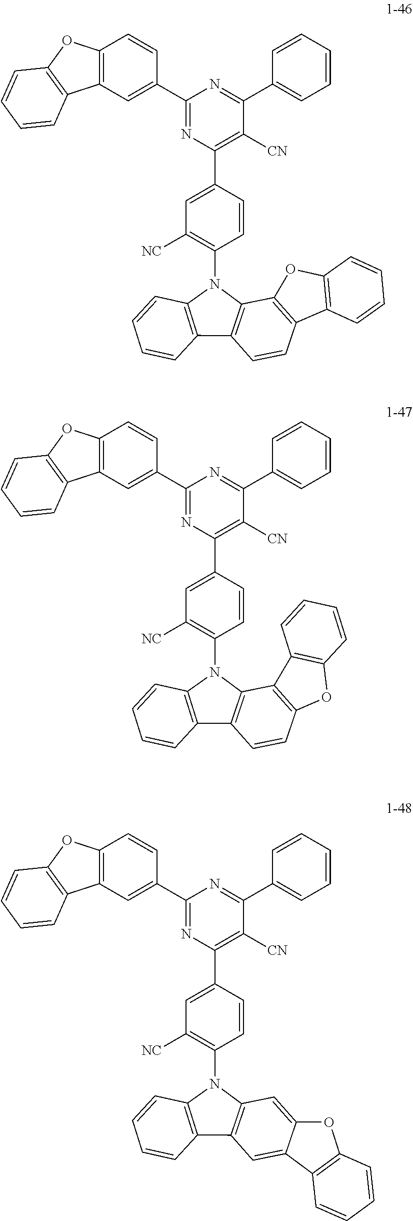 [SMILES: N#Cc1cc(-c2nc(-c3ccc4oc5ccccc5c4c3)nc(-c3ccccc3)c2C#N)ccc1-n1c2ccccc2c2ccc3c4ccccc4oc3c21 ; N#Cc1cc(-c2nc(-c3ccc4oc5ccccc5c4c3)nc(-c3ccccc3)c2C#N)ccc1-n1c2ccccc2c2ccc3oc4ccccc4c3c21 ; N#Cc1cc(-c2nc(-c3ccc4oc5ccccc5c4c3)nc(-c3ccccc3)c2C#N)ccc1-n1c2ccccc2c2cc3c(cc21)oc1ccccc13]

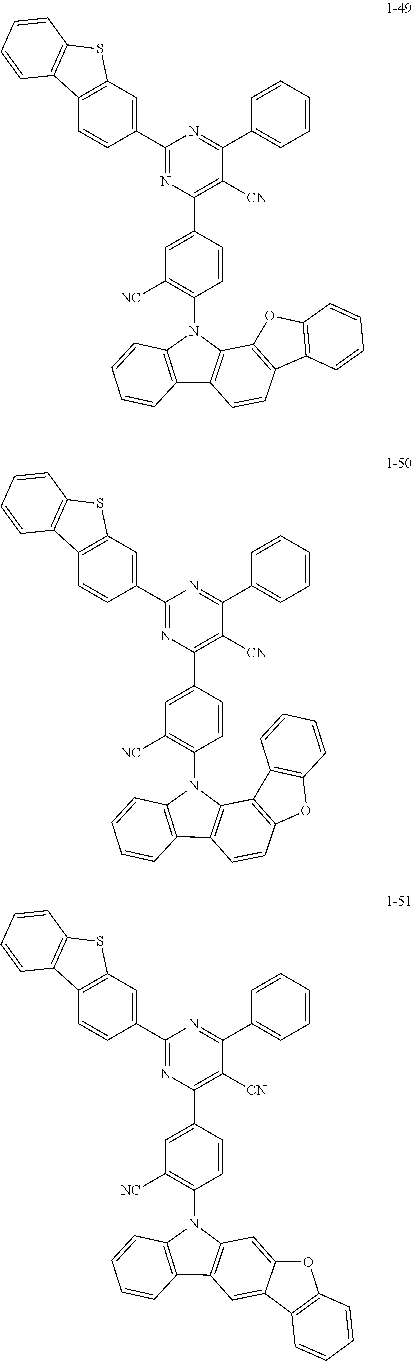 [SMILES: N#Cc1cc(-c2nc(-c3ccc4c(c3)sc3ccccc34)nc(-c3ccccc3)c2C#N)ccc1-n1c2ccccc2c2ccc3c4ccccc4oc3c21 ; N#Cc1cc(-c2nc(-c3ccc4c(c3)sc3ccccc34)nc(-c3ccccc3)c2C#N)ccc1-n1c2ccccc2c2ccc3oc4ccccc4c3c21 ; N#Cc1cc(-c2nc(-c3ccc4c(c3)sc3ccccc34)nc(-c3ccccc3)c2C#N)ccc1-n1c2ccccc2c2cc3c(cc21)oc1ccccc13]

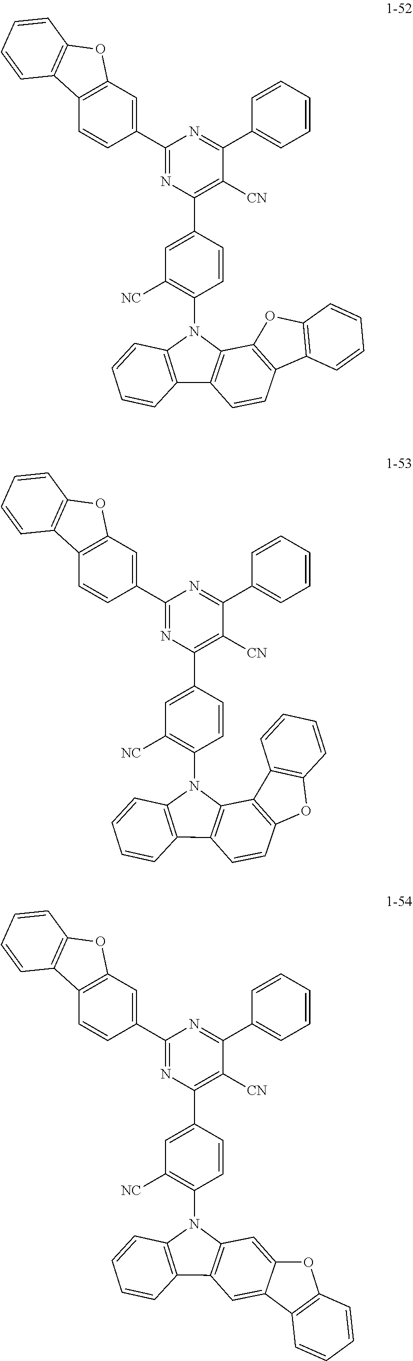 [SMILES: N#Cc1cc(-c2nc(-c3ccc4c(c3)oc3ccccc34)nc(-c3ccccc3)c2C#N)ccc1-n1c2ccccc2c2ccc3c4ccccc4oc3c21 ; N#Cc1cc(-c2nc(-c3ccc4c(c3)oc3ccccc34)nc(-c3ccccc3)c2C#N)ccc1-n1c2ccccc2c2ccc3oc4ccccc4c3c21 ; N#Cc1cc(-c2nc(-c3ccc4c(c3)oc3ccccc34)nc(-c3ccccc3)c2C#N)ccc1-n1c2ccccc2c2cc3c(cc21)oc1ccccc13]

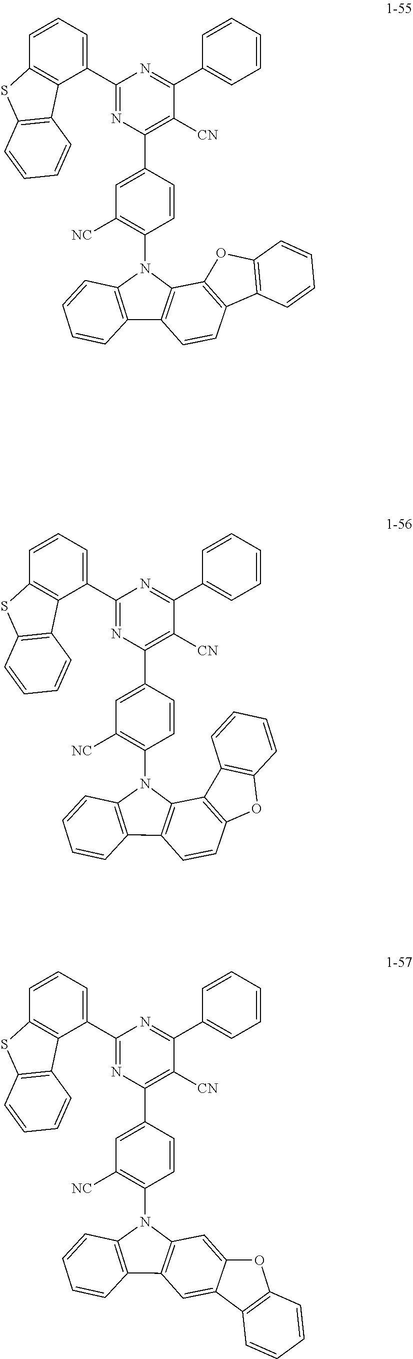 [SMILES: N#Cc1cc(-c2nc(-c3cccc4sc5ccccc5c34)nc(-c3ccccc3)c2C#N)ccc1-n1c2ccccc2c2ccc3c4ccccc4oc3c21 ; N#Cc1cc(-c2nc(-c3cccc4sc5ccccc5c34)nc(-c3ccccc3)c2C#N)ccc1-n1c2ccccc2c2ccc3oc4ccccc4c3c21 ; N#Cc1cc(-c2nc(-c3cccc4sc5ccccc5c34)nc(-c3ccccc3)c2C#N)ccc1-n1c2ccccc2c2cc3c(cc21)oc1ccccc13]

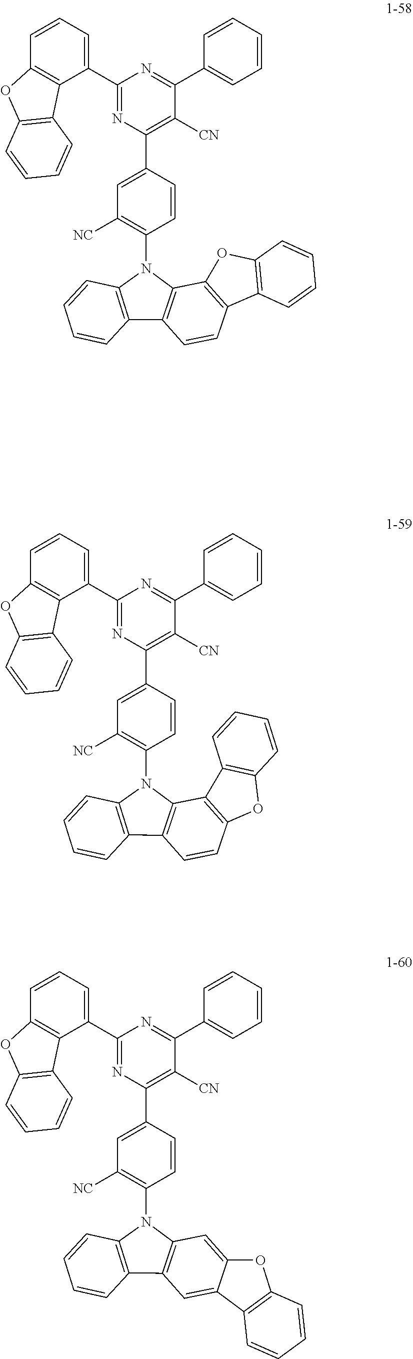 [SMILES: N#Cc1cc(-c2nc(-c3cccc4oc5ccccc5c34)nc(-c3ccccc3)c2C#N)ccc1-n1c2ccccc2c2ccc3c4ccccc4oc3c21 ; N#Cc1cc(-c2nc(-c3cccc4oc5ccccc5c34)nc(-c3ccccc3)c2C#N)ccc1-n1c2ccccc2c2ccc3oc4ccccc4c3c21 ; N#Cc1cc(-c2nc(-c3cccc4oc5ccccc5c34)nc(-c3ccccc3)c2C#N)ccc1-n1c2ccccc2c2cc3c(cc21)oc1ccccc13]

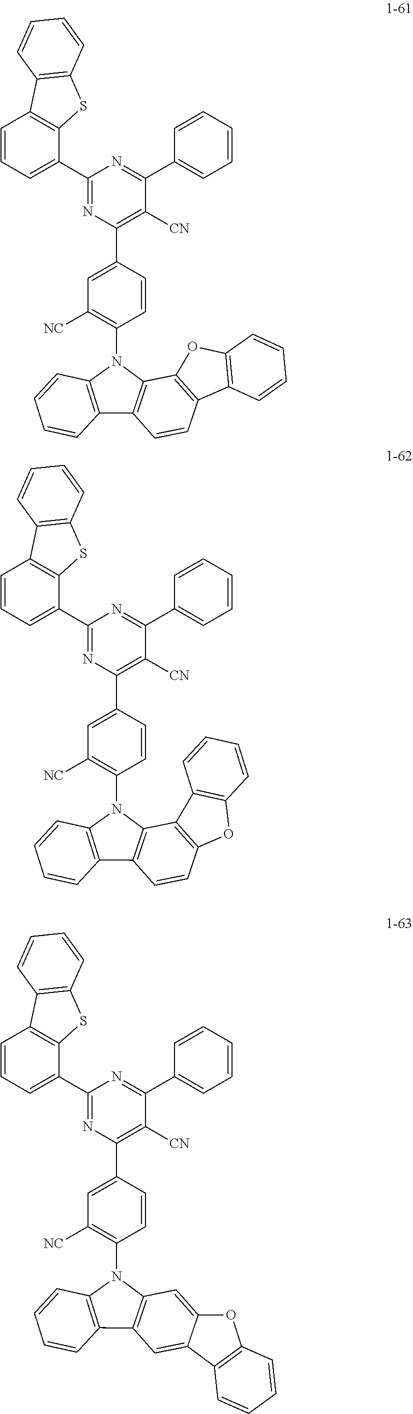

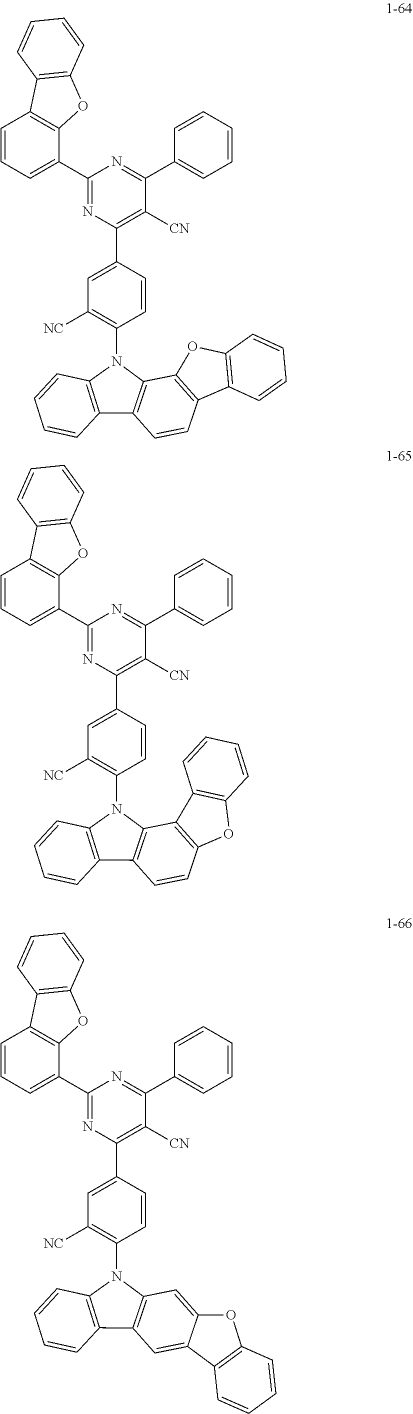

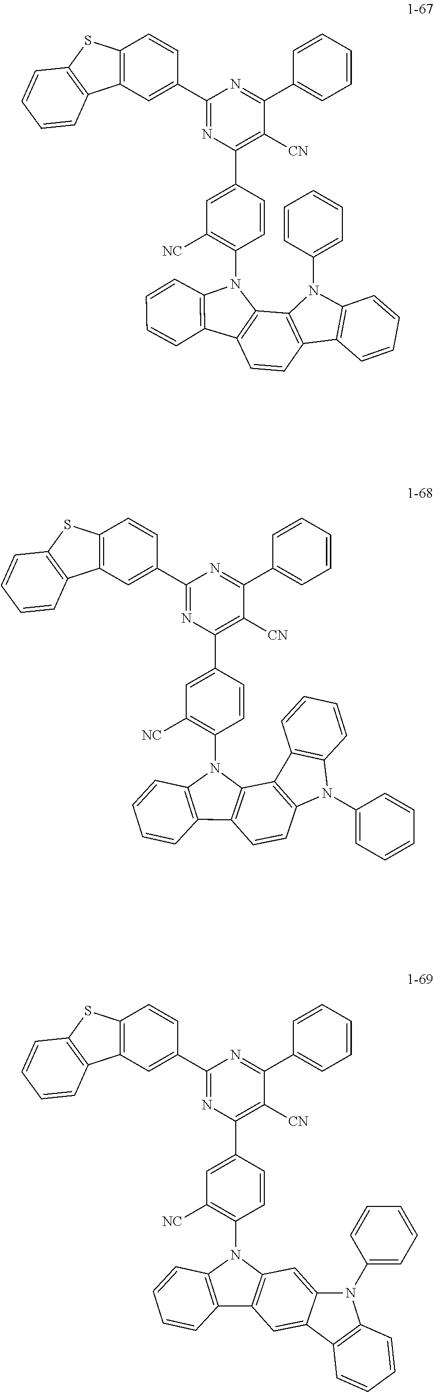 [SMILES: N#Cc1cc(-c2nc(-c3ccc4sc5ccccc5c4c3)nc(-c3ccccc3)c2C#N)ccc1-n1c2ccccc2c2ccc3c4ccccc4n(-c4ccccc4)c3c21 ; N#Cc1cc(-c2nc(-c3ccc4sc5ccccc5c4c3)nc(-c3ccccc3)c2C#N)ccc1-n1c2ccccc2c2ccc3c(c4ccccc4n3-c3ccccc3)c21 ; N#Cc1cc(-c2nc(-c3ccc4sc5ccccc5c4c3)nc(-c3ccccc3)c2C#N)ccc1-n1c2ccccc2c2cc3c4ccccc4n(-c4ccccc4)c3cc21]

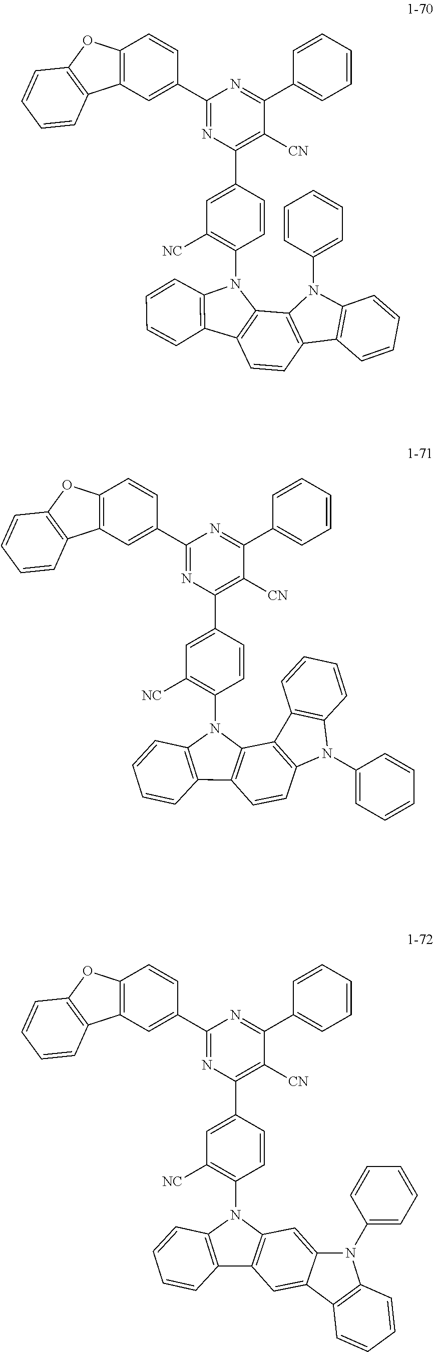 [SMILES: N#Cc1cc(-c2nc(-c3ccc4oc5ccccc5c4c3)nc(-c3ccccc3)c2C#N)ccc1-n1c2ccccc2c2ccc3c4ccccc4n(-c4ccccc4)c3c21 ; N#Cc1cc(-c2nc(-c3ccc4oc5ccccc5c4c3)nc(-c3ccccc3)c2C#N)ccc1-n1c2ccccc2c2ccc3c(c4ccccc4n3-c3ccccc3)c21 ; N#Cc1cc(-c2nc(-c3ccc4oc5ccccc5c4c3)nc(-c3ccccc3)c2C#N)ccc1-n1c2ccccc2c2cc3c4ccccc4n(-c4ccccc4)c3cc21]

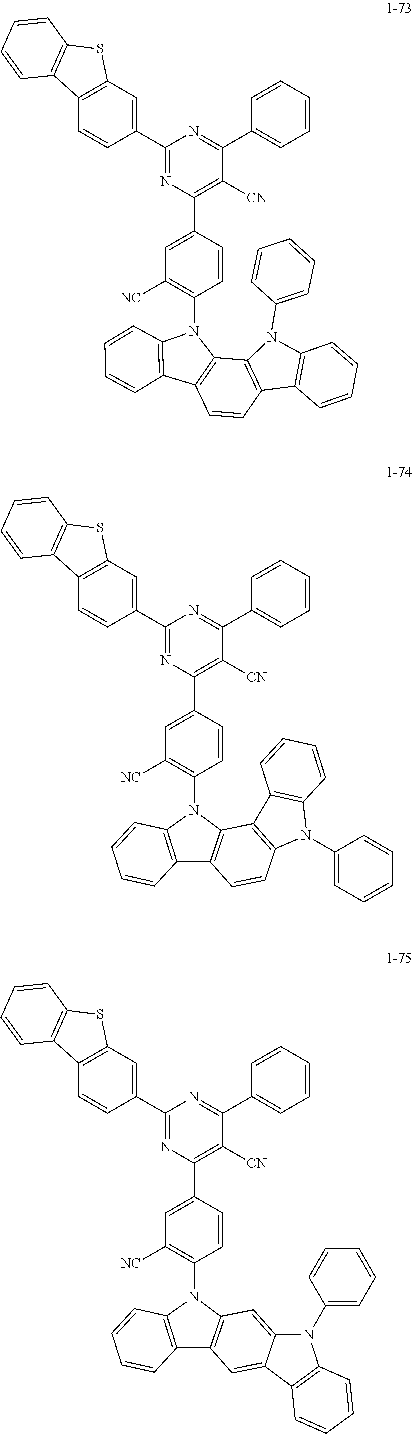 [SMILES: N#Cc1cc(-c2nc(-c3ccc4c(c3)sc3ccccc34)nc(-c3ccccc3)c2C#N)ccc1-n1c2ccccc2c2ccc3c4ccccc4n(-c4ccccc4)c3c21 ; N#Cc1cc(-c2nc(-c3ccc4c(c3)sc3ccccc34)nc(-c3ccccc3)c2C#N)ccc1-n1c2ccccc2c2ccc3c(c4ccccc4n3-c3ccccc3)c21 ; N#Cc1cc(-c2nc(-c3ccc4c(c3)sc3ccccc34)nc(-c3ccccc3)c2C#N)ccc1-n1c2ccccc2c2cc3c4ccccc4n(-c4ccccc4)c3cc21]

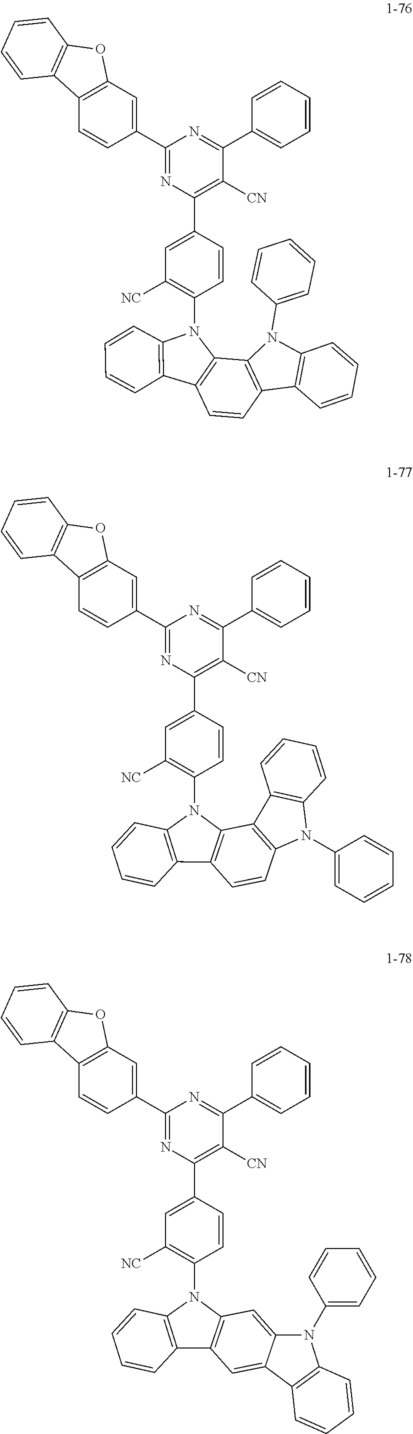 [SMILES: N#Cc1cc(-c2nc(-c3ccc4c(c3)oc3ccccc34)nc(-c3ccccc3)c2C#N)ccc1-n1c2ccccc2c2ccc3c4ccccc4n(-c4ccccc4)c3c21 ; N#Cc1cc(-c2nc(-c3ccc4c(c3)oc3ccccc34)nc(-c3ccccc3)c2C#N)ccc1-n1c2ccccc2c2ccc3c(c4ccccc4n3-c3ccccc3)c21 ; N#Cc1cc(-c2nc(-c3ccc4c(c3)oc3ccccc34)nc(-c3ccccc3)c2C#N)ccc1-n1c2ccccc2c2cc3c4ccccc4n(-c4ccccc4)c3cc21]

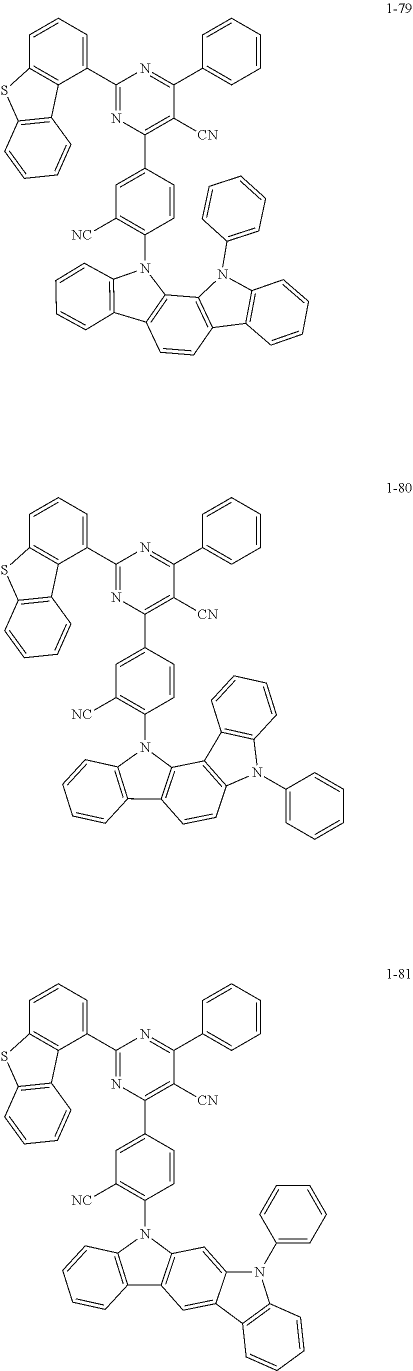 [SMILES: N#Cc1cc(-c2nc(-c3cccc4sc5ccccc5c34)nc(-c3ccccc3)c2C#N)ccc1-n1c2ccccc2c2ccc3c4ccccc4n(-c4ccccc4)c3c21 ; N#Cc1cc(-c2nc(-c3cccc4sc5ccccc5c34)nc(-c3ccccc3)c2C#N)ccc1-n1c2ccccc2c2ccc3c(c4ccccc4n3-c3ccccc3)c21 ; N#Cc1cc(-c2nc(-c3cccc4sc5ccccc5c34)nc(-c3ccccc3)c2C#N)ccc1-n1c2ccccc2c2cc3c4ccccc4n(-c4ccccc4)c3cc21]

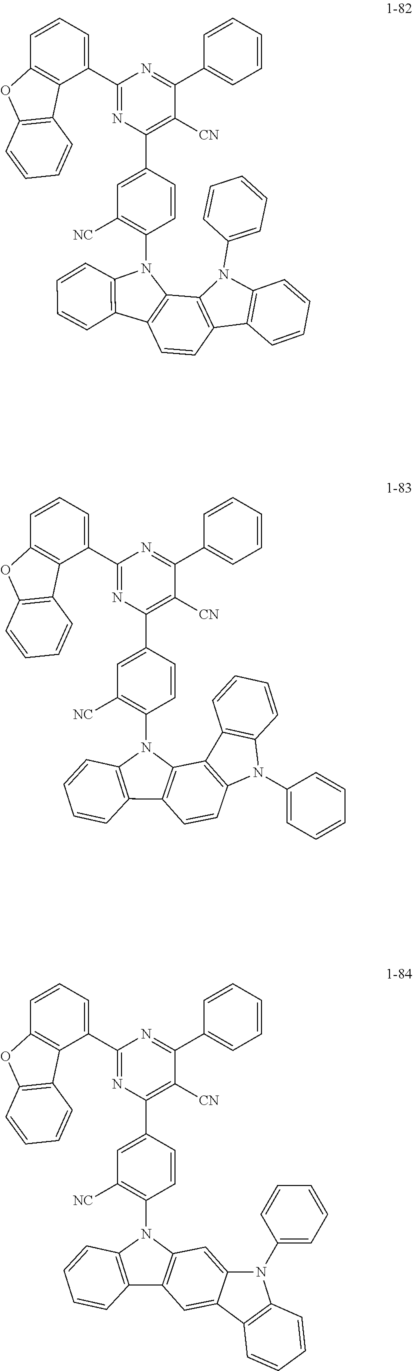 [SMILES: N#Cc1cc(-c2nc(-c3cccc4oc5ccccc5c34)nc(-c3ccccc3)c2C#N)ccc1-n1c2ccccc2c2ccc3c4ccccc4n(-c4ccccc4)c3c21 ; N#Cc1cc(-c2nc(-c3cccc4oc5ccccc5c34)nc(-c3ccccc3)c2C#N)ccc1-n1c2ccccc2c2ccc3c(c4ccccc4n3-c3ccccc3)c21 ; N#Cc1cc(-c2nc(-c3cccc4oc5ccccc5c34)nc(-c3ccccc3)c2C#N)ccc1-n1c2ccccc2c2cc3c4ccccc4n(-c4ccccc4)c3cc21]

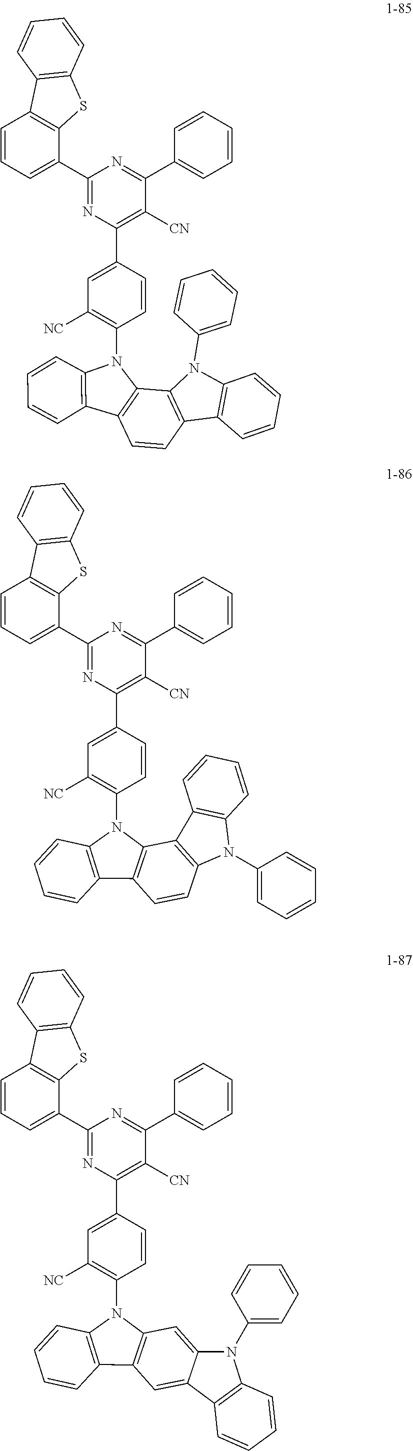 [SMILES: N#Cc1cc(-c2nc(-c3cccc4c3sc3ccccc34)nc(-c3ccccc3)c2C#N)ccc1-n1c2ccccc2c2ccc3c4ccccc4n(-c4ccccc4)c3c21 ; N#Cc1cc(-c2nc(-c3cccc4c3sc3ccccc34)nc(-c3ccccc3)c2C#N)ccc1-n1c2ccccc2c2ccc3c(c4ccccc4n3-c3ccccc3)c21 ; N#Cc1cc(-c2nc(-c3cccc4c3sc3ccccc34)nc(-c3ccccc3)c2C#N)ccc1-n1c2ccccc2c2cc3c4ccccc4n(-c4ccccc4)c3cc21]

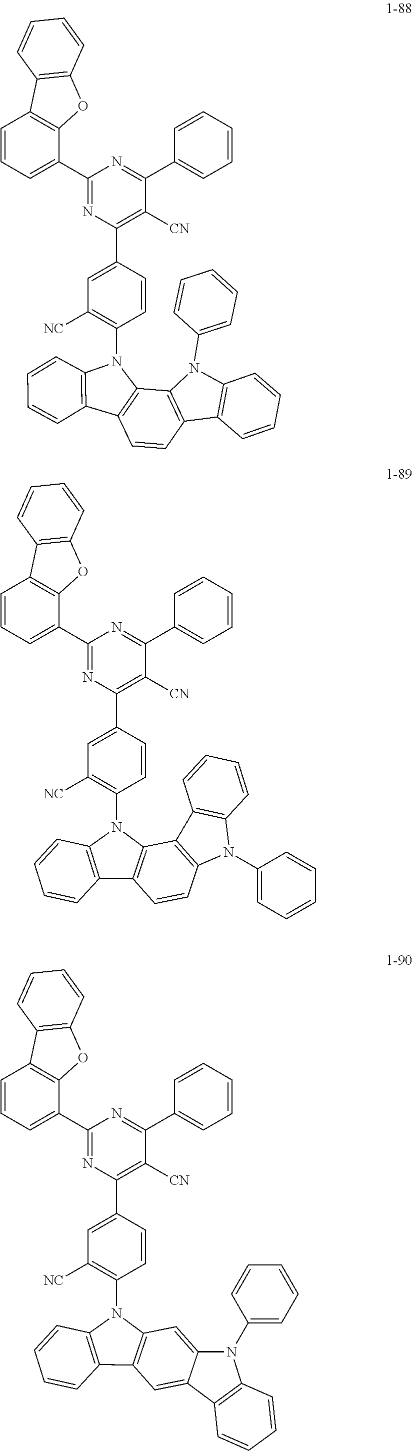 [SMILES: N#Cc1cc(-c2nc(-c3cccc4c3oc3ccccc34)nc(-c3ccccc3)c2C#N)ccc1-n1c2ccccc2c2ccc3c4ccccc4n(-c4ccccc4)c3c21 ; N#Cc1cc(-c2nc(-c3cccc4c3oc3ccccc34)nc(-c3ccccc3)c2C#N)ccc1-n1c2ccccc2c2ccc3c(c4ccccc4n3-c3ccccc3)c21 ; N#Cc1cc(-c2nc(-c3cccc4c3oc3ccccc34)nc(-c3ccccc3)c2C#N)ccc1-n1c2ccccc2c2cc3c4ccccc4n(-c4ccccc4)c3cc21]

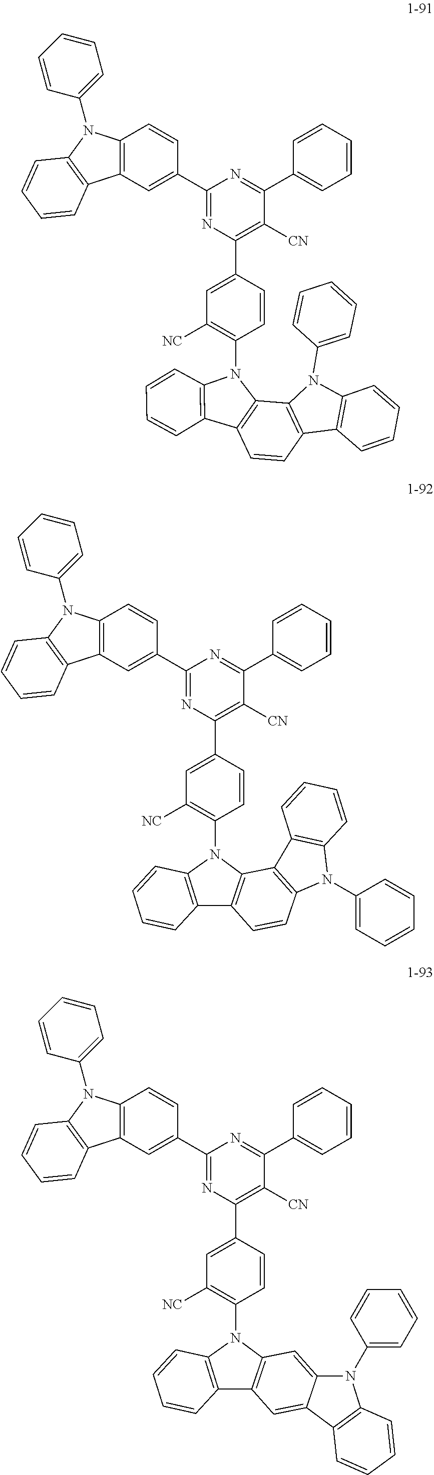 [SMILES: N#Cc1cc(-c2nc(-c3ccc4c(c3)c3ccccc3n4-c3ccccc3)nc(-c3ccccc3)c2C#N)ccc1-n1c2ccccc2c2ccc3c4ccccc4n(-c4ccccc4)c3c21 ; N#Cc1cc(-c2nc(-c3ccc4c(c3)c3ccccc3n4-c3ccccc3)nc(-c3ccccc3)c2C#N)ccc1-n1c2ccccc2c2ccc3c(c4ccccc4n3-c3ccccc3)c21 ; N#Cc1cc(-c2nc(-c3ccc4c(c3)c3ccccc3n4-c3ccccc3)nc(-c3ccccc3)c2C#N)ccc1-n1c2ccccc2c2cc3c4ccccc4n(-c4ccccc4)c3cc21]

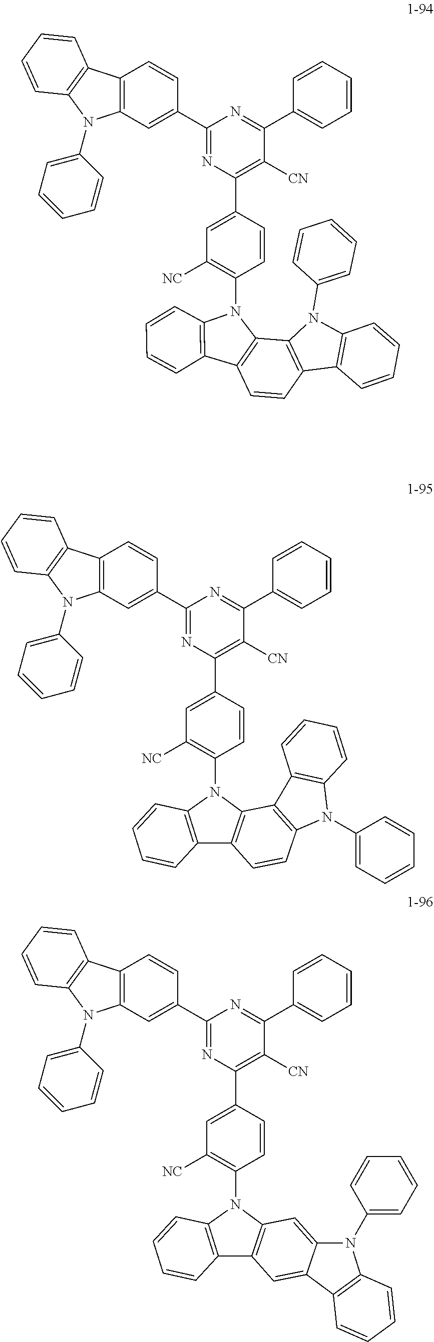 [SMILES: N#Cc1cc(-c2nc(-c3ccc4c5ccccc5n(-c5ccccc5)c4c3)nc(-c3ccccc3)c2C#N)ccc1-n1c2ccccc2c2ccc3c4ccccc4n(-c4ccccc4)c3c21 ; N#Cc1cc(-c2nc(-c3ccc4c5ccccc5n(-c5ccccc5)c4c3)nc(-c3ccccc3)c2C#N)ccc1-n1c2ccccc2c2ccc3c(c4ccccc4n3-c3ccccc3)c21 ; N#Cc1cc(-c2nc(-c3ccc4c5ccccc5n(-c5ccccc5)c4c3)nc(-c3ccccc3)c2C#N)ccc1-n1c2ccccc2c2cc3c4ccccc4n(-c4ccccc4)c3cc21]

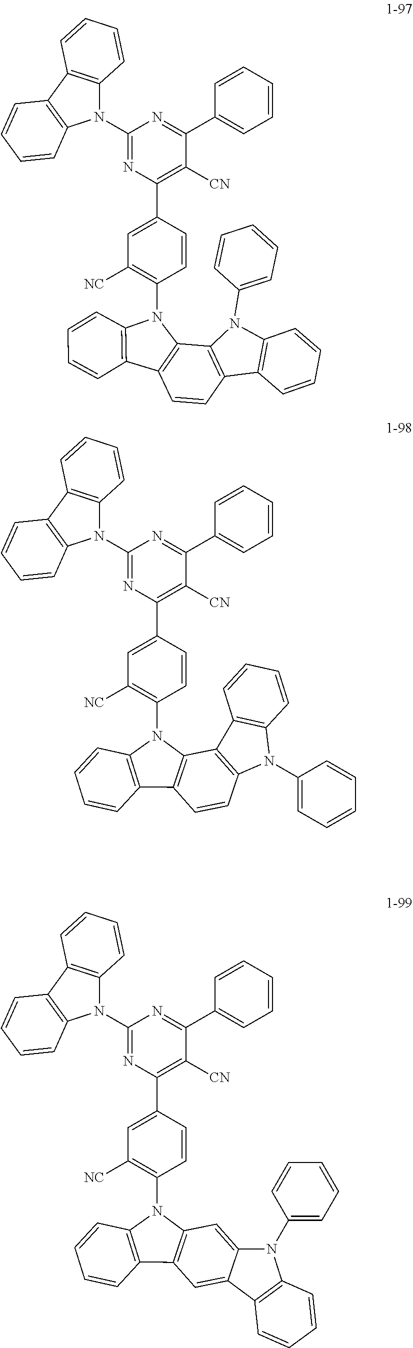 [SMILES: N#Cc1cc(-c2nc(-n3c4ccccc4c4ccccc43)nc(-c3ccccc3)c2C#N)ccc1-n1c2ccccc2c2ccc3c4ccccc4n(-c4ccccc4)c3c21 ; N#Cc1cc(-c2nc(-n3c4ccccc4c4ccccc43)nc(-c3ccccc3)c2C#N)ccc1-n1c2ccccc2c2ccc3c(c4ccccc4n3-c3ccccc3)c21 ; N#Cc1cc(-c2nc(-n3c4ccccc4c4ccccc43)nc(-c3ccccc3)c2C#N)ccc1-n1c2ccccc2c2cc3c4ccccc4n(-c4ccccc4)c3cc21]

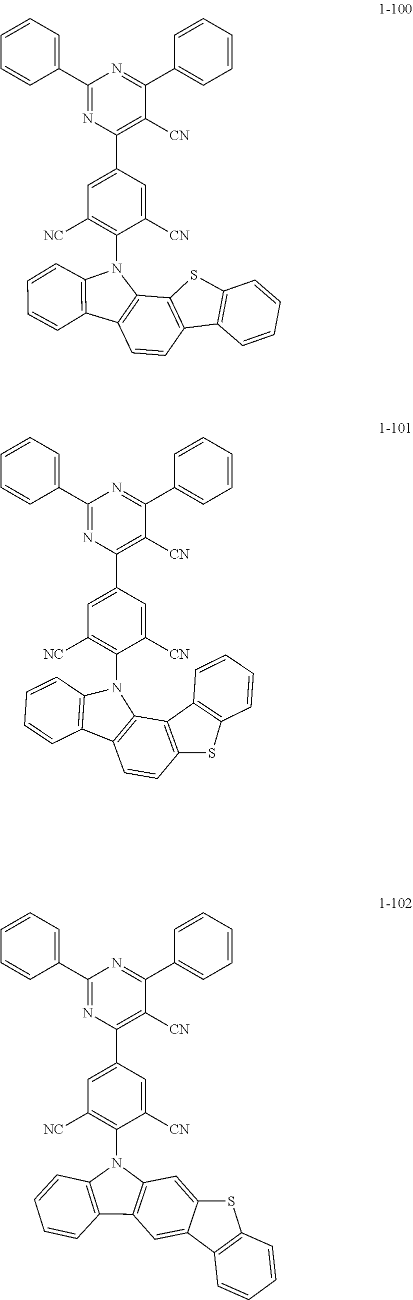 [SMILES: N#Cc1cc(-c2nc(-c3ccccc3)nc(-c3ccccc3)c2C#N)cc(C#N)c1-n1c2ccccc2c2ccc3c4ccccc4sc3c21 ; N#Cc1cc(-c2nc(-c3ccccc3)nc(-c3ccccc3)c2C#N)cc(C#N)c1-n1c2ccccc2c2ccc3sc4ccccc4c3c21 ; N#Cc1cc(-c2nc(-c3ccccc3)nc(-c3ccccc3)c2C#N)cc(C#N)c1-n1c2ccccc2c2cc3c(cc21)sc1ccccc13]

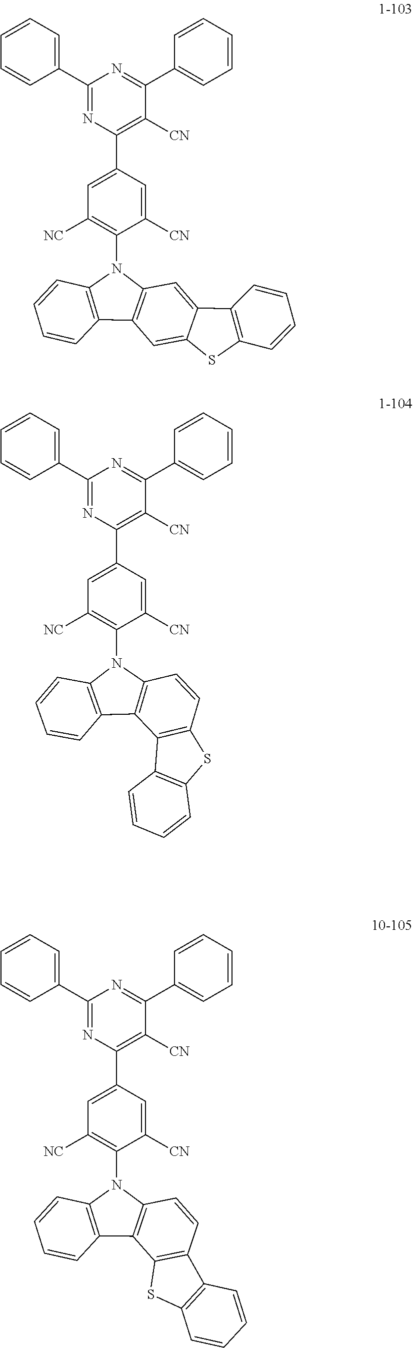 [SMILES: N#Cc1cc(-c2nc(-c3ccccc3)nc(-c3ccccc3)c2C#N)cc(C#N)c1-n1c2ccccc2c2cc3sc4ccccc4c3cc21 ; N#Cc1cc(-c2nc(-c3ccccc3)nc(-c3ccccc3)c2C#N)cc(C#N)c1-n1c2ccccc2c2c3c(ccc21)sc1ccccc13 ; N#Cc1cc(-c2nc(-c3ccccc3)nc(-c3ccccc3)c2C#N)cc(C#N)c1-n1c2ccccc2c2c3sc4ccccc4c3ccc21]

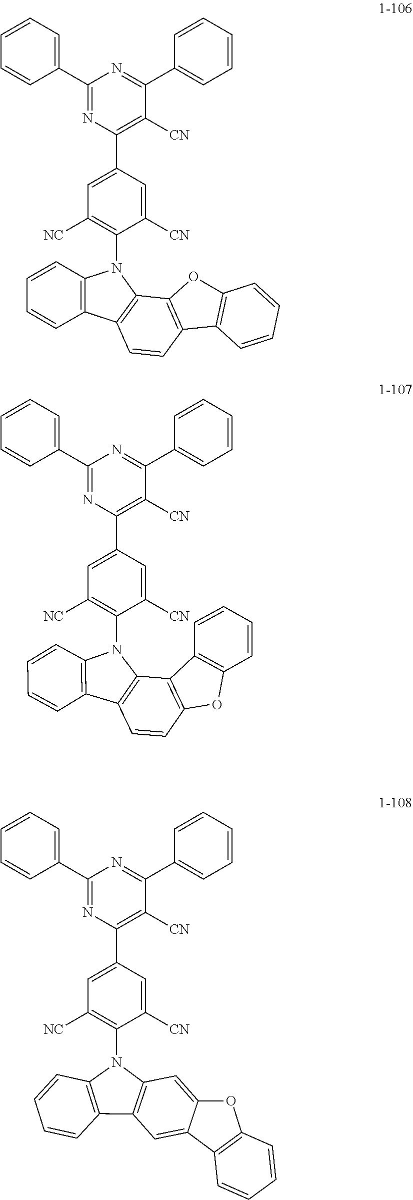 [SMILES: N#Cc1cc(-c2nc(-c3ccccc3)nc(-c3ccccc3)c2C#N)cc(C#N)c1-n1c2ccccc2c2ccc3c4ccccc4oc3c21 ; N#Cc1cc(-c2nc(-c3ccccc3)nc(-c3ccccc3)c2C#N)cc(C#N)c1-n1c2ccccc2c2ccc3oc4ccccc4c3c21 ; N#Cc1cc(-c2nc(-c3ccccc3)nc(-c3ccccc3)c2C#N)cc(C#N)c1-n1c2ccccc2c2cc3c(cc21)oc1ccccc13]

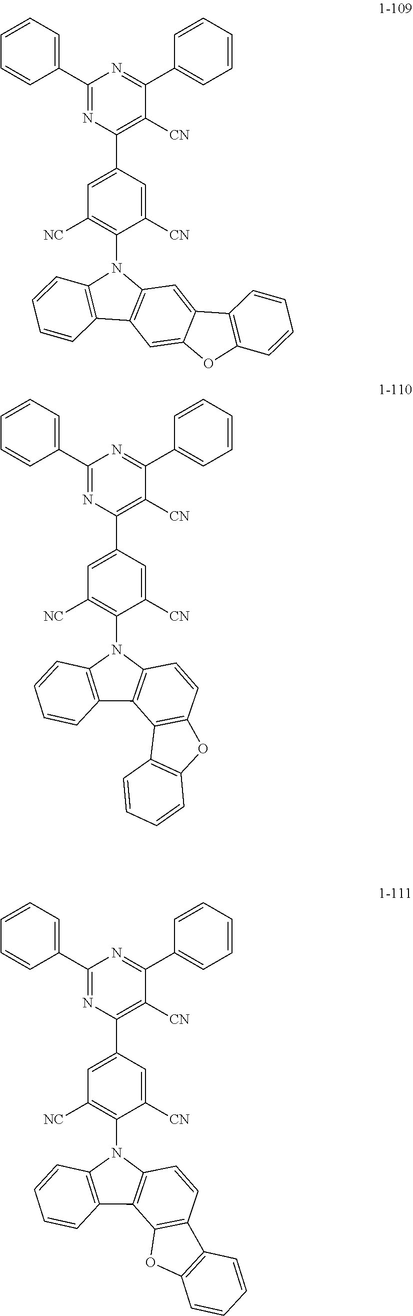

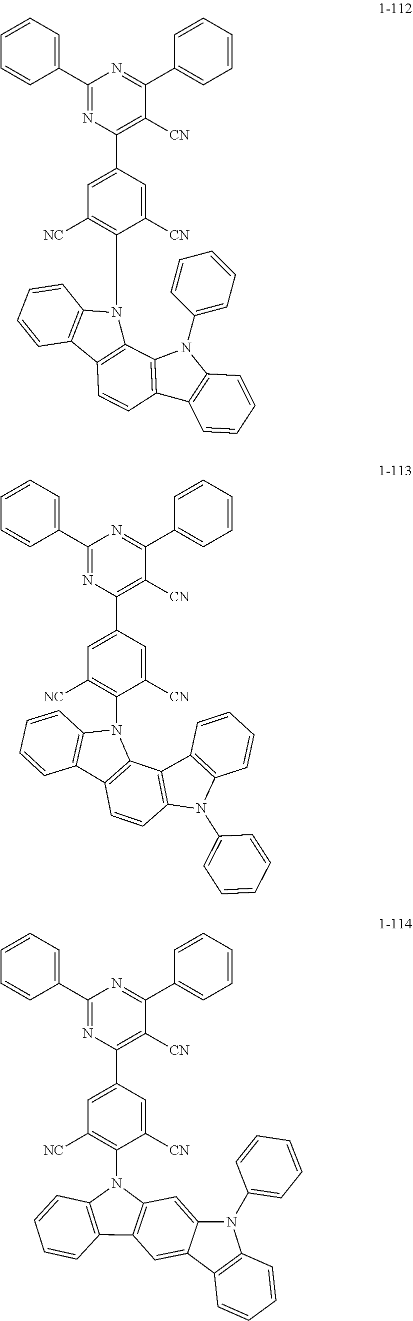

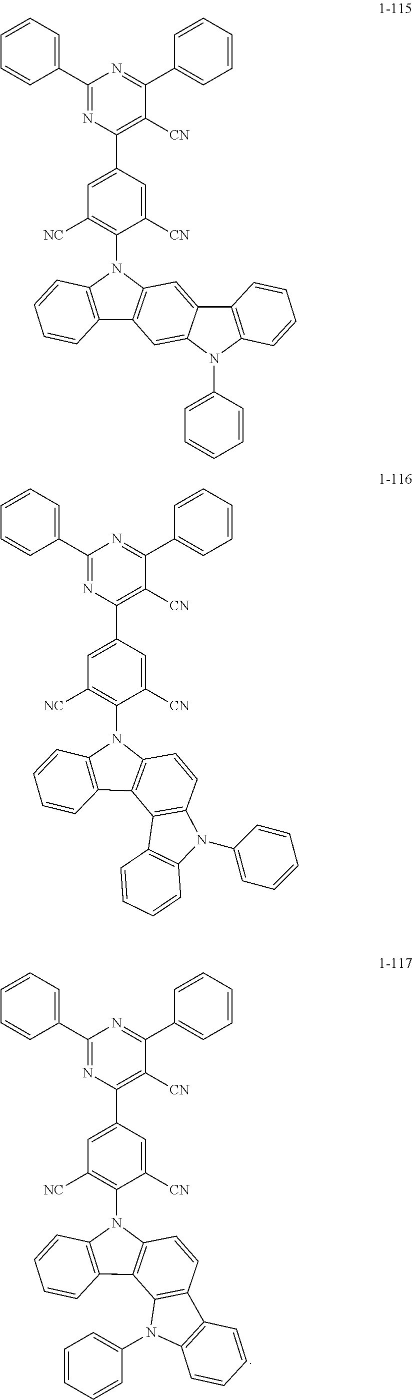

The first compound DF having the structure of Formulae 1 to 4 has delayed fluorescent property as well as a singlet energy level, a triplet energy level, a HOMO (Highest Occupied Molecular Orbital) energy level and a LUMO (Lowest Unoccupied Molecular Orbital) energy level sufficiently transfer exciton energies to the second compound FD, as described below. The first compound DF of the delayed fluorescent material has little energy bandgap ΔE_{ST}^{DF} between the excited singlet energy level S₁^{DF} and the excited triplet energy level T₁^{DF} of equal to or less than about 0.3 eV (FIG. 6) and shows excellent quantum efficiency because the excited triplet exciton energy of the first compound DF is converted to the excited singlet exciton of the second compound FD by RISC.

The first compound DF having the structure of Formulae 1 to 4 has a distorted chemical conformation due to the binding structure between the electron donor moiety and the electron acceptor moiety. Since the first compound DF utilizes triplet excitons, addition charge transfer transition (CT transition) is induced in the first compound DF. The first compound DF having the structure of Formulae 1 to 4 has short luminous lifespan by the luminous properties caused by the CT luminous mechanism.

The EML 240 includes the second compound FD of the fluorescent material in order to improve the shout luminous lifespan due to the first compound DF of the delayed fluorescent material and to implement hyper-fluorescence. As described above, the first compound DF of the delayed fluorescent material can utilize both the singlet exciton energy and the triplet exciton energy. When the EML 240 includes the second compound FD of the fluorescent material having proper energy levels comparted to the first compound DF of the delayed fluorescent material, the second compound FD can absorb exciton energies released from the first compound DF, and then the second compound FD can generate 100% singlet excitons utilizing the absorbed exciton energies with maximizing its luminous efficiency.

The singlet exciton energy of the first compound DF, which includes the singlet exciton energy of the first compound DF converted from its own triplet exciton energy and initial singlet exciton energy of the first compound DF in the EML 240, is transferred to the second compound FD of the fluorescent material in the same EML 240 via Forster resonance energy transfer (FRET) mechanism, and the ultimate emission is occurred at the second compound FD. Organic material having an absorption spectrum widely overlapped with a photoluminescence spectrum of the first compound DF can be used as the second compound FD so that the exciton energy generated at the first compound DF can be efficiently transferred to the second compound FD. Since the second compound FD emits light with singlet excitons shifted from the excited state to the ground state, not CT luminous mechanism, its luminous lifespan is relatively long comparted to the lifespan of the first compound DF.

The second compound FD in the EML 240 can be green fluorescent material. For example, the second compound FD can be an organic compound having a tetracene core substituted four aromatic and/or hetero aromatic groups. As an example, the second compound FD having the tetracene core can have the following structure of Formula 5:

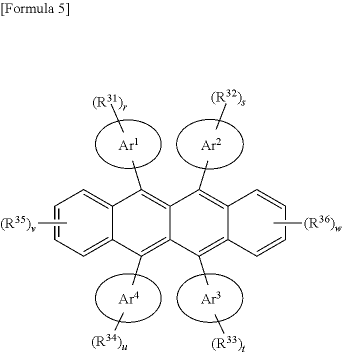

wherein, in Formula 5,

each of R³¹ to R³⁶ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when r is an integer of 2 or more, each R³¹ is identical to or different from each other, when s is an integer of 2 or more, each R³² is identical to or different from each other, when t is an integer of 2 or more, each R³³ is identical to or different from each other, when u is an integer of 2 or more, each R³⁴ is identical to or different from each other, when v is an integer of 2 or more, each R³⁵ is identical to or different from each other, when w is an integer of 2 or more, each R³⁶ is identical to or different from each other;

each of Ar¹ to Ar⁴ is independently an unsubstituted or substituted C₆-C₃₀ aromatic group or an unsubstituted or substituted C₃-C₃₀ hetero aromatic group;

each of r, s, t and u is independently an integer of 0 to 10; and

each of v and w is independently an integer of 0 to 4.

For example, each of the C₆-C₃₀ aromatic group and the C₃-C₃₀ hetero aromatic group constituting R³¹ to R³⁶ and Ar¹ to Ar⁴ in Formula 5 can be independently unsubstituted or substituted with at least one of deuterium, tritium, C₁-C₂₀ alkyl, C₆-C₃₀ aryl and/or C₃-C₃₀ hetero aryl.

The C₆-C₃₀ aromatic group constituting each of R³¹ to R³⁶ and Ar¹ to Ar⁴ in Formula 5 can independently include, but is not limited to, C₆-C₃₀ aryl, C₇-C₃₀ aryl alkyl, C₆-C₃₀ aryl oxy and C₆-C₃₀ aryl amino. The C₃-C₃₀ hetero aromatic group constituting each of R³¹ to R³⁶ and Ar¹ to Ar⁴ in Formula 5 can independently include, but is not limited to, C₃-C₃₀ hetero aryl, C₄-C₃₀ hetero aryl alkyl, C₃-C₃₀ hetero aryl oxy and C₃-C₃₀ hetero aryl amino.

In one exemplary aspect, each of Ar¹ to Ar⁴ in Formula 5 can be phenyl, and each of the C₆-C₃₀ aromatic group and the C₃-C₃₀ hetero aromatic group constituting R³¹ to R³⁶ can be the C₆-C₃₀ aryl and the C₃-C₃₀ hetero aryl, respectively. For example, the second compound FD can be a rubrene-based organic compound and can have the following structure of Formula 6:

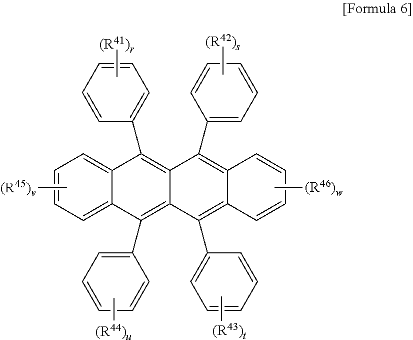

wherein, Formula 6,

each of R⁴¹ to R⁴⁶ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when r is an integer of 2 or more, each R⁴¹ is identical to or different from each other, when s is an integer of 2 or more, each R⁴² is identical to or different from each other, when t is an integer of 2 or more, each R⁴³ is identical to or different from each other, when u is an integer of 2 or more, each R⁴⁴ is identical to or different from each other, when v is an integer of 2 or more, each R⁴⁵ is identical to or different from each other, when w is an integer of 2 or more, each R⁴⁶ is identical to or different from each other; and

each of r, s, t and u is independently an integer of 0 to 5; and

each of v and w is independently an integer of 0 to 4.

For example, each of the C₆-C₃₀ aryl and the C₃-C₃₀ hetero aryl constituting R⁴¹ to R⁴⁶ in Formula 6 can be independently unsubstituted or substituted with at least one of deuterium, tritium, C₁-C₂₀ alkyl, C₆-C₃₀ aryl and/or C₃-C₃₀ hetero aryl.

More particularly, the second compound FD can be selected from, but is not limited to, the following compounds of Formula 7:

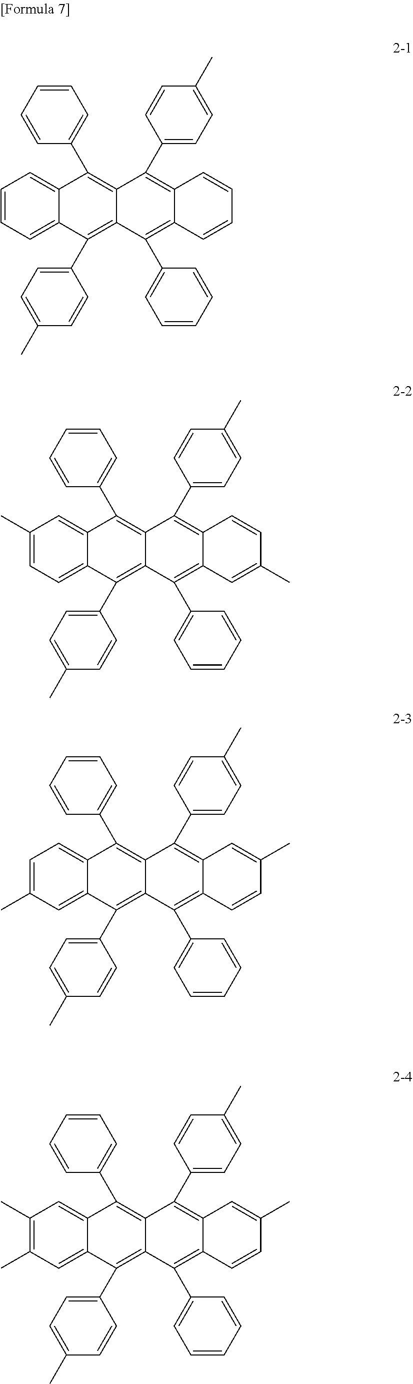

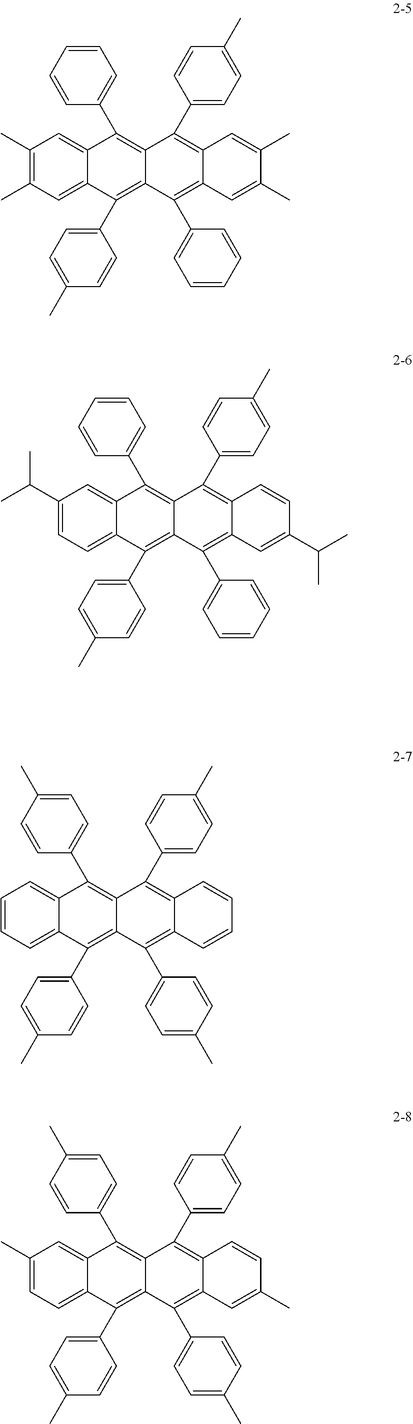

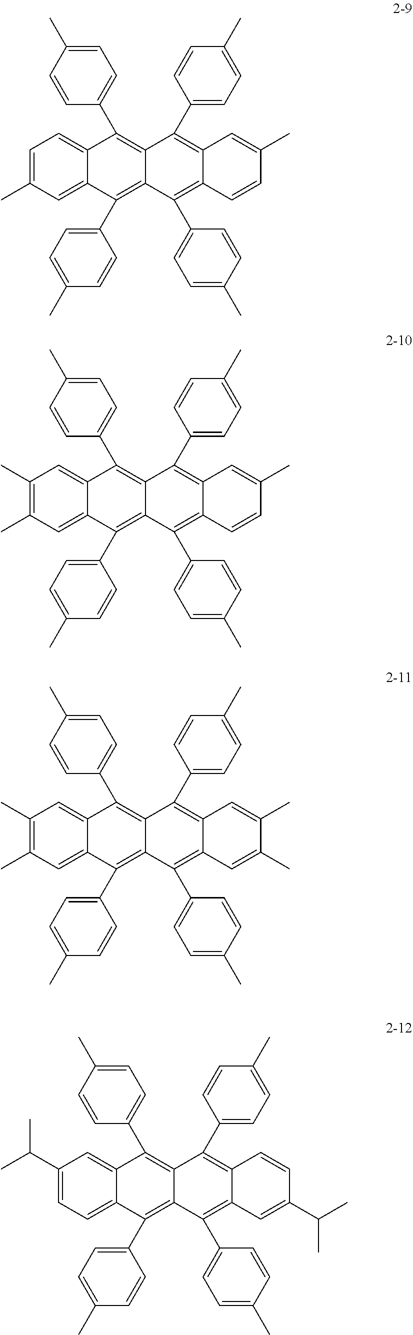

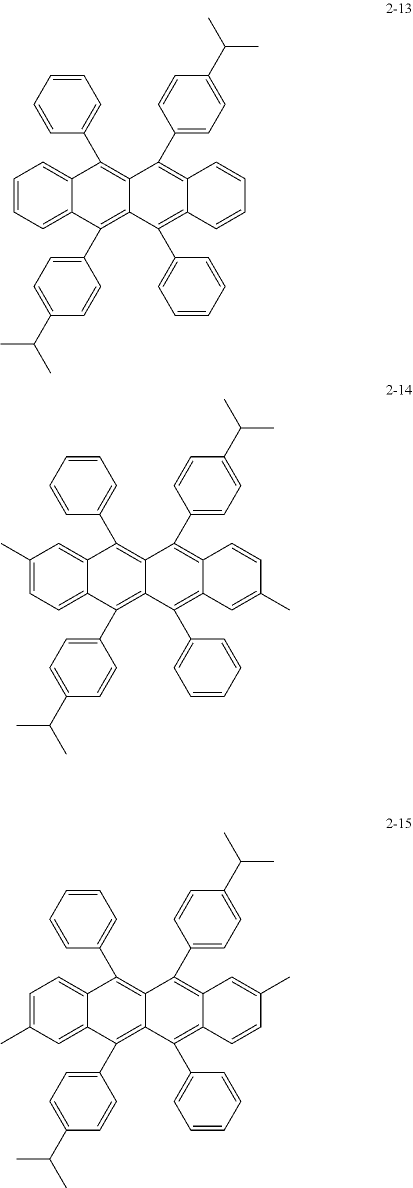

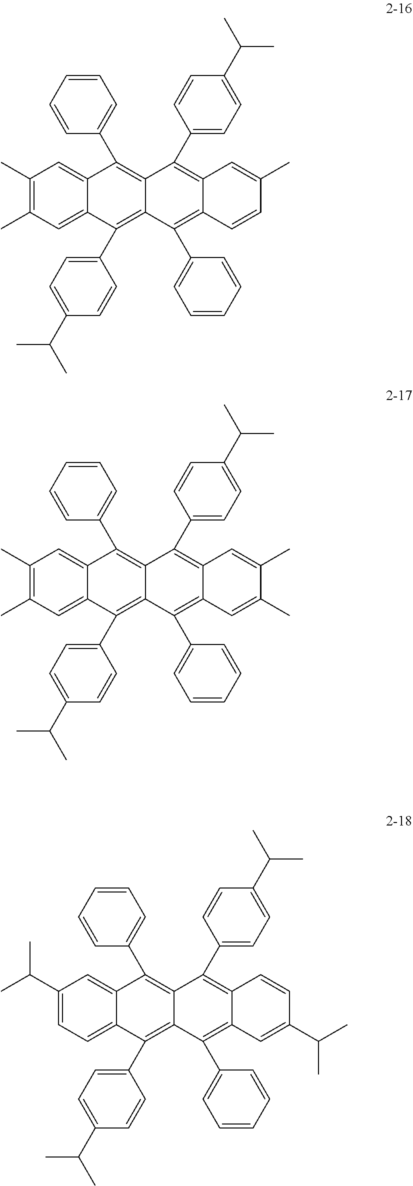

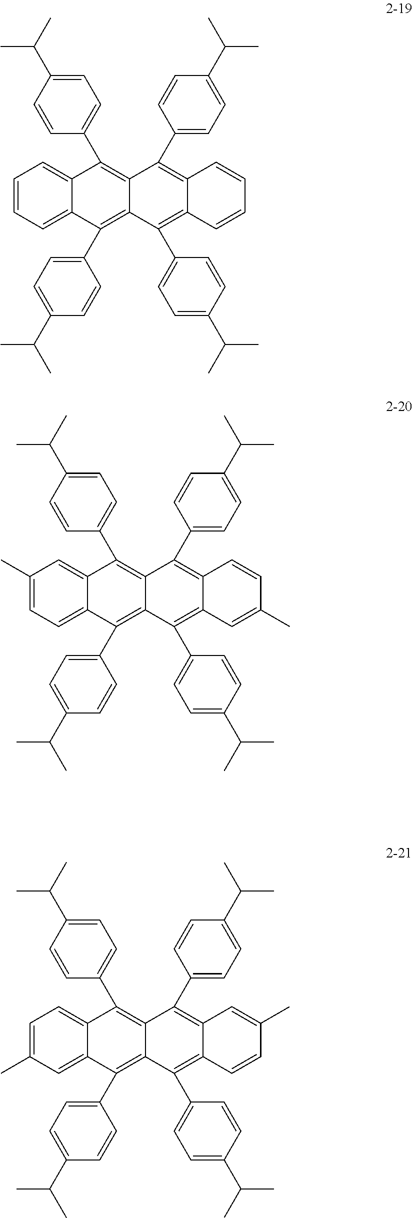

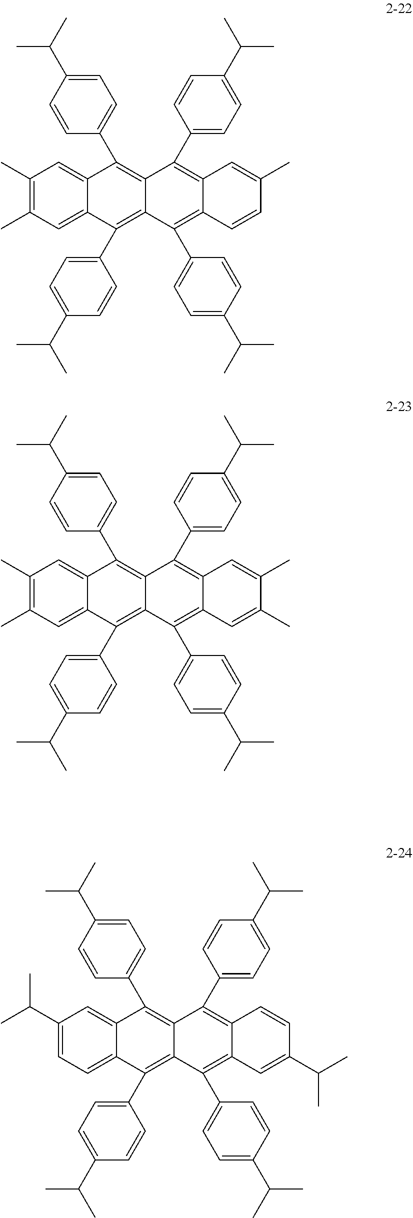

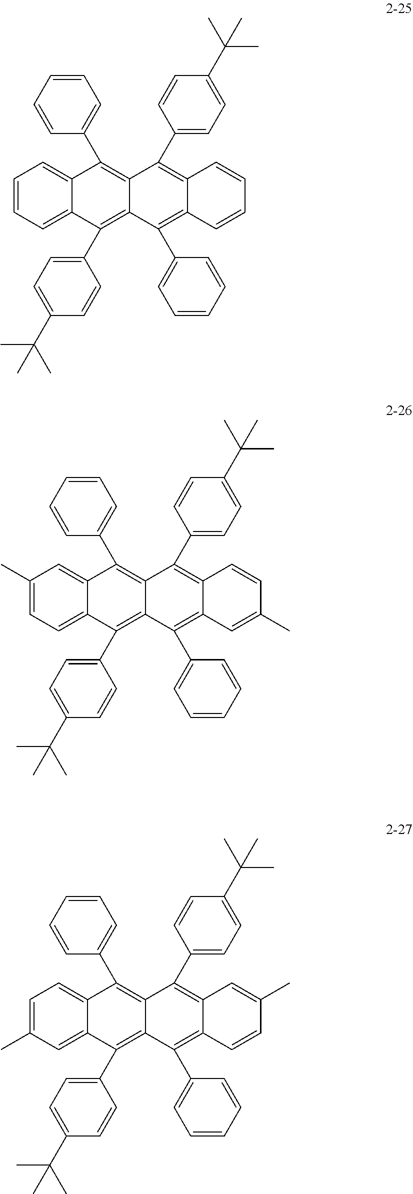

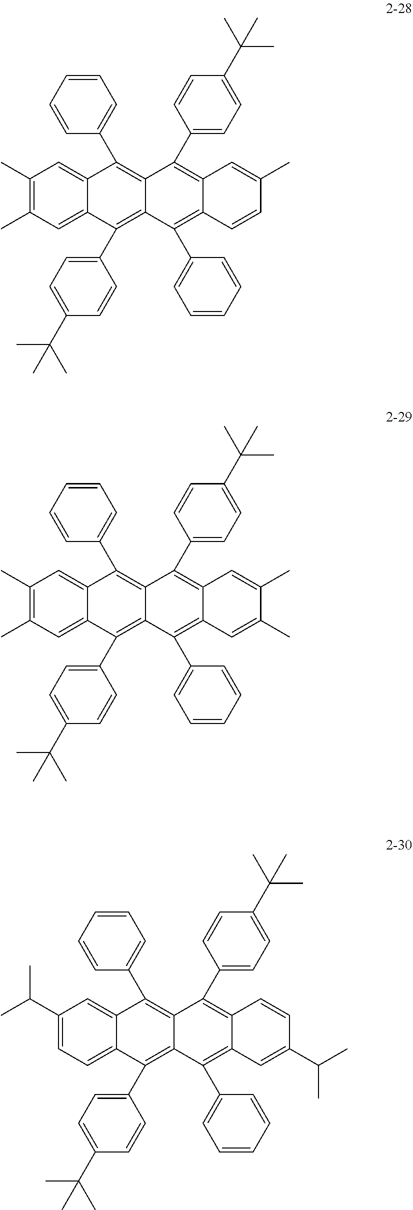

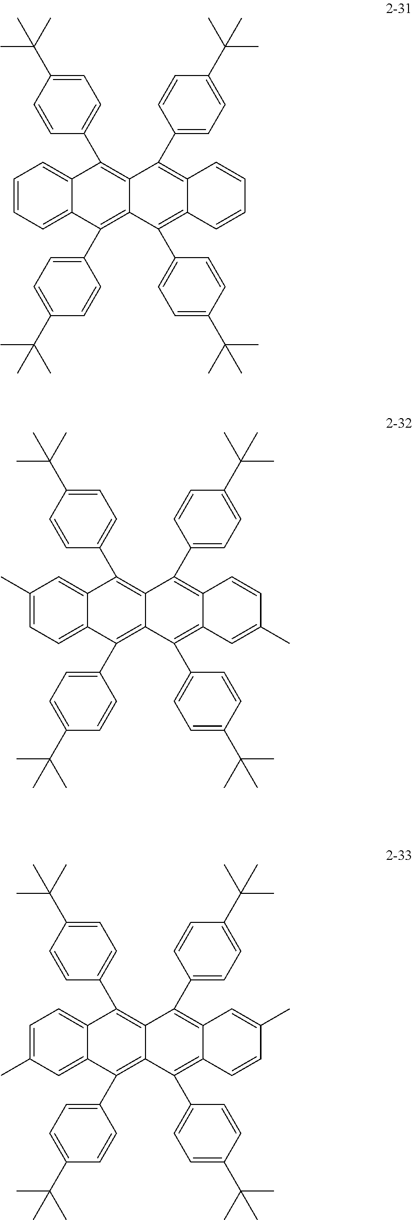

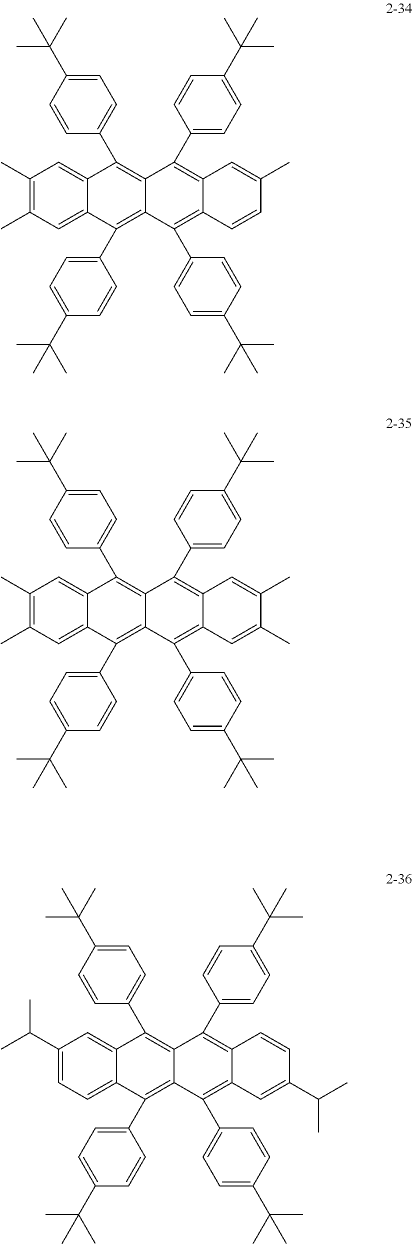

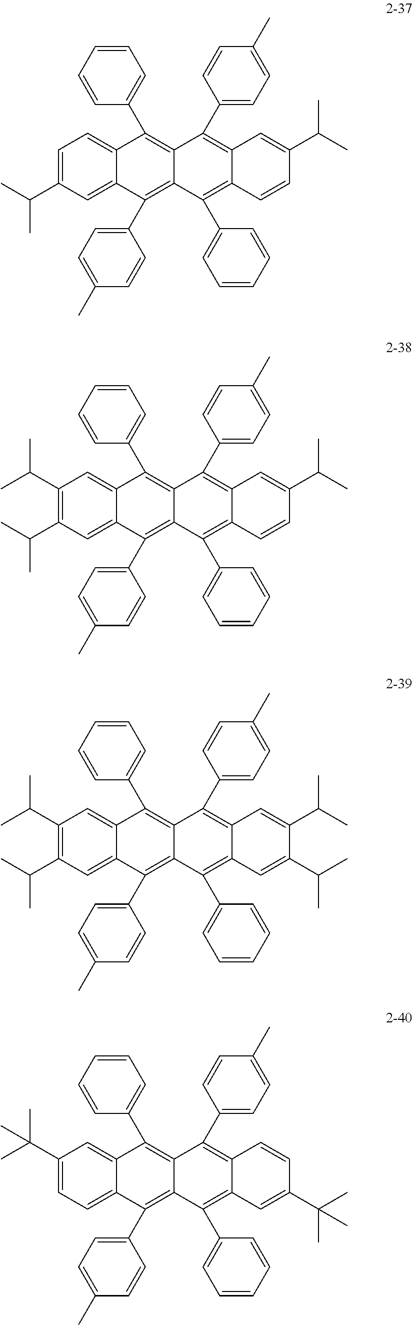

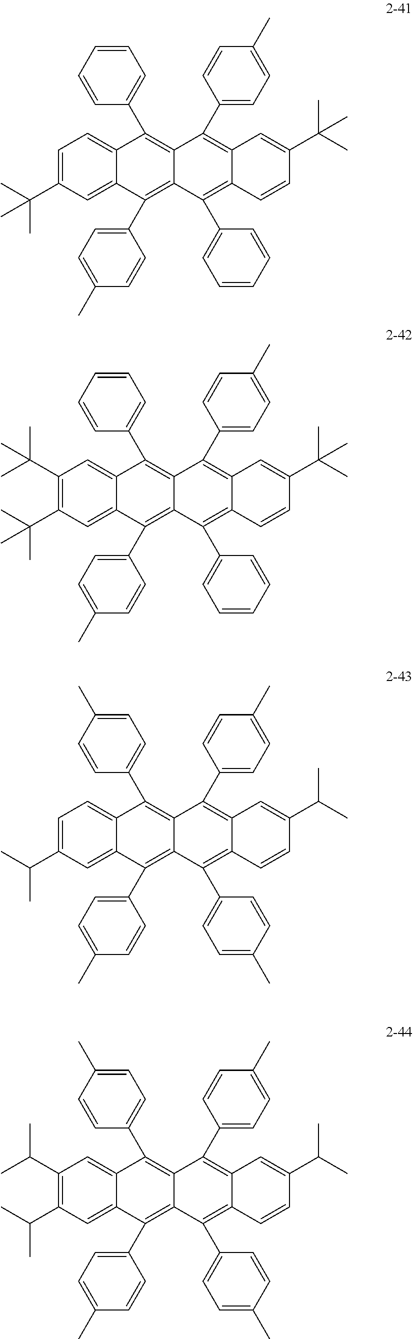

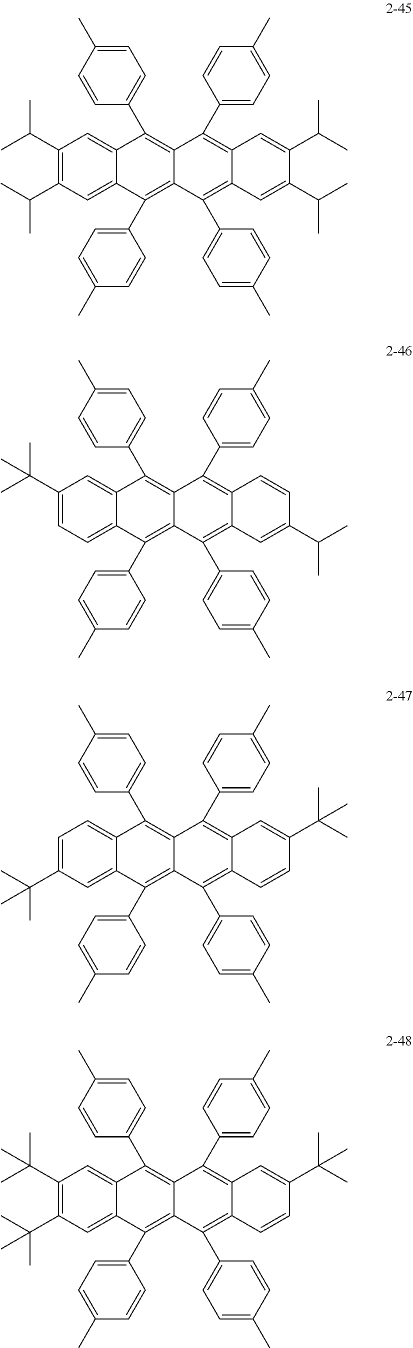

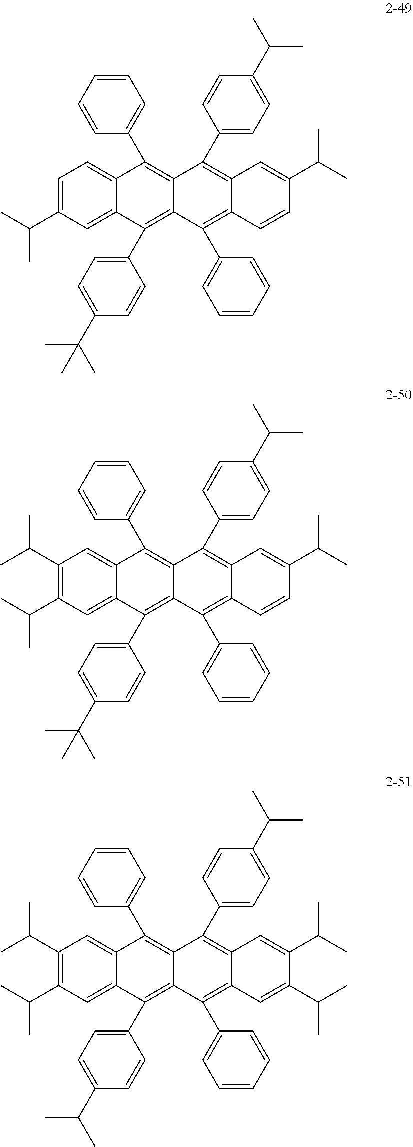

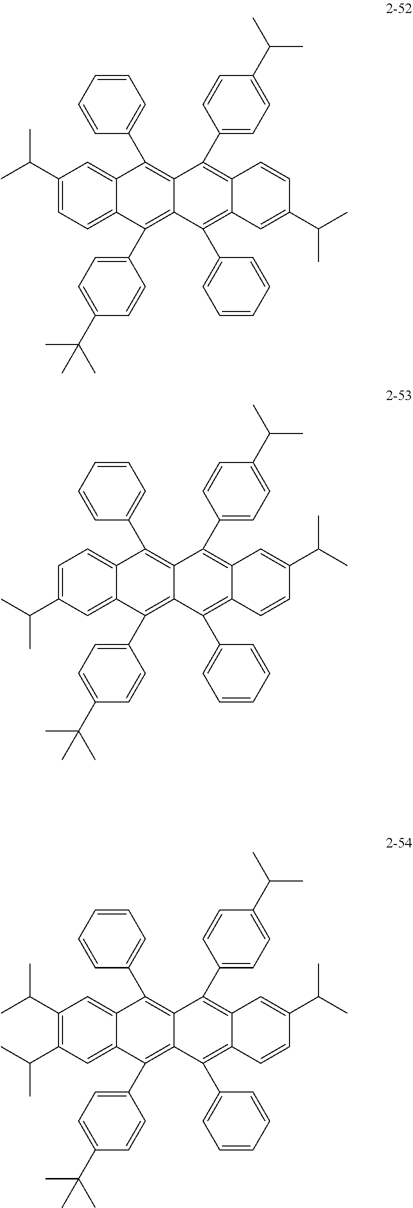

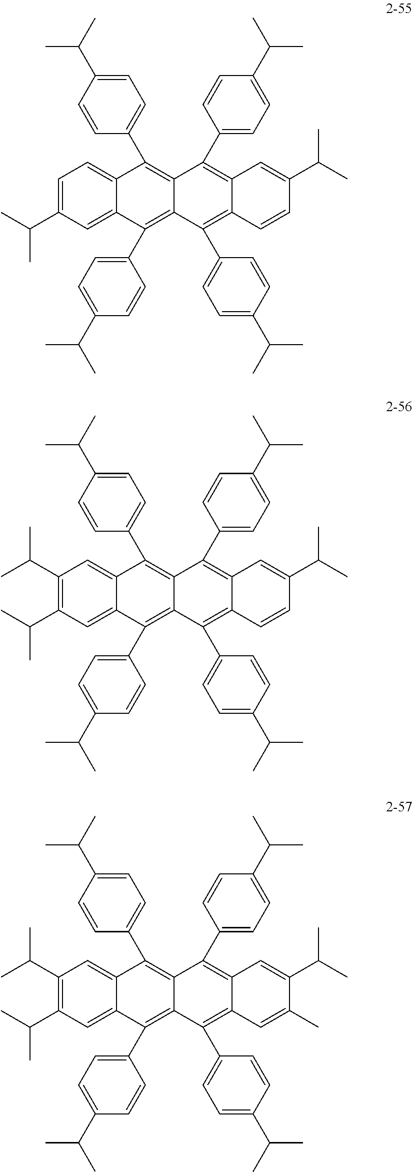

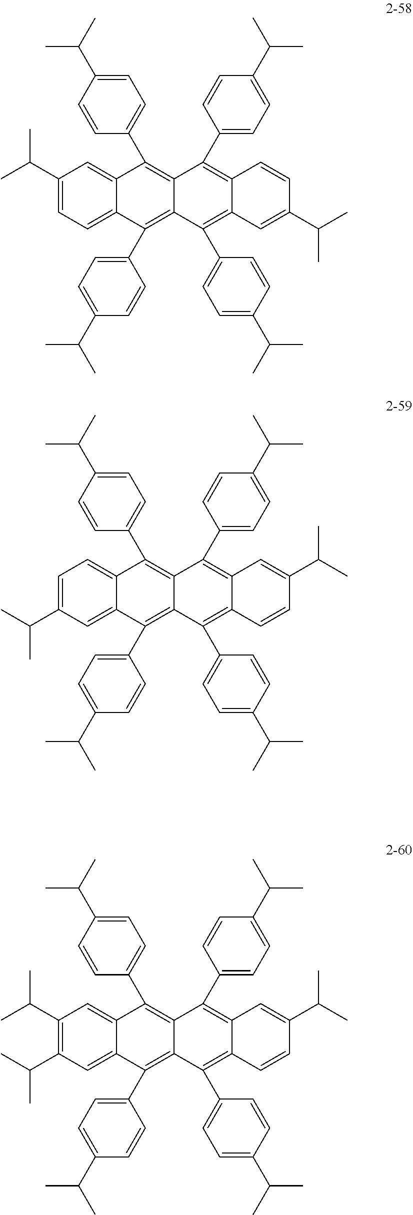

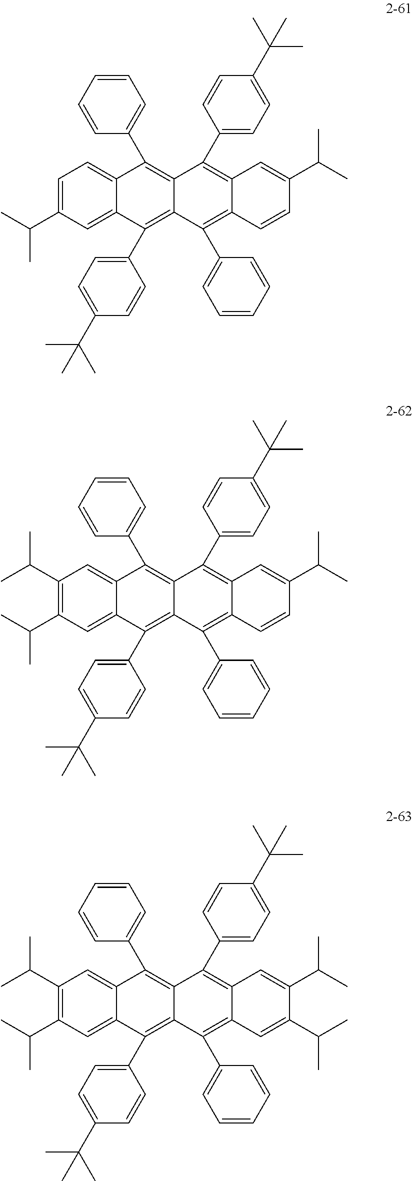

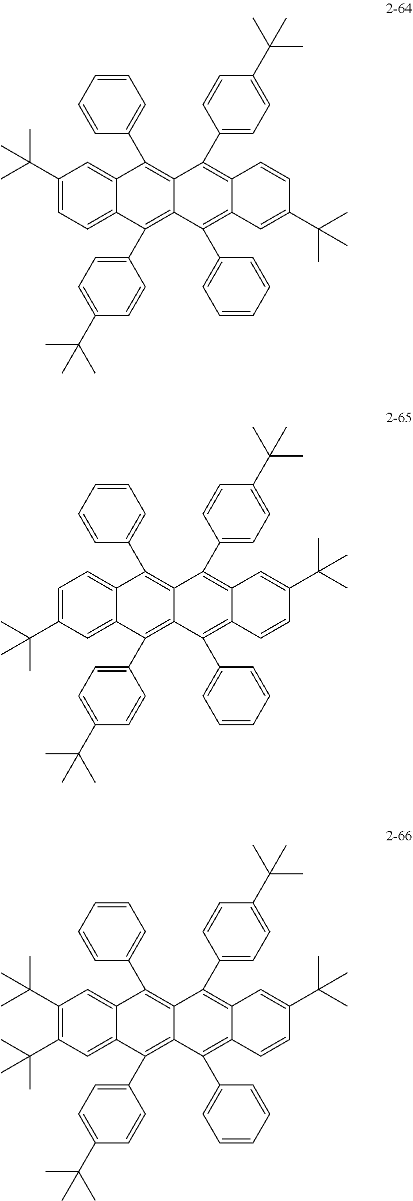

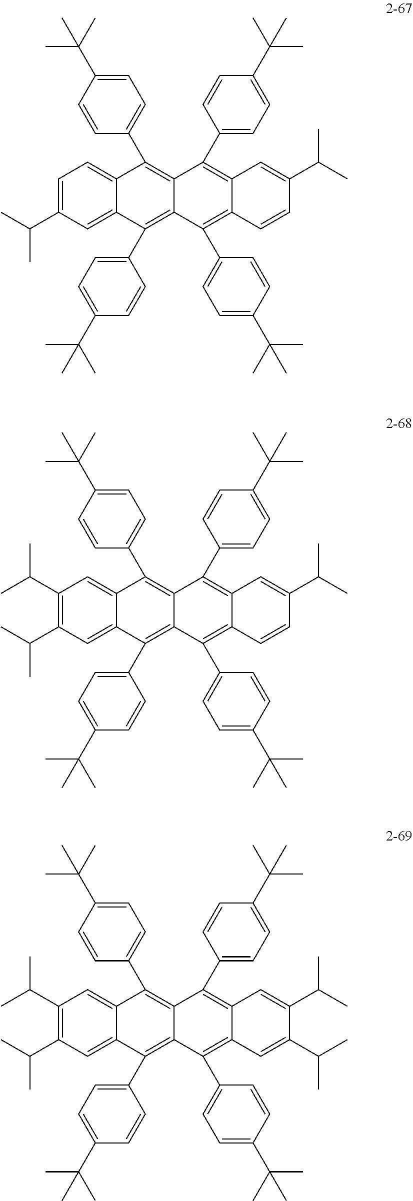

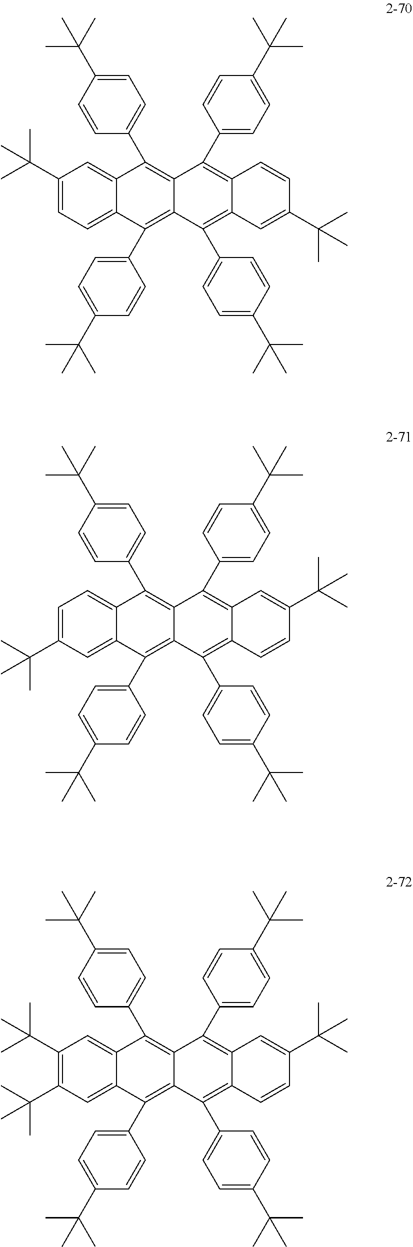

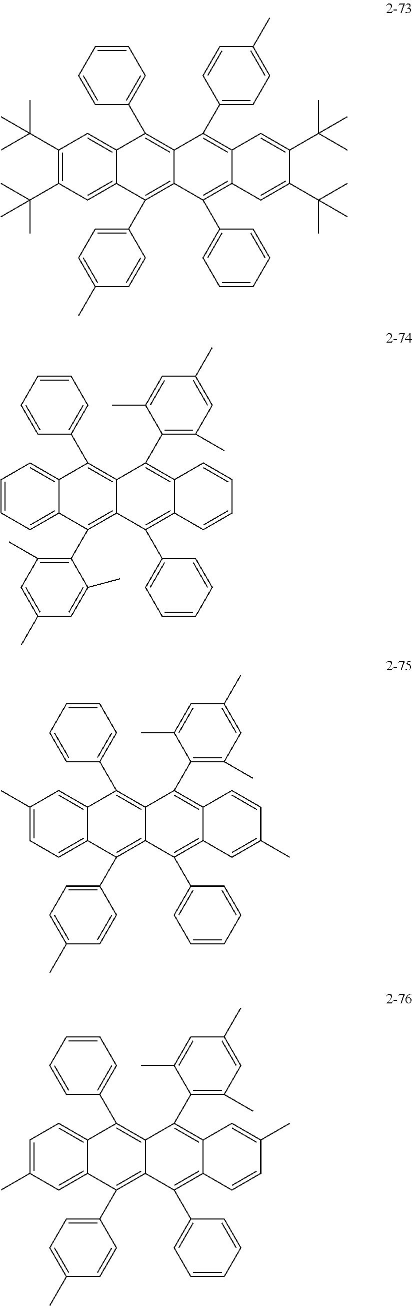

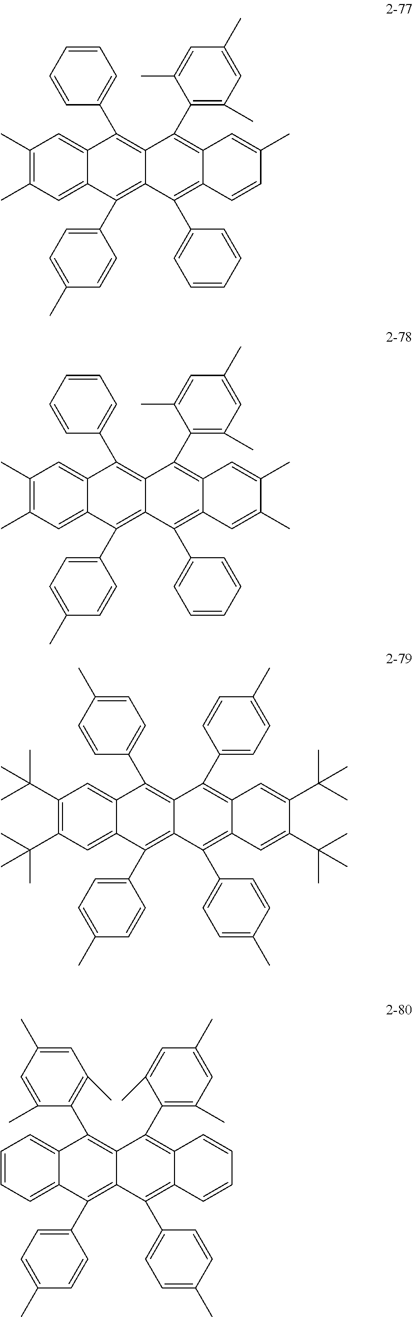

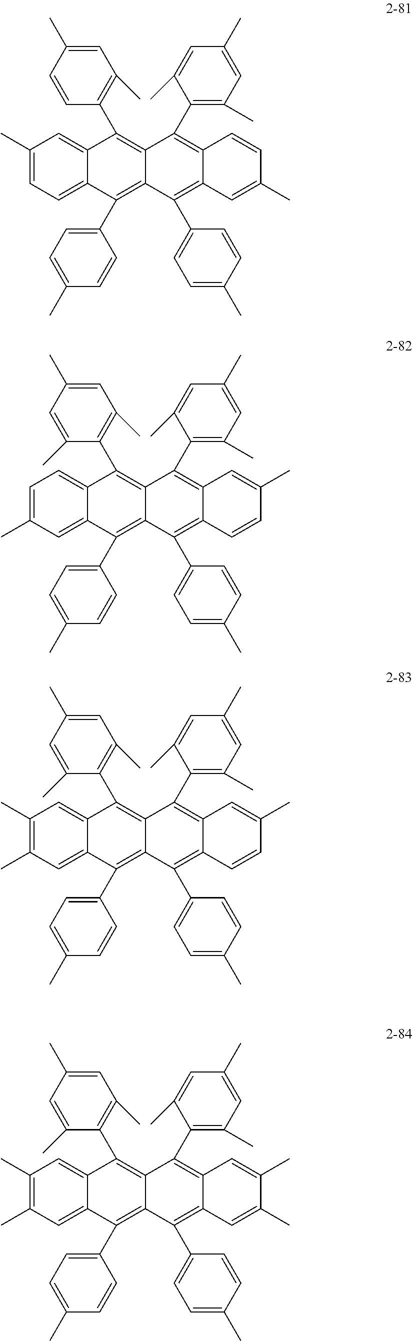

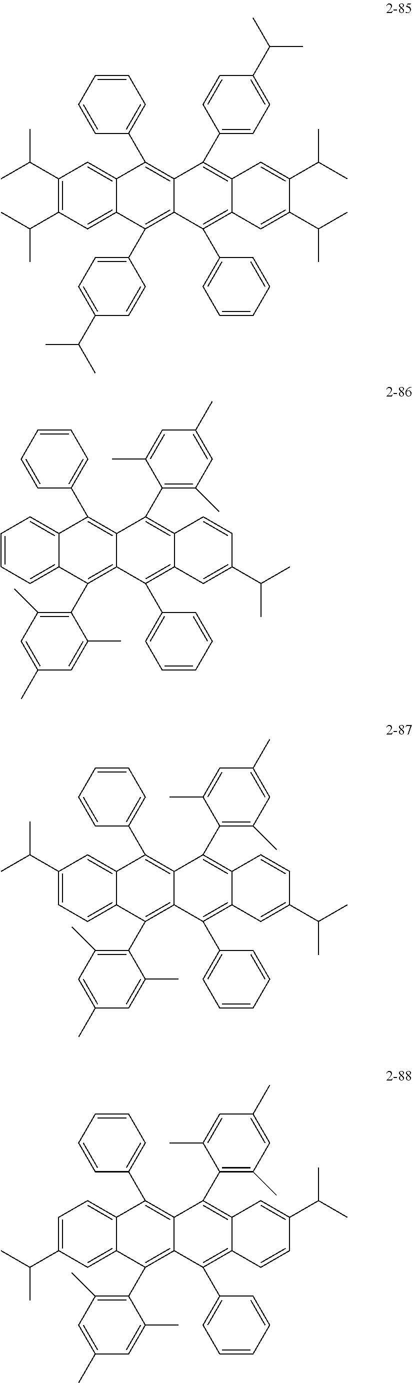

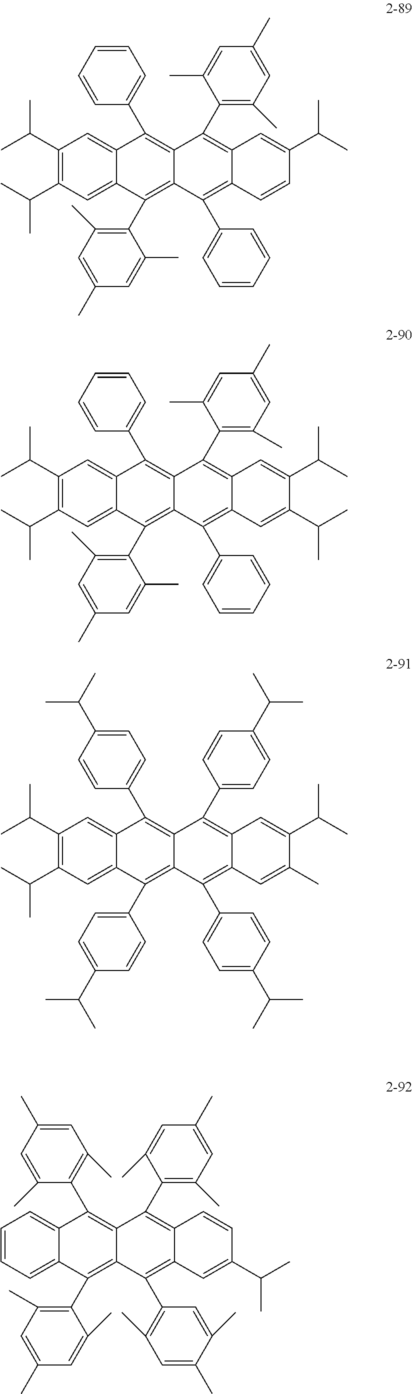

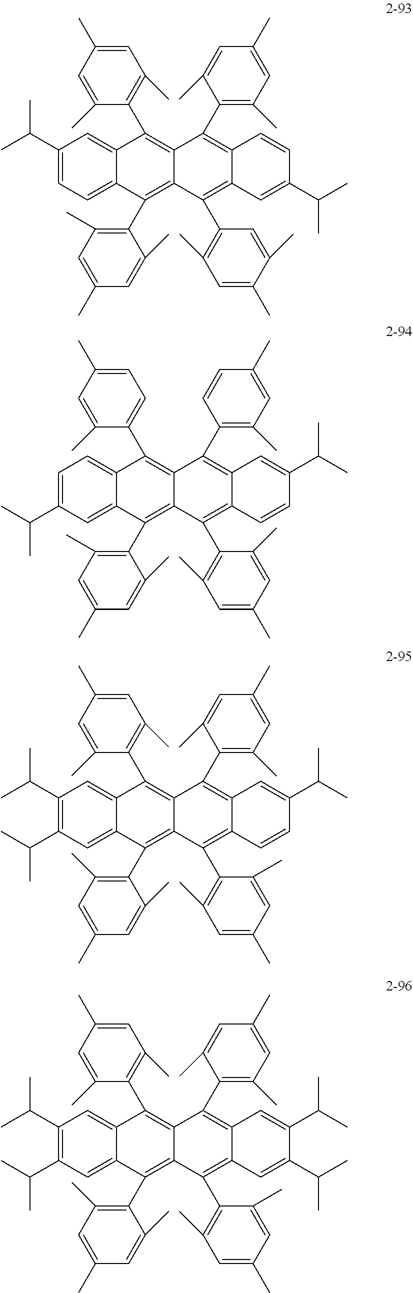

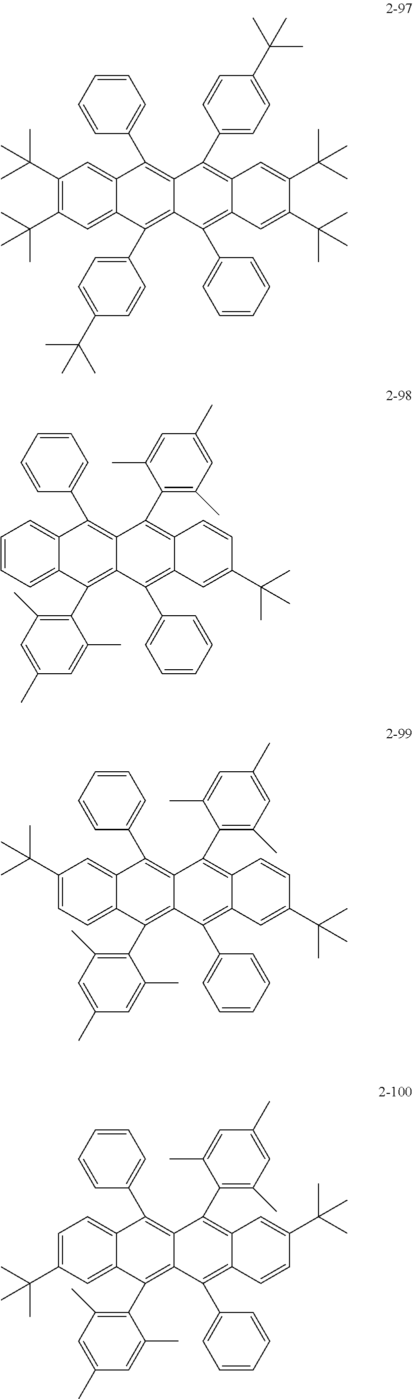

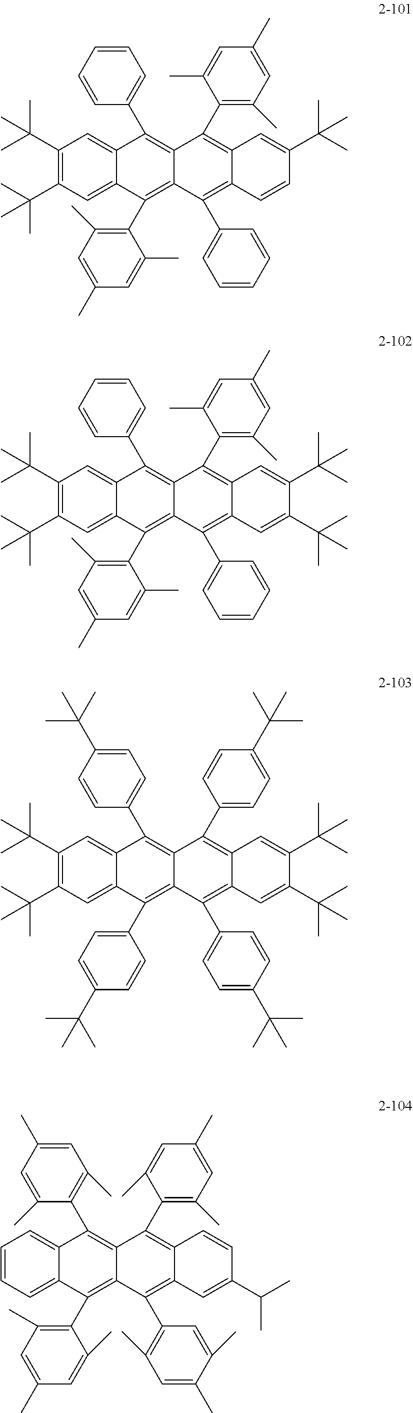

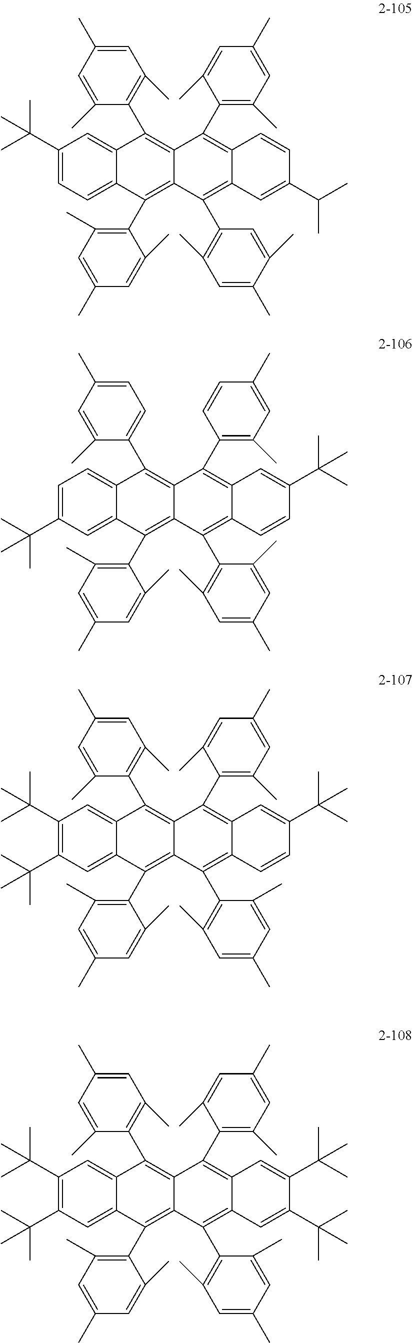

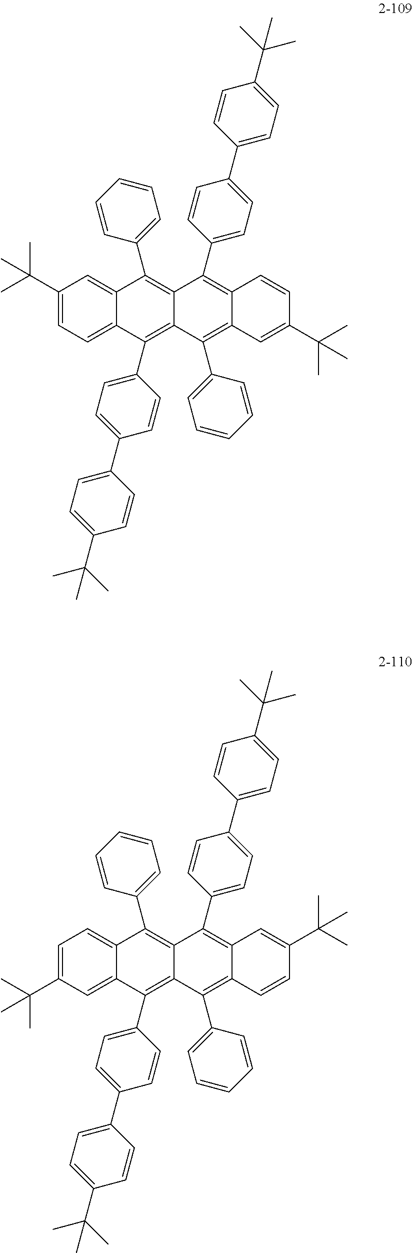

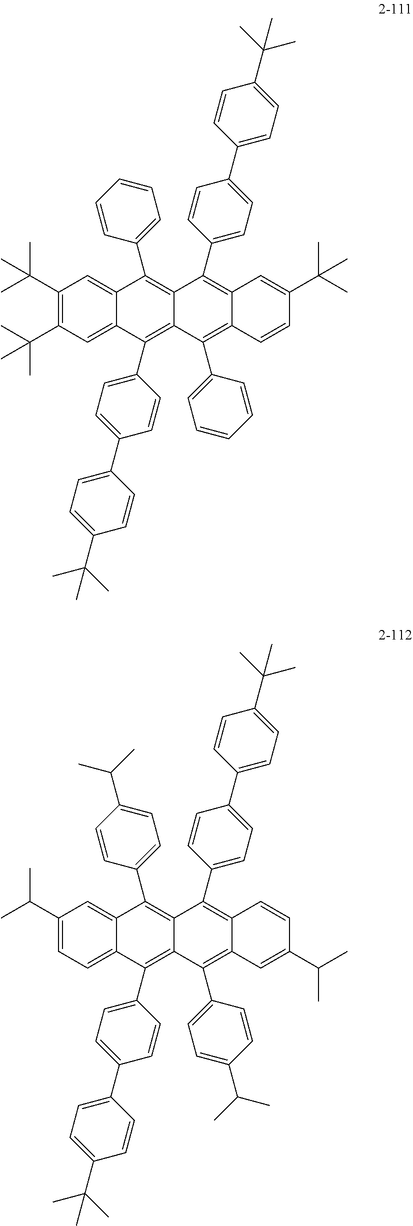

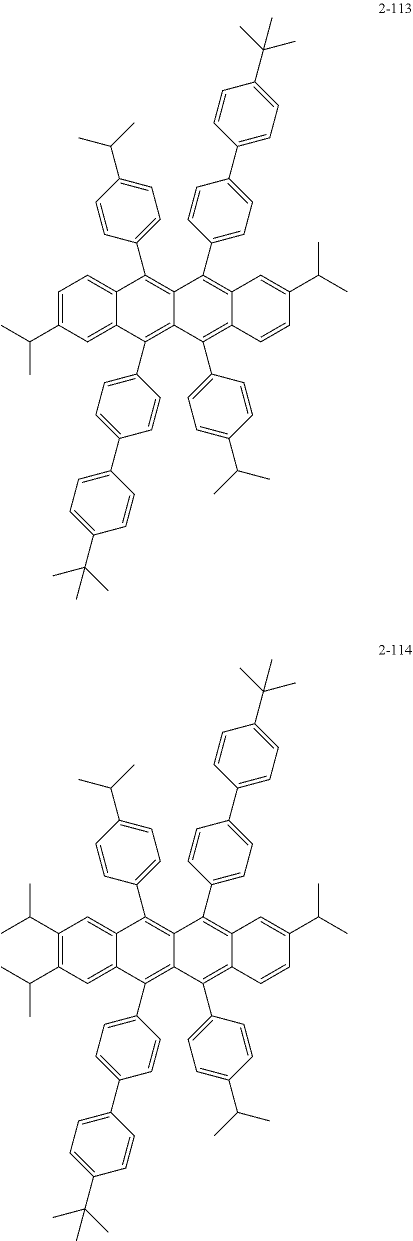

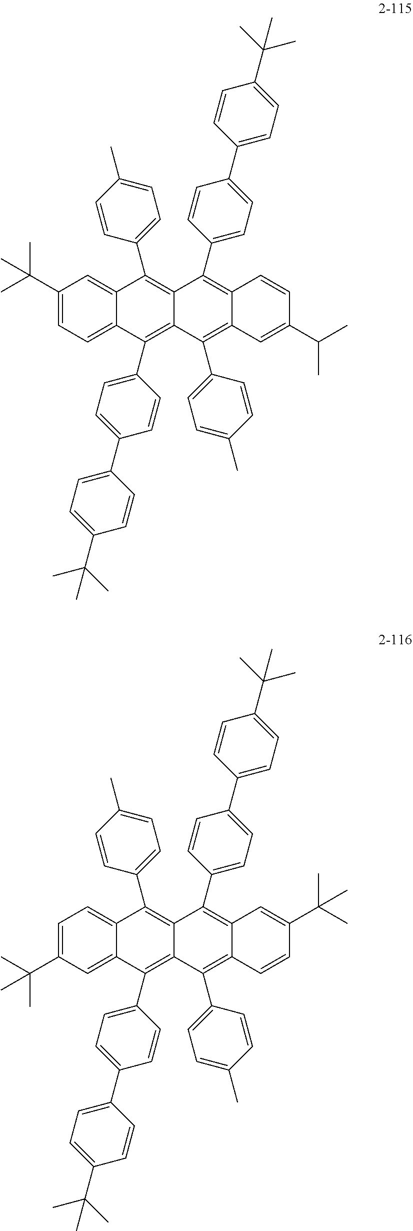

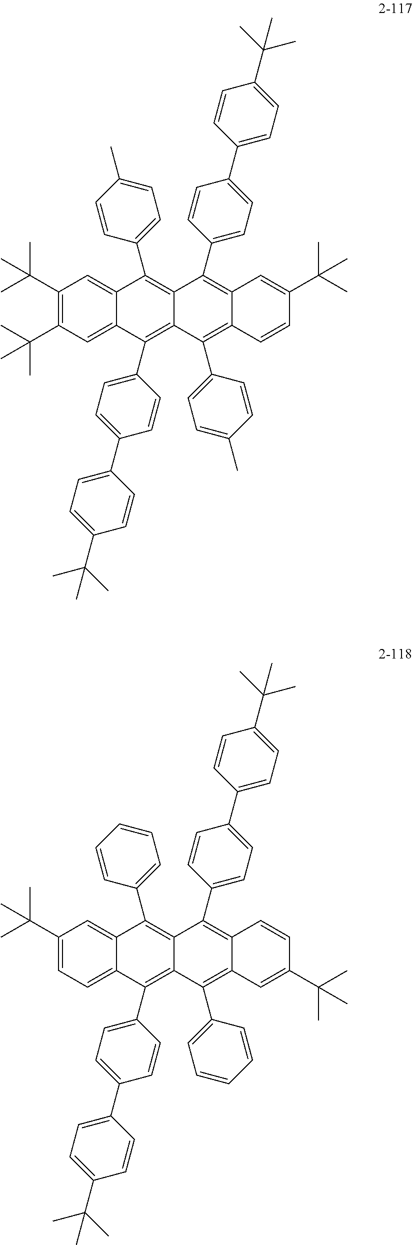

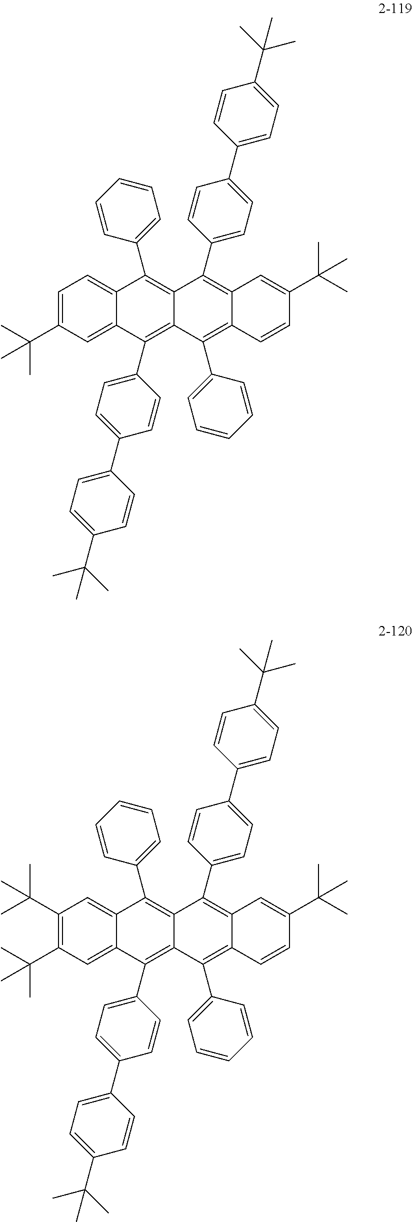

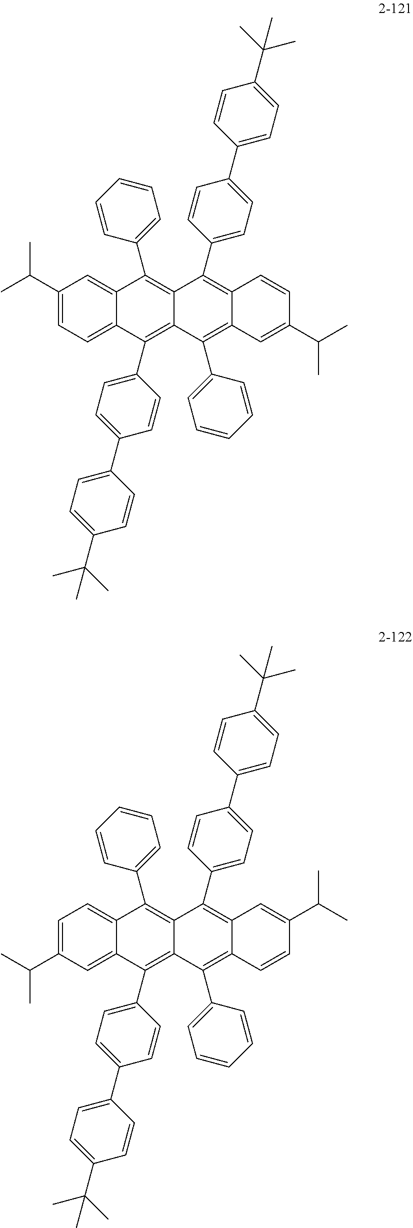

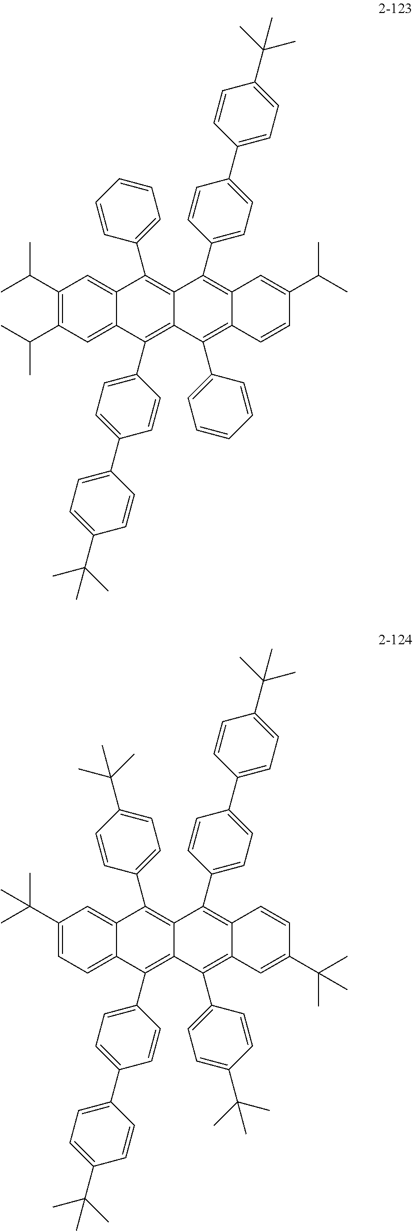

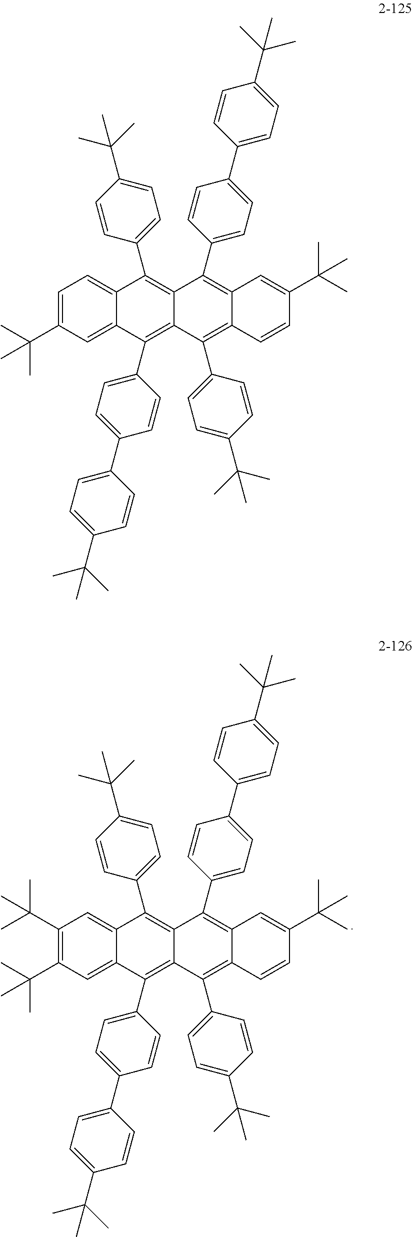

Since the second compound FD having the structure of Formulae 5 to 7 has a wide plate-like structure, the exciton energy emitted from the first compound DF can be efficiently transferred to the second compound FD, and thereby maximizing luminous efficiency. In addition, since the second compound FD can utilize only the singlet excitons, the second compound FD has relatively narrow full-width at half maximum (FWHM), which enables the second compound FD to have very excellent color purity and luminous lifespan.

The third compound H in the EML 240 can include any organic compound having wider energy level bandgap between a HOMO energy level and a LUMO energy level compared to the first compound DF and/or the second compound FD. As an example, when the EML 240 includes the third compound H of the host, the first compound DF can be a first dopant and the second compound FD can be a second dopant.

In an exemplary aspect, the third compound H, which can be included in the EML 240, can have at least one carbazolyl moiety or group and a fused hetero aromatic moiety of group linked to the carbazolyl moiety directly or via an aromatic or hetero aromatic bridging group or linker. As an example, the third compound H can have the following structure of Formula 8:

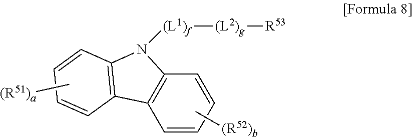

wherein, in Formula 8,

each of R⁵¹ and R⁵² is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when a is an integer of 2 or more, each R⁵¹ is identical to or different from each other, and when b is an integer of 2 or more, each R⁵² is identical to or different from each other;

R₅₃ is unsubstituted or substituted carbazolyl, unsubstituted or substituted dibenzofuranyl or unsubstituted or substituted dibenzothiophenyl;

each of L¹ and L² is independently unsubstituted or substituted C₆-C₃₀ arylene or unsubstituted or substituted C₃-C₃₀ hetero arylene; and

each of f and g is independently 0 or 1.

For example, each of the C₆-C₃₀ aryl, the C₃-C₃₀ hetero aryl, the C₆-C₃₀ arylene and the C₃-C₃₀ hetero arylene constituting or formed by R⁵¹, R⁵², L¹ and L² in Formula 8 can be independently unsubstituted or substituted with at least one of deuterium, tritium, C₁-C₂₀ alkyl, C₆-C₃₀ aryl and/or C₃-C₃₀ hetero aryl.

As an example, each of the C₆-C₃₀ aryl and the C₃-C₃₀ hetero aryl constituting R⁵¹ and R⁵² in Formula 8 can be identical to each of the C⁶-C₃₀ aryl and the C₃-C₃₀ hetero aryl defined in Formula 1, and each of the C₆-C₃₀ arylene and the C₃-C₃₀ hetero arylene constituting L1 and L2 in Formula 8 can be each of the divalent aromatic and the hetero aromatic bridging group corresponding to each of the C₆-C₃₀ aryl and the C₃-C₃₀ hetero aryl defined in Formula 1. For example, at least one of f and g in Formula 8 can be 1.

As an example, the third compound H can have the following structure of Formula 9A or Formula 9B:

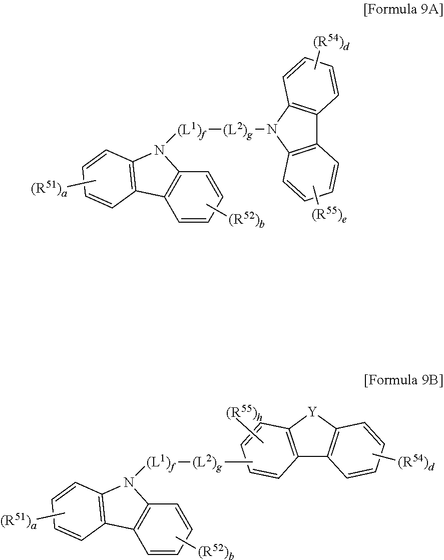

wherein, in Formulae 9A and 9B,
each of R⁵¹, R⁵², L¹, L², a, b, f and g is identical as defined in Formula 8;

each of R⁵⁴ and R⁵⁵ is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when d is an integer of 2 or more, each R⁵⁴ is identical to or different from each other, and when e or h is an integer of 2 or more, each R⁵⁵ is identical to or different from each other;

Y is O, S, or NH;

each of d and e is independently an integer of 0 to 4; and

h is an integer of 0 to 3.

For example, each of the C₆-C₃₀aryl, the C₃-C₃₀ hetero aryl, the C₆-C₃₀ arylene, the C₃-C₃₀ hetero arylene constituting R⁵¹, R³², R⁵⁴, R⁵⁵, L¹ and L² can be independently unsubstituted or substituted with at least one of deuterium, tritium, C₁-C₂₀ alkyl, C₆-C₃₀ aryl and/or C₃-C₃₀ hetero aryl.

More particularly, the third compound H can be selected from, but is not limited to, the following compound of Formula 10:

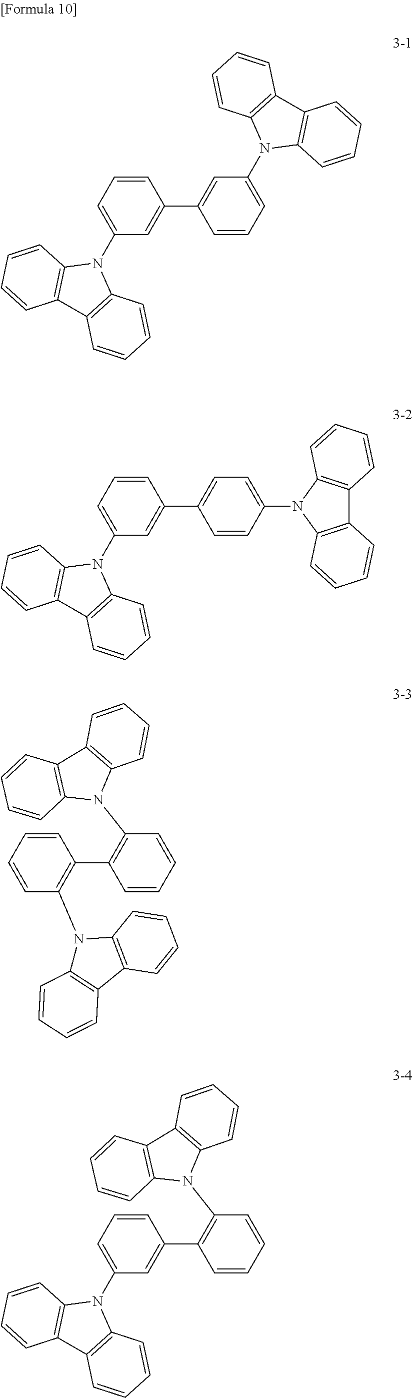

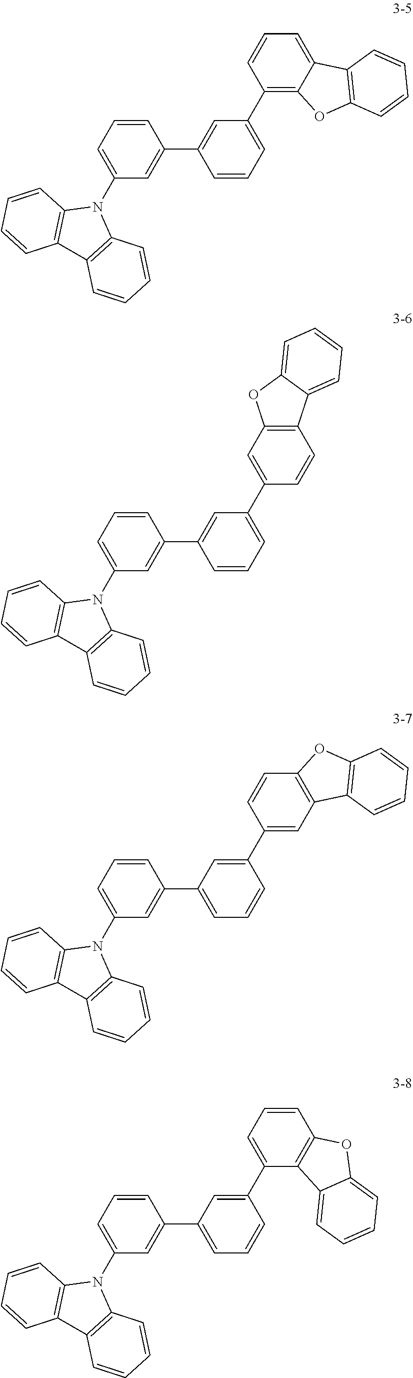 [SMILES: c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2ccc3c(c2)oc2ccccc23)c1 ; c1cc(-c2cccc(-n3c4ccccc4c4ccccc43)c2)cc(-c2ccc3oc4ccccc4c3c2)c1 ; c1cc(-c2cccc(-n3c4ccccc4c4ccccc43)c2)cc(-c2cccc3oc4ccccc4c23)c1]

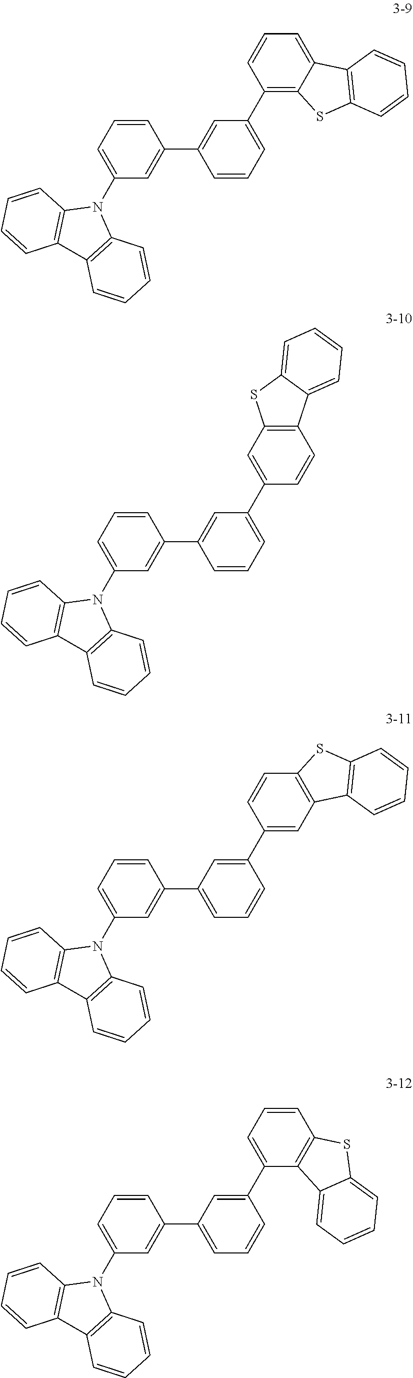 [SMILES: c1cc(-c2cccc(-n3c4ccccc4c4ccccc43)c2)cc(-c2cccc3c2sc2ccccc23)c1 ; c1cc(-c2cccc(-n3c4ccccc4c4ccccc43)c2)cc(-c2ccc3c(c2)sc2ccccc23)c1 ; c1cc(-c2cccc(-n3c4ccccc4c4ccccc43)c2)cc(-c2ccc3sc4ccccc4c3c2)c1 ; c1cc(-c2cccc(-n3c4ccccc4c4ccccc43)c2)cc(-c2cccc3sc4ccccc4c23)c1]

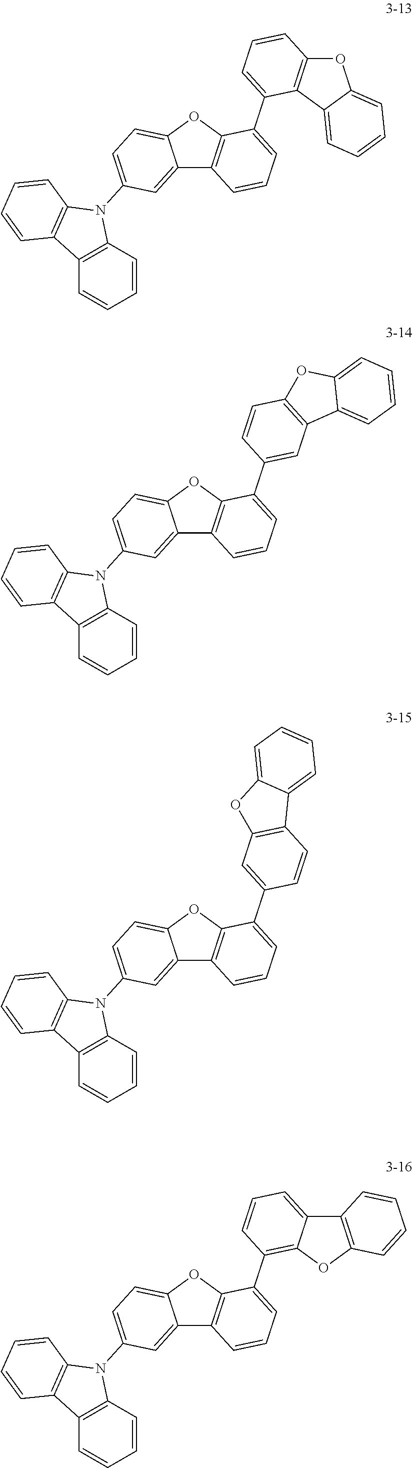 [SMILES: c1ccc2c(c1)oc1cccc(-c3cccc4c3oc3ccc(-n5c6ccccc6c6ccccc65)cc34)c12 ; c1ccc2c(c1)oc1ccc(-c3cccc4c3oc3ccc(-n5c6ccccc6c6ccccc65)cc34)cc12 ; c1ccc2c(c1)oc1cc(-c3cccc4c3oc3ccc(-n5c6ccccc6c6ccccc65)cc34)ccc12 ; c1ccc2c(c1)oc1c(-c3cccc4c3oc3ccc(-n5c6ccccc6c6ccccc65)cc34)cccc12]

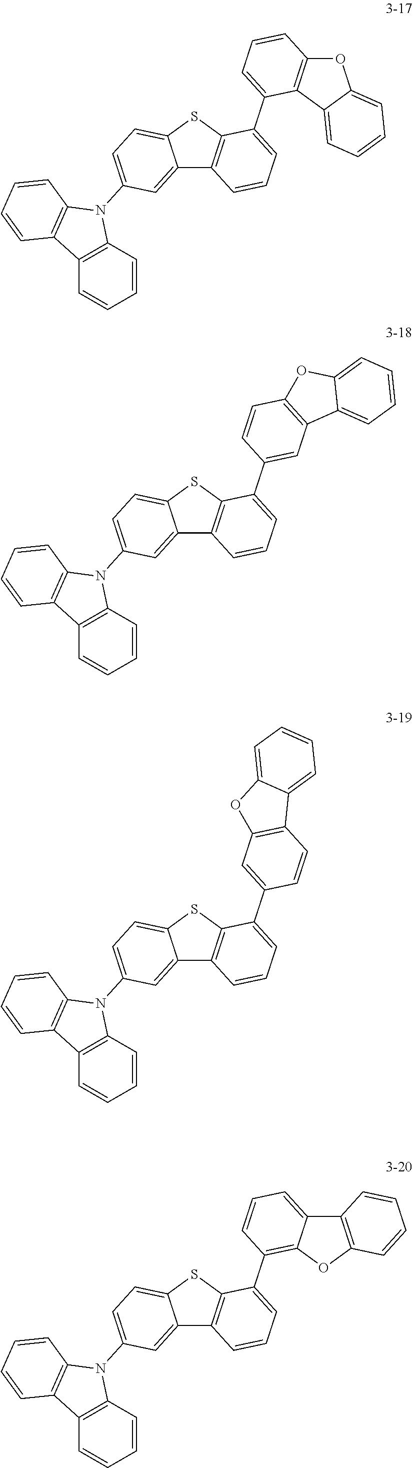 [SMILES: c1ccc2c(c1)oc1cccc(-c3cccc4c3sc3ccc(-n5c6ccccc6c6ccccc65)cc34)c12 ; c1ccc2c(c1)oc1ccc(-c3cccc4c3sc3ccc(-n5c6ccccc6c6ccccc65)cc34)cc12 ; c1ccc2c(c1)oc1cc(-c3cccc4c3sc3ccc(-n5c6ccccc6c6ccccc65)cc34)ccc12 ; c1ccc2c(c1)oc1c(-c3cccc4c3sc3ccc(-n5c6ccccc6c6ccccc65)cc34)cccc12]

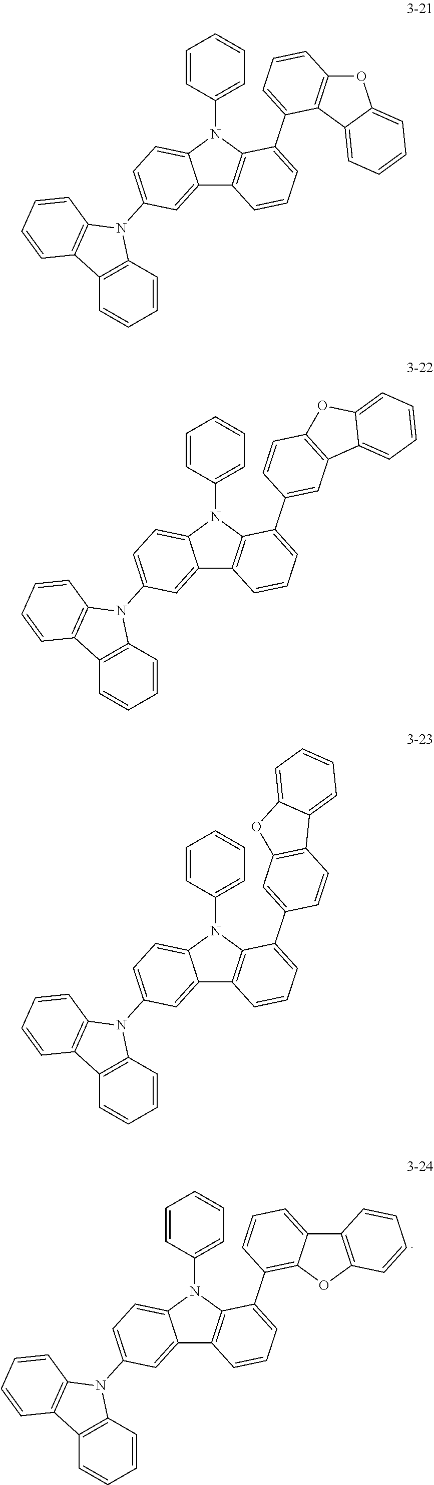 [SMILES: c1ccc(-n2c3ccc(-n4c5ccccc5c5ccccc54)cc3c3cccc(-c4cccc5oc6ccccc6c45)c32)cc1 ; c1ccc(-n2c3ccc(-n4c5ccccc5c5ccccc54)cc3c3cccc(-c4ccc5oc6ccccc6c5c4)c32)cc1 ; c1ccc(-n2c3ccc(-n4c5ccccc5c5ccccc54)cc3c3cccc(-c4ccc5c(c4)oc4ccccc45)c32)cc1 ; c1ccc(-n2c3ccc(-n4c5ccccc5c5ccccc54)cc3c3cccc(-c4cccc5c4oc4ccccc45)c32)cc1]

In an exemplary aspect, when the EML 240 includes the first compound DF, the second compound FD and the third compound H, the contents of the third compound H in the EML 240 can be larger than the contents of the first compound DF in the EML 240, and the contents of the first compound DF in the EML 240 can be larger than the contents of the second compound FD in the EML 240. When the contents of the first compound DF is larger than the contents of the second compound FD, exciton energy can be effectively transferred from the first compound DF to the second compound FD via FRET mechanism. For example, the contents of the third compound H in the EML 240 can be about 65 wt. % to about 85 wt. %, for example, about 65 wt. % to about 75 wt. %, the contents of the first compound DF in the EML 240 can be about 5 wt. % to about 30 wt. %, for example, about 15 wt. % to about 35 wt. %, and the contents of the second compound FD in the EML 240 can be about 0.1 wt. % to about 5 wt. %, for example, about 0.1 wt. % to about 2 wt. %, but is not limited thereto.

In one exemplary aspect, HOMO energy levels and/or LUMO energy levels among the third compound H of the host, the first compound DF of the delayed fluorescent material and the second compound FD of the fluorescent material must be properly adjusted. For example, the host must induce the triplet excitons generated at the delayed fluorescent material to be involved in the luminescence process without quenching as non-radiative recombination in order to implement hyper fluorescence. To this end, the energy levels among the third compound H of the host, the first compound DF of the delayed fluorescent material and the second compound FD of the fluorescent material should be adjusted.

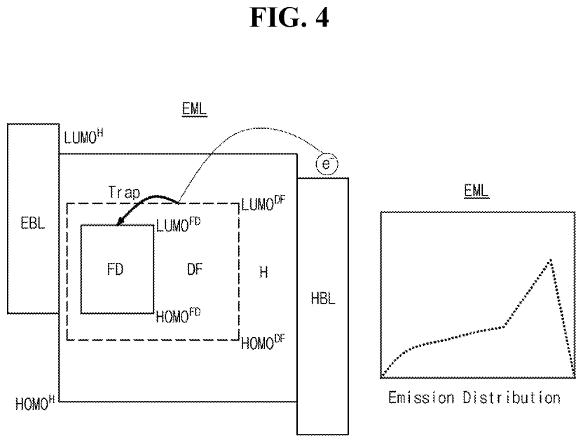

FIG. 4 is a schematic diagram illustrating a state in which electrons are trapped in the second compound and emission zone in an EML is not formed uniformly when the LUMO energy levels among the first and second compounds are not properly adjusted. As illustrated in FIG. 4, when a LUMO energy level LUMO^{DF} of the first compound DF is shallower than a LUMO energy level LUMO^{FD} of the second compound FD (LUMO^{FD}<LUMO^{DF}), the electrons transported from the HBL adjacently disposed to the EML are trapped in the second compound FD. Electrons, which can form excitons in the EML, are injected in delay due to the electron traps. Accordingly, the emission area in the EML is biased to the HBL in which electrons are injected as the excessive holes are injected to the EML compared to the electrons.

In this case, since triplet-triplet annihilation (TTA) and/or triplet-polaron annihilation (TPA) phenomena is generated in the EML, electrons are trapped in the second compound FD that has relatively low molecular dissociation energy, which causes the luminous materials to be deteriorated. As a result, the luminous lifespan of the luminous materials and lifespan of the OLED are greatly reduced.

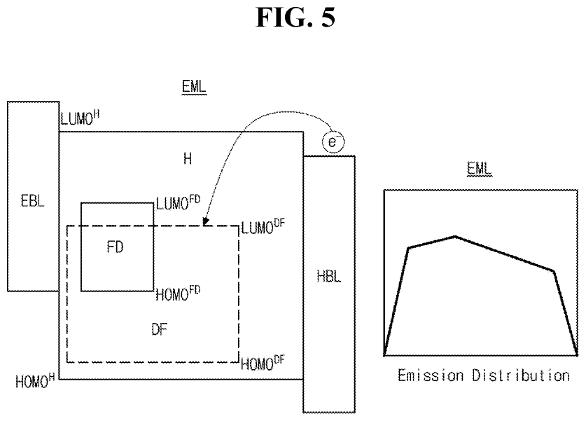

FIG. 5 is a schematic diagram illustrating a state in which the LUMO energy levels among the first and second compounds are adjusted, holes and electrons are injected into an EML in balance, and therefore, emission zone is formed uniformly in the EML in accordance with an exemplary aspect of the present disclosure. As illustrated in FIG. 5, in case of designing the EML in which the LUMO energy level LUMO^{DF} of the first compound DF is identical to or deeper than the LUMO energy level LUMO^{FD} of the second compound FD, the electrons transported from the HBL are not trapped in the second compound FD. Since holes and electrons are injected into the EML in balance, the emission area in the EML is distributed uniformly. The material deterioration owing to the electron traps in the second compound FD is minimized. Accordingly, it is possible to improve with great the luminous lifespans of the luminous materials and the lifespan of the OLED D1.

In one exemplary aspect, the LUMO energy level LUMO^{DF} of the first compound DF and the LUMO energy level LUMO^{FD} of the second compound FD can satisfy the following relationship in Equation (1):

LUMO^{FD}≥LUMO^{DF} (1)

wherein LUMO^{FD} is a LUMO energy level of the second compound and LUMO^{DF} is a LUMO energy level of the first compound.

As an example, the LUMO energy level LUMO^{DF} of the first compound DF can be designed to be identical to or deeper than at most about 1.0 eV, for example at most about 0.5 eV the LUMO energy level LUMO^{FD} of the second compound FD.

In an alternative aspect, the HOMO energy level HOMO^{DF} of the first compound DF and the HOMO energy level HOMO^{FD} of the second compound FD can satisfy the following relationship in Equation (2):

HOMO^{FD}≥HOMO^{DF} (2)

wherein HOMO^{FD} is a HOMO energy level of the second compound and HOMO^{DF} is a HOMO energy level of the first compound.

When the HOMO energy level HOMO^{DF} of the first compound DF and the HOMO energy level HOMO^{FD} of the second compound FD satisfy the relationship in Equation (2), i.e., when the HOMO energy level HOMO^{DF} of the first compound DF is designed to be identical to or deeper than the HOMO energy level HOMO^{FD} of the second compound, holes are not trapped in the second compound FD. Holes injected into the EML are transferred to the first compound DF, which can utilize the singlet exciton energies as well as the triplet exciton energies, and form excitons.

As an example, the HOMO energy level HOMO^{DF} of the first compound DF can be designed to be identical to or deeper than at most about 1.0 eV, for example at most about 0.5 eV the HOMO energy level HOMO^{FD} of the second compound FD.

When the LUMO energy level LUMO^{DF} and/or the HOMO energy level HOMO^{DF} of the first compound DF and the LUMO energy level LUMO_{FD} and/or the HOMO energy level HOMO^{FD} of the second compound FD satisfy the relationship in Equation (1) and/or (2), holes and electrons injected into the EML are transferred to the first compound DF. Since the excitons can be recombined in the first compound that can utilize both the singlet excitons and triplet excitons, it is possible to implement 100% of internal quantum efficiency using RISC mechanism. The excited singlet exciton energies generated at the first compound DF through RISC are transferred to the second compound FD of the fluorescent material via FRET, and then efficient light emission can be occurred at the second compound FD.

As an example, the first compound DF can have, but is not limited to, the LUMO energy level LUMO^{DF} between about −3.0 eV and about −3.5 eV and the HOMO energy level HOMO^{DF} between about −5.6 eV and about −6.0 eV. The second compound FD can have, but is not limited to, the LUMO energy level LUMO between about −2.8 eV and about −3.0 eV and the HOMO energy level HOMO^{FD} between about −5.2 eV and about −5.5 eV.

In addition, an energy bandgap Eg^{DF} between the LUMO energy level LUMO^{DF} and the HOMO energy level HOMO^{DF} of the first compound DF can satisfy the following relationship in Equation (3):

2.0 eV≤Eg^{DF}≤3.0 eV (3)

wherein Eg^{DF} is an energy bandgap between a HOMO energy level and a LUMO energy level of the first compound.

As an example, the energy bandgap Eg^{DF} between the LUMO energy level LUMODF and the HOMO energy level HOMO^{DF} of the first compound DF can be equal to or more than 2.0 eV and equal to or less than 2.6 eV.

In addition, a LUMO energy level LUMO^{H} of the third compound H can be shallower than the LUMO energy level LUMO^{DF} of the first compound DF and the LUMO energy level LUMO^{FD} of the second compound FD, and a HOMO energy level HOMO^{H} of the third compound H can be deeper than the HOMO energy level HOMO^{DF} of the first compound DF and the HOMO energy level HOMO^{FD} of the second compound FD. An energy bandgap Eg^{H} between the HOMO energy level HOMO^{H} and the LUMO energy level LUMO^{H} of the third compound H can be wider than the energy bandgap Eg^{DF} between the HOMO energy level HOMO^{DF} and the LUMO energy level LUMO^{DF} of the first compound DF.

As an example, an energy level bandgap (|HOMO^{H}−HOMO^{DF}|) between the HOMO energy level (HOMO^{H}) of the third compound H and the HOMO energy level (HOMO^{DF}) of the first compound DF, or an energy level bandgap (|LUMO^{H}−LUMO^{DF}|) between the LUMO energy level (LUMO^{H}) of the third compound H and the LUMO energy level (LUMO^{DF}) of the first compound DF can be equal to or less than about 0.5 eV, for example, between about 0.1 eV to about 0.5 eV. In this case, the charges can be transported efficiently from the third compound H to the first compound DF and thereby enhancing the ultimate luminous efficiency in the OLED D1.

Now, we will describe the luminous mechanism in the EML 240. FIG. 6 is a schematic diagram illustrating luminous mechanism by singlet and triplet energy levels among luminous materials in an EML in accordance with one exemplary aspect of the present disclosure. As schematically illustrated in FIG. 6, the singlet energy level S₁^{H} of the third compound H, which can be the host in the EML 240, is higher than the singlet energy level S₁^{DF} of the first compound DF having the delayed fluorescent property. In addition, the triplet energy level T₁^{H} of the third compound H can be higher than the triplet energy level T₁^{DF} of the first compound DF. As an example, the triplet energy level T₁^{H} of the third compound H can be higher than the triplet energy level T₁^{DF} of the first compound DF by at least about 0.2 eV, for example, at least about 0.3 eV such as at least about 0.5 eV.

When the triplet energy level T₁^{H} and/or the singlet energy level S₁^{H} of the third compound H is not high enough than the triplet energy level T₁^{DF} and/or the singlet energy level S₁^{DF} of the first compound DF, the excitons at the triplet energy level T₁^{DF} of the first compound DF can be reversely transferred to the triplet energy level T₁^{H} of the third compound H. In this case, the triplet exciton reversely transferred to the third compound H where the triplet exciton cannot be emitted is quenched as non-emission so that the triplet exciton energy of the first compound DF having the delayed fluorescent property cannot contribute to luminescence. As an example, the first compound DF having the delayed fluorescent property can have the energy level bandgap ΔE_{ST} between the singlet energy level S₁^{DF} and the triplet energy level T₁^{DF} equal to or less than about 0.3 eV, for example between about 0.05 eV and about 0.3 eV.

In addition, the singlet exciton energy, which is generated at the first compound DF of the delayed fluorescent material for example converted to ICT complex by RISC in the EML 240, should be efficiently transferred to the second compound FD of the fluorescent material so as to implement OLED D1 having high luminous efficiency and high color purity. To this end, the singlet energy level S₁^{DF} of the first compound DF of the delayed fluorescent material is higher than the singlet energy level Sim of the second compound FD of the fluorescent material. Optionally, the triplet energy level T₁^{DF} of the first compound DF can be higher than the triplet energy level Tim of the second compound FD.

Returning to FIG. 3, the HIL 250 is disposed between the first electrode 210 and the HTL 260 and improves an interface property between the inorganic first electrode 210 and the organic HTL 260. In one exemplary aspect, the HIL 250 can include, but is not limited to, 4,4′4″-Tris(3-methylphenylamino)triphenylamine (MTDATA), 4,4′,4″-Tris(N,N-diphenyl-amino)triphenylamine (NATA), 4,4′,4″-Tris(N-(naphthalene-1-yl)-N-phenyl-amino)triphenylamine (1T-NATA), 4,4′,4″-Tris(N-(naphthalene-2-yl)-N-phenyl-amino)triphenylamine (2T-NATA), Copper phthalocyanine (CuPc), Tris(4-carbazoyl-9-yl-phenyl)amine (TCTA), N,N′-Diphenyl-N,N′-bis(1-naphthyl)-1,1′-biphenyl-4,4″-diamine (NPB; NPD), 1,4,5,8,9,11-Hexaazatriphenylenehexacarbonitrile (Dipyrazino[2,3-f:2′3′-h]quinoxaline-2,3,6,7,10,11-hexacarbonitrile; HAT-CN), 1,3,5-tris[4-(diphenylamino)phenyl]benzene (TDAPB), poly(3,4-ethylenedioxythiphene)polystyrene sulfonate (PEDOT/PSS), N-(biphenyl-4-yl)-9,9-dimethyl-N-(4-(9-phenyl-9H-carbazol-3-yl)phenyl)-9H-fluoren-2-amine and combination thereof. The HIL 250 can be omitted in compliance with a structure of the OLED D1.

The HTL 260 is disposed between the HIL 250 and the EML 240. In one exemplary aspect, the HTL 260 can include, but is not limited to, N,N′-Diphenyl-N,N′-bis(3-methylphenyl)-1,1′-biphenyl-4,4′-diamine (TPD), NPB, 4,4′-bis(carbazol-9-yl)biphenyl (CBP), Poly[N,N′-bis(4-butylphenyl)-N,N′-bis(phenyl)-benzidine] (Poly-TPD), Poly[(9,9-dioctylfluorenyl-2,7-diyl)-co-(4,4′-(N-(4-sec-butylphenyl)diphenylamine))] (TFB), Di-[4-(N,N-di-p-tolyl-amino)-phenyl]cyclohexane (TAPC), 5-Di(9H-carbazol-9-yl)-N,N-diphenylaniline(DCDPA), N-(biphenyl-4-yl)-9,9-dimethyl-N-(4-(9-phenyl-9H-carbazol-3-yl)phenyl)-9H-fluoren-2-amine, N-(biphenyl-4-yl)-N-(4-(9-phenyl-9H-carbazol-3-yl)phenyl)biphenyl-4-amine and combination thereof.

The ETL 270 and the EIL 280 can be laminated sequentially between the EML 240 and the second electrode 230. The ETL 270 includes material having high electron mobility so as to provide electrons stably with the EML 240 by fast electron transportation. In one exemplary aspect, the ETL 270 can include, but is not limited to, any one of oxadiazole-based compounds, triazole-based compounds, phenanthroline-based compounds, benzoxazole-based compounds, benzothiazole-based compounds, benzimidazole-based compounds, triazine-based compounds, and the like.

As an example, the ETL 270 can include, but is not limited to, tris-(8-hydroxyquinoline aluminum (Alq₃), 2-biphenyl-4-yl-5-(4-t-butylphenyl)-1,3,4-oxadiazole (PBD), spiro-PBD, lithium quinolate (Liq), 1,3,5-Tris(N-phenylbenzimidazol-2-yl)benzene (TPBi), Bis(2-methyl-8-quinolinolato-N1,O8)-(1,1′-biphenyl-4-olato)aluminum (BAlq), 4,7-diphenyl-1,10-phenanthroline (Bphen), 2,9-Bis(naphthalene-2-yl)4,7-diphenyl-1,10-phenanthroline (NBphen), 2,9-Dimethyl-4,7-diphenyl-1,10-phenaathroline (BCP), 3-(4-Biphenyl)-4-phenyl-5-tert-butylphenyl-1,2,4-triazole (TAZ), 4-(Naphthalen-1-yl)-3,5-diphenyl-4H-1,2,4-triazole (NTAZ), 1,3,5-Tri(p-pyrid-3-yl-phenyl)benzene (TpPyPB), 2,4,6-Tris(3′-(pyridin-3-yl)biphenyl-3-yl)1,3,5-triazine (TmPPPyTz), Poly[9,9-bis(3′-(N,N-dimethyl)-N-ethylammonium)-propyl)-2,7-fluorene]-alt-2,7-(9,9-dioctylfluorene)] (PFNBr), tris(phenylquinoxaline) (TPQ), diphenyl[4-(triphenylsilyl)phenyl]phosphine oxide (TSPO1) and combination thereof.

The EIL 280 is disposed between the second electrode 230 and the ETL 270, and can improve physical properties of the second electrode 230 and therefore, can enhance the luminous lifespan of the OLED D1. In one exemplary aspect, the EIL 280 can include, but is not limited to, an alkali metal halide or an alkaline earth metal halide such as LiF, CsF, NaF, BaF₂ and the like, and/or an organic metal compound such as lithium quinolate, lithium benzoate, sodium stearate, and the like.

When holes are transferred to the second electrode 230 via the EML 240 and/or electrons are transferred to the first electrode 210 via the EML 240, the OLED D1 can have short lifespan and reduced luminous efficiency. In order to prevent these phenomena, the OLED D1 in accordance with this aspect of the present disclosure can have at least one exciton blocking layer adjacent to the EML 240.

For example, the OLED D1 of the exemplary aspect includes the EBL 265 between the HTL 260 and the EML 240 so as to control and prevent electron transfers. In one exemplary aspect, the EBL 265 can comprise, but is not limited to, TCTA, Tris[4-(diethylamino)phenyl]amine, N-(biphenyl-4-yl)-9,9-dimethyl-N-(4-(9-phenyl-9H-carbazol-3-yl)phenyl)-9H-fluorene-2-amine, TAPC, MTDATA, 1,3-bis(N-carbazolyl)benzene (mCP), 3,3-di(9H-carbazol-9-yl)biphenyl (mCBP), CuPc, N,N′-bis[4-(bis(3-methylphenyl)amino)phenyl]-N,N′-diphenyl-[1,1′-biphenyl]-4,4′-diamine (DNTPD), TDAPB, 3,6-bis(N-carbazolyl)-N-phenyl-carbazole and combination thereof.

In addition, the OLED D1 can further include the HBL 275 as a second exciton blocking layer between the EML 240 and the ETL 270 so that holes cannot be transferred from the EML 240 to the ETL 270. In one exemplary aspect, the HBL 275 can comprise, but is not limited to, any one of oxadiazole-based compounds, triazole-based compounds, phenanthroline-based compounds, benzoxazole-based compounds, benzothiazole-based compounds, benzimidazole-based compounds, and triazine-based compounds each of which can be used in the ETL 270.

For example, the HBL 275 can include a compound having a relatively low HOMO energy level compared to the HOMO energy level of the luminescent materials in EML 240. The HBL 275 can include, but is not limited to, BCP, BAlq, Alq₃, PBD, spiro-PBD, Liq, Bis-4,5-(3,5-di-3-pyridylphenyl)-2-methylpyrimidine (B3PYMPM), bis[2-(diphenylphosphino)phenyl] ether oxide (DPEPO), 9-(6-(9H-carbazol-9-yl)pyridine-3-yl)-9H-3,9′-bicarbazole and combination thereof.

In the above aspect, the first compound having the delayed fluorescent material and the second compound having the fluorescent material are included within the same EML. Unlike that aspect, the first compound and the second compound are included in separate EMLs.

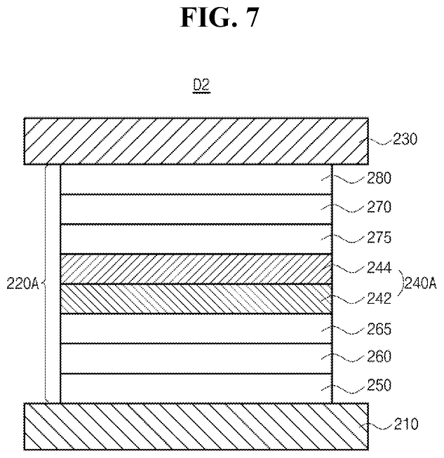

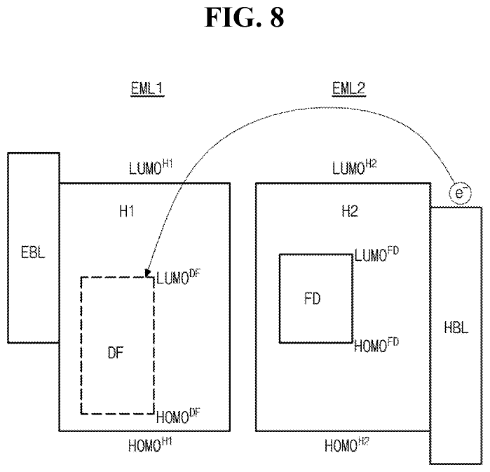

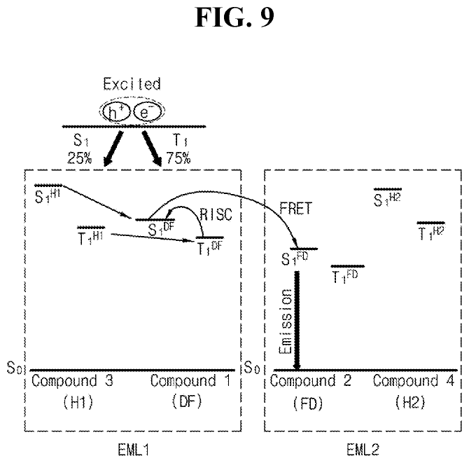

FIG. 7 is a schematic cross-sectional view illustrating an OLED in accordance with another exemplary aspect of the present disclosure. FIG. 8 is a schematic diagram illustrating a state in which the LUMO energy levels among the first and second compounds are adjusted, and therefore, electrons are not trapped in the second compound in accordance with another exemplary aspect of the present disclosure. FIG. 9 is a schematic diagram illustrating luminous mechanism by singlet and triplet energy levels among luminous materials in EMLs in accordance with another exemplary aspect of the present disclosure.

As illustrated in FIG. 8, the OLED D2 includes first and second electrodes 210 and 230 facing each other and an emissive layer 220A having single emitting part disposed between the first and second electrodes 210 and 230. The organic light emitting display device 100 (FIG. 2) includes a red pixel region, a green pixel region and a blue pixel region, and the OLED D2 can be disposed in the green pixel region.

In one exemplary aspect, the emissive layer 220A includes an EML 240A. Also, the emissive layer 220A can include at least one of an HTL 260 disposed between the first electrode 210 and the EML 240A and an ETL 270 disposed between the second electrode 230 and the EML 240A. Also, the emissive layer 220A can further comprise at least one of an HIL 250 disposed between the first electrode 210 and the HTL 260 and an EIL 280 disposed between the second electrode 230 and the ETL 270. Alternatively, the emissive layer 220A can further comprise an EBL 265 disposed between the HTL 260 and the EML 240A and/or an HBL 275 disposed between the EML 240A and the ETL 270. The configuration of the first and second electrodes 210 and 230 as well as other layers except the EML 240A in the emissive layer 220A can be substantially identical to the corresponding electrodes and layers in the OLED D1.

The EML 240A includes a first EML (EML1, lower EML, first layer) 242 disposed between the EBL 265 and the HBL 275 and a second EML (EML2, upper EML, second layer) 244 disposed between the EML1 242 and the HBL 275. Alternatively, the EML2244 can be disposed between the EBL 265 and the EML1 242.

One of the EML1 242 and the EML2 244 includes the first compound (first dopant) DF of the delayed fluorescent material, and the other of the EML1 242 and the EML2 244 includes the second compound (second dopant) FD of the fluorescent material. Also, each of the EML1 242 and the EML2 244 includes a third compound (Compound 3) H1 of a first host and a fourth compound (Compound 4) H2 of a second host. As an example, the EML1 242 can include the first compound DF and the third compound H1, and the EML2 244 can include the second compound FD and the fourth compound H2.

The first compound DF in the EML1 242 can include any delayed fluorescent material having the structure of Formulae 1 to 4. The triplet exciton energy of the first compound DF having delayed fluorescent property can be converted upwardly to its own singlet exciton energy via RISC mechanism. While the first compound DF has high internal quantum efficiency, but it has poor color purity and short luminous lifespan.

The EML 2244 includes the second compound FD of the florescent material. The second compound FD includes any organic compound having the structure of Formulae 5 to 7. While the second compound FD of the fluorescent material having the structure of Formulae 5 to 7 has an advantage in terms of color purity due to its narrow FWHM and luminous lifespan, but its internal quantum efficiency is low because its triplet exciton cannot be involved in the luminescence process.

However, in this exemplary aspect, the singlet exciton energy as well as the triplet exciton energy of the first compound DF having the delayed fluorescent property in the EML1242 can be transferred to the second compound FD in the EML2244 disposed adjacently to the EML1242 by FRET mechanism, and the ultimate light emission occurs in the second compound FD within the EML2244.

In other words, the triplet exciton energy of the first compound DF is converted upwardly to its own singlet exciton energy in the EML1242 by RISC mechanism. Then, both the initial singlet exciton energy and the converted singlet exciton energy of the first compound DF is transferred to the singlet exciton energy of the second compound FD in the EML2244. The second compound FD in the EML2244 can emit light using the triplet exciton energy as well as the singlet exciton energy. As the singlet exciton energy generated at the first compound DF in the EML1242 is efficiently transferred to the second compound FD in the EML2244, the OLED D2 can implement hyper fluorescence. In this case, while the first compound DF having the delayed fluorescent property only acts as transferring exciton energy to the second compound FD, substantial light emission is occurred in the EML2244 including the second compound FD. The OLED D2 can enhance its luminous efficiency, color purity and luminous lifespan.

Each of the EML1 242 and the EML2 244 includes the third compound H1 and the fourth compound H2, respectively. The third compound H1 can be identical to or different from the fourth compound H2. For example, each of the third compound H1 and the fourth compound H2 can include, but is not limited to, the organic compound having the structure of Formulae 8 to 10.

Similar to the first aspect, the LUMO energy level LUMO^{DF} of the first compound DF and the LUMO energy level LUMO^{FD} of the second compound FD can satisfy the requirement defined in Equation (1). The HOMO energy level HOMO^{DF} of the first compound DF and the HOMO energy level HOMO^{FD} of the second compound FD can satisfy the requirement defined in Equation (2). In addition, the energy bandgap Eg^{DF} between the LUMO energy level LUMO^{DF} and the HOMO energy level HOMO^{DF} of the first compound DF can satisfy the requirement defined in Equation (3). In this case, holes and electrons are injected into the EML, the luminous area is distributed uniformly in the EML, and therefore, the luminous lifespan of the luminous materials and lifespan of the OLED D2 can be improved greatly.

Also, an energy level bandgap (|HOMO^{H}−HOMO^{DF}|) between the HOMO energy levels (HOMO^{H1} and HOMO^{H2}) of the third and fourth compounds H1 and H2 and the HOMO energy level (HOMO^{DF}) of the first compound DF, or an energy level bandgap (|LUMO^{H}−LUMO^{DF}|) between the LUMO energy levels (LUMO^{H1} and LUMO^{H2}) of the third and fourth compounds H1 and H2 and the LUMO energy level (LUMO^{DF}) of the first compound DF can be equal to or less than about 0.5 eV. The HOMO or LUMO energy level bandgap between the third and fourth compounds and the first compound does not satisfy that condition, the exciton energy at the first compound DF can be quenched as a non-radiative recombination, or exciton energies may not be transferred efficiently to the first compound DF and/or the second compound FD from the third and fourth compounds H1 and H2, thus the internal quantum efficiency in the OLED D2 can be reduced.

Also, each of the exciton energies generated in each of the third compound H1 in the EML1 242 and the fourth compound H2 in the EML2 244 should be transferred primarily to the first compound DF of the delayed florescent material and then to the second compound FD of the fluorescent material in order to realize efficient light emission. As illustrated in FIG. 9, each of the singlet energy levels S₁^{H1} and S₁^{H2} of the third and fourth compounds H1 and H2 is higher than the singlet energy level S₁^{DF} of the first compound DF having the delayed fluorescent property. Also, each of the triplet energy levels T₁^{H1} and T₁^{H2} of the third and fourth compounds H1 and H2 can be higher than the triplet energy level T₁^{DF} of the first compound DF. For example, the triplet energy levels T₁^{H1} and T₁^{H2} of the third and fourth compound H1 and H2 can be higher than the triplet energy level T₁^{DF} of the first compound DF by at least about 0.2 eV, for example, by at least 0.3 eV such as by at least 0.5 eV.

Also, the singlet energy level S₁^{H2} of the fourth compound H2 of the second host is higher than the singlet energy level S₁^{FD} of the second compound FD of the fluorescent material. Optionally, the triplet energy level T₁^{H2} of the fourth compound H2 can be higher than the triplet energy level Tim of the second compound FD. In this case, the singlet exciton energy generated at the fourth compound H2 can be transferred to the singlet energy of the second compound FD.

In addition, the singlet exciton energy, which is generated at the first compound DF having the delayed fluorescent property that is converted to ICT complex by RISC in the EML1 242, should be efficiently transferred to the second compound FD of the fluorescent material in the EML2 244. To this end, the singlet energy level S₁^{DF} of the first compound DF of the delayed fluorescent material in the EML1 242 is higher than the singlet energy level Sim of the second compound FD of the fluorescent material in the EML2 244. Optionally, the triplet energy level T₁^{DF} of the first compound DF in the EML1 242 can be higher than the triplet energy level T₁^{FD} of the second compound FD in the EML2 244.

Each of the contents of the third and fourth compounds H1 and H2 in the EML1242 and the EML2244 can be larger than or identical to each of the contents of the first and second compounds DF and FD in the same layer, respectively. Also, the contents of the first compound DF in the EML1 242 can be larger than the contents of the second compound FD in the EML2 244. In this case, exciton energy is efficiently transferred from the first compound DF to the second compound FD via FRET mechanism. As an example, the EML1 242 can include the first compound DF between about 1 wt. % and about 50 wt. %, for example, about 10 wt. % and about 40 wt. % such as about 20 wt. % and about 40 wt. %. The EML2 244 can include the second compound FD between about 1 wt. % and about 10 wt. %, for example, about 1 wt. % and 5 wt. %.

In one exemplary aspect, when the EML2 244 is disposed adjacently to the HBL 275, the fourth compound H2 in the EML2244 can be the same material as the HBL 275. In this case, the EML2244 can have a hole blocking function as well as an emission function. In other words, the EML2244 can act as a buffer layer for blocking holes. In one aspect, the HBL 275 can be omitted where the EML2 244 can be a hole blocking layer as well as an emitting material layer.

In another exemplary aspect, when the EML2 244 is disposed adjacently to the EBL 265, the fourth compound H2 in the EML2244 can be the same as the EBL 265. In this case, the EML2 244 can have an electron blocking function as well as an emission function. In other words, the EML2 244 can act as a buffer layer for blocking electrons. In one aspect, the EBL 265 can be omitted where the EML2 244 can be an electron blocking layer as well as an emitting material layer.

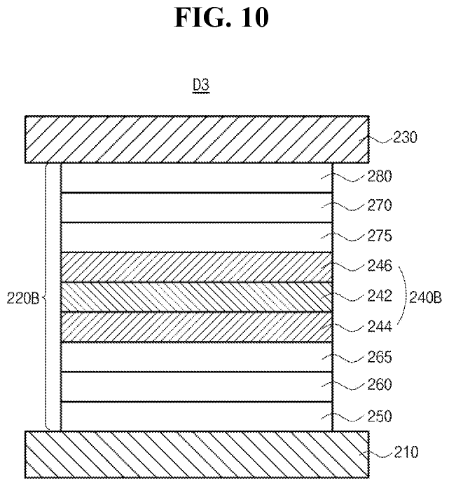

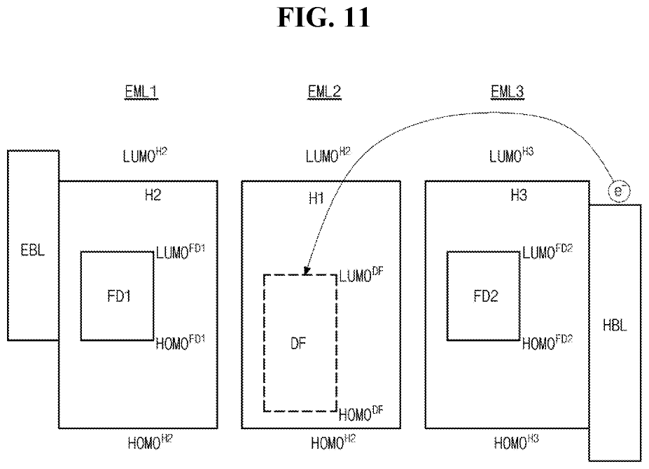

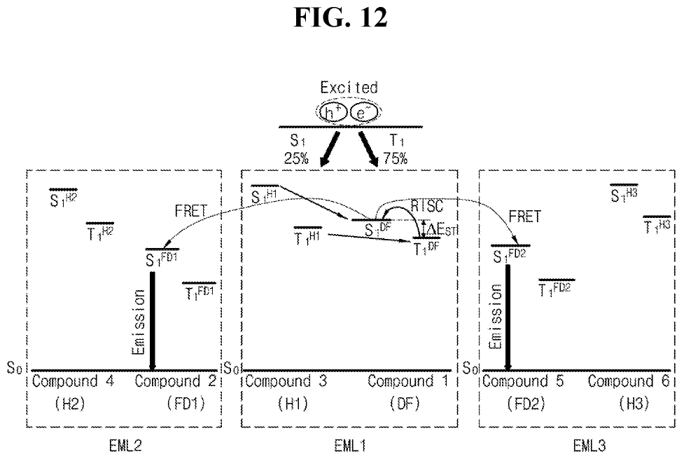

An OLED having a triple-layered EML will be explained. FIG. 10 is a schematic cross-sectional view illustrating an OLED in accordance with still another exemplary aspect of the present disclosure. FIG. 11 is a schematic diagram illustrating a state in which the LUMO energy levels among the first, second and fifth compounds are adjusted, and therefore, electrons are not trapped in the second and fifth compounds in accordance with still another exemplary aspect of the present disclosure. FIG. 12 is a schematic diagram illustrating luminous mechanism by singlet and triplet energy levels among luminous materials in EMLs in accordance with still another exemplary aspect of the present disclosure.

As illustrated in FIG. 11, the OLED D3 includes first and second electrodes 210 and 230 facing each other and an emissive layer 220B disposed between the first and second electrodes 210 and 230. The organic light emitting display device 100 (FIG. 2) includes a red pixel region, a green pixel region and a blue pixel region, and the OLED D3 can be disposed in the green pixel region.

In one exemplary aspect, the emissive layer 220B having single emitting part includes a triple-layered EML 240B. The emissive layer 220B can include at least one of an HTL 260 disposed between the first electrode 210 and the EML 240B and an ETL 270 disposed between the second electrode 230 and the EML 240B. Also, the emissive layer 220B can further include at least one of an HIL 250 disposed between the first electrode 210 and the HTL 260 and an EIL 280 disposed between the second electrode 230 and the ETL 270. Alternatively, the emissive layer 220B can further include an EBL 265 disposed between the HTL 260 and the EML 240B and/or an HBL 275 disposed between the EML 240B and the ETL 270. The configurations of the first and second electrodes 210 and 230 as well as other layers except the EML 240B in the emissive layer 220B is substantially identical to the corresponding electrodes and layers in the OLEDs D1 and D2.

The EML 240B includes a first EML (EML1, middle EML, first layer) 242, a second EML (EML2, lower EML, second layer) 244 and a third EML (EML3, upper EML, third layer) 246. The EML1 242 is disposed between the EBL 265 and the HBL 275, the EML2 244 is disposed between the EBL 265 and the EML1 242 and the EML3 246 is disposed between the EML1 242 and the HBL 275.

The EML1 242 includes the first compound (first dopant) DF of the delayed fluorescent material. Each of the EML2244 and the EML3 246 includes the second compound (second dopant) FD1 and a fifth compound (Compound 5, third dopant) FD2 each of which is the fluorescent material, respectively. Also, each of the EML 1242, the EML2 244 and the EML3 246 includes the third compound H1 of the first host, the fourth compound H2 of the second host and a sixth compound (Compound 6) H3 of a third host, respectively.

In accordance with this aspect, both the singlet energy as well as the triplet energy of the first compound DF of the delayed fluorescent material in the EML1242 can be transferred to the second and fifth compounds FD1 and FD2 of the fluorescent materials each of which is included in the EML2244 and EML3 246 disposed adjacently to the EML1242 by FRET energy transfer mechanism. Accordingly, the ultimate emission occurs in the second and fifth compounds FD1 and FD2 in the EML2 244 and the EML3 246.

In other words, the triplet exciton energy of the first compound DF having the delayed fluorescent property in the EML1242 is converted upwardly to its own singlet exciton energy by RISC mechanism, then the singlet exciton energy including the initial and converted singlet exciton energy of the first compound DF is transferred to the singlet exciton energy of the second and fifth compounds FD1 and FD2 in the EML2244 and the EML3 246 because the first compound DF has the singlet energy level S₁^{DF} higher than each of the singlet energy levels S₁^{FD1} and S₁^{FD2} of the second and fifth compounds FD1 and FD2 (FIG. 12). The singlet exciton energy of the first compound DF in the EML1242 is transferred to the second and fifth compounds FD1 and FD2 in the EML2244 and the EML3 246 which are disposed adjacently to the EML1242 by FRET mechanism.

Both the second and fifth compounds FD1 and FD2 in the EML2244 and EML3 246 can emit light using the singlet exciton energy as well as the triplet exciton energy derived from the first compound DF. Each of the second and fifth compounds FD1 and FD2 has excellent color purity and luminous lifespan compared to the first compound DF. In this aspect, the OLED D3 can improve its quantum efficiency, color purity and luminous lifespan. The ultimate emission occurs in the EML2 244 and the EML3 246 each of which includes the second compound FD1 and the fifth compound FD2, respectively.

The first compound DF of the delayed fluorescent material includes any organic compound having the structure of Formulae 1 to 4. Each of the second and fifth compounds FD1 and FD2 of the fluorescent material includes independently any organic compound having the structure of Formulae 5 to 7. The third compound H1, the fourth compound H2 and the sixth compound H3 can be identical to or different from each other. For example, each of the third compound H1, the fourth compound H2 and the sixth compound H3 can independently include, but is not limited to, the organic compound having the structure of Formulae 8 to 10, respectively.

Similar to the first and second aspects, the LUMO energy level LUMO^{DF} of the first compound DF and the LUMO energy level LUMO^{FD} of the second compound FD can satisfy the requirement defined in Equation (1). The HOMO energy level HOMO^{DF} of the first compound DF and the HOMO energy level HOMO of the second compound FD can satisfy the requirement defined in Equation (2). In addition, the energy bandgap Eg^{DF} between the LUMO energy level LUMO^{DF} and the HOMO energy level HOMO^{DF} of the first compound DF can satisfy the requirement defined in Equation (3). In this case, holes and electrons are injected into the EML, the luminous area is distributed uniformly in the EML, and therefore, the luminous lifespan of the luminous materials and lifespan of the OLED D3 can be improved greatly.

Also, an energy level bandgap (|HOMO^{H}−HOMO^{DF}|) between the HOMO energy levels (HOMO^{H1}, HOMO^{H2} and HOMO^{H3}) of the third, fourth and sixth compounds H1, H2 and H3 and the HOMO energy level (HOMO^{DF}) of the first compound DF, or an energy level bandgap (|LUMO^{H}−LUMO^{DF}|) between the LUMO energy levels (LUMO^{H1}, LUMO^{H2} and LUMO^{H3}) of the third, fourth and sixth compounds H1, H2 and H3 and the LUMO energy level (LUMO^{DF}) of the first compound DF can be equal to or less than about 0.5 eV.

The singlet and triplet energy levels among the luminous materials should be properly adjusted in order to implement efficient luminescence. Referring to FIG. 12, each of the singlet energy levels S₁^{H1}, S₁^{H2} and S₁^{H3} of the third, fourth and sixth compounds H1, H2 and H3 of the first to third hosts is higher than the singlet energy level S₁^{DF} of the first compound DF having the delayed fluorescent property. Also, each of the triplet energy levels T₁^{H1}, T₁^{H2} and T₁^{H3} of the third, fourth and sixth compounds H1, H2 and H3 can be higher than the triplet energy level T₁^{DF} of the first compound DF.

In addition, the singlet exciton energy, which is generated at the first compound DF having the delayed fluorescent property that is converted to ICT complex by RISC in the EML1 242, should be efficiently transferred to each of second and fifth compounds FD1 and FD2 of the fluorescent material in the EML2 244 and the EML3 246. To this end, the singlet energy level S₁^{DF} of the first compound DF of the delayed fluorescent material in the EML1 242 is higher than each of the singlet energy levels S₁^{FD1} and S₁^{FD2} of the second and fifth compounds FD1 and FD2 of the fluorescent material in the EML2 244 and the EML3 246. Optionally, the triplet energy level T₁^{DF} of the first compound DF in the EML1 242 can be higher than each of the triplet energy levels T₁^{FD1} and T₁^{FD2} of the second and fifth compounds FD1 and FD2 in the EML2 244 and the EML3 246.

In addition, exciton energy transferred to each of the second and fifth compounds FD1 and FD2 from the first compound DF should not be transferred to each of the fourth and sixth compounds H2 and H3 in order to realize efficient luminescence. To this end, each of the singlet energy levels S₁^{H2} and S₁^{H3} of the fourth and sixth compounds H2 and H3, each of which can be the second host and the third host, is higher than each of the singlet energy levels S₁^{FD1} and S₁^{FD2} of the third and sixth compounds FD1 and FD2 of the fluorescent material, respectively. Optionally, each of the triplet energy levels T₁^{H2} and T₁^{H3} of the fifth and seventh compounds H2 and H3 is higher than each of the triplet energy levels T₁^{FD1} and T₁^{FD2} of the third and sixth compounds FD1 and FD2, respectively.

Each of the contents of the first and second compounds DF1 and DF2 in the EML1 442 can be larger than each of the contents of the third and sixth compounds FD1 and FD2 in the EML2 444 or the EML3 446. In this case, exciton energy can be transferred sufficiently from the first and second compounds DF1 and DF2 in the EML1 442 to each of the third and sixth compounds FD1 and FD2 in the EML2 444 and the EML3 446 via FRET mechanism. As an example, the EML1 442 can include each of the first and second compounds DF1 and DF2 between about 10 wt. % and about 40 wt. %. Each of the EML2 444 and the EML3 446 can include the third and sixth compound FD1 and FD2 between about 1 wt. % and about 10 wt. %, for example, about 1 wt. % and 5 wt. %.

In one exemplary aspect, when the EML2 244 is disposed adjacently to the EBL 265, the fourth compound H2 in the EML2244 can be the same material as the EBL 265. In this case, the EML2244 can have an electron blocking function as well as an emission function. In other words, the EML2244 can act as a buffer layer for blocking electrons. In one aspect, the EBL 265 can be omitted where the EML2244 can be an electron blocking layer as well as an emitting material layer.

When the EML3 246 is disposed adjacently to the HBL 275, the sixth compound H3 in the EML3 246 can be the same material as the HBL 275. In this case, the EML3 246 can have a hole blocking function as well as an emission function. In other words, the EML3 246 can act as a buffer layer for blocking holes. In one aspect, the HBL 275 can be omitted where the EML3 246 can be a hole blocking layer as well as an emitting material layer.

In still another exemplary aspect, the fourth compound H2 in the EML2244 can be the same material as the EBL 265 and the sixth compound H3 in the EML3 246 can be the same material as the HBL 275. In this aspect, the EML2244 can have an electron blocking function as well as an emission function, and the EML3 246 can have a hole blocking function as well as an emission function. In other words, each of the EML2244 and the EML3 246 can act as a buffer layer for blocking electrons or hole, respectively. In one aspect, the EBL 265 and the HBL 275 can be omitted where the EML2244 can be an electron blocking layer as well as an emitting material layer and the EML3 246 can be a hole blocking layer as well as an emitting material layer.

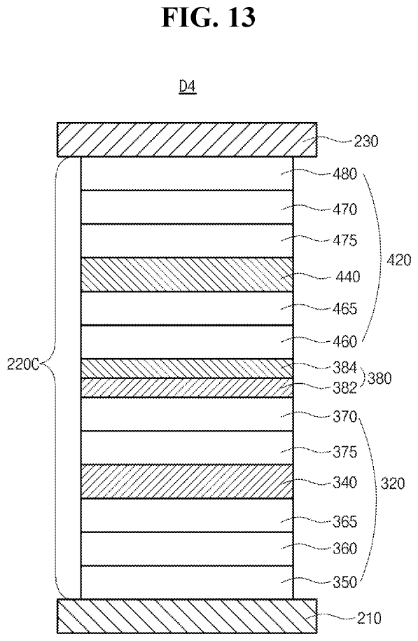

In an alternative aspect, an OLED can include multiple emitting parts. FIG. 13 is a schematic cross-sectional view illustrating an OLED in accordance with still another exemplary aspect of the present disclosure.

As illustrated in FIG. 13, the OLED D4 includes first and second electrodes 210 and 230 facing each other and an emissive layer 220C with two emitting parts disposed between the first and second electrodes 210 and 230. The organic light emitting display device 100 (FIG. 1) includes a red pixel region, a green pixel region and a blue pixel region, and the OLED D4 can be disposed in the green pixel region. The first electrode 210 can be an anode and the second electrode 230 can be a cathode.

The emissive layer 220C includes a first emitting part 320 that includes a first EML (EML1) 340 and a second emitting part 420 that includes a second EML (EML2) 440. Also, the emissive layer 220C can further include a charge generation layer (CGL) 380 disposed between the first emitting part 320 and the second emitting part 420.

The CGL 380 is disposed between the first and second emitting parts 320 and 420 so that the first emitting part 320, the CGL 380 and the second emitting part 420 are sequentially disposed on the first electrode 210. In other words, the first emitting part 320 is disposed between the first electrode 210 and the CGL 380 and the second emitting part 420 is disposed between the second electrode 230 and the CGL 380.

The first emitting part 320 includes the EML1 340. The first emitting part 320 can further includes at least one of an HIL 350 disposed between the first electrode 210 and the EML1 340, a first HTL (HTL1) 360 disposed between the HIL 350 and the EML1340 and a first ETL (ETL1) 370 disposed between the EML1 340 and the CGL 380. Alternatively, the first emitting part 320 can further include a first EBL (EBL1) 365 disposed between the HTL1 360 and the EML1 340 and/or a first HBL (HBL1) 375 disposed between the EML1 340 and the ETL1 370.

The second emitting part 420 includes the EML2 440. The second emitting part 420 can further include at least one of a second HTL (HTL2) 460 disposed between the CGL 380 and the EML2 440, a second ETL (ETL2) 470 disposed between the EML2 440 and the second electrode 230 and an EIL 480 disposed between the ETL2 470 and the second electrode 230. Alternatively, the second emitting part 420 can further include a second EBL (EBL2) 465 disposed between the HTL2 460 and the EML2 440 and/or a second HBL (HBL2) 475 disposed between the EML2 440 and the ETL2 470.

The CGL 380 is disposed between the first emitting part 320 and the second emitting part 420. The first emitting part 320 and the second emitting part 420 are connected via the CGL 380. The CGL 380 can be a PN-junction CGL that junctions an N-type CGL (N-CGL) 382 with a P-type CGL (P-CGL) 384.

The N-CGL 382 is disposed between the ETL1 370 and the HTL2 460 and the P-CGL 384 is disposed between the N-CGL 382 and the HTL2 460. The N-CGL 382 transports electrons to the EML1 340 of the first emitting part 320 and the P-CGL 384 transport holes to the EML2 440 of the second emitting part 420.

In this aspect, each of the EML1 340 and the EML2 440 can be a green emitting material layer. For example, at least one of the EML1 340 and the EML2 440 can include the first compound DF of the delayed fluorescent material, the second compound FD of the fluorescent material, and optionally the third compound H of the host.

As an example, when the EML1 340 and/or the EML2 440 includes the first to third compounds, the contents of the third compound H can be larger than or equal to the contents of the first compound DF, and the contents of the first compound DF can be larger than the contents of the second compound FD. In this case, exciton energy can be transferred efficiently from the first compound DF to the second compound FD.

In one exemplary aspect, the EML2 440 can include the first and second compounds DF and FD, and optionally the third compound H as the same as the EML1 340. Alternatively, the EML2 440 can include another compound for example different from at least one of the first compound DF and the second compound FD in the EML1 340, and thus the EML2 440 can emit light different from the light emitted from the EML1 340 or can have different luminous efficiency different from the luminous efficiency of the EML1 340.

In FIG. 13, each of the EML1 340 and the EML2 440 has a single-layered structure. Alternatively, each of the EML1 340 and the EML2 440, each of which can include the first to third compounds, can have a double-layered structure (FIG. 7) or a triple-layered structure (FIG. 10), respectively.

The LUMO energy level LUMO^{DF} and/or the HOMO energy level HOMO^{DF} of the first compound DF and the LUMO energy level LUMO^{FD} and/or the HOMO energy level HOMO^{FD} of the second compound FD are properly regulated as indicated in the above aspects (FIGS. 5, 8 and 11). The emission area in the EML1 340 and EML2 440 are distributed uniformly, and therefore, the luminous lifespan of the luminous materials and lifetime of the OLED D4 can be improved.

In the OLED D4, the singlet exciton energy of the first compound DF of the delayed fluorescent material is transferred to the second compound FD of fluorescent material, and the ultimate emission is occurred at the second compound FD. Accordingly, the OLED D4 can improve its color purity and luminous lifespan. Moreover, since the OLED D4 has a double stack structure of a green emitting material layer, the OLE4 D4 can further improve its color sense or optimize its luminous efficiency.

FIG. 14 is a schematic cross-sectional view illustrating an organic light emitting display device in accordance with another exemplary aspect of the present disclosure. As illustrated in FIG. 14, an organic light emitting display device 500 includes a substrate 510 that defines first to third pixel regions P1, P2 and P3, a thin film transistor Tr disposed over the substrate 510 and an OLED D disposed over the thin film transistor Tr and connected to the thin film transistor Tr. As an example, the first pixel region P1 can be a green pixel region, the second pixel region P2 can be a red pixel region and the third pixel region P3 can be a blue pixel region.

The substrate 510 can be a glass substrate or a flexible substrate. For example, the flexible substrate can be any one of a P1 substrate, a PES substrate, a PEN substrate, a PET substrate and a PC substrate. A buffer layer 512 is disposed over the substrate 510 and the thin film transistor Tr is disposed over the buffer layer 512. The buffer layer 512 can be omitted. As illustrated in FIG. 2, the thin film transistor Tr includes a semiconductor layer, a gate electrode, a source electrode and a drain electrode and acts as a driving element.

A passivation layer 550 is disposed over the thin film transistor Tr. The passivation layer 550 has a flat top surface and includes a drain contact hole 552 that exposes a drain electrode of the thin film transistor Tr.

The OLED D is disposed over the passivation layer 550, and includes a first electrode 610 that is connected to the drain electrode of the thin film transistor Tr, and an emissive layer 620 and a second electrode 630 each of which is disposed sequentially on the first electrode 610. The OLED D is disposed in each of the first to third pixel regions P1, P2 and P3 and emits different light in each pixel region. For example, the OLED D in the first pixel region P1 can emit green light, the OLED D in the second pixel region P2 can emit red light and the OLED D in the third pixel region P3 can emit blue light.

The first electrode 610 is separately formed for each of the first to third pixel regions P1, P2 and P3, and the second electrode 630 corresponds to the first to third pixel regions P1, P2 and P3 and is formed integrally.

The first electrode 610 can be one of an anode and a cathode, and the second electrode 630 can be the other of the anode and the cathode. In addition, one of the first electrode 610 and the second electrode 630 can be a transmissive (or semi-transmissive) electrode and the other of the first electrode 610 and the second electrode 630 can be a reflective electrode.

For example, the first electrode 610 can be an anode and can include conductive material having a relatively high work function value, i.e., a transparent conductive oxide layer of transparent conductive oxide (TCO). The second electrode 630 can be a cathode and can include conductive material having relatively low work function value, i.e., a metal material layer of low-resistant metal. For example, the first electrode 610 can include any one of ITO, IZO, ITZO, SnO, ZnO, ICO and AZO, and the second electrode 630 can include Al, Mg, Ca, Ag, alloy thereof (e.g. Mg—Ag) or combination thereof.

When the organic light emitting display device 500 is a bottom-emission type, the first electrode 610 can have a single-layered structure of a transparent conductive oxide layer. Alternatively, when the organic light emitting display device 500 is a top-emission type, a reflective electrode or a reflective layer can be disposed under the first electrode 610. For example, the reflective electrode or the reflective layer can include, but is not limited to, Ag or APC alloy. In the OLED D of the top-emission type, the first electrode 610 can have a triple-layered structure of ITO/Ag/ITO or ITO/APC/ITO. Also, the second electrode 630 is thin so as to have light-transmissive (or semi-transmissive) property.

A bank layer 560 is disposed on the passivation layer 550 in order to cover edges of the first electrode 610. The bank layer 560 corresponds to each of the first to third pixel regions P1, P2 and P3 and exposes a center of the first electrode 610.

An emissive layer 620 is disposed on the first electrode 610. In one exemplary aspect, the emissive layer 620 can have a single-layered structure of an EML. Alternatively, the emissive layer 620 can include at least one of an HIL, an HTL, and an EBL disposed sequentially between the first electrode 610 and the EML and/or an HBL, an ETL and an EIL disposed sequentially between the EML and the second electrode 630.

In one exemplary aspect, the EML of the emissive layer 630 in the first pixel region P1 of the green pixel region can include the first compound DF of the delayed fluorescent material having the structure of Formulae 1 to 4, the second compound FD of the fluorescent material having the structure of Formula 5 to 7, and optionally the third compound H of the host having the structure of Formulae 8 to 10.

An encapsulation film 570 is disposed over the second electrode 630 in order to prevent outer moisture from penetrating into the OLED D. The encapsulation film 570 can have, but is not limited to, a triple-layered structure of a first inorganic insulating film, an organic insulating film and a second inorganic insulating film.

The organic light emitting display device 500 can have a polarizer in order to decrease external light reflection. For example, the polarizer can be a circular polarizer. When the organic light emitting display device 500 is a bottom-emission type, the polarizer can be disposed under the substrate 510. Alternatively, when the organic light emitting display device 500 is a top emission type, the polarizer can be disposed over the encapsulation film 570.

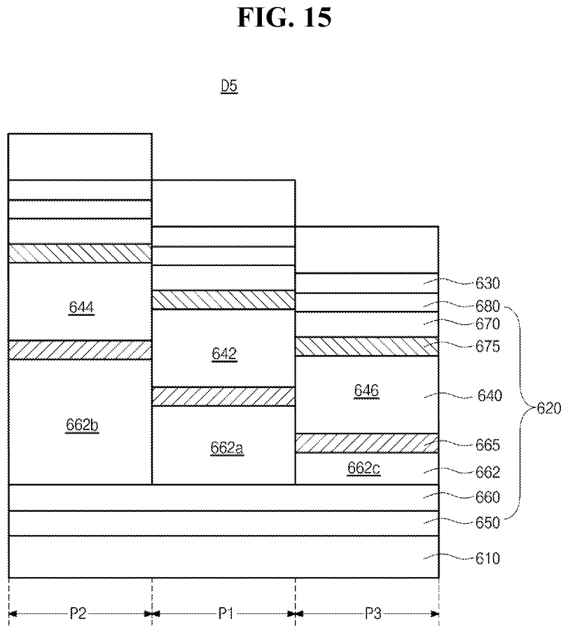

FIG. 15 is a schematic cross-sectional view illustrating an OLED in accordance with still another exemplary aspect of the present disclosure. As illustrated in FIG. 15, the OLED D5 includes a first electrode 610, a second electrode 630 facing the first electrode 610 and an emissive layer 620 disposed between the first and second electrodes 610 and 630.

The first electrode 610 can be an anode and the second electrode 630 can be a cathode. As an example, the first electrode 610 can be a reflective electrode and the second electrode 630 can be a transmissive (or semi-transmissive) electrode.

The emissive layer 620 includes an EML 640. The emissive layer 620 can include at least one of an HTL 660 disposed between the first electrode 610 and the EML 640 and an ETL 670 disposed between the EML 640 and the second electrode 630. Also, the emissive layer 620 can further include at least one of an HIL 650 disposed between the first electrode 610 and the HTL 660 and an EIL 680 disposed between the ETL 670 and the second electrode 630. In addition, the emissive layer 620 can further include at least one of an EBL 665 disposed between the HTL 660 and the EML 640 and an HBL 675 disposed between the EML 640 and the ETL 670.

In addition, the emissive layer 620 can further include an auxiliary hole transport layer (auxiliary HTL) 662 disposed between the HTL 660 and the EBL 665. The auxiliary HTL 662 can include a first auxiliary HTL 662a located in the first pixel region P1, a second auxiliary HTL 662b located in the second pixel region P2 and a third auxiliary HTL 662c located in the third pixel region P3.

The first auxiliary HTL 662a has a first thickness, the second auxiliary HTL 662b has a second thickness and the third auxiliary HTL 662c has a third thickness. The first thickness is less than the second thickness and is more than the third thickness. Accordingly, the OLED D5 has a micro-cavity structure.

Owing to the first to third auxiliary HTLs 662a, 662b and 662c having different thickness to each other, the distance between the first electrode 610 and the second electrode 630 in the first pixel region P1 emitting light in the first wavelength range (greenlight) is smaller than the distance between the first electrode 610 and the second electrode 630 in the second pixel region P2 emitting light in the second wavelength range (red light), which is longer than the first wave length range, but is longer than the distance between the first electrode 610 and the second electrode 630 in the third pixel region P3 emitting light in the third wavelength range (blue light), which is shorter than the first wave length range. Accordingly, the OLED D5 has improved luminous efficiency.

In FIG. 15, the third auxiliary HTL 662c is located in the third pixel region P3. Alternatively, the OLED D5 can implement the micro-cavity structure without the third auxiliary HTL 662c. In addition, a capping layer can be disposed over the second electrode 630 in order to improve out-coupling of the light emitted from the OLED D5.

The EML 640 includes a first EML (EML1) 642 located in the first pixel region P1, a second EML (EML2) 644 located in the second pixel region P2 and a third EML (EML3) 646 located in the third pixel region P3. Each of the EML1 642, the EML2 644 and the EML3 646 can be a green EML, a red EML and a blue EML, respectively.

In one exemplary aspect, the EML1 642 located in the first pixel region P1 can include the first compound of the delayed fluorescent material having the structure of Formulae 1 to 4, the second compound FD of the fluorescent material having the structure of Formulae 5 to 7, and optionally the third compound H of the host having the structure of Formulae 8 to 10. The EML1 642 can have a single-layered structure, a double-layered structure (FIG. 7) or a triple-layered structure (FIG. 10).

When the EML1 642 includes the first to third compounds DF, FD and H, the contents of the third compound H can be larger than or equal to the contents of the first compound DF, and the contents of the first compound DF can be larger than the contents of the second compound FD. In this case, exciton energy can be transferred efficiently from the first compound DF to the second compound FD.

The EML2 644 located in the second pixel region P2 can include a host and a red dopant and the EML3 646 located in the third pixel region P3 can include a host and a blue dopant. For example, the red dopant in the EML2 644 can include at least one of red phosphorescent material, red fluorescent material and red delayed fluorescent material. The blue dopant in the EML3 646 can include at least one of blue phosphorescent material, blue fluorescent material and blue delayed fluorescent material.

As an example, the host in the EML1 644 can include, but is not limited to, 9,9′-Diphenyl-9H,9′H-3,3′-bicarbazole (BCzPh), CBP, 1,3,5-Tris(carbazole-9-yl)benzene (TCP), TCTA, 4,4′-Bis(carbazole-9-yl)-2,2′-dimethylbipheyl (CDBP), 2,7-Bis(carbazole-9-yl)-9,9-dimethylfluorene (DMFL-CBP), 2,2′,7,7′-Tetrakis(carbazole-9-yl)-9,9-spiorofluorene (spiro-CBP), DPEPO, 4′-(9H-carbazol-9-yl)biphenyl-3,5-dicarbonitrile (PCzB-2CN), 3′-(9H-carbazol-9-yl)biphenyl-3,5-dicarbonitrile (mCzB-2CN), 3,6-Bis(carbazole-9-yl)-9-(2-ethyl-hexyl)-9H-carbazole (TCz1), Bis[2-(2-hydroxyphenyl)-pyridine] beryllium (Bepp₂), Bis(10-hydroxylbenzo[h]quinolinato)beryllium (Bebq₂) [symbol] 1,3,5-Tris(1-pyrenyl)benzene (TPB3) and combination thereof.

The red dopant in the EML2 644 can include, but is not limited to, red phosphorescent dopant and/or red fluorescent dopant such as [Bis(2-(4,6-dimethyl)phenylquinoline)](2,2,6,6-tetramethylheptane-3,5-dionate)iridium(III), Bis[2-(4-n-hexylphenyl)quinoline](acetylacetonate)iridium(III) (Hex-Ir(phq)₂(acac)), Tris[2-(4-n-hexylphenyl)quinoline]iridium(III) (Hex-Ir(phq)₃), Tris[2-phenyl-4-methylquinoline]iridium(III) (Ir(Mphq)₃), Bis(2-phenylquinolinex2,2,6,6-tetramethylheptene-3,5-dionate)iridium(III)(Ir(dpm)PQ₂), Bis(phenylisoquinoline)(2,2,6,6-tetramethylheptene-3,5-dionate)iridium(III) (Ir(dpm)(piq)₂), Bis[(4-n-hexylphenyl)isoquinoline](acetylacetonate)iridium(III) (Hex-Ir(piq)₂(acac)), Tris[2-(4-n-hexylphenyl)quinoline]iridium(III) (Hex-Ir(piq)₃), Tris(2-(3-methylphenyl)-7-methyl-quinolato)iridium (Ir(dmpq)₃), Bis[2-(2-methylphenyl)-7-methyl-quinoline](acetylacetonate)iridium(III) (Ir(dmpq)₂(acac)), Bis[2-(3,5-dimethylphenyl)-4-methyl-quinoline](acetylacetonate)iridium(III) (Ir(mphmq)₂(acac)), Tris(dibenzoylmethane)mono(1,10-phenanthroline)europium(III) (Eu(dbm)₃(phen) and combination thereof.

The host in the EML3 646 can include, but is not limited to, mCP, 9-(3-(9H-carbazol-8-yl)phenyl)-9H-carbazole-3-carbonitrile (mCP-CN), mCBP, CBP-CN, 9-(3-(9H-Carbazol-9-yl)phenyl)-3-(diphenylphosphoryl)-9H-carbazole (mCPPO1) 3,5-Di(9H-carbazol-9-yl)biphenyl (Ph-mCP), TSPO1, 9-(3′-(9H-carbazol-9-yl)-[1,1′-biphenyl]-3-yl)-9H-pyrido[2,3-b]indole (CzBPCb), Bis(2-methylphenyl)diphenylsilane (UGH-1), 1,4-Bis(triphenylsilyl)benzene (UGH-2), 1,3-Bis(triphenylsilyl)benzene (UGH-3), 9,9-Spiorobifluoren-2-yl-diphenyl-phosphine oxide (SPPO1), 9,9′-(5-(Triphenylsilyl)-1,3-phenylene)bis(9H-carbazole) (SimCP) and combination thereof.

The blue dopant in the EML3 646 can include, but is not limited to, blue phosphorescent dopant and/or blue fluorescent dopant such as perylene, 4,4′-Bis[4-(di-p-tolylamino)styryl]biphenyl (DPAVBi), 4-(Di-p-tolylamino)-4-4′-[(di-p-tolylamino)styryl]stilbene (DPAVB), 4,4′-Bis[4-(diphenylamino)styryl]biphenyl (BDAVBi), 2,7-Bis(4-diphenylamino)styryl)-9,9-spiorfluorene (spiro-DPVBi), 11,4-bis[2-[4-[N,N-di(p-tolyl)amino]phenyl]vinyl] benzene (DSB), 1-4-di-[4-(N,N-diphenyl)amino]styryl-benzene (DSA), 2,5,8,11-Tetra-tetr-butylperylene (TBPe), Bepp₂, 9-(9-Phenylcarbazole-3-yl)-10-(naphthalene-1-yl)anthracene (PCAN), mer-Tris(1-phenyl-3-methylimidazolin-2-ylidene-C,C(2)′iridium(III) (mer-Ir(pmi)₃), fac-Tris(1,3-diphenyl-benzimidazolin-2-ylidene-C,C(2)′iridium(III) (fac-Ir(dpbic)₃), Bis(3,4,5-trifluoro-2-(2-pyridyl)phenyl-(2-carboxypyridyl)iridium(III) (Ir(tfpd)₂pic), tris(2-(4,6-difluorophenyl)pyridine))iridium(III) (Ir(Fppy)₃), Bis[2-(4,6-difluorophenyl)pyridinato-C²,N](picolinato)iridium(III) (FIrpic) and combination thereof.

The OLED D5 emits green light, red light and blue light in each of the first to third pixel regions P1, P2 and P3 so that the organic light emitting display device 500 (FIG. 14) can implement a full-color image.

The organic light emitting display device 500 can further include a color filter layer corresponding to the first to third pixel regions P1, P2 and P3 for improving color purity of the light emitted from the OLED D. As an example, the color filter layer can comprise a first color filter layer (green color filter layer) corresponding to the first pixel region P1, the second color filter layer (red color filter layer) corresponding to the second pixel region P2 and the third color filter layer (blue color filter layer) corresponding to the third pixel region P3.

When the organic light emitting display device 500 is a bottom-emission type, the color filter layer can be disposed between the OLED D and the substrate 510. Alternatively, when the organic light emitting display device 500 is a top-emission type, the color filter layer can be disposed over the OLED D.

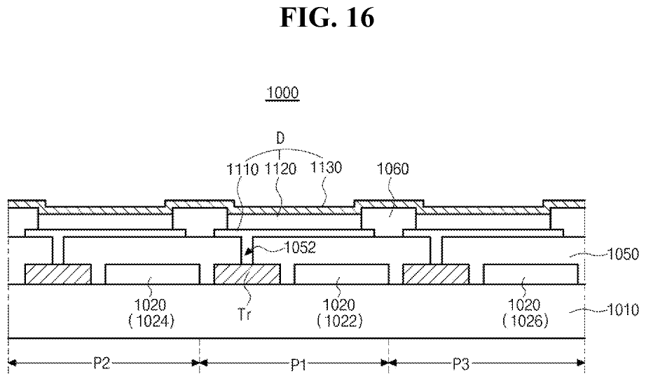

FIG. 16 is a schematic cross-sectional view illustrating an organic light emitting display device in accordance with still another exemplary aspect of the present disclosure. As illustrated in FIG. 16, the organic light emitting display device 1000 includes a substrate 1010 defining a first pixel region P1, a second pixel region P2 and a third pixel region P3, a thin film transistor Tr disposed over the substrate 1010, an OLED D disposed over the thin film transistor Tr and connected to the thin film transistor Tr and a color filter layer 1020 corresponding to the first to third pixel regions P1, P2 and P3. As an example, the first pixel region P1 can be a green pixel region, the second pixel region P2 can be a red pixel region and the third pixel region P3 can be a blue pixel region.

The substrate 1010 can be a glass substrate or a flexible substrate. For example, the flexible substrate can be any one of a PI substrate, a PES substrate, a PEN substrate, a PET substrate and a PC substrate. The thin film transistor Tr is located over the substrate 1010. Alternatively, a buffer layer can be disposed over the substrate 1010 and the thin film transistor Tr can be disposed over the buffer layer. As illustrated in FIG. 2, the thin film transistor Tr includes a semiconductor layer, a gate electrode, a source electrode and a drain electrode and acts as a driving element.

The color filter layer 1020 is located over the substrate 1010. As an example, the color filter layer 1020 can include a first color filter pattern 1022 corresponding to the first pixel region P1, a second color filter pattern 1024 corresponding to the second pixel region P2 and a third color filter pattern 1026 corresponding to the third pixel region P3. The first color filter pattern 1022 can be a green color filter pattern, the second color filter pattern 1024 can be a red color filter pattern and the third color filter pattern 1026 can be a blue color filter pattern. For example, the first color filter pattern 1022 can include at least one of green dye or green pigment, the second color filter pattern 1024 can include at least one of red dye or red pigment and the third color filter pattern 1026 can include at least one of blue dye or blue pigment.

A passivation layer 1050 is disposed over the thin film transistor Tr and the color filter layer 1020. The passivation layer 1050 has a flat top surface and includes a drain contact hole 1052 that exposes a drain electrode of the thin film transistor Tr.

The OLED D is disposed over the passivation layer 1050 and corresponds to the color filter layer 1020. The OLED D includes a first electrode 1110 that is connected to the drain electrode of the thin film transistor Tr, and an emissive layer 1120 and a second electrode 1130 each of which is disposed sequentially on the first electrode 1110. The OLED D emits white light in the first to third pixel regions P1, P2 and P3.

The first electrode 1110 is separately formed for each of the first to third pixel regions P1, P2 and P3, and the second electrode 1130 corresponds to the first to third pixel regions P1, P2 and P3 and is formed integrally. The first electrode 1110 can be one of an anode and a cathode, and the second electrode 1130 can be the other of the anode and the cathode. In addition, the first electrode 1110 can be a transmissive (or semi-transmissive) electrode and the second electrode 1130 can be a reflective electrode.

For example, the first electrode 1110 can be an anode and can include conductive material having a relatively high work function value, i.e., a transparent conductive oxide layer of transparent conductive oxide (TCO). The second electrode 1130 can be a cathode and can include conductive material having relatively low work function value, i.e., a metal material layer of low-resistant metal. For example, the transparent conductive oxide layer of the first electrode 1110 can include any one of ITO, IZO, ITZO, SnO, ZnO, ICO and AZO, and the second electrode 1130 can include Al, Mg, Ca, Ag, alloy thereof (e.g., Mg—Ag) or combination thereof.

The emissive layer 1120 is disposed on the first electrode 1110. The emissive layer 1120 includes at least two emitting parts emitting different colors. Each of the emitting part can have a single-layered structure of an EML. Alternatively, each of the emitting parts can include at least one of an HIL, an HTL, an EBL, an HBL, an ETL and an EIL. In addition, the emissive layer 1120 can further comprise a CGL disposed between the emitting parts.

At least one of the at least two emitting parts can include the first compound DF of the delayed fluorescent material having the structure of Formulae 1 to 4, the second compound FD of the boron-based fluorescent material having the structure of Formulae 5 to 7, and optionally the third compound H of the host having the structure of Formulae 8 to 10.

A bank layer 1060 is disposed on passivation layer 1050 in order to cover edges of the first electrode 1110. The bank layer 1060 corresponds to each of the first to third pixel regions P1, P2 and P3 and exposes a center of the first electrode 1110. As described above, since the OLED D emits white light in the first to third pixel regions P1, P2 and P3, the emissive layer 1120 can be formed as a common layer without being separated in the first to third pixel regions P1, P2 and P3. The bank layer 1060 is formed to prevent current leakage from the edges of the first electrode 1110, and the bank layer 1060 can be omitted.

Moreover, the organic light emitting display device 1000 can further include an encapsulation film disposed on the second electrode 1130 in order to prevent outer moisture from penetrating into the OLED D. In addition, the organic light emitting display device 1000 can further comprise a polarizer disposed under the substrate 1010 in order to decrease external light reflection.

In the organic light emitting display device 1000 in FIG. 16, the first electrode 1110 is a transmissive electrode, the second electrode 1130 is a reflective electrode, and the color filter layer 1020 is disposed between the substrate 1010 and the OLED D. That is, the organic light emitting display device 1000 is a bottom-emission type. Alternatively, the first electrode 1110 can be a reflective electrode, the second electrode 1120 can be a transmissive electrode (or semi-transmissive electrode) and the color filter layer 1020 can be disposed over the OLED D in the organic light emitting display device 1000.

In the organic light emitting display device 1000, the OLED D located in the first to third pixel regions P1, P2 and P3 emits white light, and the white light passes through each of the first to third pixel regions P1, P2 and P3 so that each of a green color, a red color and a blue color is displayed in the first to third pixel regions P1, P2 and P3, respectively.

A color conversion film can be disposed between the OLED D and the color filter layer 1020. The color conversion film corresponds to the first to third pixel regions P1, P2 and P3, and includes a green color conversion film, a red color conversion film and a blue color conversion film each of which can convert the white light emitted from the OLED D into green light, red light and blue light, respectively. For example, the color conversion film can include quantum dots. Accordingly, the organic light emitting display device 1000 can further enhance its color purity. Alternatively, the color conversion film can displace the color filter layer 1020.

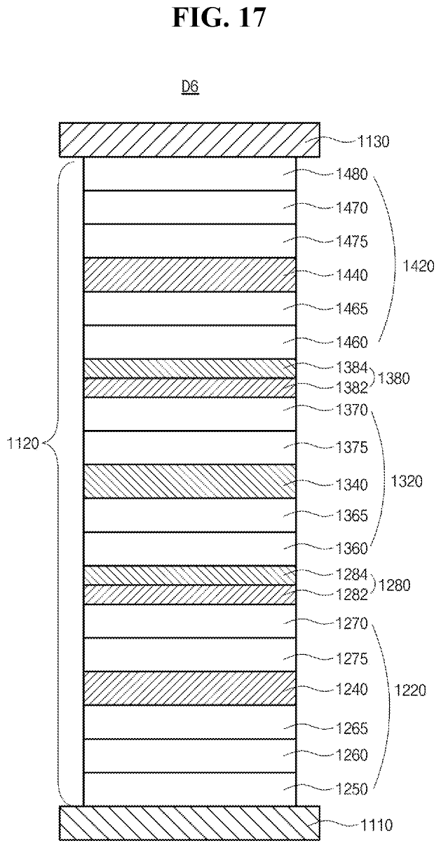

FIG. 17 is a schematic cross-sectional view illustrating an OLED in accordance with still another exemplary aspect of the present disclosure. As illustrated in FIG. 17, the OLED D6 includes first and second electrodes 1110 and 1130 facing each other and an emissive layer 1120 disposed between the first and second electrodes 1110 and 1130. The first electrode 1110 can be an anode and the second electrode 1130 can be a cathode. For example, the first electrode 1100 can be a transmissive electrode and the second electrode 1130 can be a reflective electrode.

The emissive layer 1120 includes a first emitting part 1220 including a first EML (lower EML, EML1) 1240, a second emitting part 1320 comprising a second EML (middle EML, EML2) 1340 and a third emitting part 1420 comprising a third EML (upper EML, EML3) 1440. In addition, the emissive layer 1120 can further includes a first charge generation layer (CGL1) 1280 disposed between the first emitting part 1220 and the second emitting part 1320 and a second charge generation layer (CGL2) 1380 disposed between the second emitting part 1320 and the third emitting part 1420. Accordingly, the first emitting part 1220, the CGL1 1280, the second emitting part 1320, the CGL2 1380 and the third emitting part 1420 are disposed sequentially on the first electrode 1110.

The first emitting part 1220 can further include at least one of an HIL 1250 disposed between the first electrode 1110 and the EML1 1240, a first HTL (HTL1) 1260 disposed between the EML1 1240 and the HIL 1250 and a first ETL (ETL1) 1270 disposed between the EML1 1240 and the CGL1 1280. Alternatively, the first emitting part 1220 can further include at least one of a first EBL (EBL1) 1265 disposed between the HTL1 1260 and the EML1 1240 and a first HBL (HBL1) 1275 disposed between the EML1 1240 and the ETL1 1270.

The second emitting part 1320 can further include at least one of a second HTL (HTL2) 1360 disposed between the CGL1 1280 and the EML2 1340, a second ETL (ETL2) 1370 disposed between the EML2 1340 and the CGL2 1380. Alternatively, the second emitting part 1320 can further include a second EBL (EBL2) 1365 disposed between the HTL2 1360 and the EML2 1340 and/or a second HBL (HBL2) 1375 disposed between the EML2 1340 and the ETL2 1370.

The third emitting part 1420 can further include at least one of a third HTL (HTL3) 1460 disposed between the CGL2 1380 and the EML3 1440, a third ETL (ETL3) 1470 disposed between the EML3 1440 and the second electrode 1130 and an EIL 1480 disposed between the ETL3 1470 and the second electrode 1130. Alternatively, the third emitting part 1420 can further comprise a third EBL (EBL3) 1465 disposed between the HTL3 1460 and the EML3 1440 and/or a third HBL (HBL3) 1475 disposed between the EML3 1440 and the ETL3 1470.

The CGL1 1280 is disposed between the first emitting part 1220 and the second emitting part 1320. For example, the first emitting part 1220 and the second emitting part 1320 are connected via the CGL1 1280. The CGL1 1280 can be a PN-junction CGL that junctions a first N-type CGL (N-CGL1) 1282 with a first P-type CGL (P-CGL1) 1284.

The N-CGL1 1282 is disposed between the ETL1 1270 and the HTL2 1360 and the P-CGL1 1284 is disposed between the N-CGL1 1282 and the HTL2 1360. The N-CGL1 1282 transports electrons to the EML1 1240 of the first emitting part 1220 and the P-CGL1 1284 transport holes to the EML2 1340 of the second emitting part 1320.

The CGL2 1380 is disposed between the second emitting part 1320 and the third emitting part 1420. For example, the second emitting part 1320 and the third emitting part 1420 are connected via the CGL2 1380. The CGL2 1380 can be a PN-junction CGL that junctions a second N-type CGL (N-CGL2) 1382 with a second P-type CGL (P-CGL2) 1384.

The N-CGL2 1382 is disposed between the ETL2 1370 and the HTL3 1460 and the P-CGL2 1384 is disposed between the N-CGL2 1382 and the HTL3 1460. The N-CGL2 1382 transports electrons to the EML2 1340 of the second emitting part 1320 and the P-CGL2 1384 transport holes to the EML3 1440 of the third emitting part 1420.

In this aspect, one of the first to third EMLs 1240, 1340 and 1440 can be a blue EML, another of the first to third EMLs 1240, 1340 and 1440 can be a green EML and the third of the first to third EMLs 1240, 1340 and 1440 can be a red EML.

As an example, the EML1 1240 can be a blue EML, the EML2 1340 can be a green EML and the EML3 1440 can be a red EML. Alternatively, the EML1 1240 can be a red EML, the EML2 1340 can be a green EML and the EML3 1440 can be a blue EML1.

The EML1 1240 can include a host and a blue dopant (or red dopant), the EML3 1440 can include a host and a red dopant (or blue dopant). As an example, the host in each of the EML1 1240 and the EML3 1440 can include the blue or red host as described above, the dopant in each of the EML1 1240 and the EML3 1440 can include at least one of blue or red phosphorescent material, blue or red fluorescent material and blue or red delayed fluorescent material as described above.

The EML2 1340 can include the first compound DF of the delayed fluorescent material having the structure of Formulae 1 to 4, the second compound FD of the fluorescent material having the structure of Formulae 5 to 7, and optionally the third compound H of the host having the structure of Formulae 8 to 10. The EML2 1340 can have a single-layered structure, a double-layered structure (FIG. 7) or a triple-layered structure (FIG. 10).

In the EML2 1340, the contents of the third compound H can be larger than or equal to the contents of the first compound DF, and the contents of the first compound DF is larger than the contents of the second compound FD. In this case, exciton energy can be transferred efficiently from the first compound DF to the second compound FD.

The OLED D6 emits white light in each of the first to third pixel regions P1, P2 and P3 and the white light passes though the color filter layer 1020 (FIG. 16) correspondingly disposed in the first to third pixel regions P1, P2 and P3. Accordingly, the organic light emitting display device 1000 (FIG. 16) can implement a full-color image.

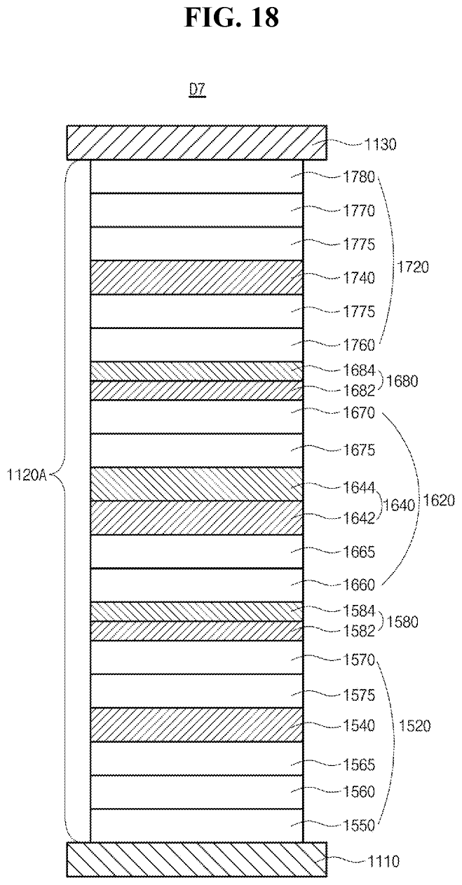

FIG. 18 is a schematic cross-sectional view illustrating an OLED in accordance with still another exemplary aspect of the present disclosure. As illustrated in FIG. 18, the OLED D7 includes first and second electrodes 1110 and 1130 facing each other and an emissive layer 1120A disposed between the first and second electrodes 1110 and 1130. The first electrode 1110 can be an anode and the second electrode 1130 can be a cathode. For example, the first electrode 1100 can be a transmissive electrode and the second electrode 1130 can be a reflective electrode.

The emissive layer 1120A includes a first emitting part 1520 comprising an EML1 (lower EML) 1540, a second emitting part 1620 comprising an EML2 (middle EML) 1640 and a third emitting part 1720 comprising an EML3 (upper EML) 1740. In addition, the emissive layer 1120A can further include a CGL1 1580 disposed between the first emitting part 1520 and the second emitting part 1620 and a CGL2 1680 disposed between the second emitting part 1620 and the third emitting part 1720. Accordingly, the first emitting part 1520, the CGL1 1580, the second emitting part 1620, the CGL2 1680 and the third emitting part 1720 are disposed sequentially on the first electrode 1110.

The first emitting part 1520 can further include at least one of an HIL 1550 disposed between the first electrode 1110 and the EML1 1540, an HTL1 1560 disposed between the EML1 1540 and the HIL 1550 and an ETL1 1570 disposed between the EML1 1540 and the CGL1 1580. Alternatively, the first emitting part 1520 can further comprise an EBL1 1565 disposed between the HTL1 1560 and the EML1 1540 and/or an HBL1 1575 disposed between the EML1 1540 and the ETL1 1570.

The EML2 1640 of the second emitting part 1620 includes a middle lower EML (first layer) 1642 and a middle upper EML (second layer) 1644. The middle lower EML 1642 is located adjacently to the first electrode 1110 and the upper middle EML 1644 is located adjacently to the second electrode 1130. In addition, the second emitting part 1620 can further include at least one of an HTL2 1660 disposed between the CGL1 1580 and the EML2 1640, an ETL2 1670 disposed between the EML2 1640 and the CGL2 1680. Alternatively, the second emitting part 1620 can further comprise at least one of an EBL2 1665 disposed between the HTL2 1660 and the EML2 1640 and an HBL2 1675 disposed between the EML2 1640 and the ETL2 1670.

The third emitting part 1720 can further include at least one of an HTL3 1760 disposed between the CGL2 1680 and the EML3 1740, an ETL3 1770 disposed between the EML3 1740 and the second electrode 1130 and an EIL 1780 disposed between the ETL3 1770 and the second electrode 1130. Alternatively, the third emitting part 1720 can further include an EBL3 1765 disposed between the HTL3 1760 and the EML3 1740 and/or an HBL3 1775 disposed between the EML3 1740 and the ETL3 1770.

The CGL1 1580 is disposed between the first emitting part 1520 and the second emitting part 1620. For example, the first emitting part 1520 and the second emitting part 1620 are connected via the CGL1 1580. The CGL1 1580 can be a PN-junction CGL that junctions an N-CGL1 1582 with a P-CGL1 1584. The N-CGL1 1582 is disposed between the ETL1 1570 and the HTL2 1660 and the P-CGL1 1584 is disposed between the N-CGL1 1582 and the HTL2 1560.

The CGL2 1680 is disposed between the second emitting part 1620 and the third emitting part 1720. For example, the second emitting part 1620 and the third emitting part 1720 are connected via the CGL2 1680. The CGL2 1680 can be a PN-junction CGL that junctions an N-CGL2 1682 with a P-CGL2 1684. The N-CGL2 1682 is disposed between the ETL2 1570 and the HTL3 1760 and the P-CGL2 1684 is disposed between the N-CGL2 1682 and the HTL3 1760.

In this aspect, each of the EML1 1540 and the EML3 1740 can be a blue EML. In an exemplary aspect, each of the EML1 1540 and the EML3 1740 can include a host and a blue dopant. The host in each of the EML1 1540 and the EML3 1740 can include the blue host as described above, and the blue dopant in each of the EML1 1540 and the EMl3 1740 can include at least one of the blue phosphorescent material, the blue fluorescent material and the blue delayed fluorescent material as described above. The host and/or the blue dopant in the EML1 1540 can be identical to or different from the host and/or the blue dopant in the EML3 1740. As an example, the blue dopant in the EML1 1540 can have different luminous efficiency and/or emission peak different from the luminous efficiency and/or emission peak of the blue dopant in the EML3 1740.

One of the middle lower EML 1642 and the middle upper EML 1644 of the EML2 1640 can be a green EML and the other of the middle lower EML 1642 and the middle upper EML 1644 of the EML2 1640 can be a red EML. The green EML and the red EML are sequentially disposed to form the EML2 1640.

As an example, the middle lower EML 1642 of the green EML can include the first compound of DF of the delayed fluorescent material having the structure of Formulae 1 to 4, the second compound FD of the fluorescent material having the structure of Formulae 5 to 7, and optionally the third compound H of the host having the structure of Formulae 8 to 10. The middle lower EML 1642 can have a single-layered structure, a double-layered structure (FIG. 7) or a triple-layered structure (FIG. 10).

The middle upper EML 1644 of the red EML can include a host and a red dopant. The host in the middle upper EML 1644 can include the red host as described above, and the red dopant in the middle upper EML 1644 can include at least one of the red phosphorescent materials, the red fluorescent materials and the red delayed fluorescent materials as described above.

As an example, when the middle lower EML 1644 include the first compound DF, the second compound FD and the third compound H, the contents of the third compound H can be larger than or equal to the contents of the first compound DF and the contents of the first compound DF can be larger than the contents of the second compound FD.

The OLED D7 emits white light in each of the first to third pixel regions P1, P2 and P3 and the white light passes though the color filter layer 1020 (FIG. 16) correspondingly disposed in the first to third pixel regions P1, P2 and P3. Accordingly, the organic light emitting display device 1000 (FIG. 16) can implement a full-color image.

In FIG. 18, the OLED D7 has a three-stack structure including the first to third emitting parts 1520, 1620 and 1720 which includes the EML1 1540 and the EML3 1740 as a blue EML. Alternatively, the OLED D7 can have a two-stack structure where one of the first emitting part 1520 and the third emitting part 1720 each of which includes the EML1 1540 and the EML3 1740 as a blue EML is omitted.

### Example 1 (Ex. 1): Fabrication of OLED

An OLED in which an EML includes Compound 1-3 of Formula 4 (LUMO: −3.1 eV, HOMO: −5.7 eV) as the first compound DF and Compound 3-1 of Formula 10 (mCBP, LUMO: −2.5 eV, HOMO: −6.0 eV) was fabricated. The ITO substrate was washed by UV-Ozone treatment before using, and was transferred to a vacuum chamber for depositing emission layer. Subsequently, an anode, an emission layer and a cathode were deposited by evaporation from a heating boat under 10⁻⁷ torr vacuum condition with setting deposition rate of 1 Å/s in the following order:

An anode (ITO, 50 nm); an HIL (HAT-CN, 7 nm); an HTL (NPB, 45 nm); an EBL (TAPC, 10 nm), an EML (mCBP (65 wt %), Compound 1-3 (35 wt. %), 40 nm); an HBL (B3PYMPM, 10 nm); an ETL (TPBi, 25 nm), an EIL (LiF); and a cathode (Al).

The charge injection or transport materials used in the HIL, HTL, EBL, HBL and ETL are indicated below.

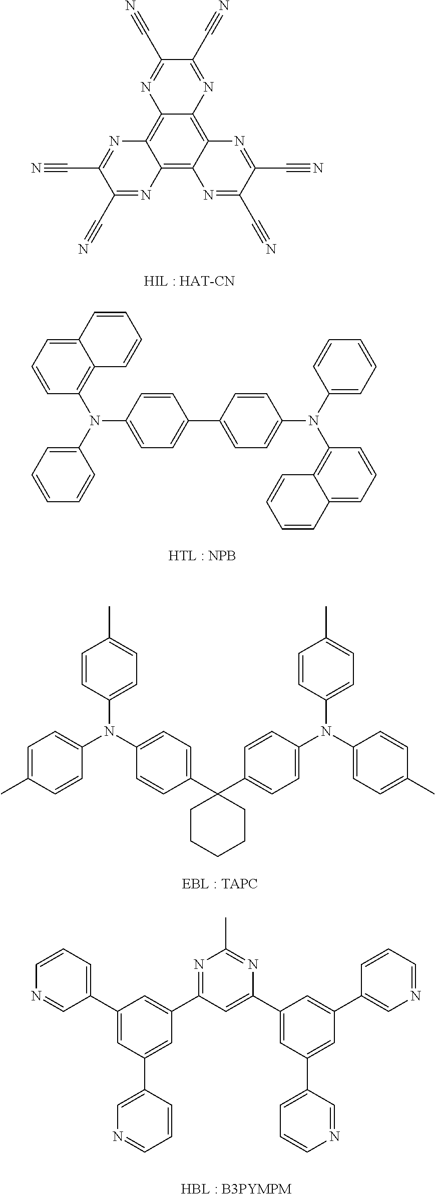

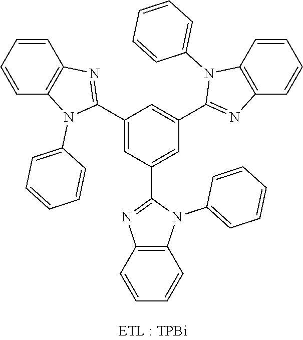 [SMILES: c1ccc(-n2c(-c3cc(-c4nc5ccccc5n4-c4ccccc4)cc(-c4nc5ccccc5n4-c4ccccc4)c3)nc3ccccc32)cc1]

### Example 2-8 (Ex. 2-8): Fabrication of OLED

An OLED was fabricated using the same materials as Example 1, except that each of Compound 1-4 of Formula 4 (LUMO: −3.1 eV, HOMO: −5.7 eV, Ex. 2), Compound 1-9 of Formula 4 (LUMO: −3.2 eV, HOMO: −5.8 eV, Ex. 3), Compound 1-10 of Formula 4 (LUMO: −3.2 eV, HOMO: −5.8 eV, Ex. 4), Compound 1-14 of Formula 4 (LUMO: −3.1 eV, HOMO: −5.7 eV, Ex. 5), Compound 1-15 of Formula 4 (LUMO: −3.1 eV, HOMO: −5.7 eV, Ex. 6), Compound 1-108 of Formula 4 (LUMO: −3.4 eV, HOMO: −5.8 eV, Ex. 7) and Compound 1-114 of Formula 4 (LUMO: −3.4 eV, HOMO: −5.7 eV, Ex. 8) as the first compound instead of the Compound 1-3 was used in the EML.

### Comparative Example 1-7 (Ref. 1-7: Fabrication of OLED

An OLED was fabricated using the same materials as Example 1, except that each of the following Com. 1 (LUMO: −2.8 eV, HOMO: −5.8 eV, Ref. 1), Com. 2 (LUMO: −2.9 eV, HOMO: −5.8 eV, Ref. 2), Com. 3 (LUMO: −3.0 eV, HOMO: −5.8 eV, Ref. 3), Com. 4 (LUMO: −2.9 eV, HOMO: −5.7 eV, Ref. 4), Com. 5 (LUMO: −2.9 eV, HOMO: −5.8 eV, Ref. 5), Com. 6 (LUMO: −2.8 eV, HOMO: −5.8 eV, Ref. 6) and Com. 6 (LUMO: −2.8 eV, HOMO: −5.8 eV, Ref. 7) as the first compound instead of the Compound 1-3 was used in the EML.

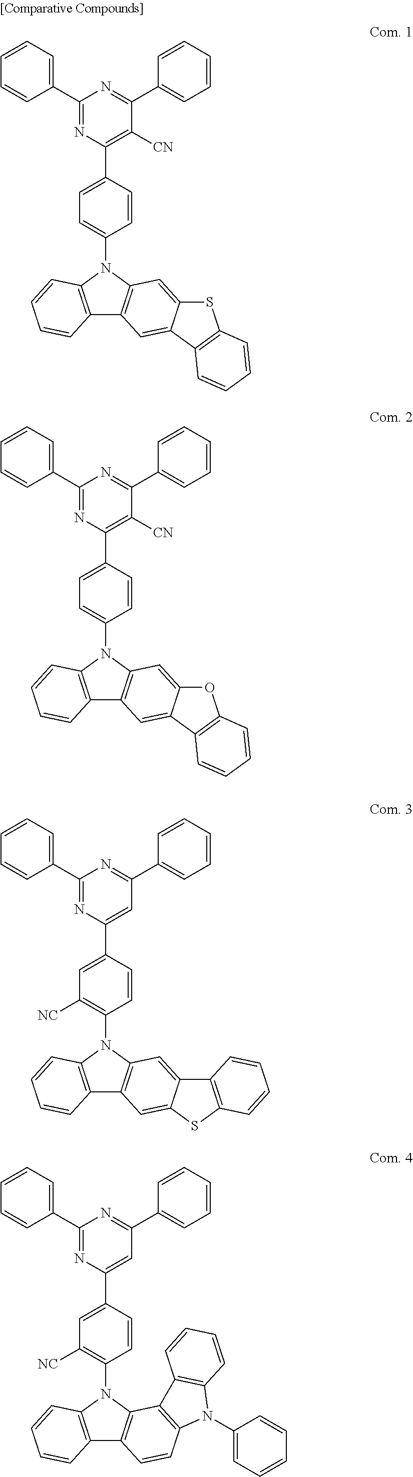 [SMILES: N#Cc1c(-c2ccccc2)nc(-c2ccccc2)nc1-c1ccc(-n2c3ccccc3c3cc4c(cc32)sc2ccccc24)cc1 ; N#Cc1c(-c2ccccc2)nc(-c2ccccc2)nc1-c1ccc(-n2c3ccccc3c3cc4c(cc32)oc2ccccc24)cc1 ; N#Cc1cc(-c2cc(-c3ccccc3)nc(-c3ccccc3)n2)ccc1-n1c2ccccc2c2cc3sc4ccccc4c3cc21 ; N#Cc1cc(-c2cc(-c3ccccc3)nc(-c3ccccc3)n2)ccc1-n1c2ccccc2c2ccc3c(c4ccccc4n3-c3ccccc3)c21]

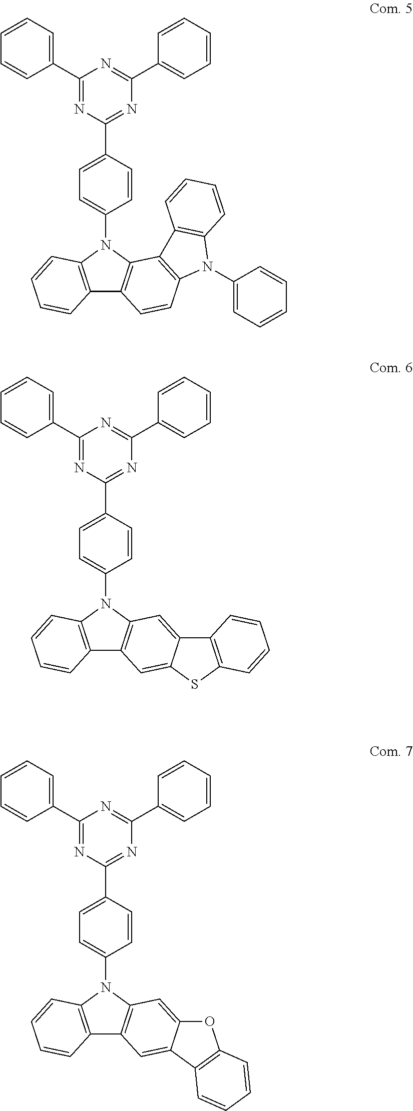 [SMILES: c1ccc(-c2nc(-c3ccccc3)nc(-c3ccc(-n4c5ccccc5c5ccc6c(c7ccccc7n6-c6ccccc6)c54)cc3)n2)cc1 ; c1ccc(-c2nc(-c3ccccc3)nc(-c3ccc(-n4c5ccccc5c5cc6sc7ccccc7c6cc54)cc3)n2)cc1 ; c1ccc(-c2nc(-c3ccccc3)nc(-c3ccc(-n4c5ccccc5c5cc6c(cc54)oc4ccccc46)cc3)n2)cc1]

### Example 9 (Ex. 9): Fabrication of OLED

An OLED was fabricated using the same materials as Example 1, except that mCBP as the third compound, the Compound 1-3 of Formula 4 as the first compound and Compound 2-64 of Formula 7 (LUMO: −3.0 eV, HOMO: −5.3 eV) as the second compound was mixed with a weight ratio of 64:35:1 in the EML.

### Examples 10-16 (Ex. 10-16: Fabrication of OLED

An OLED was fabricated using the same materials as Example 9, except that each of Compound 1-4 of Formula 4 (Ex. 10), Compound 1-9 of Formula 4 (Ex. 11), Compound 1-10 of Formula 4 (Ex. 12), Compound 1-14 of Formula 4 (Ex. 13), Compound 1-15 of Formula 4 (Ex. 14), Compound 1-108 of Formula 4 (Ex. 15) and Compound 1-114 of Formula 4 (Ex. 16) as the first compound instead of the Compound 1-3 was used in the EML.

### Example 17 (Ex. 17): Fabrication of OLED

An OLED was fabricated using the same materials as Example 9, except that Compound 1-4 of Formula 4 as the first compound instead of the Compound 1-3 and Compound 2-85 of Formula 7 (LUMO: −3.0 eV, HOMO: −5.3 eV) as the second compound instead of the Compound 2-64 were used in the EML.

### Example 18 (Ex. 18): Fabrication of OLED

An OLED was fabricated using the same materials as Example 17, except that Compound 1-14 as the first compound instead of the Compound 1-4 was used in the EML. Example 19 (Ex. 19): Fabrication of OLED

An OLED was fabricated using the same materials as Example 9, except that Compound 1-4 of Formula 4 as the first compound instead of the Compound 1-3 and Compound 2-109 of Formula 7 (LUMO: −3.0 eV, HOMO: −5.3 eV) as the second compound instead of the Compound 2-64 were used in the EML.

### Example 20 (Ex. 20): Fabrication of OLED

An OLED was fabricated using the same materials as Example 19, except that Compound 1-14 as the first compound instead of the Compound 1-4 was used in the EML.

### Comparative Examples 8-14 (Ref. 8-14): Fabrication of OLED

An OLED was fabricated using the same materials as Example 9, except that each of the Com. 1 (Ref. 8), Com. 2 (Ref. 9), Com. 3 (Ref. 10), Com. 4 (Ref. 11), Com. 5 (Ref. 12), Com. 6 (Ref. 13) and Com. 6 (Ref. 14 as the first compound instead of the Compound 1-3 was used in the EML.

### Comparative Examples 15-16 (Ref. 15-16): Fabrication of OLED

An OLED was fabricated using the same materials as Example 17, except that each of the Com. 3 (Ref. 15) and Com. 5 (Ref. 16) as the first compound instead of the Compound 1-4 was used in the EML.

### Comparative Examples 17-18 (Ref. 17-18): Fabrication of OLED

An OLED was fabricated using the same materials as Example 19, except that each of the Com. 3 (Ref. 17) and Com. 5 (Ref. 18) as the first compound instead of the Compound 1-4 was used in the EML.

### Experimental Example 1: Measurement of Luminous Properties of OLED

Each of the OLED fabricated in Ex. 1-20 and Ref. 1-18 was connected to an external power source and then luminous properties for all the diodes were evaluated using a constant current source (KEITHLEY) and a photometer PR650 at room temperature. In particular, driving voltage (V), current efficiency (cd/A), external quantum efficiency (EQE, %) and maximum electroluminescence wavelength (λₘₐₓ, nm) at 6.0 mA/cm² current density, and lifespan (LT95, h) at 12.0 mA/cm² current density of the OLEDs were measured. The measurement results for the OLEDs are shown in the following tables 1 and 2:

TABLE 1
Luminous Properties of OLED

| | | | 6.0 | | | | 12.0 |
|---|---|---|---|---|---|---|---|
| | | | mA/cm² | | | | mA/cm² |
| | EML | | | | EQE | | LT₉₅ |
| Sample | Compound 1 | Compound 2 | V | cd/A | (%) | λₘₐₓ | (h) |
| Ref. 1 | Com. 1 | — | 3.8 | 45.0 | 16.1 | 540 | 77 |
| Ref. 2 | Com. 2 | | 3.8 | 45.7 | 16.0 | 548 | 78 |
| Ref. 3 | Com. 3 | | 3.7 | 55.8 | 17.3 | 548 | 84 |
| Ref. 4 | Com. 4 | | 3.8 | 51.1 | 16.0 | 545 | 80 |
| Ref. 5 | Com. 5 | | 3.9 | 55.8 | 17.3 | 548 | 78 |
| Ref. 6 | Com. 6 | | 4.0 | 50.2 | 18.4 | 528 | 62 |
| Ref. 7 | Com. 7 | | 3.9 | 49.2 | 17.9 | 520 | 68 |
| Ex. 1 | 1-3 | — | 3.8 | 56.2 | 17.5 | 542 | 52 |
| Ex. 2 | 1-4 | | 3.7 | 46.7 | 16.2 | 544 | 50 |
| Ex. 3 | 1-9 | | 3.8 | 56.2 | 16.8 | 546 | 75 |
| Ex. 4 | 1-10 | | 3.8 | 54.2 | 17.0 | 544 | 65 |
| Ex. 5 | 1-14 | | 3.7 | 54.8 | 16.8 | 548 | 58 |
| Ex. 6 | 1-15 | | 3.7 | 54.2 | 16.8 | 548 | 70 |
| Ex. 7 | 1-108 | | 3.8 | 38.2 | 10.2 | 580 | 210 |
| Ex. 8 | 1-114 | | 3.9 | 36.4 | 9.8 | 585 | 250 |

TABLE 2
Luminous Properties of OLED

| | | | 6.0 | | | | 12.0 |
|---|---|---|---|---|---|---|---|
| | | | mA/cm² | | | | mA/cm² |
| | EML | | | | EQE | | LT₉₅ |
| Sample | Compound 1 | Compound 2 | V | cd/A | (%) | λₘₐₓ | (h) |
| Ref. 8 | Com. 1 | 2-64 | 3.8 | 44.3 | 14.0 | 558 | 53 |
| Ref. 9 | Com. 2 | | 3.9 | 44.8 | 13.6 | 554 | 60 |
| Ref. 10 | Com. 3 | | 3.8 | 44.6 | 14.3 | 558 | 53 |
| Ref. 11 | Com. 4 | | 3.9 | 43.6 | 13.6 | 558 | 60 |
| Ref. 12 | Com. 5 | | 3.9 | 44.8 | 13.6 | 554 | 55 |
| Ref. 13 | Com. 6 | | 3.8 | 44.5 | 14.2 | 556 | 42 |
| Ref. 14 | Com. 7 | | 3.9 | 45.6 | 14.5 | 558 | 48 |
| Ref. 15 | Com. 3 | 2-85 | 3.8 | 45.6 | 14.8 | 558 | 65 |
| Ref. 16 | Com. 5 | | 3.8 | 44.6 | 14.4 | 558 | 60 |
| Ref. 17 | Com. 3 | 2-109 | 3.8 | 44.6 | 15.2 | 562 | 57 |
| Ref. 18 | Com. 5 | | 3.9 | 44.8 | 14.8 | 564 | 62 |
| Ex. 9 | 1-3 | 2-64 | 3.8 | 47.3 | 14.8 | 554 | 120 |
| Ex. 10 | 1-4 | | 3.7 | 48.2 | 15.0 | 556 | 111 |
| Ex. 11 | 1-9 | | 3.7 | 45.2 | 15.2 | 556 | 130 |
| Ex. 12 | 1-10 | | 3.7 | 47.0 | 15.0 | 554 | 140 |
| Ex. 13 | 1-14 | | 3.7 | 46.8 | 14.8 | 554 | 152 |
| Ex. 14 | 1-15 | | 3.8 | 48.2 | 14.9 | 554 | 142 |
| Ex. 15 | 1-108 | | 3.1 | 20.1 | 7.5 | 640 | 310 |
| Ex. 16 | 1-114 | | 3.0 | 20.4 | 6.2 | 680 | 350 |
| Ex. 17 | 1-4 | 2-85 | 3.8 | 48.8 | 15.8 | 556 | 168 |
| Ex. 18 | 1-14 | | 3.7 | 49.2 | 16.0 | 556 | 170 |
| Ex. 19 | 1-4 | 2-109 | 3.8 | 46.4 | 15.6 | 562 | 172 |
| Ex. 20 | 1-14 | | 3.7 | 46.9 | 16.0 | 562 | 182 |

As indicated in Table 1, compared to the lifespan of the OLEDs fabricated in Ref. 1-7 in which comparative compounds as the sole dopant were introduced into the EML, the lifespan of the OLEDs fabricated in Ex. 1-8 in which organic compound as the sole dopant were introduced into the EML was increased a little bit or similar. However, as indicated in Table 2, compared to lifespan of the OLEDs fabricated in Ref. 8-18 in which the comparative compounds as the first compound and the second compounds as the dopant were introduced into the EML, the lifespan of the OLEDs fabricated in Ex. 9-20 in which the organic compounds as the first compound and the second compound as the dopant were introduced into the EML were greatly increased.

More particular, as indicated in Tables 1 and 2, compared to the OLED fabricated in Refs. 1-7 in which only each of Com. 1 to Com. 7 was introduced into the EML as the sole dopant, the OLED fabricated in Refs. 8-14 in which Com. 1 to Com. 7 as the first compound and Compound 2-64, 2-85 or 2-109 as the second compound (in this case, the LUMO energy level of the first compound is shallower than the LUMO energy level of the second compound) were introduced into the EML as the dopant decreased its luminous lifespan by maximally 36.9% (Ref. 3 vs. Ref. 8). On the other hand, compared to the OLED fabricated in Ex. 1-8 in which only each of the first compounds were introduced into the EML as the sole dopant, the OLED fabricated in Ex. 9-20 in which the organic compounds as the first compounds and Compound 2-64, 2-85 or 2-109 as the second compound (in this case, the LUMO energy level of the first compound is deeper than the LUMO energy level of the second compound) were introduced into the EML as the dopant increased its luminous lifespan by maximally 213.8% (Ex. 5 vs. Ex. 20).

### Example 21 (Ex. 21): Fabrication of OLED

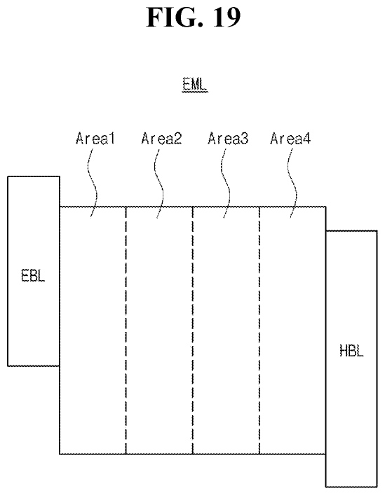

The EML were divided into four areas including area 1, area 2, area 3 and area 4 each of which has a thickness of 10 nm, as illustrated in FIG. 19, in order to certify an emission area in the EML of the OLED in which the first and second compounds are introduced into the EML. In each emission area (EML1 to EML4), mCBP: Compound 1-3: Compound 2-64 with a weight ratio of 64:35:1 were applied to form the EML. An OLED was fabricated using the same material as Example 9, except changing the thickness of the EML as indicated below:

An anode (ITO, 50 nm); an HIL (HAT-CN, 7 nm); an HTL (NPB, 45 nm); an EBL (TAPC, 10 nm), 4 EMLs (mCBP (64 wt. %), Compound 1-3 (35 wt. %), Compound 2-64 (1 wt. %), each area: 10 nm); an HBL (B3PYMPM, 10 nm); an ETL (TPBi, 25 nm), an EIL (LiF); and a cathode (Al).

### Example 22 (Ex. 22): Fabrication of OLED

An OLED was fabricated using the same materials as Example 21, except that the EML1 corresponding to Area 1 illustrated in FIG. 19, which is located closest to the EBL, includes mCBP (63.8 wt. %), the Compound 1-3 (35 wt. %), the Compound 2-64 (1 wt. %) and the following red phosphorescent material Ir(dmpq)₂(acac) (Bis(2-3,5-dimethylphenyl)quinoline-C2,N′)(acetylacetonate)irdium (III) (0.2 wt. %).

### Example 23 (Ref. 23): Fabrication of OLED

An OLED was fabricated using the same materials as Example 21, except that the EML2 corresponding to Area 2 illustrated in FIG. 19 includes mCBP (63.8 wt. %), the Compound 1-3 (35 wt. %), the Compound 2-64 (1 wt. %) and the following red phosphorescent material (0.2 wt. %).

### Example 24 (Ex. 24): Fabrication of OLED

An OLED was fabricated using the same materials as Example 21, except that the EML3 corresponding to Area 3 illustrated in FIG. 19 includes mCBP (63.8 wt. %), the Compound 1-3 (35 wt. %), the Compound 2-64 (1 wt. %) and the following red phosphorescent material (0.2 wt. %).

### Example 25 (Ex. 25): Fabrication of OLED

An OLED was fabricated using the same materials as Example 21, except that the EML4 corresponding to Area 4 illustrated in FIG. 19, which is located closest to the HBL, includes mCBP (63.8 wt. %), the Compound 1-3 (35 wt. %), the Compound 2-64 (1 wt. %) and the following red phosphorescent material (0.2 wt. %).

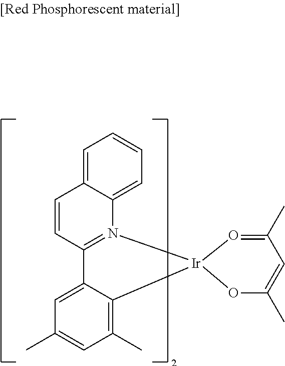

### Comparative Example 19 (Ref. 19): Fabrication of OLED

An OLED was fabricated using the same materials as Example 21, except that the Com. 3 instead of the Compound 1-3 were used in each of emission areas 1-4 each of which has a thickness of 10 nm.

### Comparative Example 20 (Ref. 20): Fabrication of OLED

An OLED was fabricated using the same materials as Ref. 19, except that the EML1 corresponding to Area 1 illustrated in FIG. 19, which is located closest to the EBL, includes mCBP (63.8 wt. %), the Compound 1-3 (35 wt. %), the Compound 2-64 (1 wt. %) and the red phosphorescent material (0.2 wt. %).

### Comparative Example 21 (Ref. 21): Fabrication of OLED

An OLED was fabricated using the same materials as Ref. 19, except that the EML2 corresponding to Area 2 illustrated in FIG. 19 includes mCBP (63.8 wt. %), the Compound 1-3 (35 wt. %), the Compound 2-64 (1 wt. %) and the red phosphorescent material (0.2 wt. %).

### Comparative Example 22 (Ref. 22): Fabrication of OLED

An OLED was fabricated using the same materials as Ref. 19, except that the EML3 corresponding to Area 3 illustrated in FIG. 19 includes mCBP (63.8 wt. %), the Compound 1-3 (35 wt. %), the Compound 2-64 (1 wt. %) and the red phosphorescent material.

### Comparative Example 23 (Ref. 23): Fabrication of OLED

An OLED was fabricated using the same materials as Ref. 19, except that the EML4 corresponding to Area 4 illustrated in FIG. 19, which is located closest to the HBL, includes mCBP (63.8 wt. %), the Compound 1-3 (35 wt. %), the Compound 2-64 (1 wt. %) and the phosphorescent material (0.2 wt. %).

### Experimental Example 2: Measurement of Emission Area in OLED

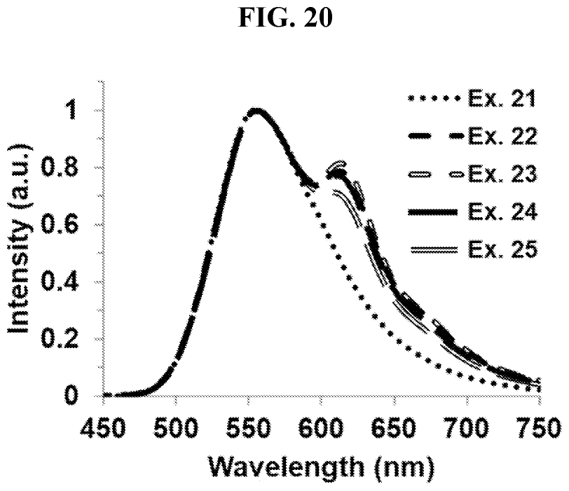

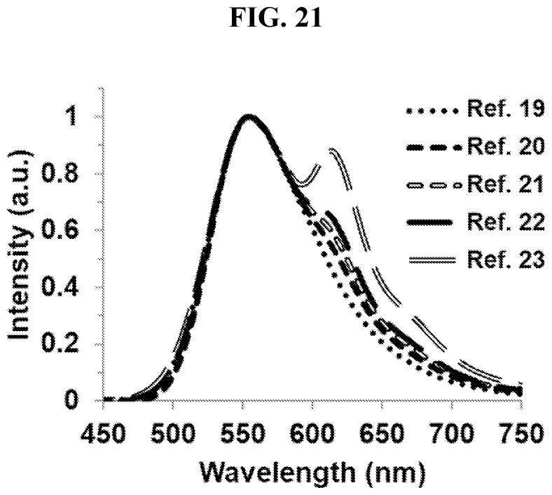

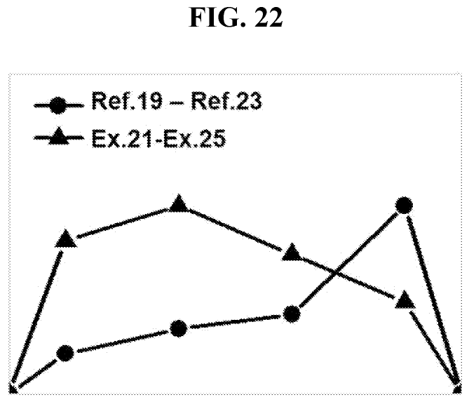

The emission peak of the red phosphorescent material in the EMLs of the OLEDs fabricated in Ex. 21 to Ex. 25 and Ref. 19 to Ref. 23 were measured. FIG. 20 is a graph illustrating measurement results of photo-luminescence (PL) spectra of the red phosphorescent material in the OLEDs fabricated in Ex. 21 to Ex. 25, and FIG. 21 is a graph illustrating measurement results of PL spectra of the red phosphorescent material in the OLEDs fabricated in Ref. 19 to Ref. 23. FIG. 22 is a graph illustrating emission peak intensities in each emission area divided in EML of OLEDs fabricated in Ex. 21 to Ex. 25 and Ref. 19 to Ref. 23.

As illustrated in FIG. 22, the emission areas in the EML are biased to the HBL in the OLEDs fabricated in Ref. 19 to Ref. 23 in which the LUMO energy level of the Com. 1 as the first compound was designed to be shallower than the LUMO energy level of the Compound 2-64 as the second compound. On the other hand, the emission areas in the EML are distributed uniformly in the OLEDs fabricated Ex. 21 to Ex. 25 in which the LUMO energy level of the Compound 1-3 as the first compound was designed to be deeper than the LUMO energy level of the Compound 2-64 as the second compound.

It will be apparent to those skilled in the art that various modifications and variations can be made in the OLED and the organic light emitting device including the OLED of the present disclosure without departing from the technical idea or scope of the disclosure. Thus, it is intended that the present disclosure cover the modifications and variations of this disclosure provided they come within the scope of the appended claims and their equivalents.

What is claimed is:

1. An organic light emitting diode comprising:
a first electrode;
a second electrode facing the first electrode; and
an emissive layer disposed between the first and second electrodes and including at least one emitting material layer,
wherein the at least one emitting material layer includes a first compound and a second compound, and
wherein the first compound has the following structure of Formula 1 and the second compound has the following structure of Formula 5: 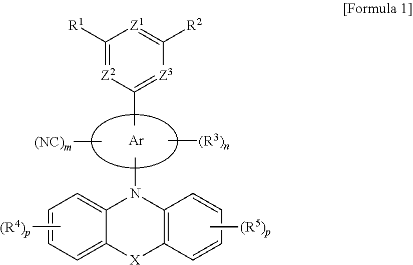
wherein, in Formula 1,
each of R¹ and R² is independently an unsubstituted or substituted C₆-C₃₀ aromatic group or an unsubstituted or substituted C₃-C₃₀ hetero aromatic group;
each of R³ to R⁵ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when n is an integer of 2 or more, each R³ is identical to or different from each other, when p is an integer of 2 or more, each R⁴ is identical to or different from each other, and when q is an integer of 2 or more, each R⁵ is identical to or different from each other;
optionally,
two adjacent elements to which R³ is attached when n is an integer of 2 or more, two adjacent elements to which R⁴ is attached when p is an integer of 2 or more, and/or two adjacent elements to which R⁵ is attached when q is an integer of 2 or more form an unsubstituted or substituted C₆-C₂₀ aromatic ring or an unsubstituted or substituted C₃-C₂₀ hetero aromatic ring;
X is a single bond, CR⁶R⁷, NR⁶, O or S, wherein each of R⁶ and R⁷ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group;
two of Z¹ to Z³ is N and other of Z¹ to Z³ is CR⁸, wherein R⁸ is —CN;
Ar is unsubstituted or substituted C₆-C₃₀ arylene or an unsubstituted or substituted C₃-C₃₀ hetero arylene;
m is an integer of 1 to 4;
n is an integer of 0 to 10; and
each of p and q is independently an integer of 0 to 4, 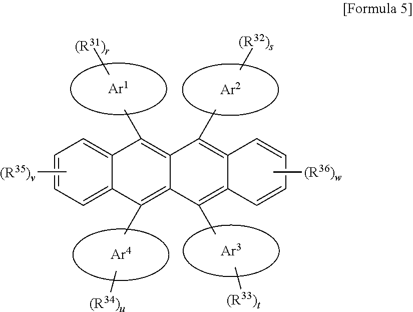
wherein, in Formula 5,
each of R³¹ to R³⁶ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when r is an integer of 2 or more, each R³¹ is identical to or different from each other, when s is an integer of 2 or more, each R³² is identical to or different from each other, when t is an integer of 2 or more, each R³³ is identical to or different from each other, when u is an integer of 2 or more, each R³⁴ is identical to or different from each other, when v is an integer of 2 or more, each R³⁵ is identical to or different from each other, when w is an integer of 2 or more, each R³⁶ is identical to or different from each other;
each of Ar¹ to Ar⁴ is independently an unsubstituted or substituted C₆-C₃₀ aromatic group or an unsubstituted or substituted C₃-C₃₀ hetero aromatic group;
each of r, s, t and u is independently an integer of 0 to 10; and
each of v and w is independently an integer of 0 to 4.

2. The organic light emitting diode of claim 1, wherein a Lowest Unoccupied Molecular Orbital (LUMO) energy level of the first compound and a LUMO energy level of the second compound satisfy the following relationship in Equation (1):
LUMO^{FD}≥LUMO^{DF} (1)
wherein LUMO^{FD} is a LUMO energy level of the second compound and LUMO^{DF} is a LUMO energy level of the first compound.

3. The organic light emitting diode of claim 1, wherein a Highest Occupied Molecular Orbital (HOMO) energy level of the first compound and a HOMO energy level of the second compound satisfy the following relationship in Equation (2):
HOMO^{FD}≥HOMO^{DF} (2)
wherein HOMO^{FD} is a HOMO energy level of the second compound and HOMO^{DF} is a HOMO energy level of the first compound.

4. The organic light emitting diode of claim 1, wherein the first compound has an energy bandgap satisfying the following relationship in Equation (3):
2.0 eV≤Eg^{DF}≤3.0 eV (3)
wherein Eg^{DF} is an energy bandgap between a HOMO energy level and a LUMO energy level of the first compound.

5. The organic light emitting diode of claim 1, wherein the first compound has the following structure of Formula 2: 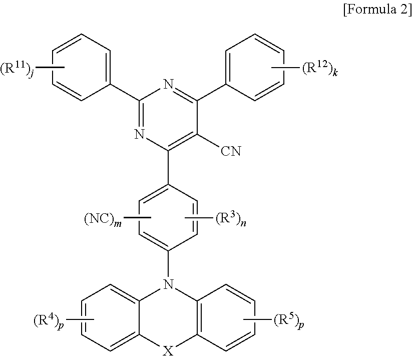
wherein, in Formula 2,
each of R³, R⁴, R⁵, R¹¹ and R¹² is independently hydrogen, protium, deuterium, tritium, unsubstituted or substituted C₁-C₂₀ alkyl, unsubstituted or substituted C₆-C₃₀ aryl or unsubstituted or substituted C₃-C₃₀ hetero aryl, when j is an integer of 2 or more, each R¹¹ is identical to or different from each other, when k is an integer of 2 or more, each R¹² is identical to or different from each other, when n is an integer of 2 or more, each R³ is identical to or different from each other, when p is an integer of 2 or more, each R⁴ is identical to or different from each other, and when q is an integer of 2 or more, each R⁵ is identical to or different from each other;
optionally,
two adjacent elements to which R¹¹ is attached when j is an integer of 2 or more, two adjacent elements to which R¹² is attached when k is an integer of 2 or more, two adjacent elements to which R³ is attached when n is an integer of 2 or more, two adjacent elements to which R⁴ is attached when p is an integer of 2 or more, and/or two adjacent elements to which R⁵ is attached when q is an integer of 2 or more form an unsubstituted or substituted C₆-C₂₀ aromatic ring or an unsubstituted or substituted C₃-C₂₀ hetero aromatic ring;
X is a single bond, CR⁶R⁷, NR⁶, O or S, wherein each of R⁶ and R⁷ is independently hydrogen, protium, deuterium, tritium, unsubstituted or substituted C₁-C₂₀ alkyl, unsubstituted or substituted C₆-C₃₀ aryl or unsubstituted or substituted C₃-C₃₀ hetero aryl;
each of j and k is independently an integer of 0 to 5;
m is an integer of 1 to 4;
n is an integer of 0 to 3; and
each of p and q is independently an integer of 0 to 4.

6. The organic light emitting diode of claim 1, wherein the first compound has the following structure of Formula 3: 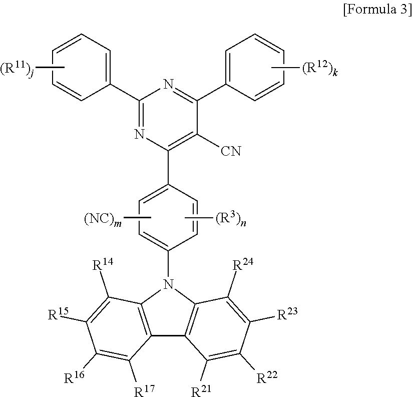
wherein, in Formula 3,
each of R¹¹ to R¹³ is independently hydrogen, protium, deuterium, tritium, unsubstituted or substituted C₁-C₂₀ alkyl, unsubstituted or substituted C₆-C₃₀ aryl or unsubstituted or substituted C₃-C₃₀ hetero aryl, when j is an integer of 2 or more, each R¹¹ is identical to or different from each other, when k is an integer of 2 or more, each R¹² is identical to or different from each other, and when n is an integer of 2 or more, each R³ is identical to or different from each other;
each of R¹⁴ to R¹⁷ is independently hydrogen, protium deuterium, tritium, unsubstituted or substituted C₁-C₂₀ alkyl, unsubstituted or substituted C₆-C₃₀ aryl or unsubstituted or substituted C₃-C₃₀ hetero aryl;
each of R²¹ to R²⁴ is independently protium deuterium, tritium, unsubstituted or substituted C₁-C₂₀ alkyl, unsubstituted or substituted C₆-C₃₀ aryl or unsubstituted or substituted C₃-C₃₀ hetero aryl, or two adjacent groups among R²¹ to R²⁴ form an unsubstituted or substituted C₆-C₂₀ aromatic ring or an unsubstituted or substituted C₃-C₂₀ hetero aromatic ring, wherein at least two adjacent groups among R²¹ to R²⁴ form an unsubstituted or substituted C₆-C₂₀ aromatic ring or an unsubstituted or substituted C₃-C₂₀ hetero aromatic ring;
m is an integer of 1 or 2; and
n is an integer of 0 to 3.

7. The organic light emitting diode of claim 1, wherein the first compound is selected form the following compounds:

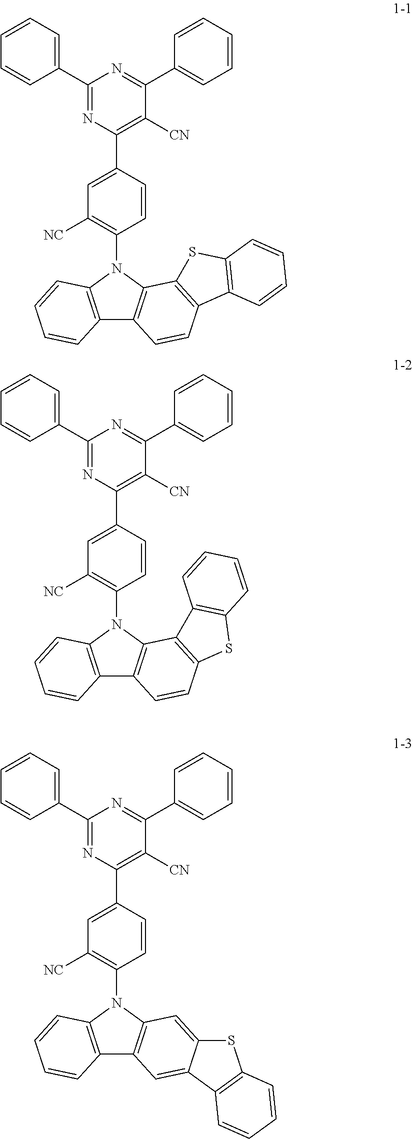

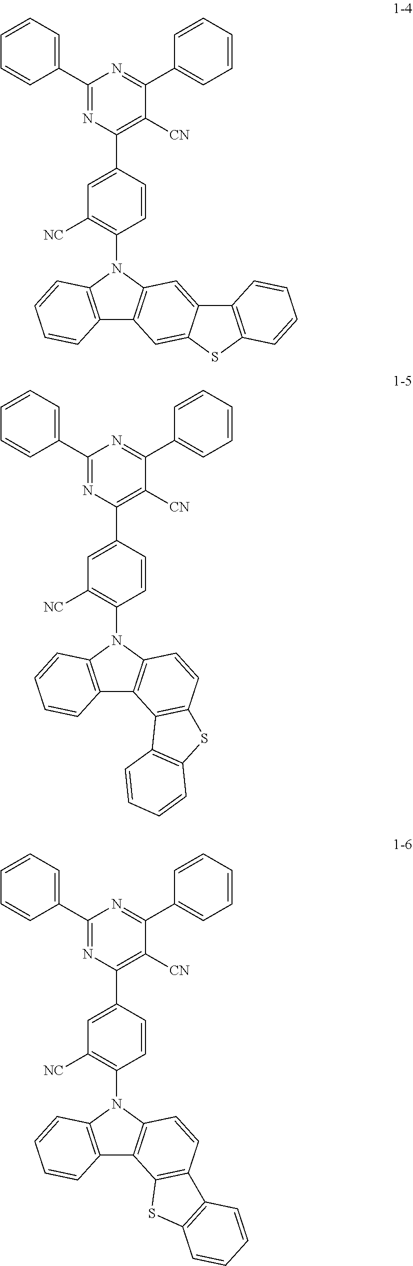

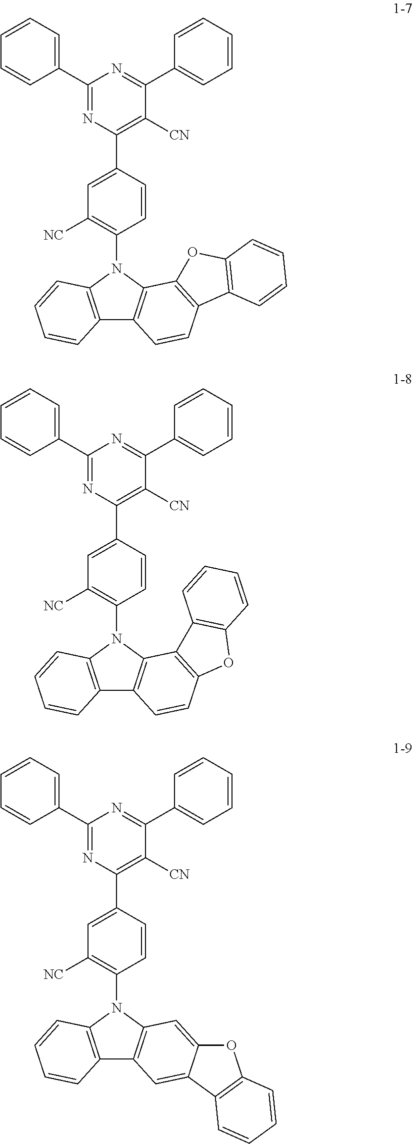 [SMILES: N#Cc1cc(-c2nc(-c3ccccc3)nc(-c3ccccc3)c2C#N)ccc1-n1c2ccccc2c2ccc3c4ccccc4oc3c21 ; N#Cc1cc(-c2nc(-c3ccccc3)nc(-c3ccccc3)c2C#N)ccc1-n1c2ccccc2c2ccc3oc4ccccc4c3c21 ; N#Cc1cc(-c2nc(-c3ccccc3)nc(-c3ccccc3)c2C#N)ccc1-n1c2ccccc2c2cc3c(cc21)oc1ccccc13]

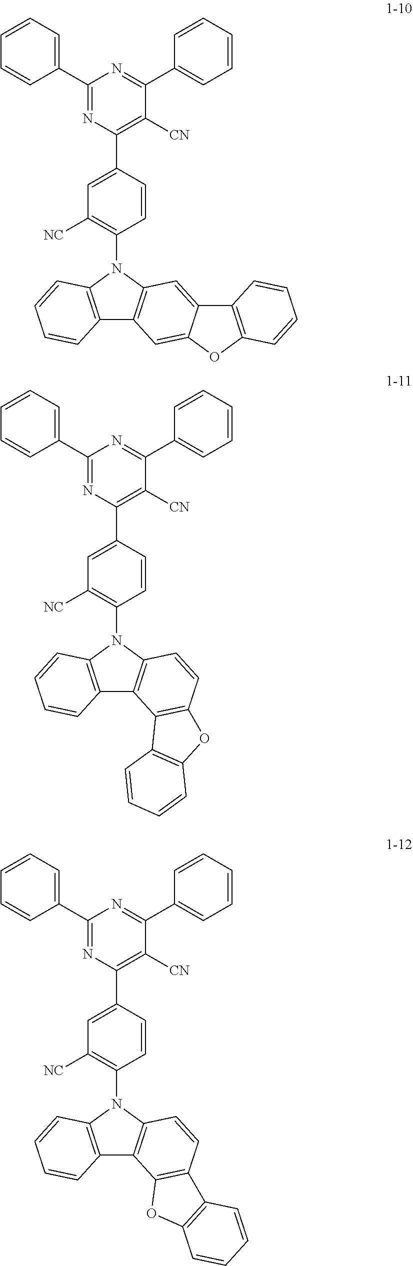

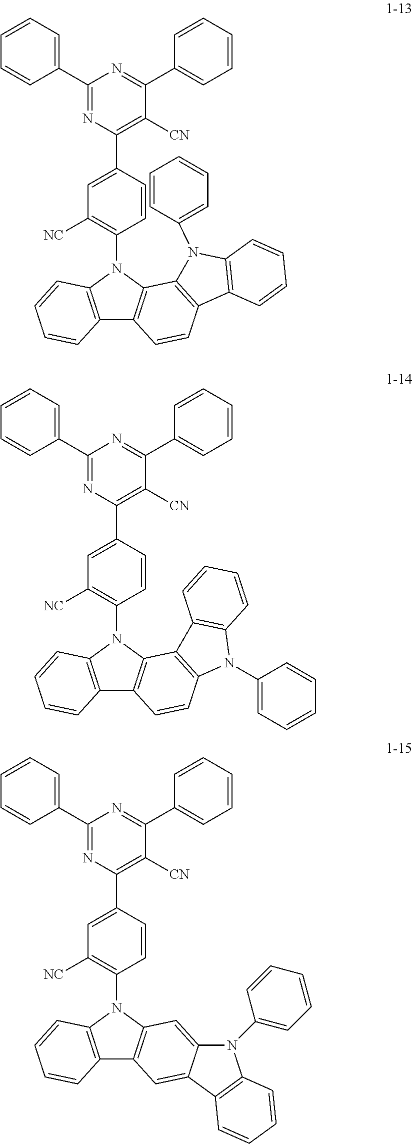 [SMILES: N#Cc1cc(-c2nc(-c3ccccc3)nc(-c3ccccc3)c2C#N)ccc1-n1c2ccccc2c2ccc3c4ccccc4n(-c4ccccc4)c3c21 ; N#Cc1cc(-c2nc(-c3ccccc3)nc(-c3ccccc3)c2C#N)ccc1-n1c2ccccc2c2ccc3c(c4ccccc4n3-c3ccccc3)c21 ; N#Cc1cc(-c2nc(-c3ccccc3)nc(-c3ccccc3)c2C#N)ccc1-n1c2ccccc2c2cc3c4ccccc4n(-c4ccccc4)c3cc21]

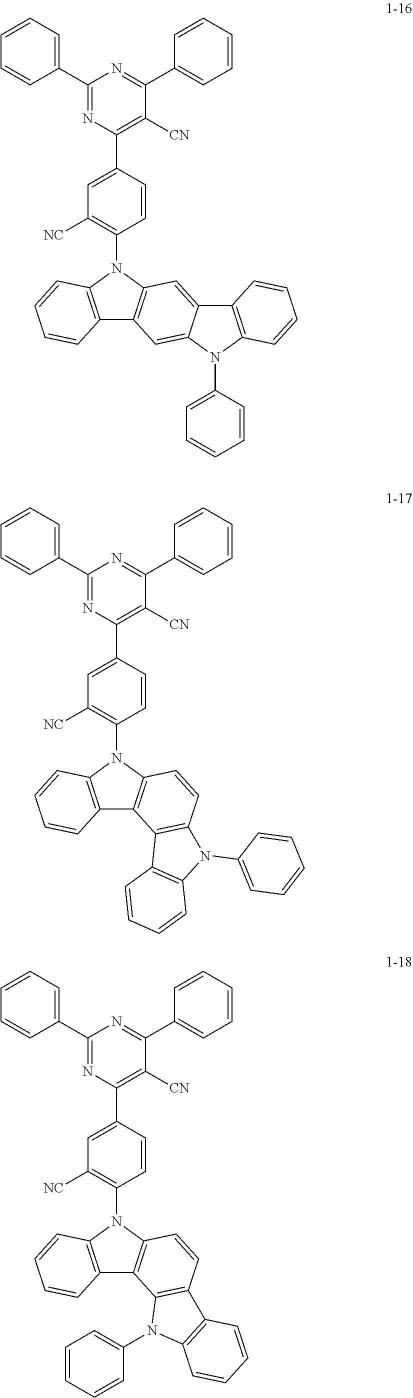 [SMILES: N#Cc1cc(-c2nc(-c3ccccc3)nc(-c3ccccc3)c2C#N)ccc1-n1c2ccccc2c2cc3c(cc21)c1ccccc1n3-c1ccccc1 ; N#Cc1cc(-c2nc(-c3ccccc3)nc(-c3ccccc3)c2C#N)ccc1-n1c2ccccc2c2c3c4ccccc4n(-c4ccccc4)c3ccc21 ; N#Cc1cc(-c2nc(-c3ccccc3)nc(-c3ccccc3)c2C#N)ccc1-n1c2ccccc2c2c1ccc1c3ccccc3n(-c3ccccc3)c12]

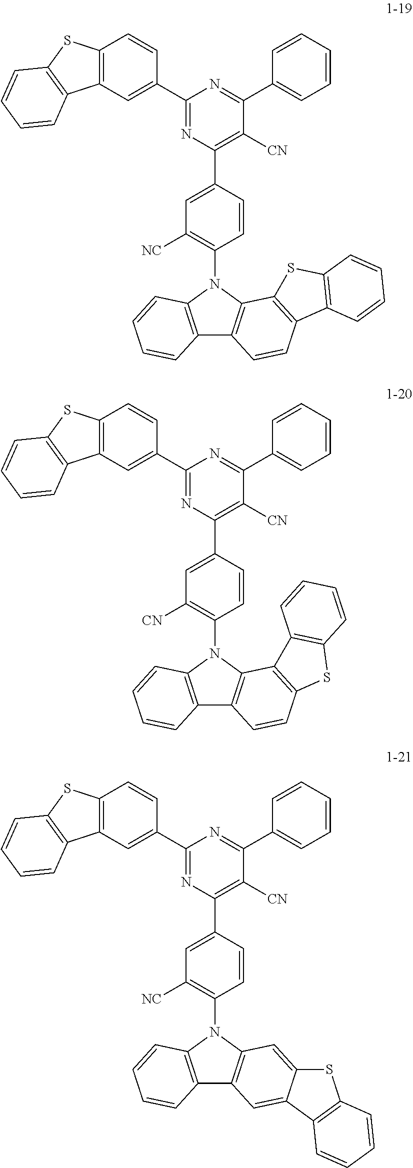

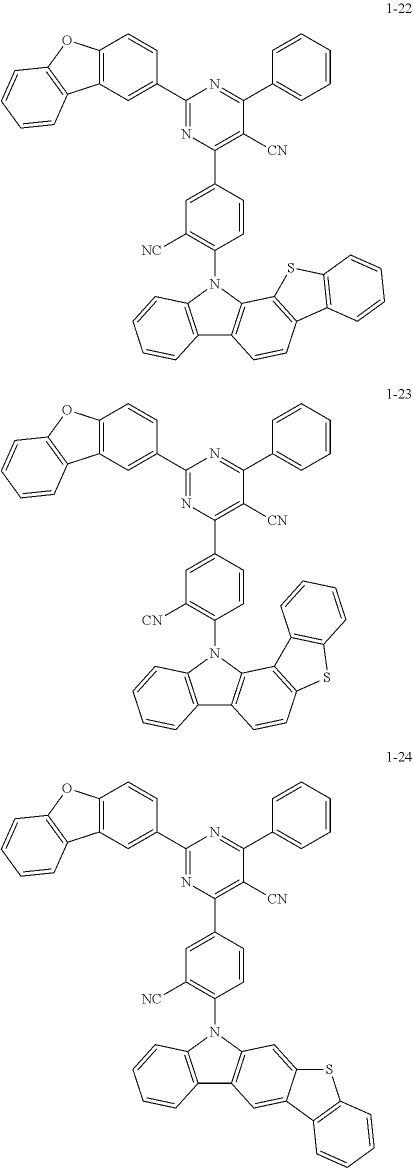

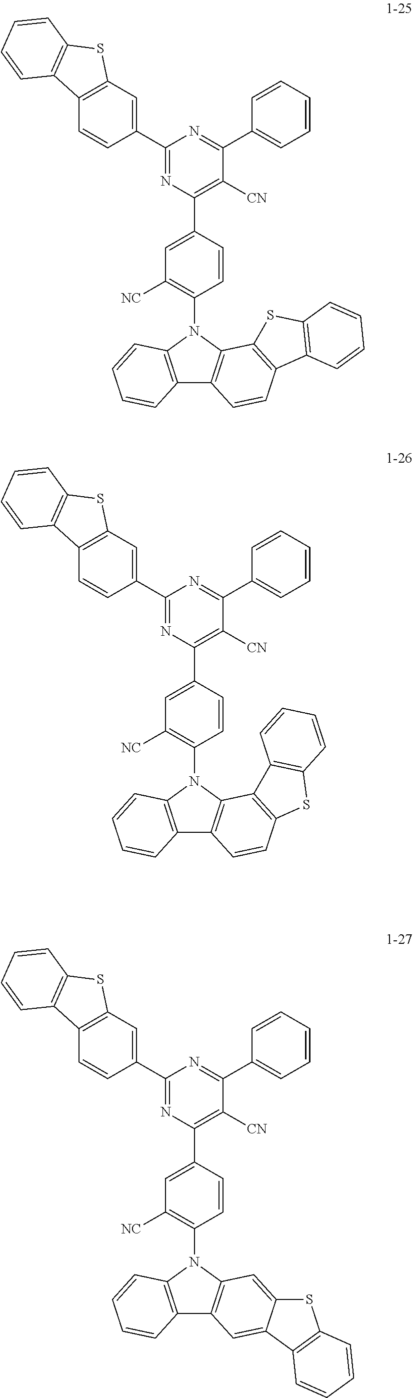 [SMILES: N#Cc1cc(-c2nc(-c3ccc4c(c3)sc3ccccc34)nc(-c3ccccc3)c2C#N)ccc1-n1c2ccccc2c2ccc3c4ccccc4sc3c21 ; N#Cc1cc(-c2nc(-c3ccc4c(c3)sc3ccccc34)nc(-c3ccccc3)c2C#N)ccc1-n1c2ccccc2c2ccc3sc4ccccc4c3c21 ; N#Cc1cc(-c2nc(-c3ccc4c(c3)sc3ccccc34)nc(-c3ccccc3)c2C#N)ccc1-n1c2ccccc2c2cc3c(cc21)sc1ccccc13]

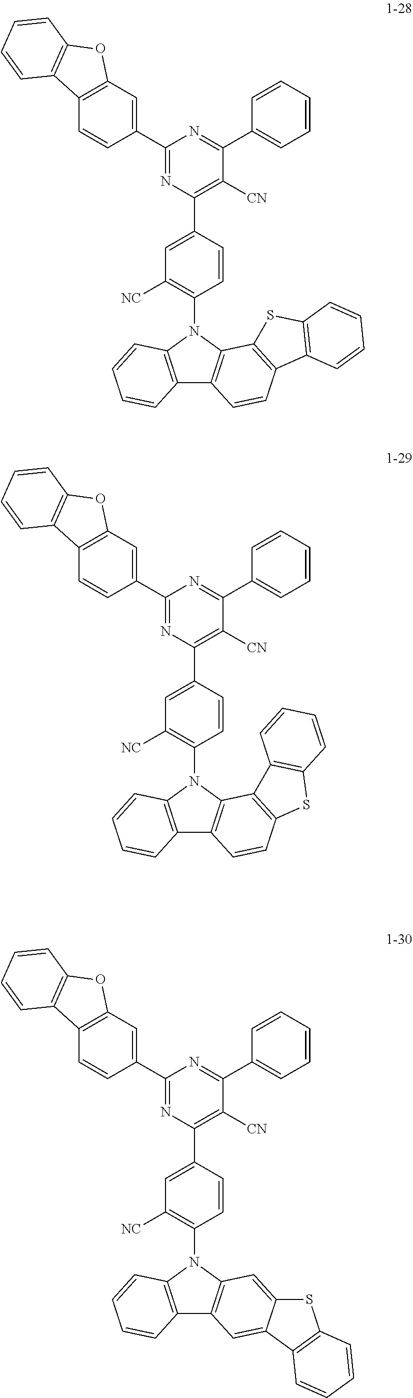 [SMILES: N#Cc1cc(-c2nc(-c3ccc4c(c3)oc3ccccc34)nc(-c3ccccc3)c2C#N)ccc1-n1c2ccccc2c2ccc3c4ccccc4sc3c21 ; N#Cc1cc(-c2nc(-c3ccc4c(c3)oc3ccccc34)nc(-c3ccccc3)c2C#N)ccc1-n1c2ccccc2c2ccc3sc4ccccc4c3c21 ; N#Cc1cc(-c2nc(-c3ccc4c(c3)oc3ccccc34)nc(-c3ccccc3)c2C#N)ccc1-n1c2ccccc2c2cc3c(cc21)sc1ccccc13]

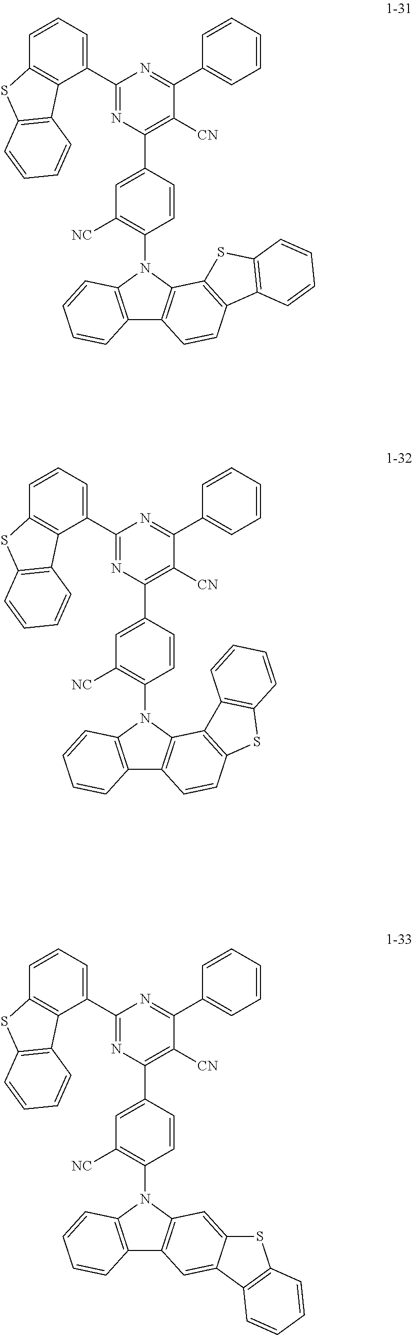 [SMILES: N#Cc1cc(-c2nc(-c3cccc4sc5ccccc5c34)nc(-c3ccccc3)c2C#N)ccc1-n1c2ccccc2c2ccc3c4ccccc4sc3c21 ; N#Cc1cc(-c2nc(-c3cccc4sc5ccccc5c34)nc(-c3ccccc3)c2C#N)ccc1-n1c2ccccc2c2ccc3sc4ccccc4c3c21 ; N#Cc1cc(-c2nc(-c3cccc4sc5ccccc5c34)nc(-c3ccccc3)c2C#N)ccc1-n1c2ccccc2c2cc3c(cc21)sc1ccccc13]

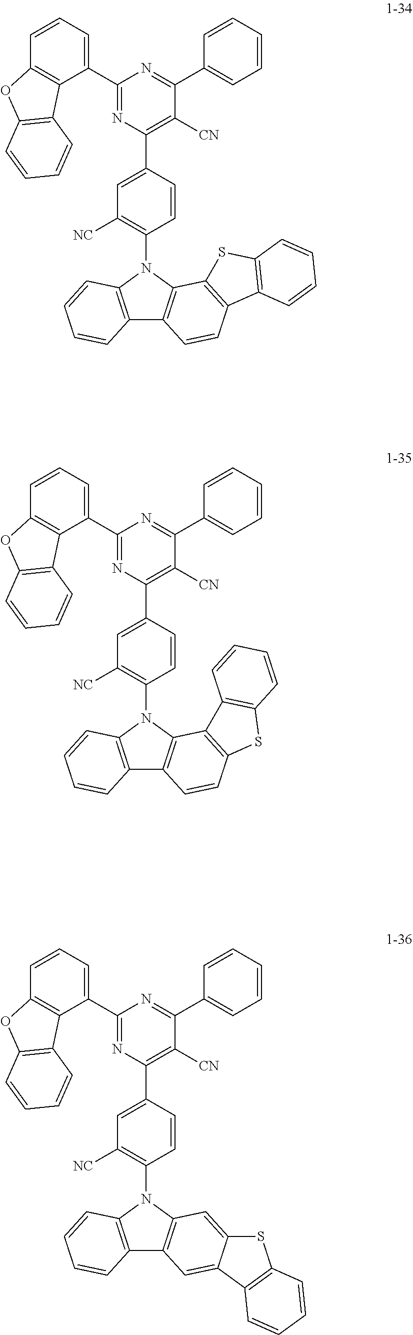 [SMILES: N#Cc1cc(-c2nc(-c3cccc4oc5ccccc5c34)nc(-c3ccccc3)c2C#N)ccc1-n1c2ccccc2c2ccc3c4ccccc4sc3c21 ; N#Cc1cc(-c2nc(-c3cccc4oc5ccccc5c34)nc(-c3ccccc3)c2C#N)ccc1-n1c2ccccc2c2ccc3sc4ccccc4c3c21 ; N#Cc1cc(-c2nc(-c3cccc4oc5ccccc5c34)nc(-c3ccccc3)c2C#N)ccc1-n1c2ccccc2c2cc3c(cc21)sc1ccccc13]

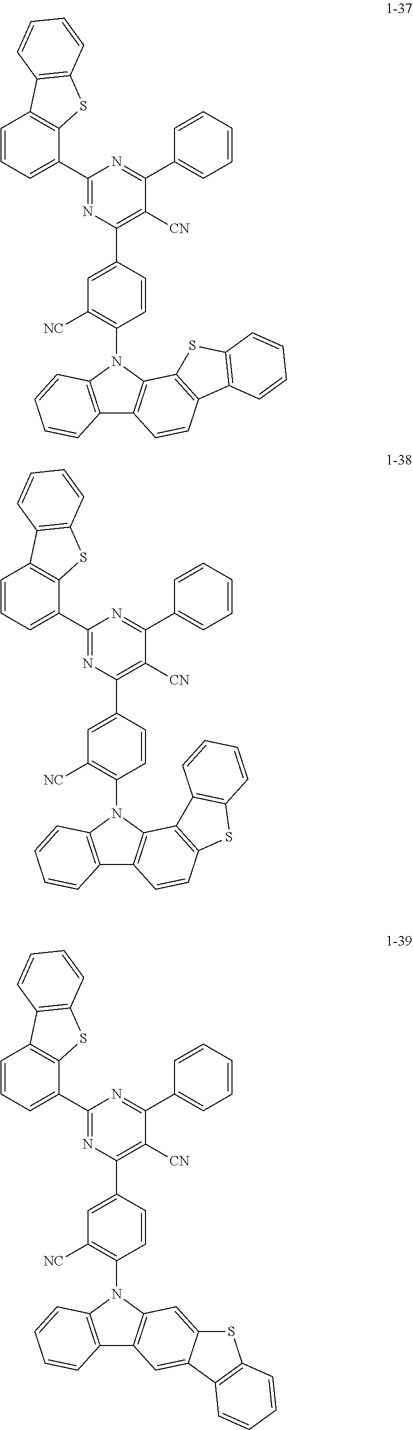

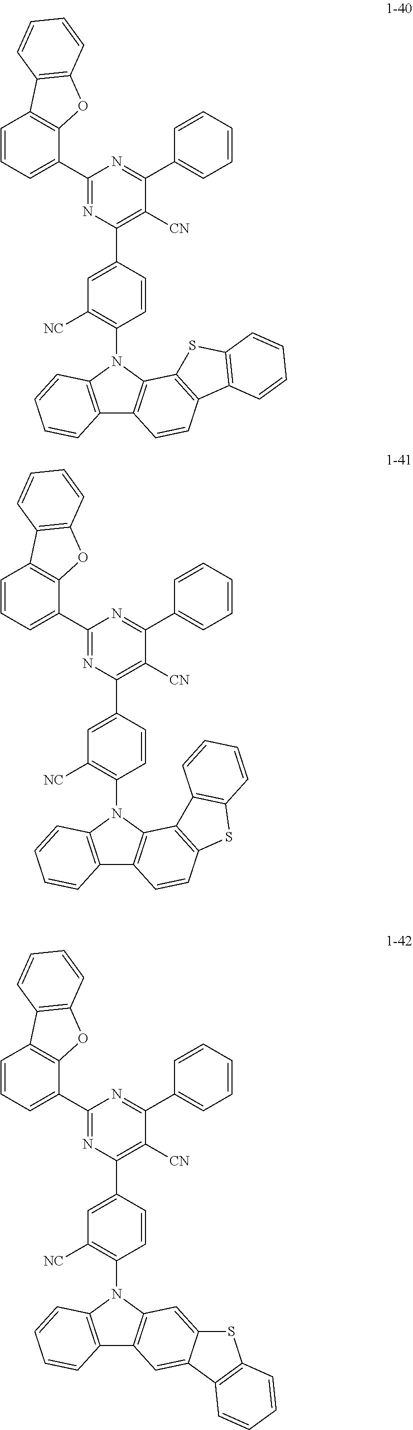

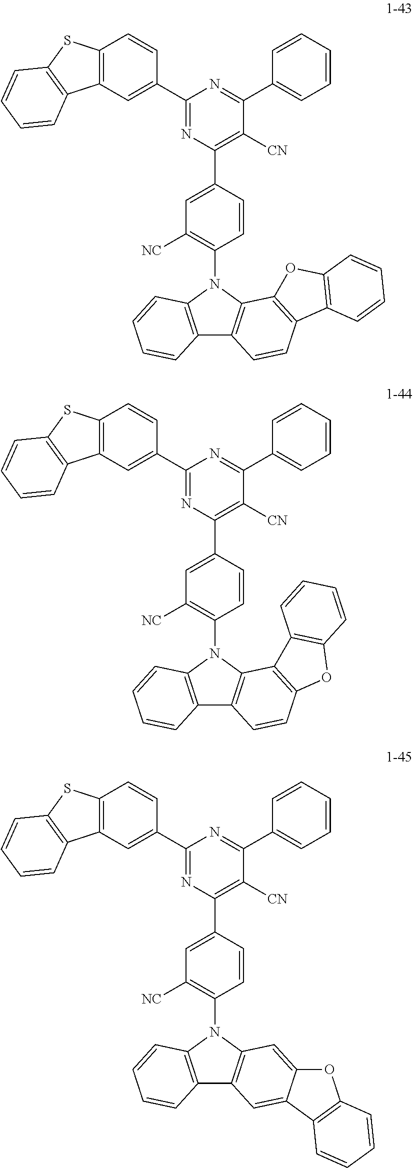 [SMILES: N#Cc1cc(-c2nc(-c3ccc4sc5ccccc5c4c3)nc(-c3ccccc3)c2C#N)ccc1-n1c2ccccc2c2ccc3c4ccccc4oc3c21 ; N#Cc1cc(-c2nc(-c3ccc4sc5ccccc5c4c3)nc(-c3ccccc3)c2C#N)ccc1-n1c2ccccc2c2ccc3oc4ccccc4c3c21 ; N#Cc1cc(-c2nc(-c3ccc4sc5ccccc5c4c3)nc(-c3ccccc3)c2C#N)ccc1-n1c2ccccc2c2cc3c(cc21)oc1ccccc13]

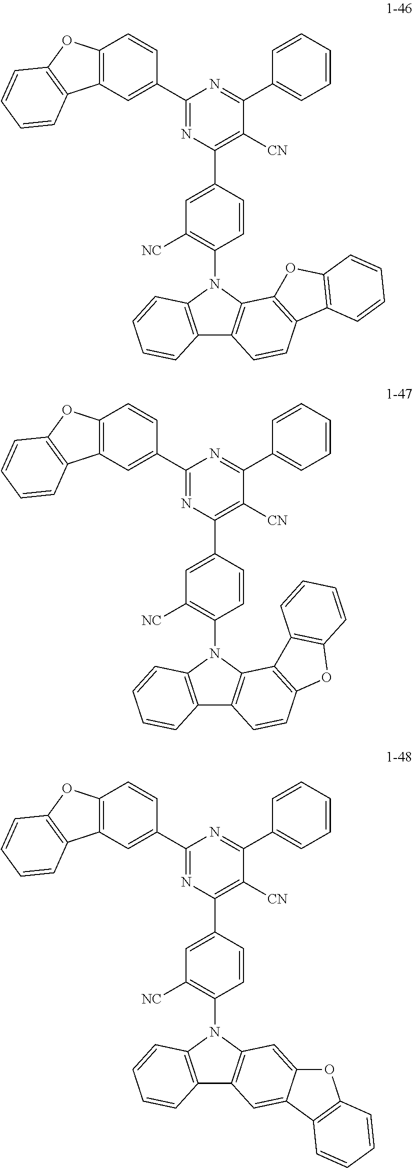 [SMILES: N#Cc1cc(-c2nc(-c3ccc4oc5ccccc5c4c3)nc(-c3ccccc3)c2C#N)ccc1-n1c2ccccc2c2ccc3c4ccccc4oc3c21 ; N#Cc1cc(-c2nc(-c3ccc4oc5ccccc5c4c3)nc(-c3ccccc3)c2C#N)ccc1-n1c2ccccc2c2ccc3oc4ccccc4c3c21 ; N#Cc1cc(-c2nc(-c3ccc4oc5ccccc5c4c3)nc(-c3ccccc3)c2C#N)ccc1-n1c2ccccc2c2cc3c(cc21)oc1ccccc13]

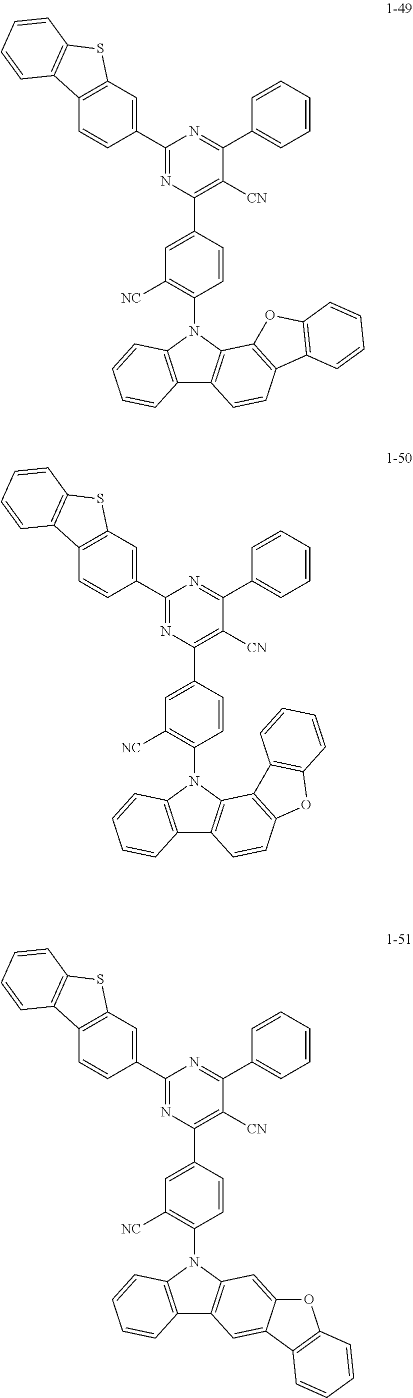 [SMILES: N#Cc1cc(-c2nc(-c3ccc4c(c3)sc3ccccc34)nc(-c3ccccc3)c2C#N)ccc1-n1c2ccccc2c2ccc3c4ccccc4oc3c21 ; N#Cc1cc(-c2nc(-c3ccc4c(c3)sc3ccccc34)nc(-c3ccccc3)c2C#N)ccc1-n1c2ccccc2c2ccc3oc4ccccc4c3c21 ; N#Cc1cc(-c2nc(-c3ccc4c(c3)sc3ccccc34)nc(-c3ccccc3)c2C#N)ccc1-n1c2ccccc2c2cc3c(cc21)oc1ccccc13]

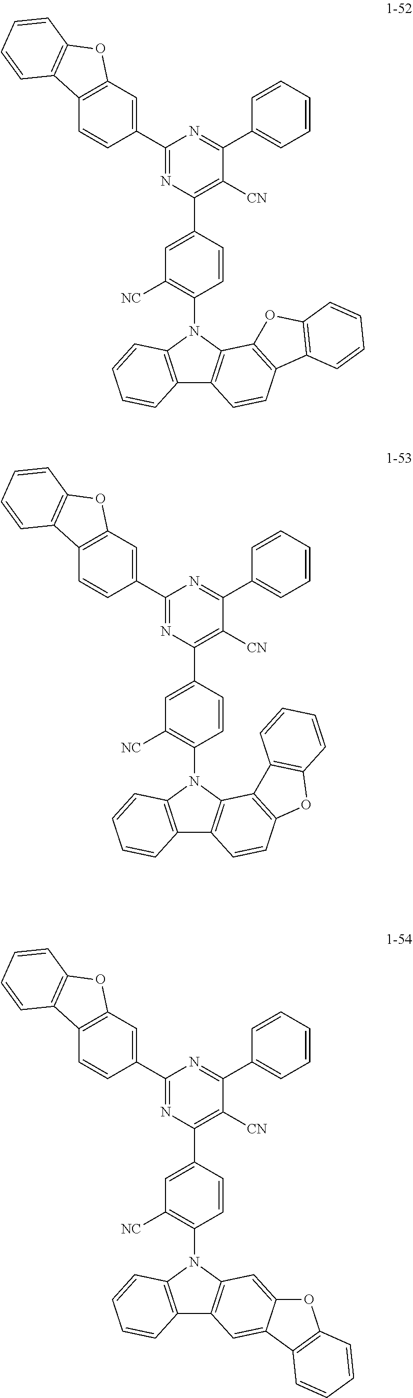 [SMILES: N#Cc1cc(-c2nc(-c3ccc4c(c3)oc3ccccc34)nc(-c3ccccc3)c2C#N)ccc1-n1c2ccccc2c2ccc3c4ccccc4oc3c21 ; N#Cc1cc(-c2nc(-c3ccc4c(c3)oc3ccccc34)nc(-c3ccccc3)c2C#N)ccc1-n1c2ccccc2c2ccc3oc4ccccc4c3c21 ; N#Cc1cc(-c2nc(-c3ccc4c(c3)oc3ccccc34)nc(-c3ccccc3)c2C#N)ccc1-n1c2ccccc2c2cc3c(cc21)oc1ccccc13]

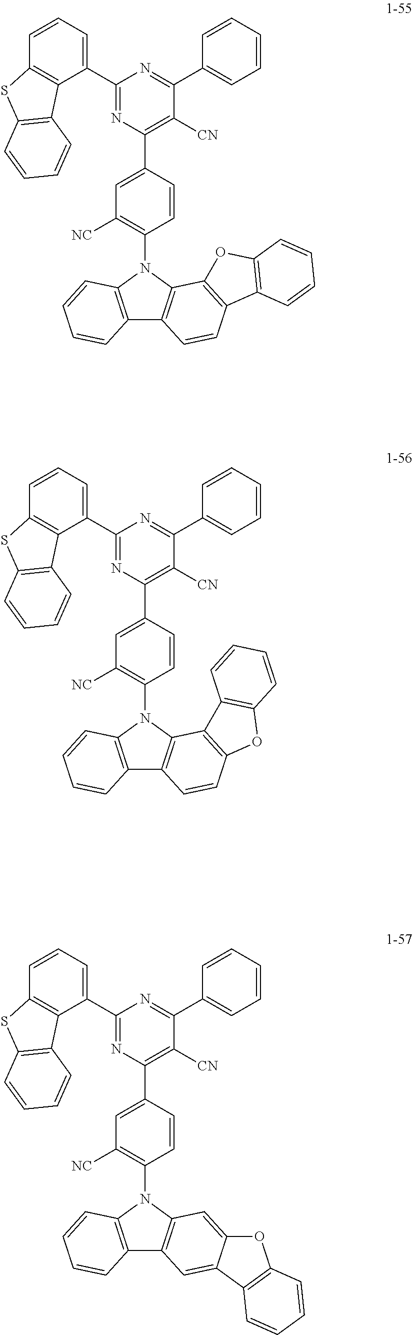 [SMILES: N#Cc1cc(-c2nc(-c3cccc4sc5ccccc5c34)nc(-c3ccccc3)c2C#N)ccc1-n1c2ccccc2c2ccc3c4ccccc4oc3c21 ; N#Cc1cc(-c2nc(-c3cccc4sc5ccccc5c34)nc(-c3ccccc3)c2C#N)ccc1-n1c2ccccc2c2ccc3oc4ccccc4c3c21 ; N#Cc1cc(-c2nc(-c3cccc4sc5ccccc5c34)nc(-c3ccccc3)c2C#N)ccc1-n1c2ccccc2c2cc3c(cc21)oc1ccccc13]

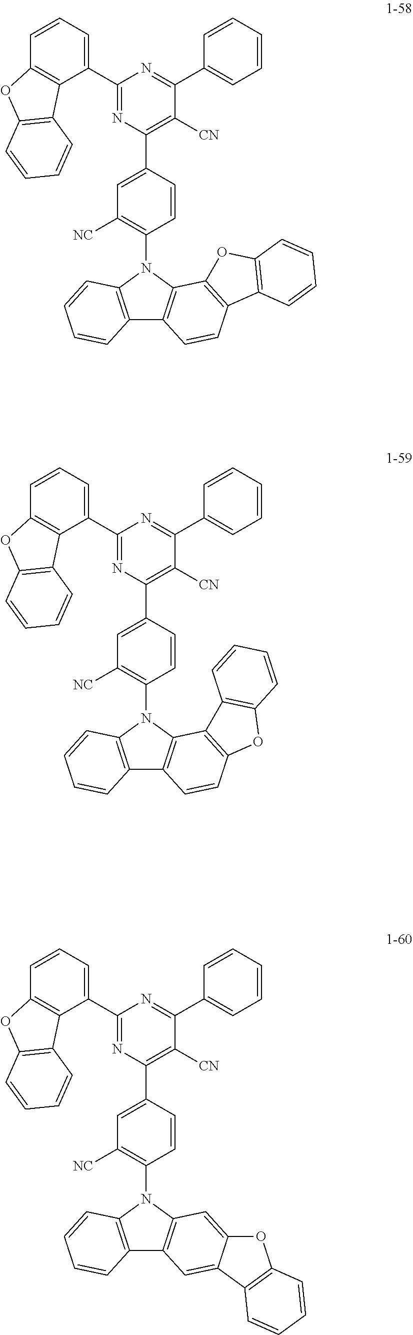 [SMILES: N#Cc1cc(-c2nc(-c3cccc4oc5ccccc5c34)nc(-c3ccccc3)c2C#N)ccc1-n1c2ccccc2c2ccc3c4ccccc4oc3c21 ; N#Cc1cc(-c2nc(-c3cccc4oc5ccccc5c34)nc(-c3ccccc3)c2C#N)ccc1-n1c2ccccc2c2ccc3oc4ccccc4c3c21 ; N#Cc1cc(-c2nc(-c3cccc4oc5ccccc5c34)nc(-c3ccccc3)c2C#N)ccc1-n1c2ccccc2c2cc3c(cc21)oc1ccccc13]

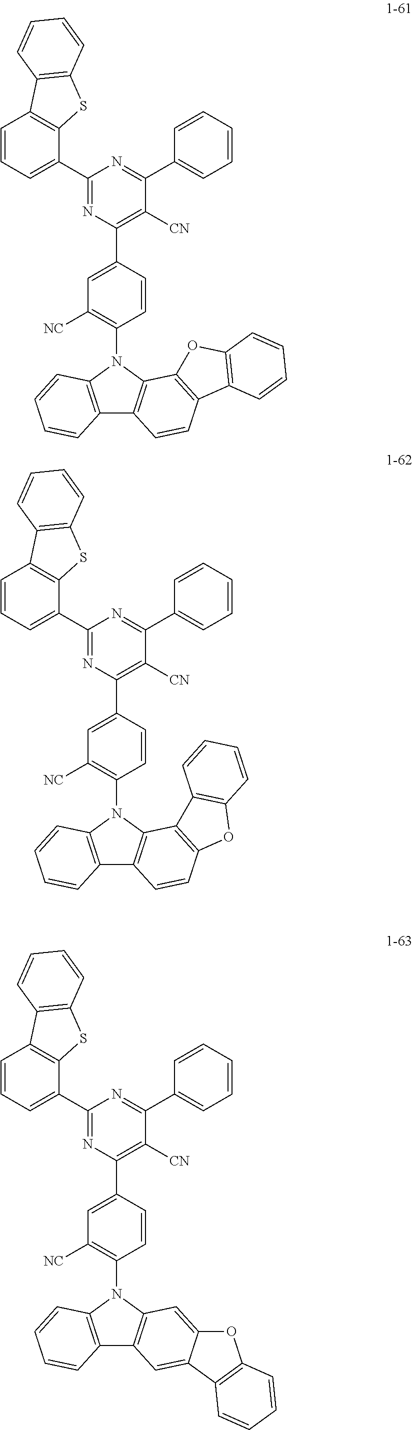

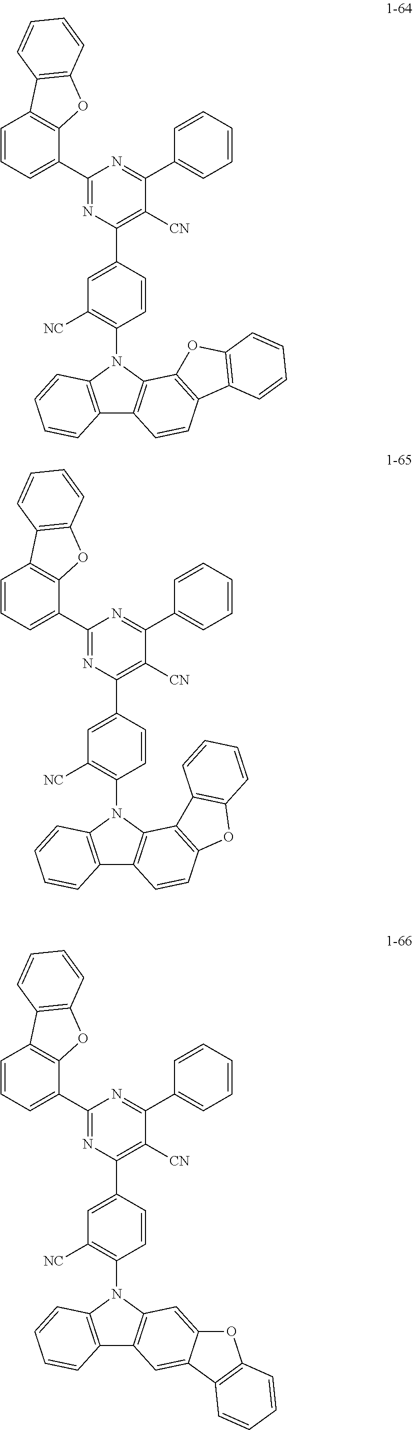

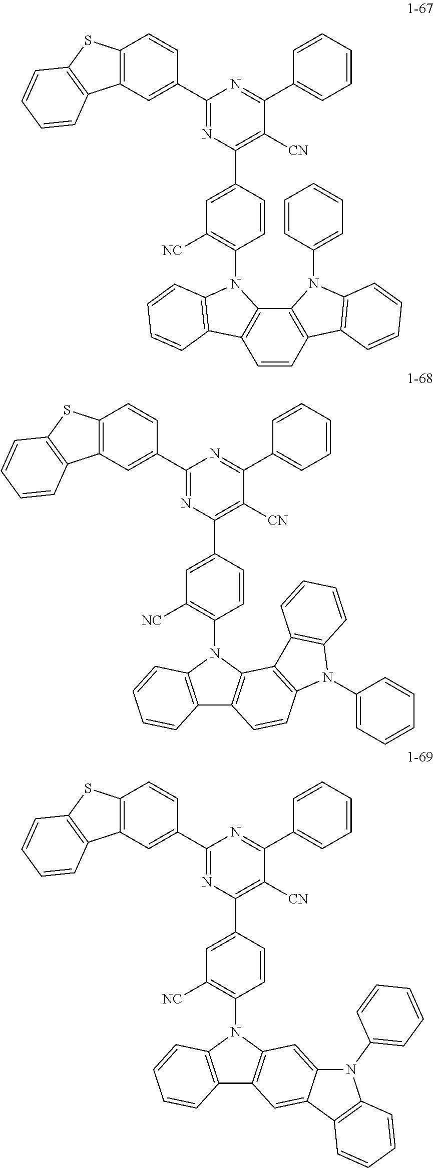

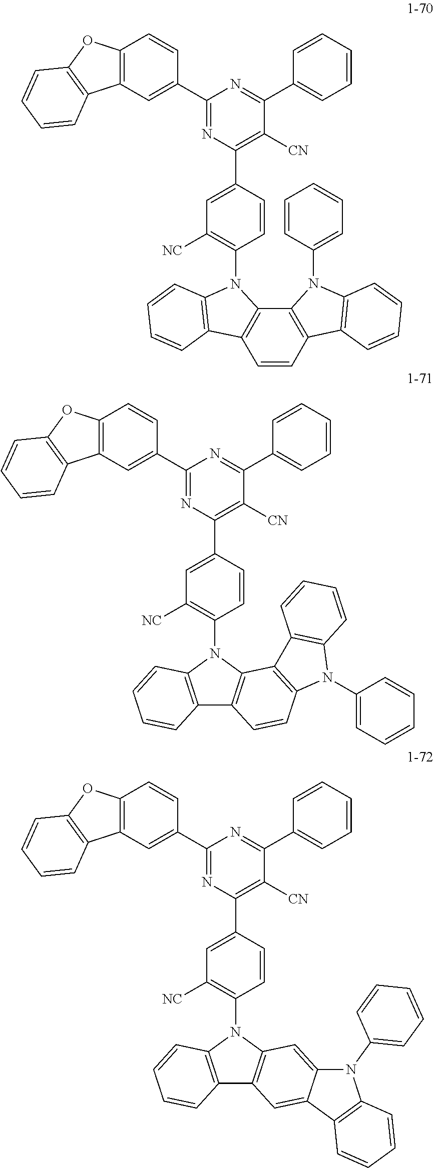

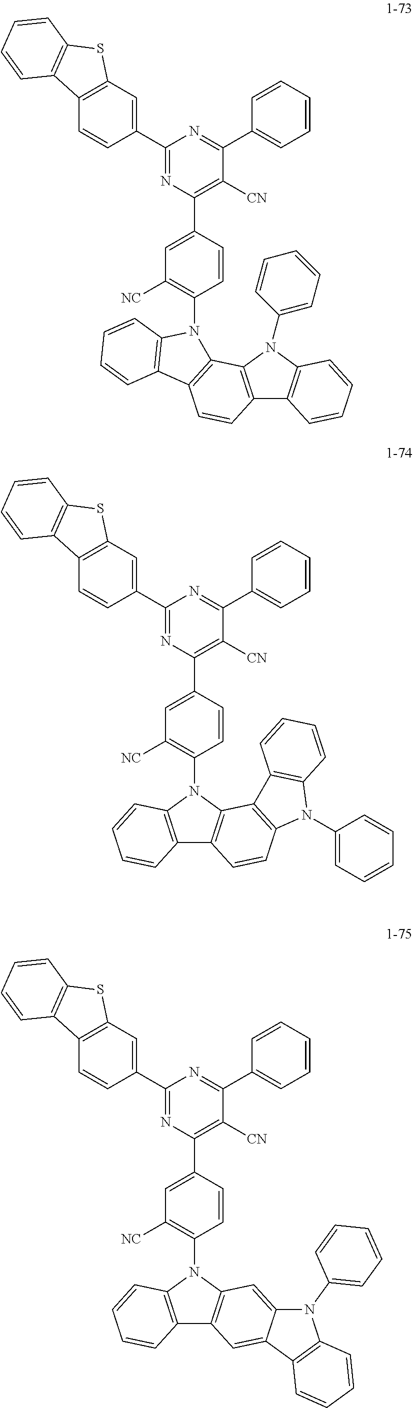 [SMILES: N#Cc1cc(-c2nc(-c3ccc4c(c3)sc3ccccc34)nc(-c3ccccc3)c2C#N)ccc1-n1c2ccccc2c2ccc3c4ccccc4n(-c4ccccc4)c3c21 ; N#Cc1cc(-c2nc(-c3ccc4c(c3)sc3ccccc34)nc(-c3ccccc3)c2C#N)ccc1-n1c2ccccc2c2ccc3c(c4ccccc4n3-c3ccccc3)c21 ; N#Cc1cc(-c2nc(-c3ccc4c(c3)sc3ccccc34)nc(-c3ccccc3)c2C#N)ccc1-n1c2ccccc2c2cc3c4ccccc4n(-c4ccccc4)c3cc21]

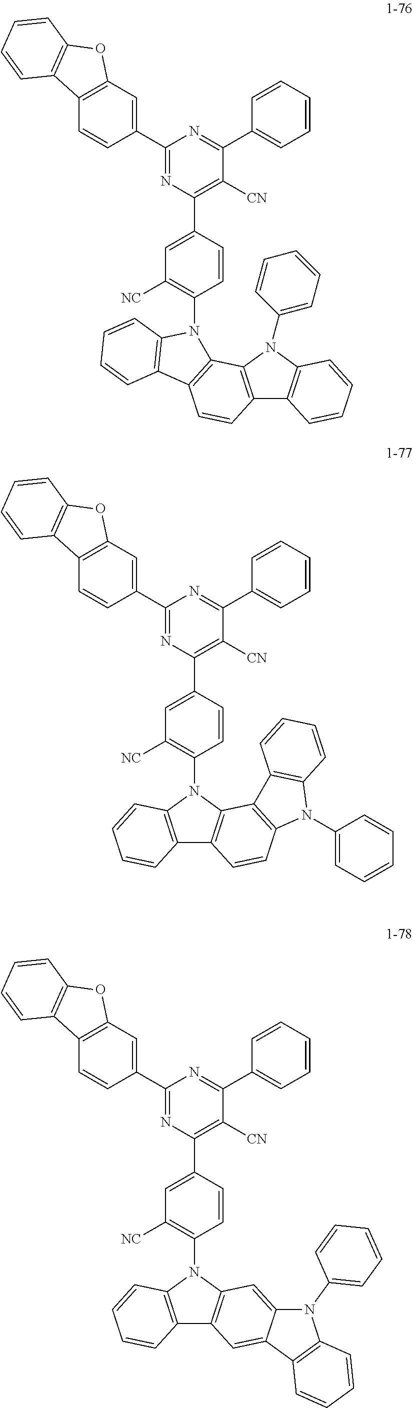 [SMILES: N#Cc1cc(-c2nc(-c3ccc4c(c3)oc3ccccc34)nc(-c3ccccc3)c2C#N)ccc1-n1c2ccccc2c2ccc3c4ccccc4n(-c4ccccc4)c3c21 ; N#Cc1cc(-c2nc(-c3ccc4c(c3)oc3ccccc34)nc(-c3ccccc3)c2C#N)ccc1-n1c2ccccc2c2ccc3c(c4ccccc4n3-c3ccccc3)c21 ; N#Cc1cc(-c2nc(-c3ccc4c(c3)oc3ccccc34)nc(-c3ccccc3)c2C#N)ccc1-n1c2ccccc2c2cc3c4ccccc4n(-c4ccccc4)c3cc21]

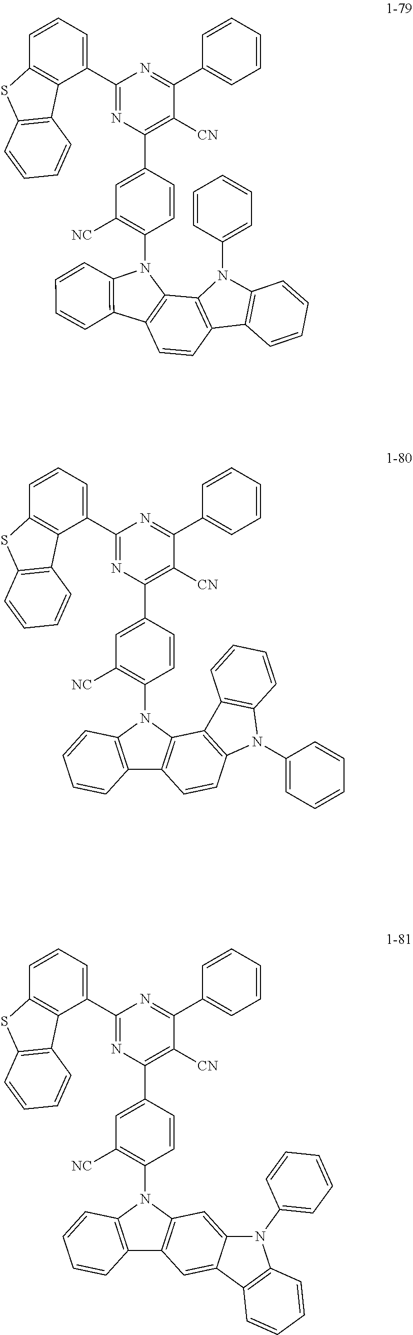 [SMILES: N#Cc1cc(-c2nc(-c3cccc4sc5ccccc5c34)nc(-c3ccccc3)c2C#N)ccc1-n1c2ccccc2c2ccc3c4ccccc4n(-c4ccccc4)c3c21 ; N#Cc1cc(-c2nc(-c3cccc4sc5ccccc5c34)nc(-c3ccccc3)c2C#N)ccc1-n1c2ccccc2c2ccc3c(c4ccccc4n3-c3ccccc3)c21 ; N#Cc1cc(-c2nc(-c3cccc4sc5ccccc5c34)nc(-c3ccccc3)c2C#N)ccc1-n1c2ccccc2c2cc3c4ccccc4n(-c4ccccc4)c3cc21]

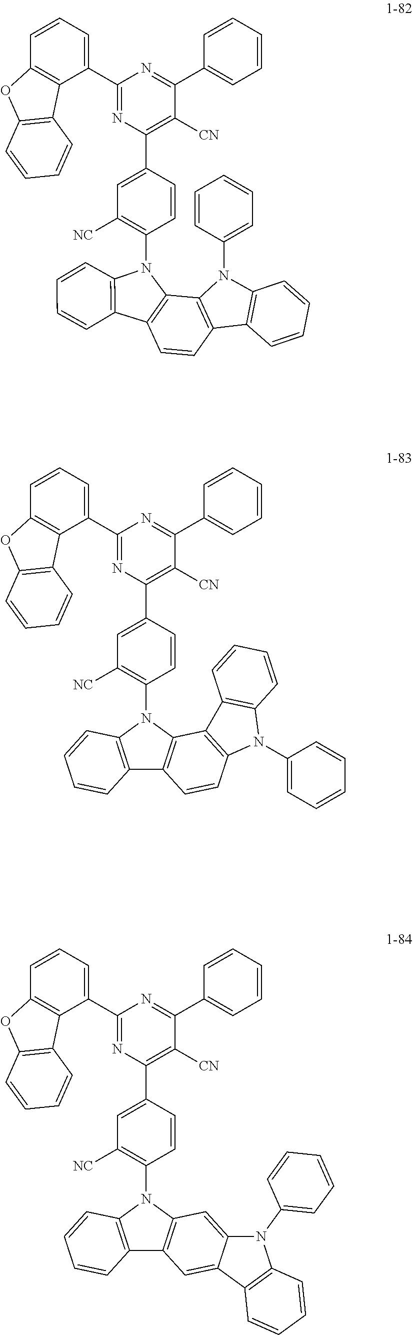 [SMILES: N#Cc1cc(-c2nc(-c3cccc4oc5ccccc5c34)nc(-c3ccccc3)c2C#N)ccc1-n1c2ccccc2c2ccc3c4ccccc4n(-c4ccccc4)c3c21 ; N#Cc1cc(-c2nc(-c3cccc4oc5ccccc5c34)nc(-c3ccccc3)c2C#N)ccc1-n1c2ccccc2c2ccc3c(c4ccccc4n3-c3ccccc3)c21 ; N#Cc1cc(-c2nc(-c3cccc4oc5ccccc5c34)nc(-c3ccccc3)c2C#N)ccc1-n1c2ccccc2c2cc3c4ccccc4n(-c4ccccc4)c3cc21]

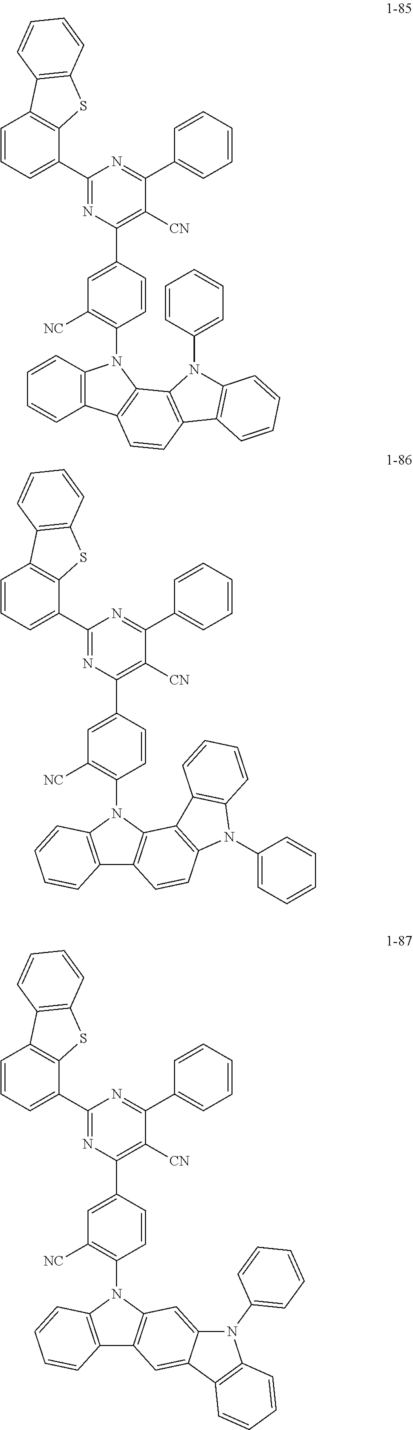 [SMILES: N#Cc1cc(-c2nc(-c3cccc4c3sc3ccccc34)nc(-c3ccccc3)c2C#N)ccc1-n1c2ccccc2c2ccc3c4ccccc4n(-c4ccccc4)c3c21 ; N#Cc1cc(-c2nc(-c3cccc4c3sc3ccccc34)nc(-c3ccccc3)c2C#N)ccc1-n1c2ccccc2c2ccc3c(c4ccccc4n3-c3ccccc3)c21 ; N#Cc1cc(-c2nc(-c3cccc4c3sc3ccccc34)nc(-c3ccccc3)c2C#N)ccc1-n1c2ccccc2c2cc3c4ccccc4n(-c4ccccc4)c3cc21]

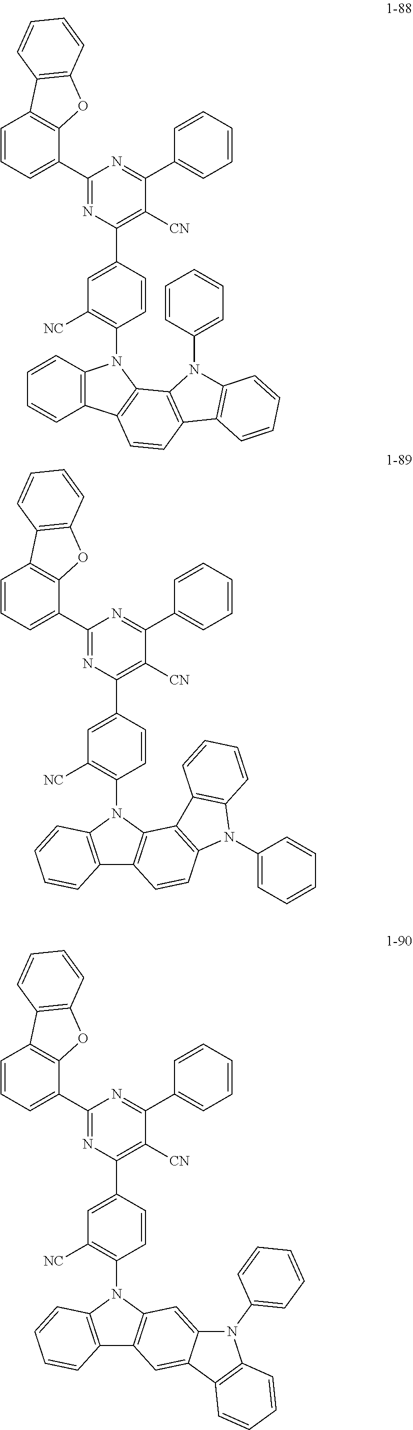 [SMILES: N#Cc1cc(-c2nc(-c3cccc4c3oc3ccccc34)nc(-c3ccccc3)c2C#N)ccc1-n1c2ccccc2c2ccc3c4ccccc4n(-c4ccccc4)c3c21 ; N#Cc1cc(-c2nc(-c3cccc4c3oc3ccccc34)nc(-c3ccccc3)c2C#N)ccc1-n1c2ccccc2c2ccc3c(c4ccccc4n3-c3ccccc3)c21 ; N#Cc1cc(-c2nc(-c3cccc4c3oc3ccccc34)nc(-c3ccccc3)c2C#N)ccc1-n1c2ccccc2c2cc3c4ccccc4n(-c4ccccc4)c3cc21]

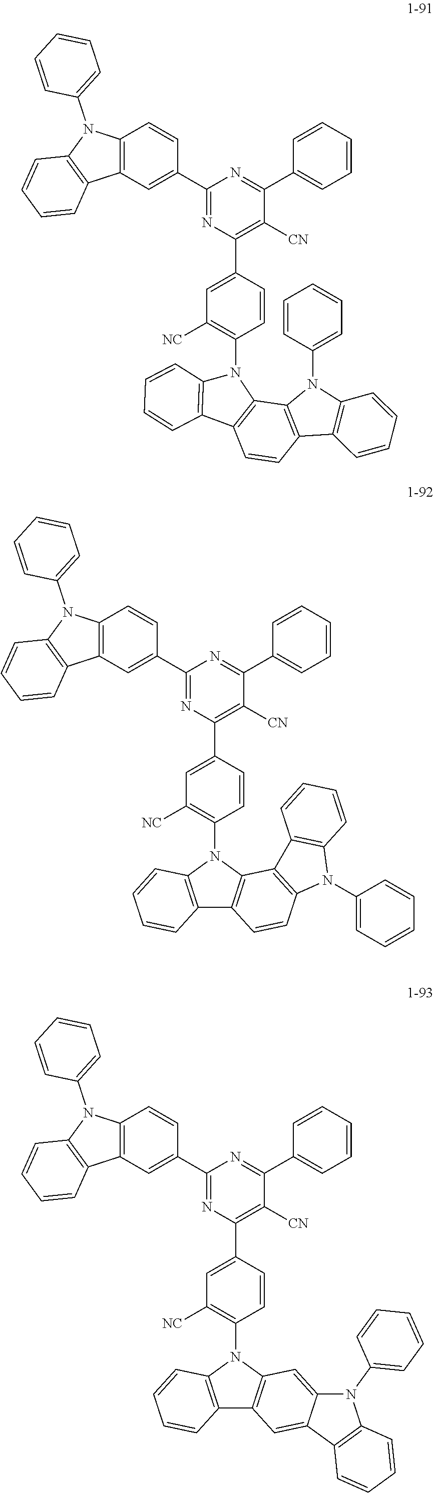 [SMILES: N#Cc1cc(-c2nc(-c3ccc4c(c3)c3ccccc3n4-c3ccccc3)nc(-c3ccccc3)c2C#N)ccc1-n1c2ccccc2c2ccc3c4ccccc4n(-c4ccccc4)c3c21 ; N#Cc1cc(-c2nc(-c3ccc4c(c3)c3ccccc3n4-c3ccccc3)nc(-c3ccccc3)c2C#N)ccc1-n1c2ccccc2c2ccc3c(c4ccccc4n3-c3ccccc3)c21 ; N#Cc1cc(-c2nc(-c3ccc4c(c3)c3ccccc3n4-c3ccccc3)nc(-c3ccccc3)c2C#N)ccc1-n1c2ccccc2c2cc3c4ccccc4n(-c4ccccc4)c3cc21]

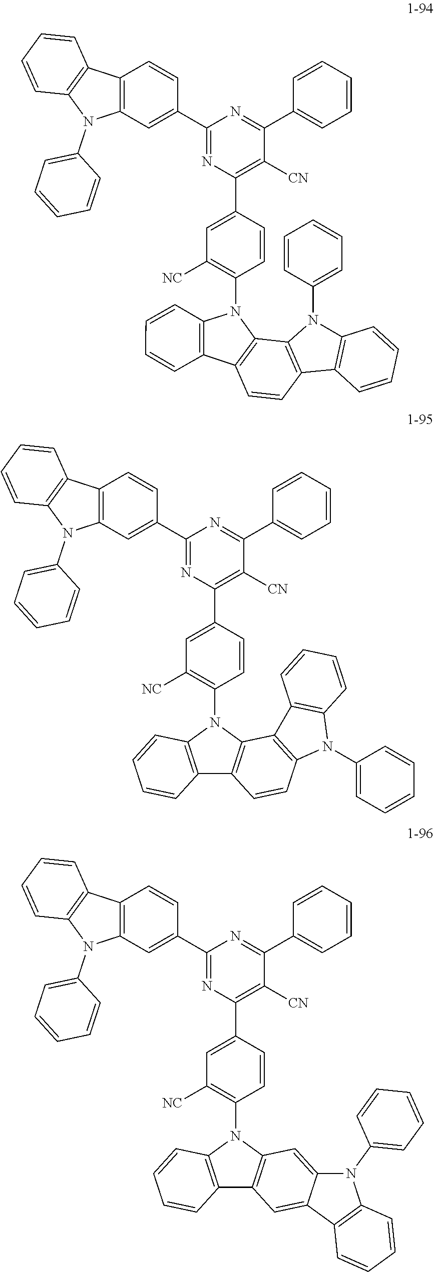 [SMILES: N#Cc1cc(-c2nc(-c3ccc4c5ccccc5n(-c5ccccc5)c4c3)nc(-c3ccccc3)c2C#N)ccc1-n1c2ccccc2c2ccc3c4ccccc4n(-c4ccccc4)c3c21 ; N#Cc1cc(-c2nc(-c3ccc4c5ccccc5n(-c5ccccc5)c4c3)nc(-c3ccccc3)c2C#N)ccc1-n1c2ccccc2c2ccc3c(c4ccccc4n3-c3ccccc3)c21 ; N#Cc1cc(-c2nc(-c3ccc4c5ccccc5n(-c5ccccc5)c4c3)nc(-c3ccccc3)c2C#N)ccc1-n1c2ccccc2c2cc3c4ccccc4n(-c4ccccc4)c3cc21]

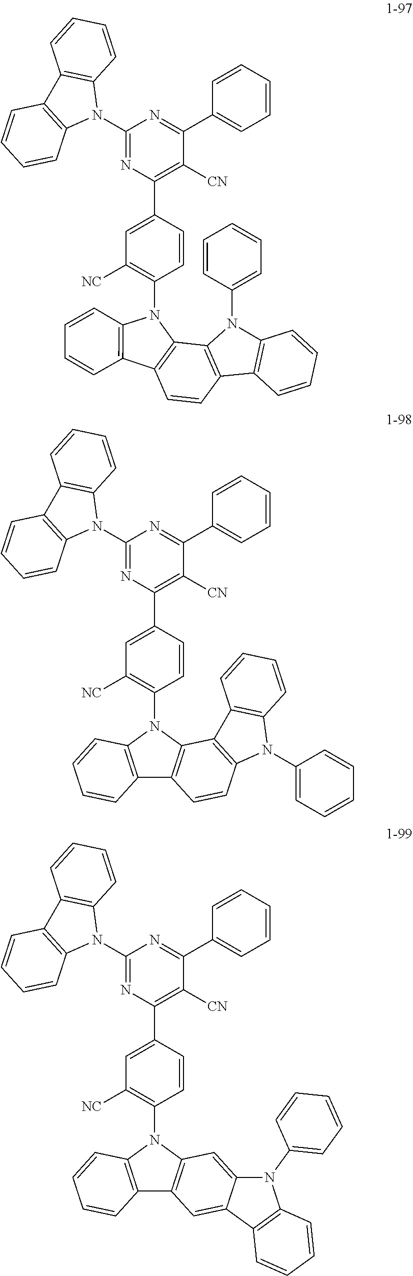 [SMILES: N#Cc1cc(-c2nc(-n3c4ccccc4c4ccccc43)nc(-c3ccccc3)c2C#N)ccc1-n1c2ccccc2c2ccc3c4ccccc4n(-c4ccccc4)c3c21 ; N#Cc1cc(-c2nc(-n3c4ccccc4c4ccccc43)nc(-c3ccccc3)c2C#N)ccc1-n1c2ccccc2c2ccc3c(c4ccccc4n3-c3ccccc3)c21 ; N#Cc1cc(-c2nc(-n3c4ccccc4c4ccccc43)nc(-c3ccccc3)c2C#N)ccc1-n1c2ccccc2c2cc3c4ccccc4n(-c4ccccc4)c3cc21]

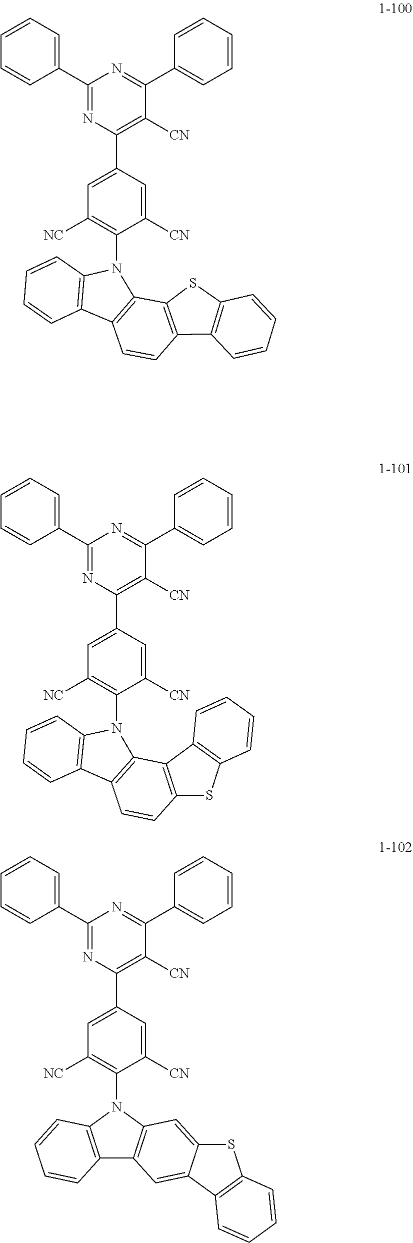 [SMILES: N#Cc1cc(-c2nc(-c3ccccc3)nc(-c3ccccc3)c2C#N)cc(C#N)c1-n1c2ccccc2c2ccc3c4ccccc4sc3c21 ; N#Cc1cc(-c2nc(-c3ccccc3)nc(-c3ccccc3)c2C#N)cc(C#N)c1-n1c2ccccc2c2ccc3sc4ccccc4c3c21 ; N#Cc1cc(-c2nc(-c3ccccc3)nc(-c3ccccc3)c2C#N)cc(C#N)c1-n1c2ccccc2c2cc3c(cc21)sc1ccccc13]

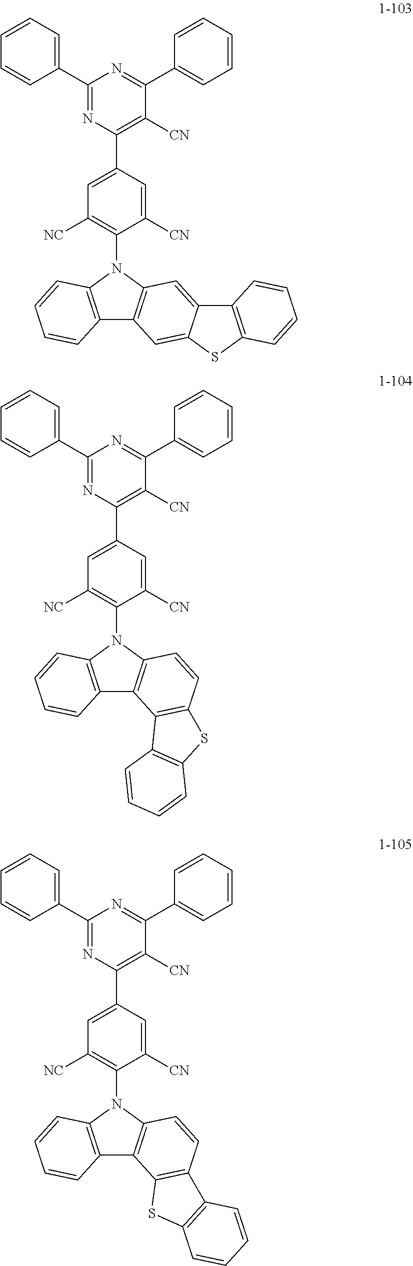

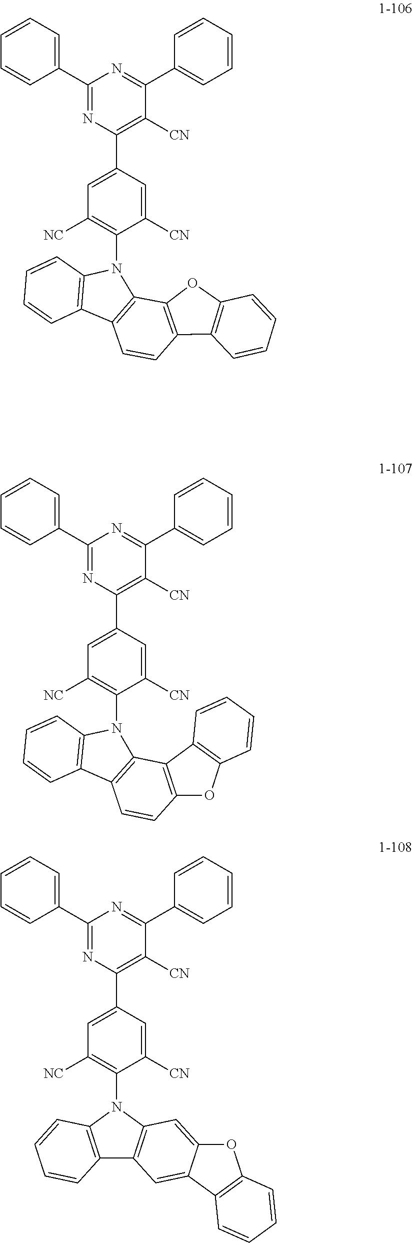 [SMILES: N#Cc1cc(-c2nc(-c3ccccc3)nc(-c3ccccc3)c2C#N)cc(C#N)c1-n1c2ccccc2c2ccc3c4ccccc4oc3c21 ; N#Cc1cc(-c2nc(-c3ccccc3)nc(-c3ccccc3)c2C#N)cc(C#N)c1-n1c2ccccc2c2ccc3oc4ccccc4c3c21 ; N#Cc1cc(-c2nc(-c3ccccc3)nc(-c3ccccc3)c2C#N)cc(C#N)c1-n1c2ccccc2c2cc3c(cc21)oc1ccccc13]

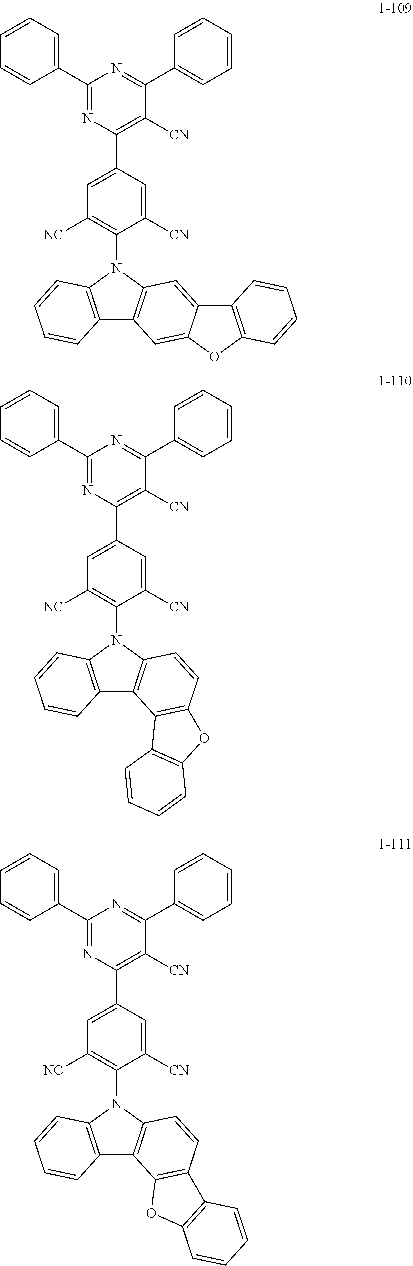

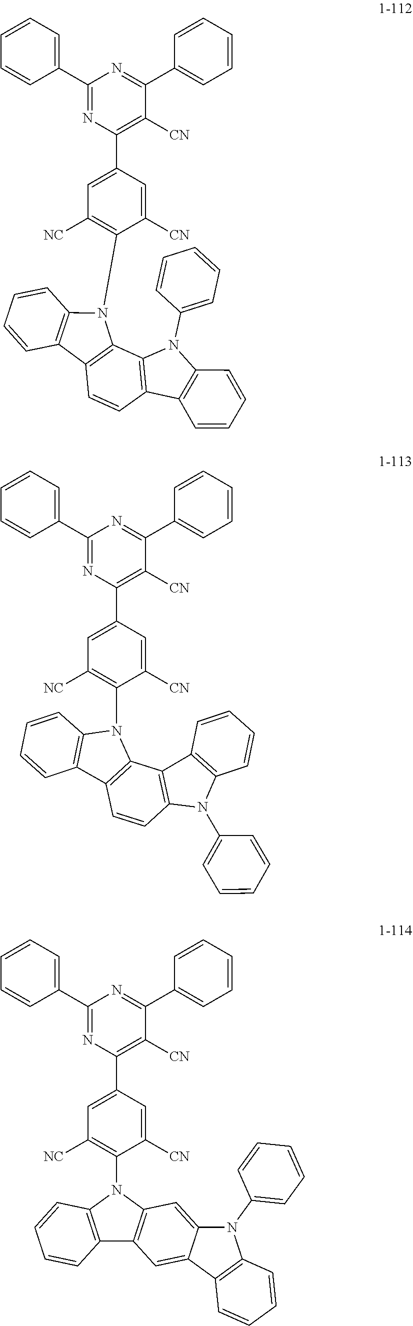

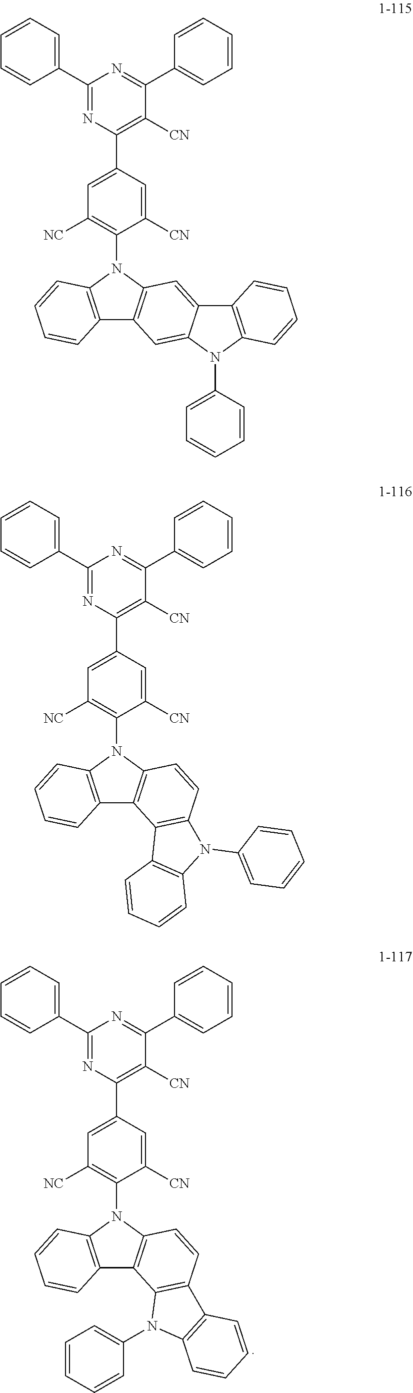

8. The organic light emitting diode of claim 1, wherein the second compound has the following structure of Formula 6: 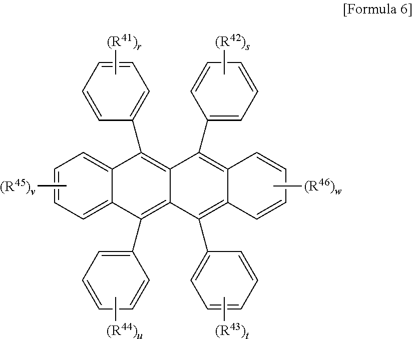
wherein, in Formula 6,
each of R⁴¹ to R⁴⁶ is independently hydrogen, protium, deuterium, tritium, unsubstituted or substituted C₁-C₂₀ alkyl, an unsubstituted or substituted C₆-C₃₀ aromatic group or an unsubstituted or substituted C₃-C₃₀ hetero aromatic group, when r is an integer of 2 or more, each R⁴¹ is identical to or different from each other, when s is an integer of 2 or more, each R⁴² is identical to or different from each other, when t is an integer of 2 or more, each R⁴³ is identical to or different from each other, when u is an integer of 2 or more, each R⁴⁴ is identical to or different from each other, when v is an integer of 2 or more, each R⁴⁵ is identical to or different from each other, when w is an integer of 2 or more, each R⁴⁶ is identical to or different from each other; and
each of r, s, t and u is independently an integer of 0 to 5; and
each of v and w is independently an integer of 0 to 4.

9. The organic light emitting diode of claim 1, wherein the second compound is selected from the following compounds:

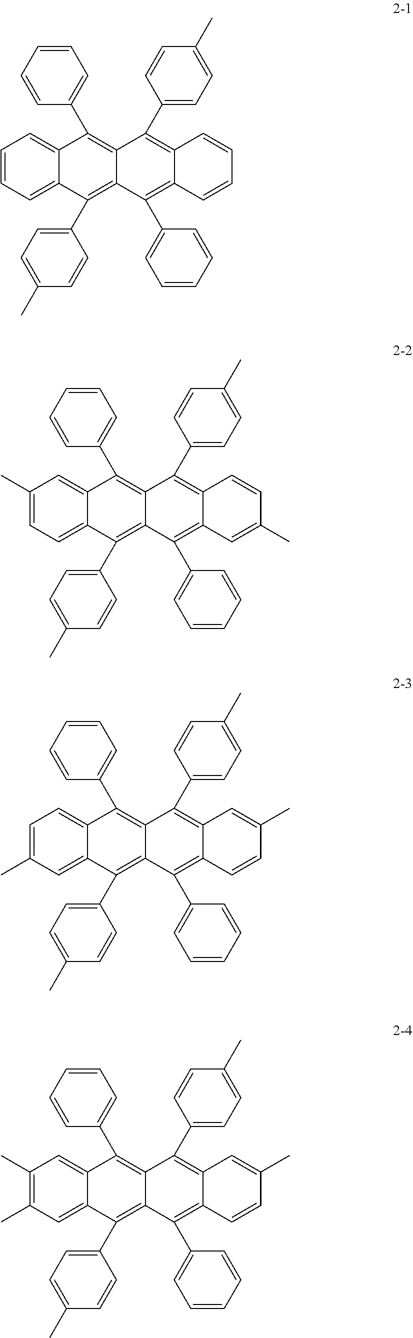

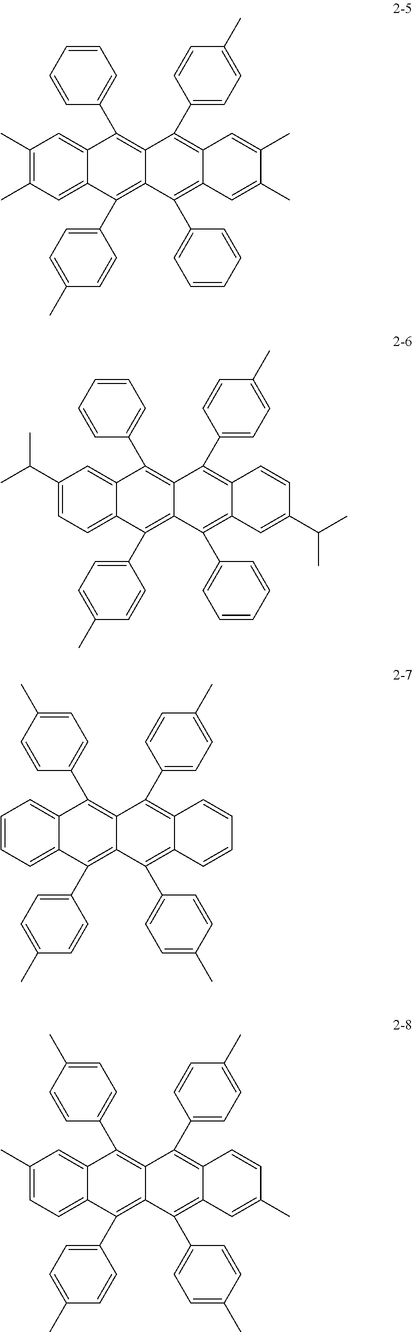

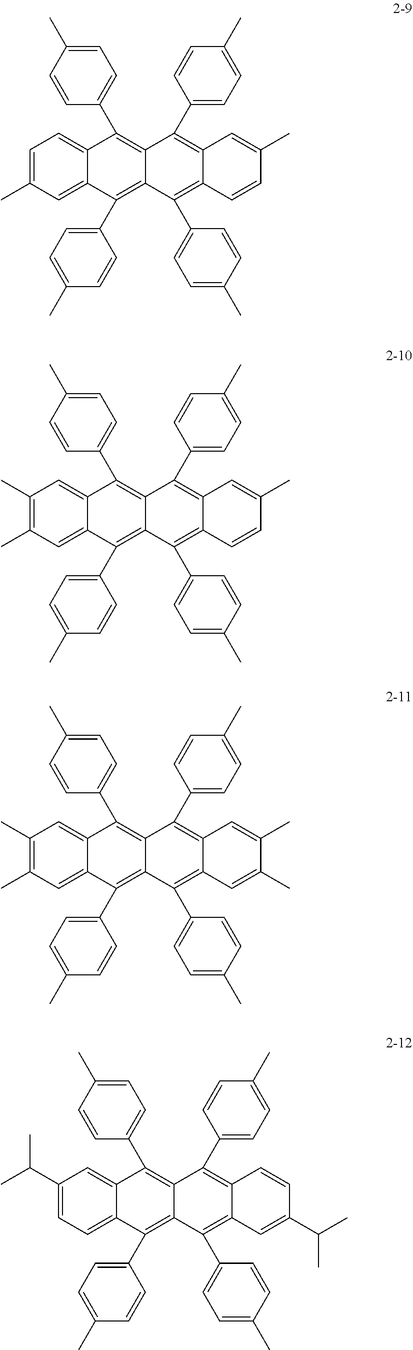

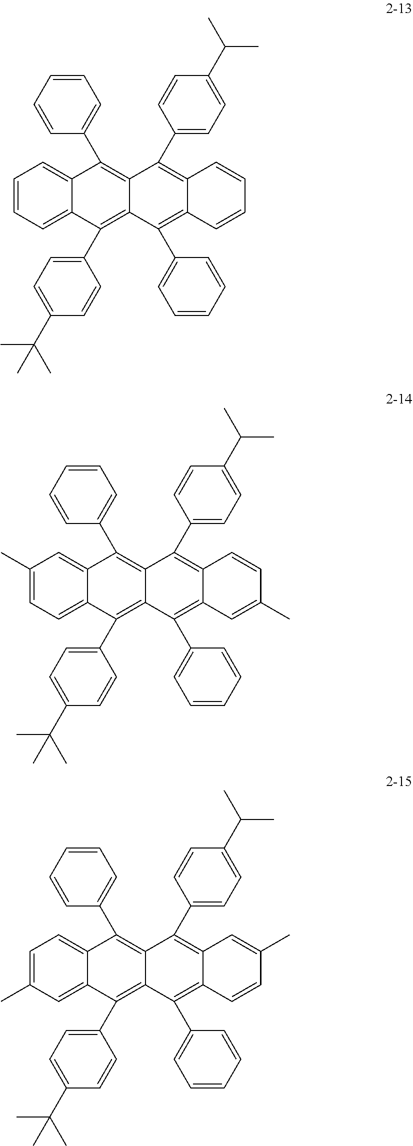

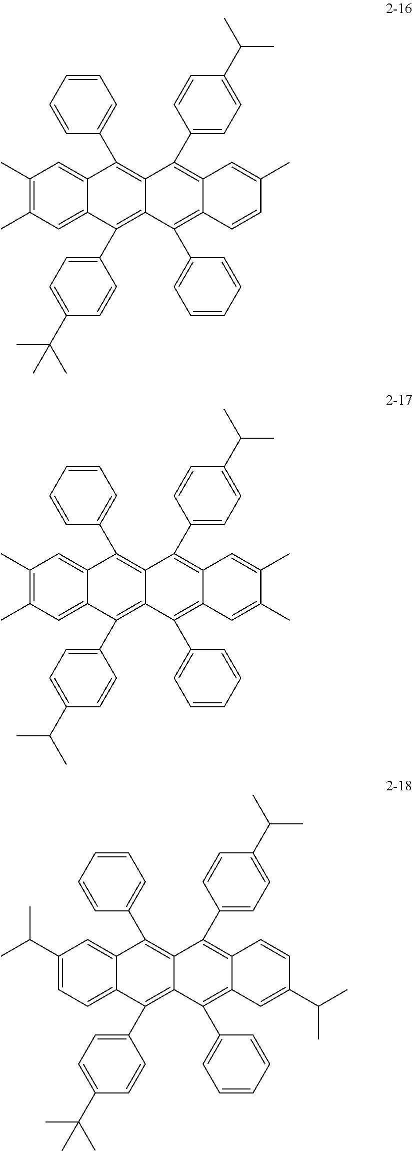

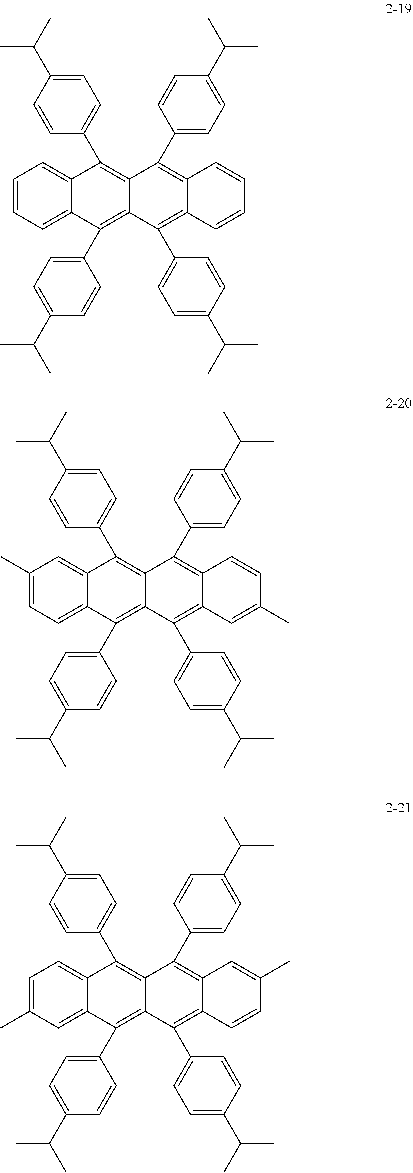

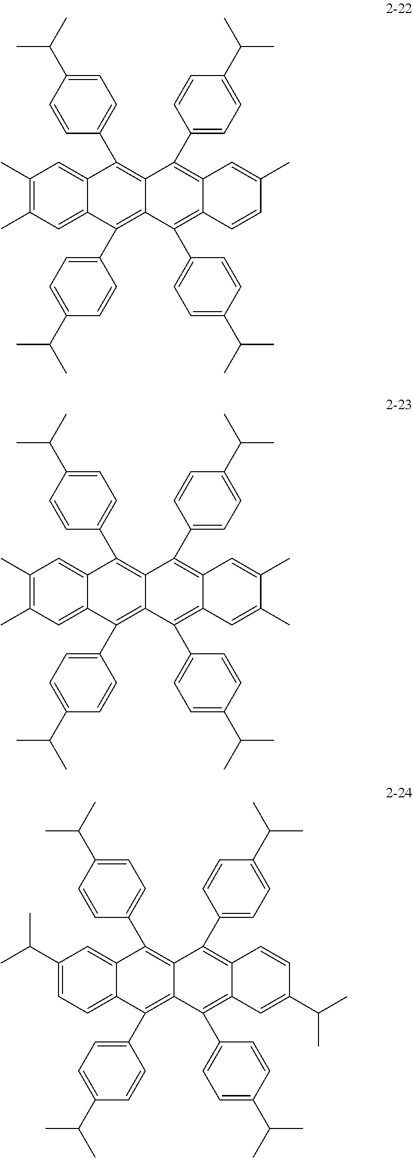

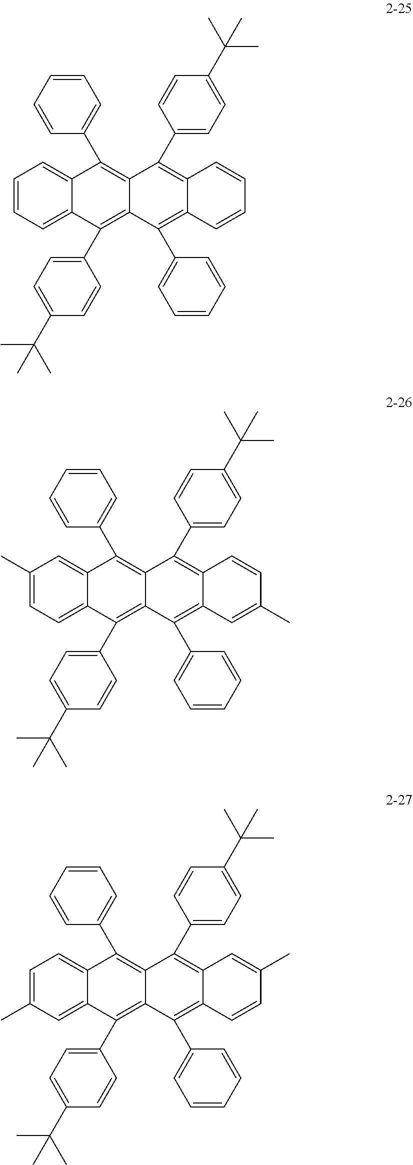

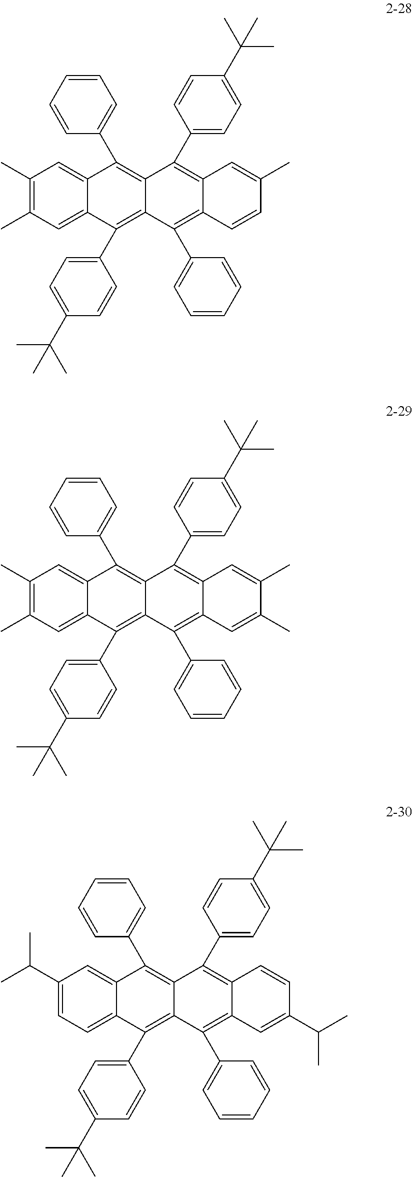

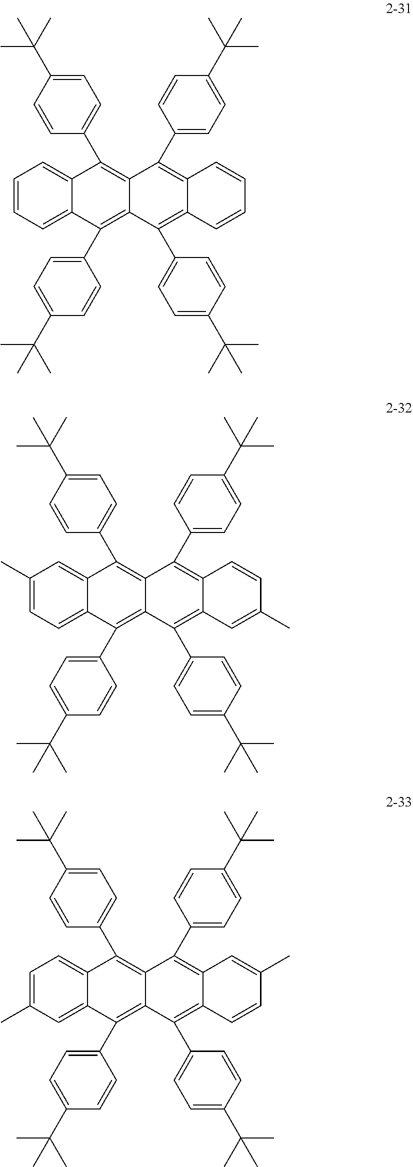

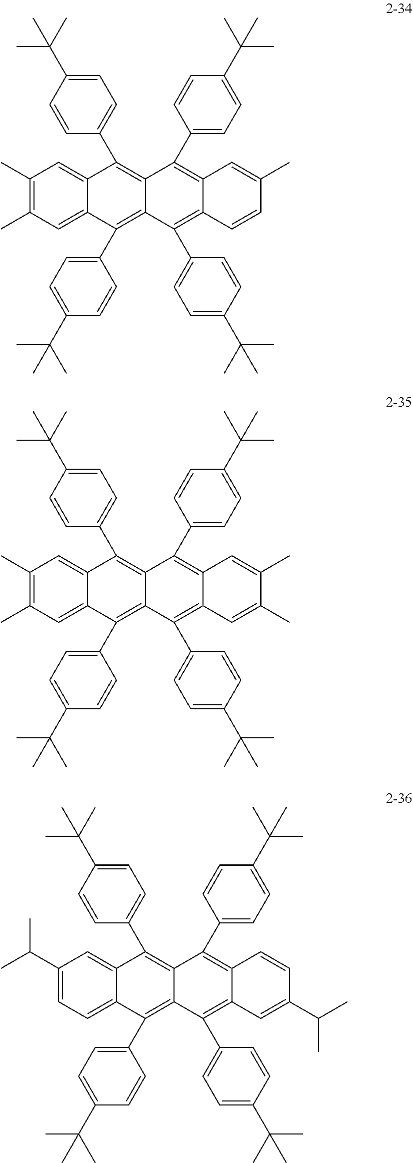

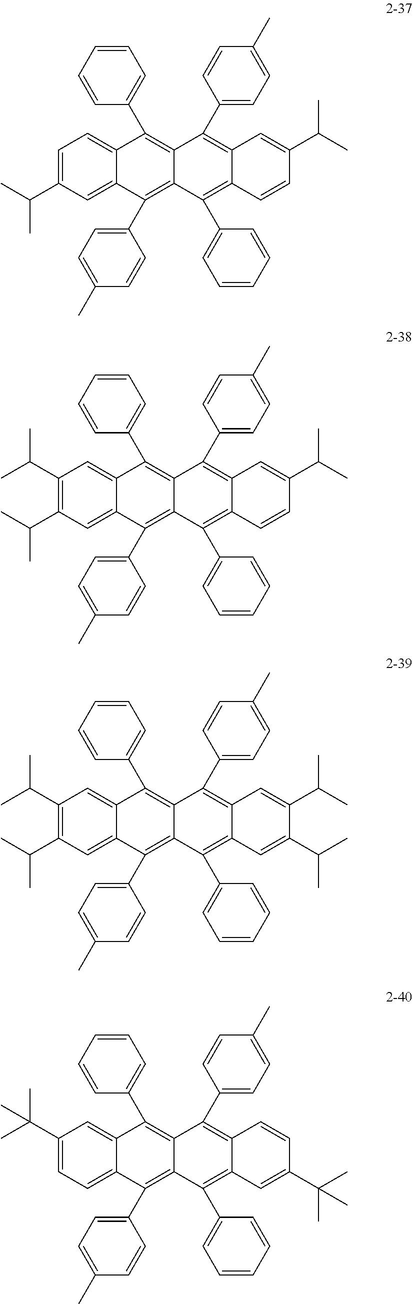

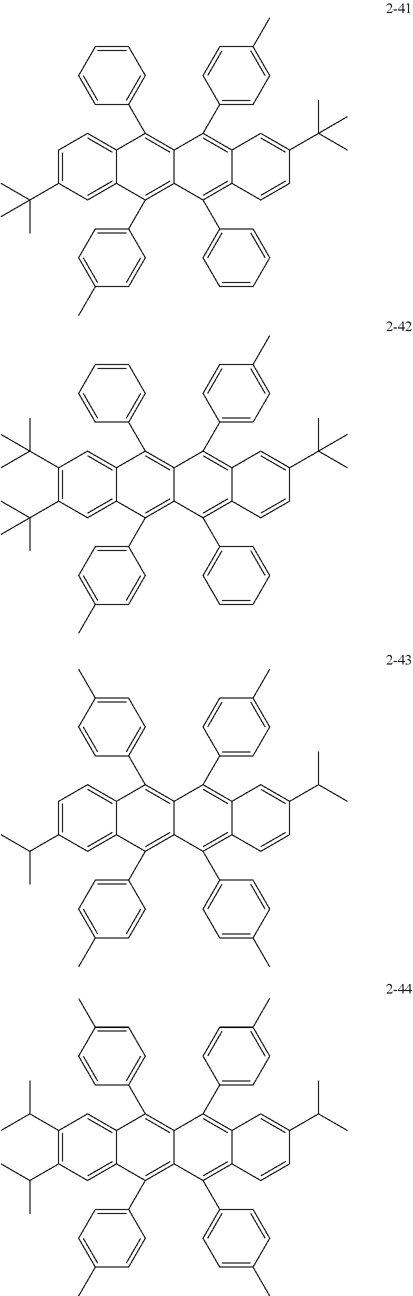

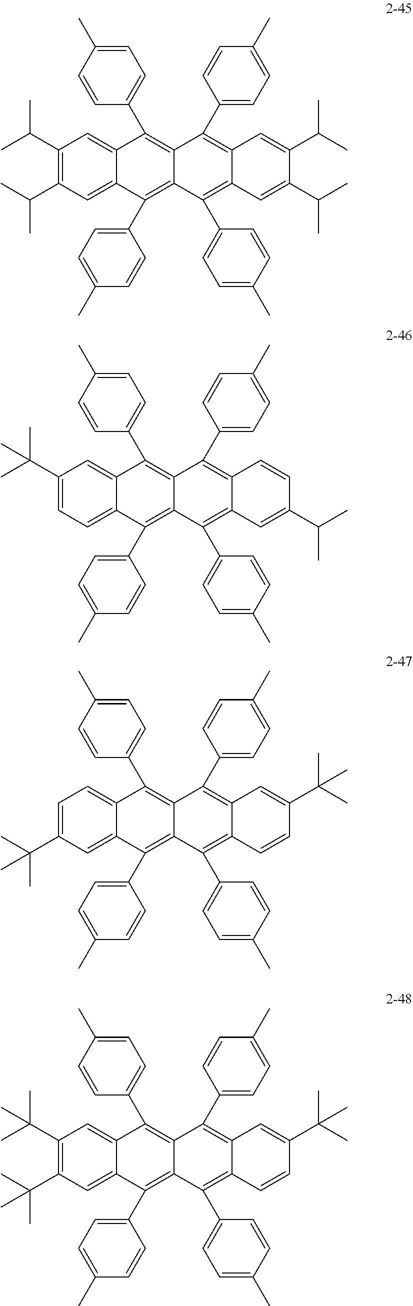

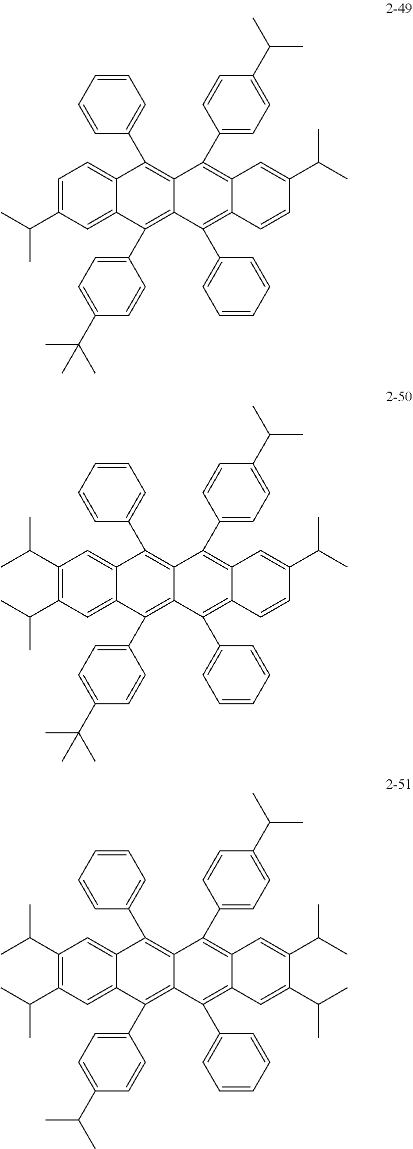

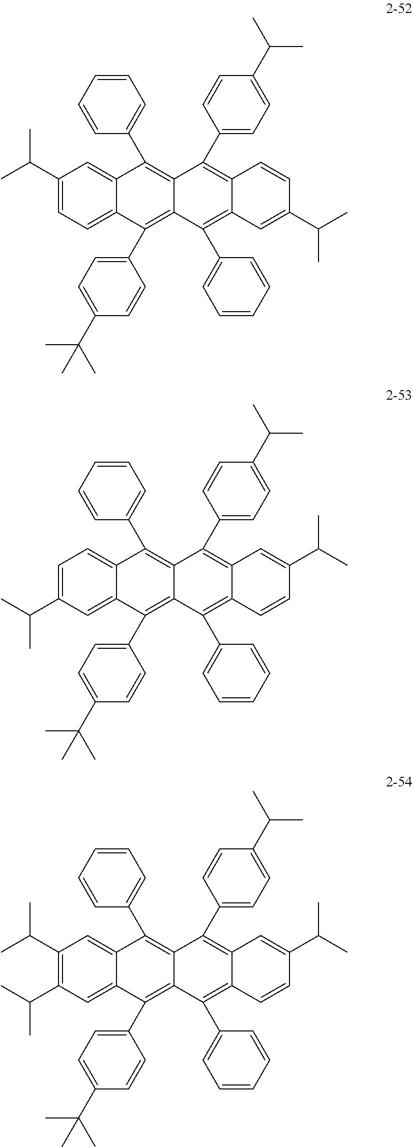

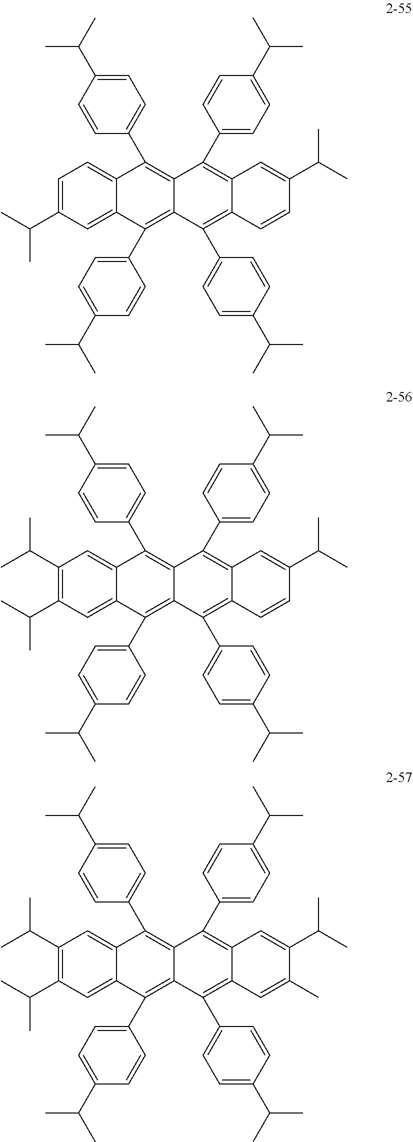

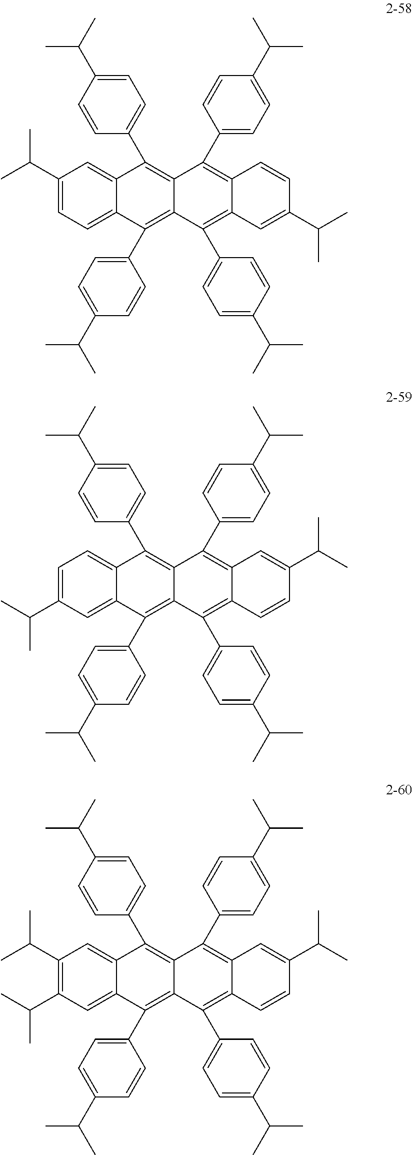

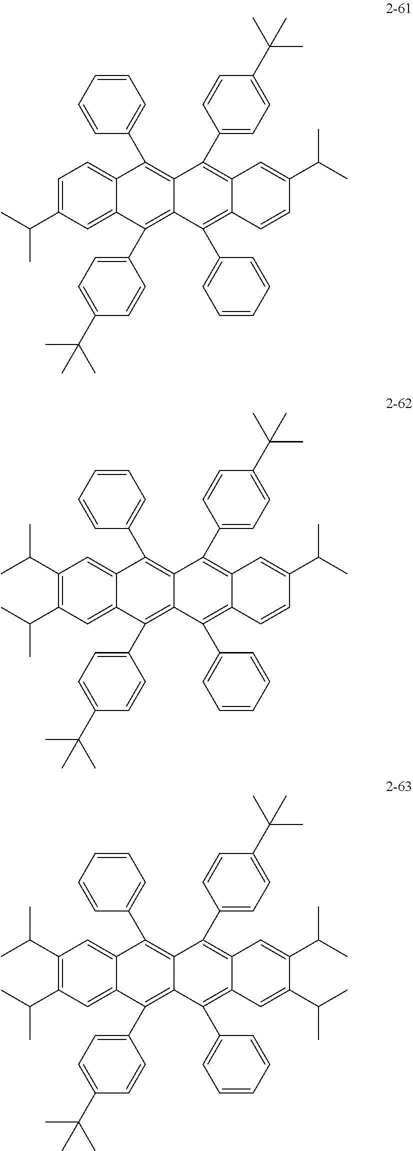

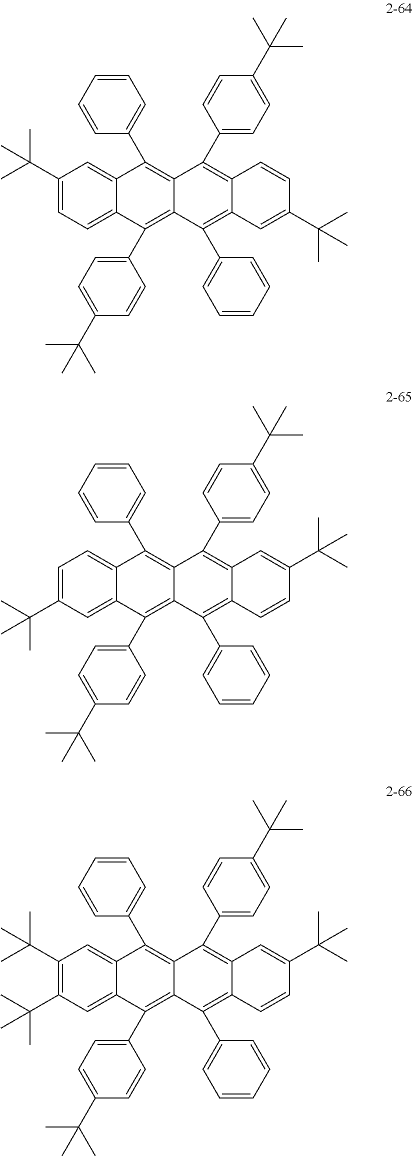

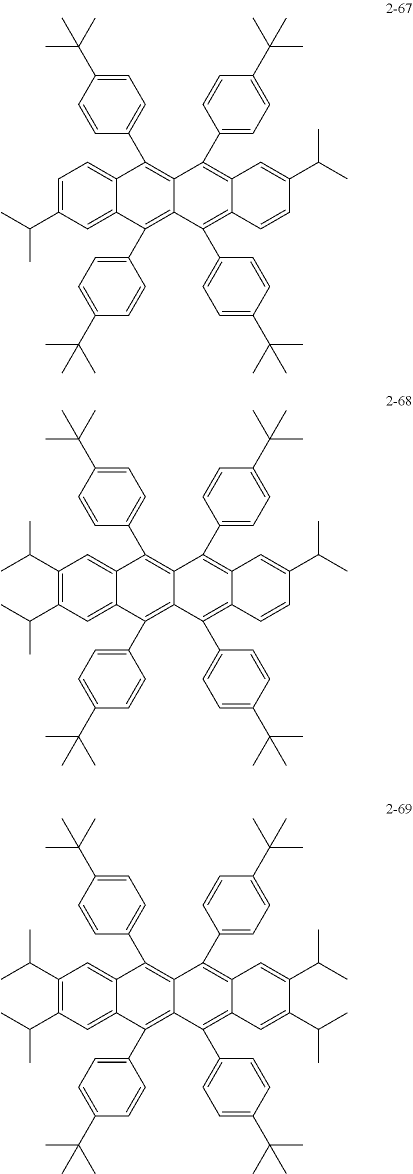

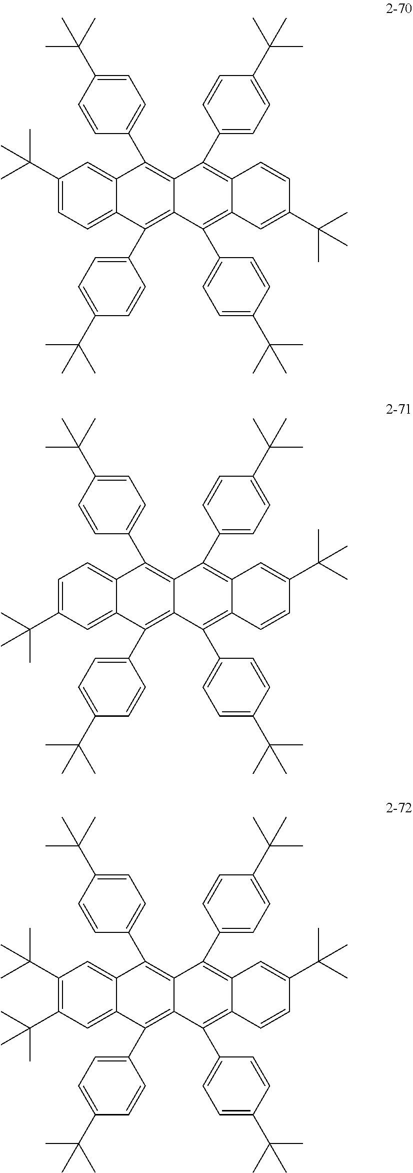

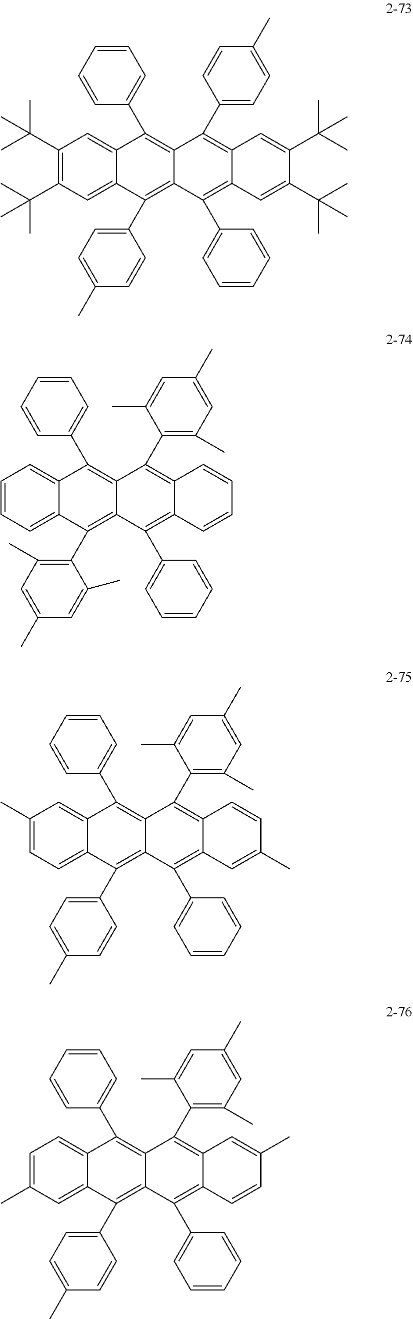

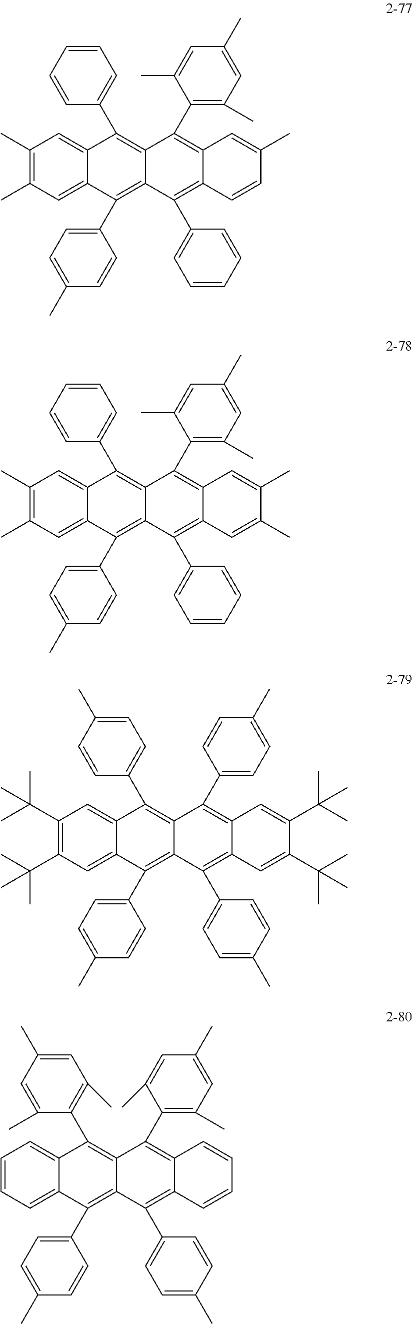

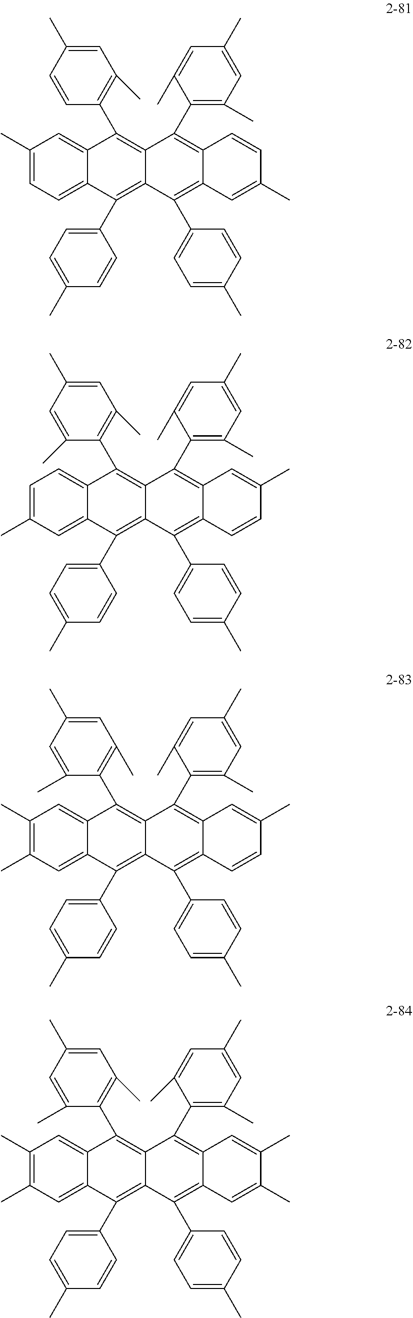

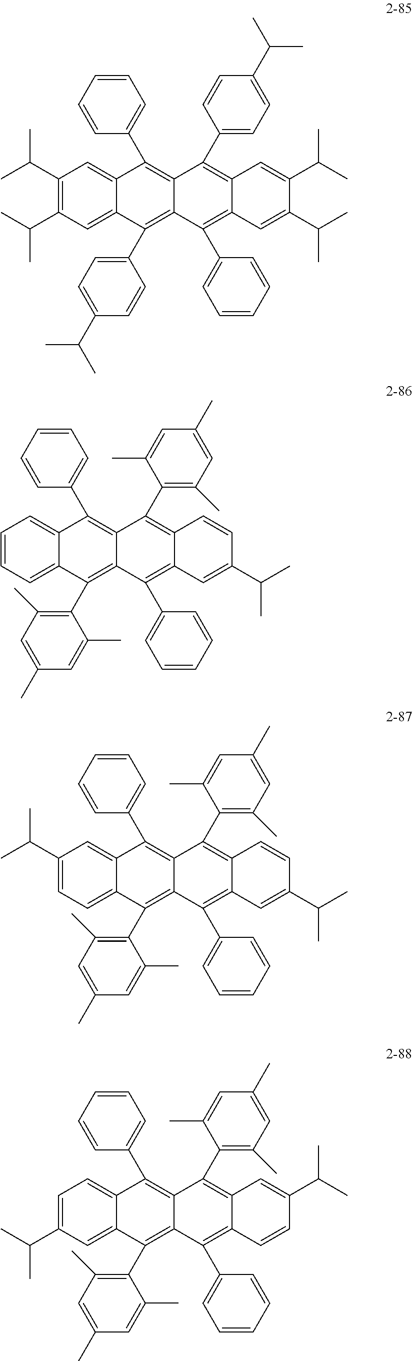

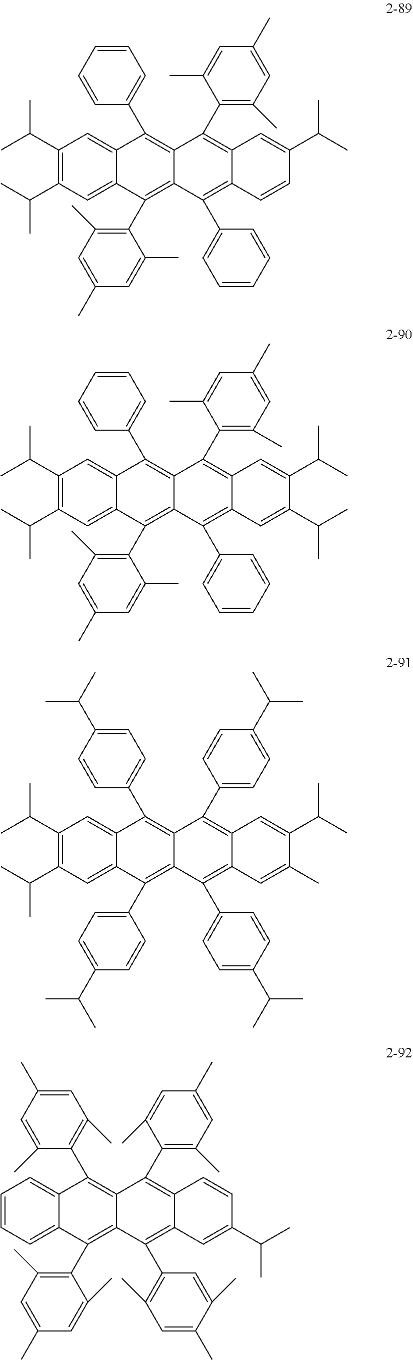

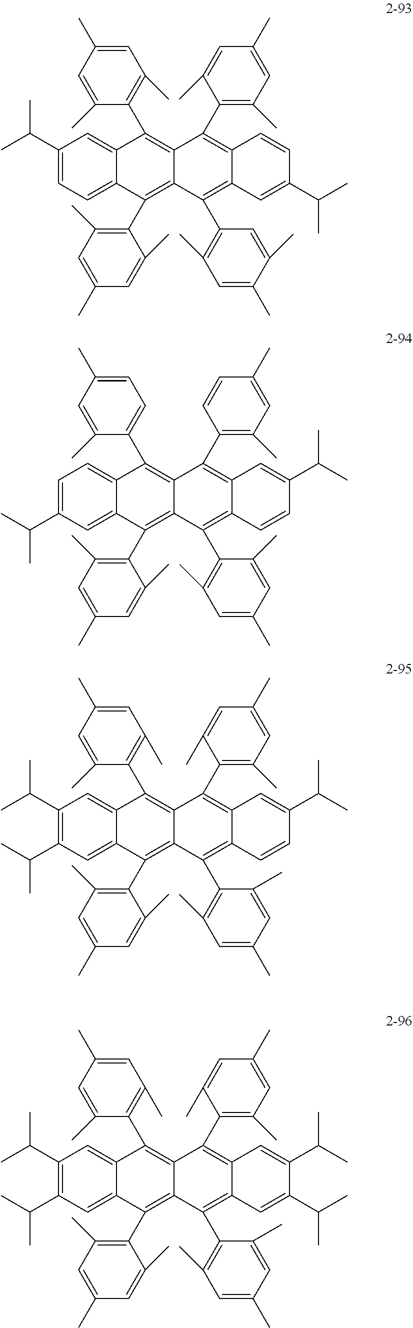

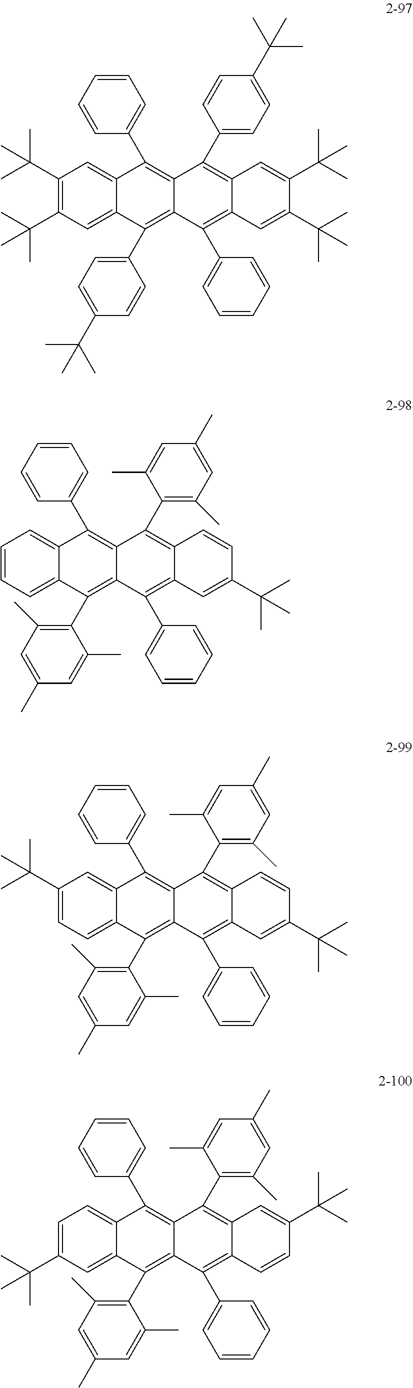

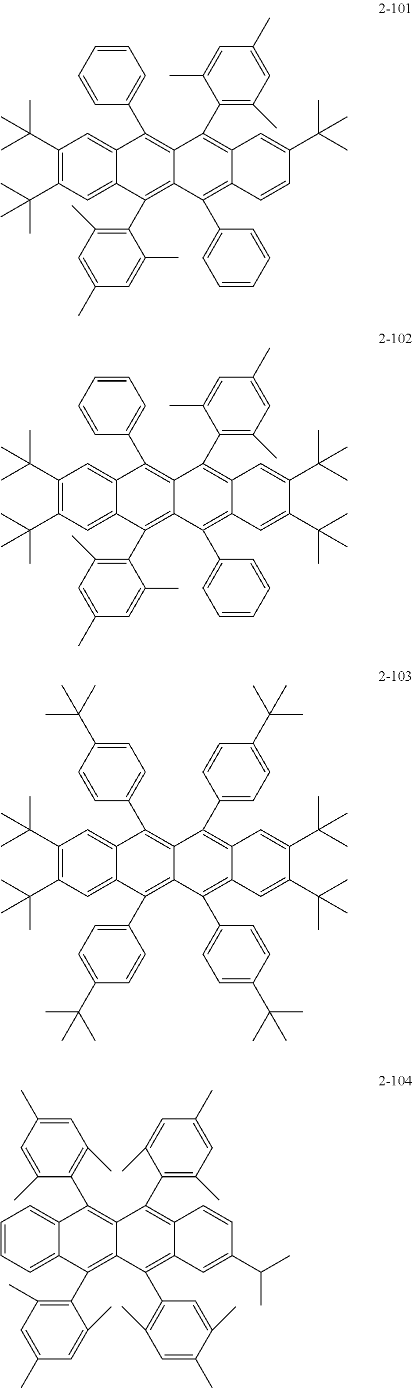

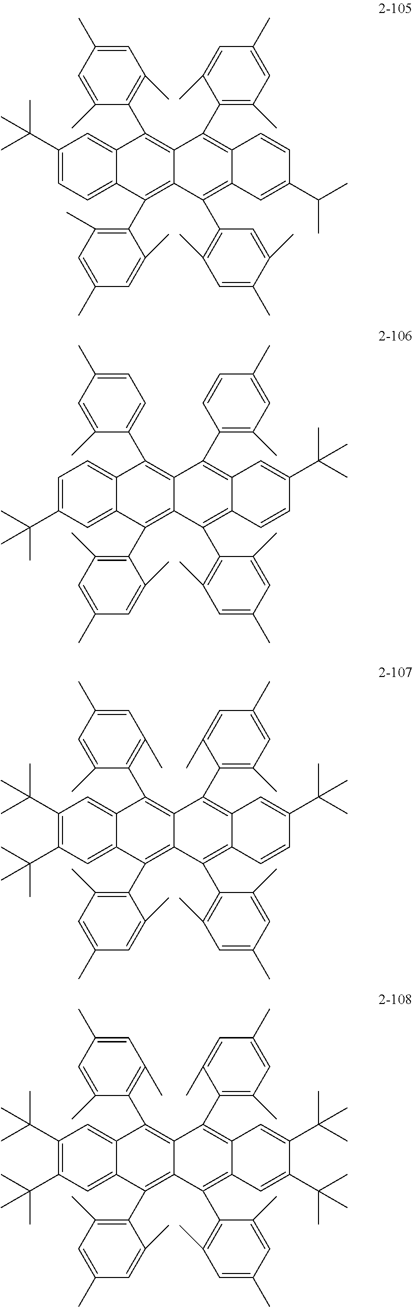

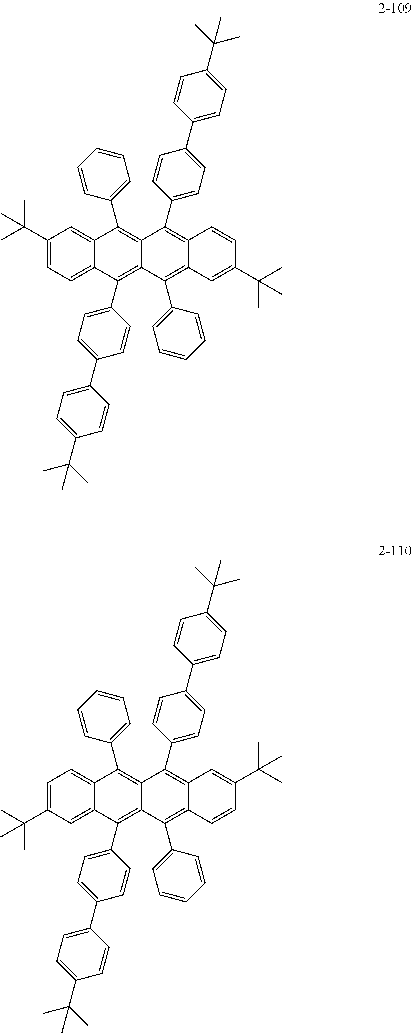

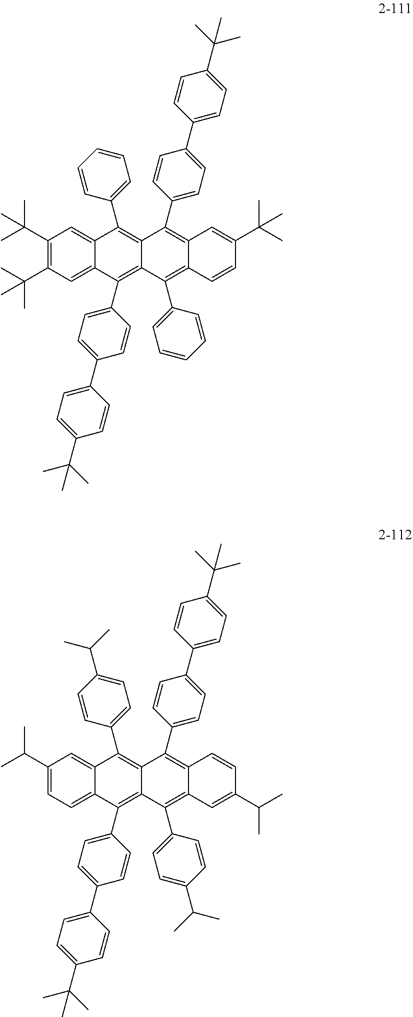

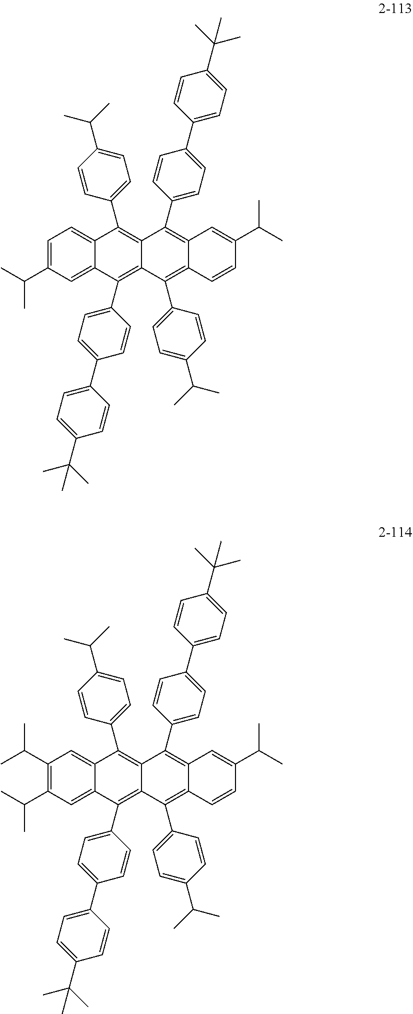

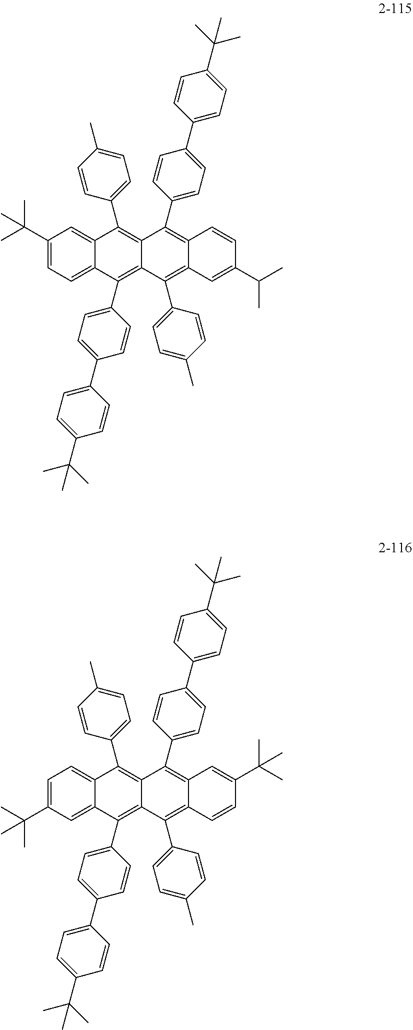

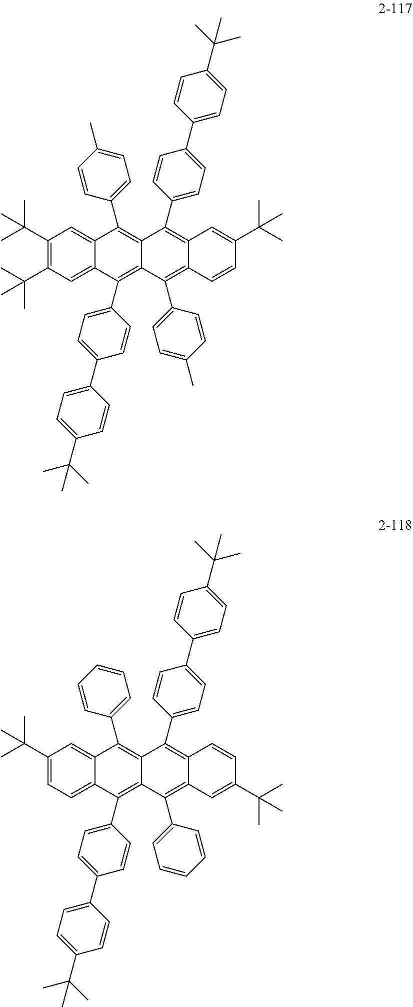

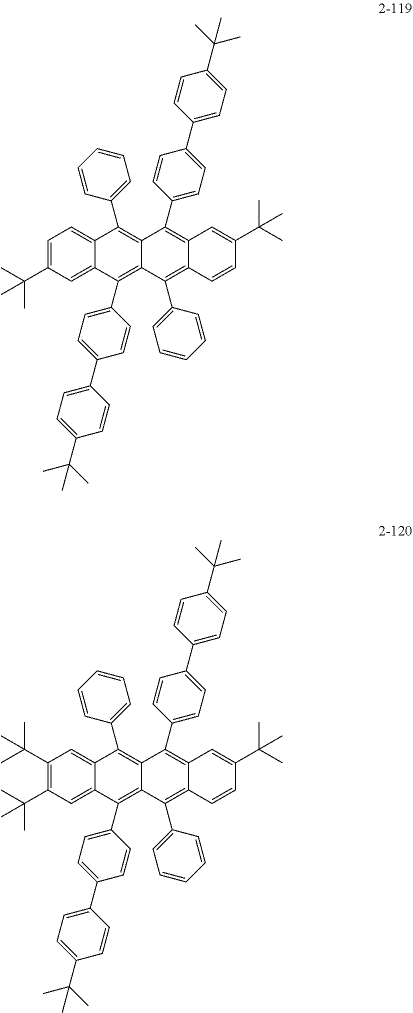

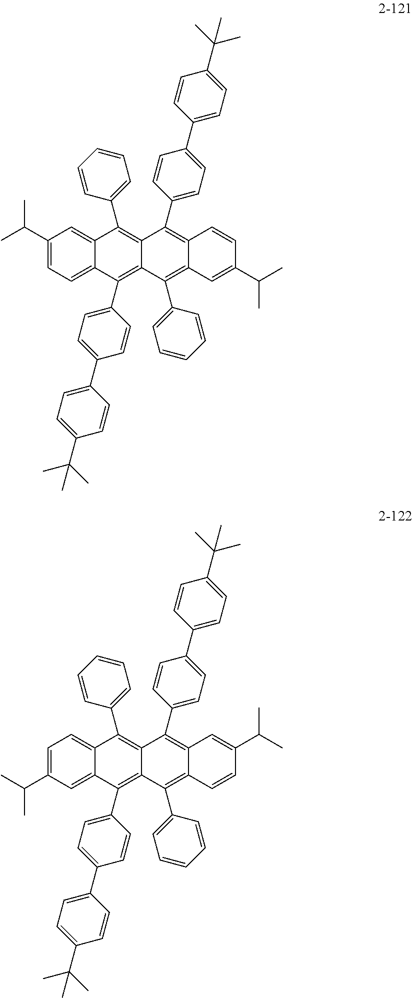

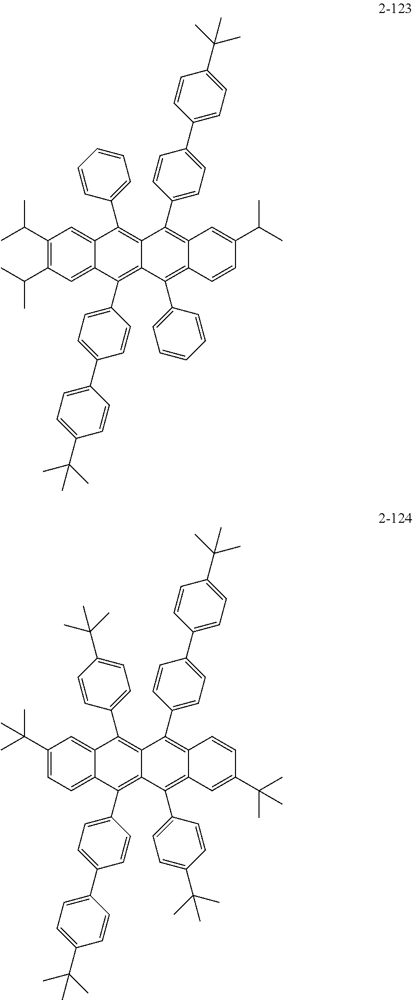

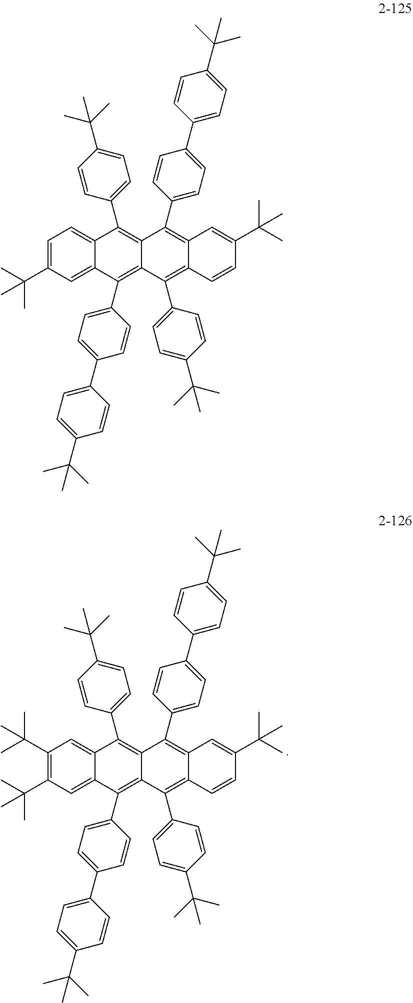

10. The organic light emitting diode of claim 1, wherein the at least one emitting material layer includes a single-layered emitting material layer.

11. The organic light emitting diode of claim 10, wherein the single-layered emitting material layer further includes a third compound.

12. The organic light emitting diode of claim 11, wherein the third compound has the following structure of Formula 8: 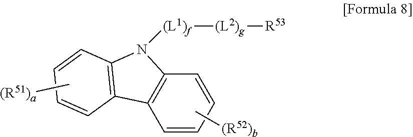
wherein, in Formula 8,
each of R⁵¹ and R⁵² is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when a is an integer of 2 or more, each R⁵¹ is identical to or different from each other, and when b is an integer of 2 or more, each R⁵² is identical to or different from each other;
R₅₃ is unsubstituted or substituted carbazolyl, unsubstituted or substituted dibenzofuranyl or unsubstituted or substituted dibenzothiophenyl;
each of L¹ and L² is independently unsubstituted or substituted C₆-C₃₀ arylene or unsubstituted or substituted C₃-C₃₀ hetero arylene; and
each of f and g is independently 0 or 1.

13. The organic light emitting diode of claim 12, wherein the third compound has the following structure of Formula 9A or Formula 9B: 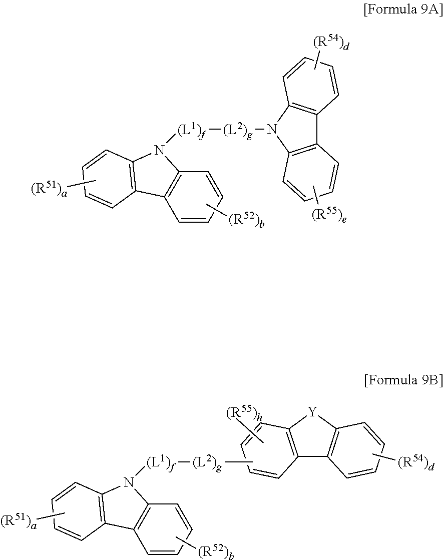
wherein, in Formulae 9A and 9B,
each of R⁵¹, R⁵², L¹, L², a, b, f and g is identical as defined in Formula 8;
each of R⁵⁴ and R⁵⁵ is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when d is an integer of 2 or more, each R⁵⁴ is identical to or different from each other, and when e or h is an integer of 2 or more, each R⁵⁵ is identical to or different from each other;
Y is O, S, or NH;
each of d and e is independently an integer of 0 to 4; and
h is an integer of 0 to 3.

14. The organic light emitting diode of claim 11, wherein the third compound is selected from the following compounds:

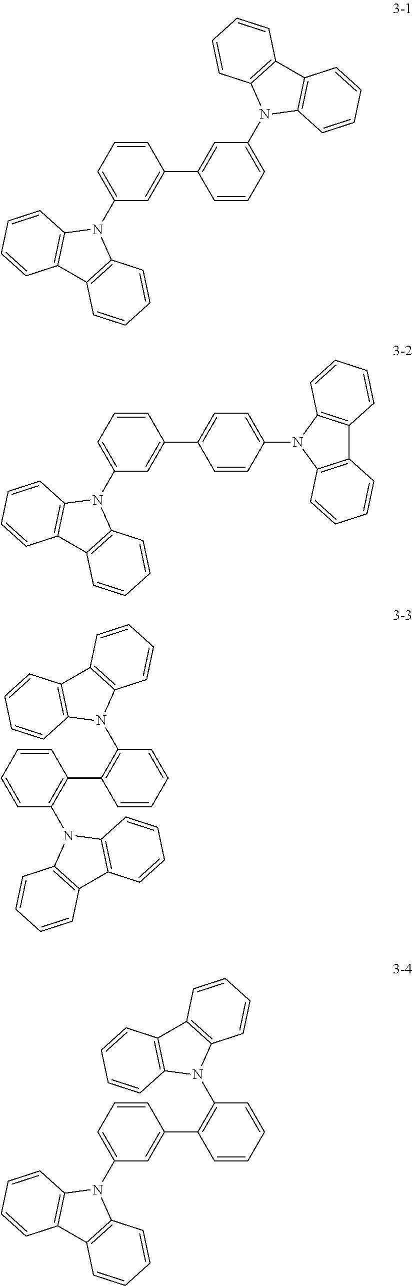

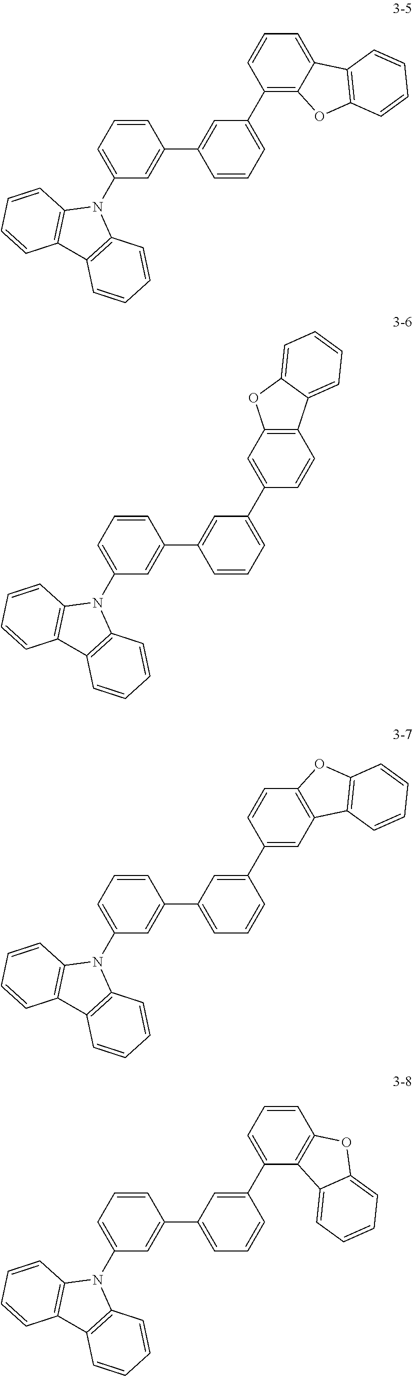 [SMILES: c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2ccc3c(c2)oc2ccccc23)c1 ; c1cc(-c2cccc(-n3c4ccccc4c4ccccc43)c2)cc(-c2ccc3oc4ccccc4c3c2)c1 ; c1cc(-c2cccc(-n3c4ccccc4c4ccccc43)c2)cc(-c2cccc3oc4ccccc4c23)c1]

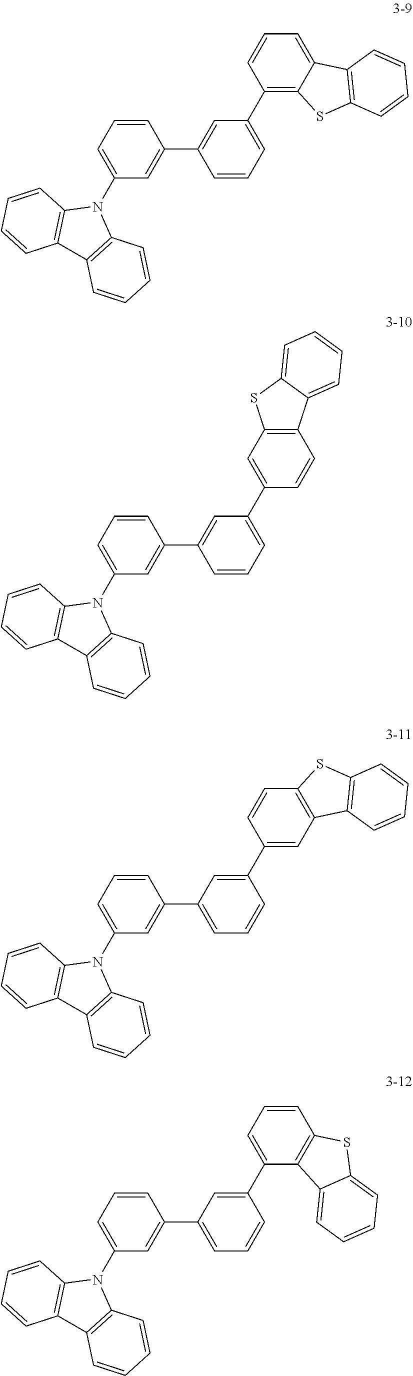 [SMILES: c1cc(-c2cccc(-n3c4ccccc4c4ccccc43)c2)cc(-c2cccc3c2sc2ccccc23)c1 ; c1cc(-c2cccc(-n3c4ccccc4c4ccccc43)c2)cc(-c2ccc3c(c2)sc2ccccc23)c1 ; c1cc(-c2cccc(-n3c4ccccc4c4ccccc43)c2)cc(-c2ccc3sc4ccccc4c3c2)c1 ; c1cc(-c2cccc(-n3c4ccccc4c4ccccc43)c2)cc(-c2cccc3sc4ccccc4c23)c1]

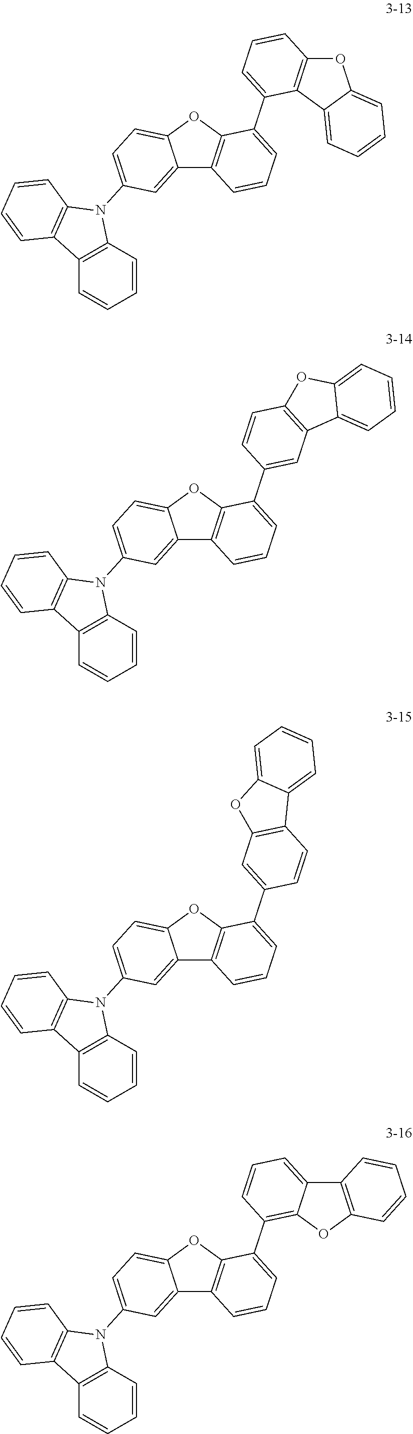 [SMILES: c1ccc2c(c1)oc1cccc(-c3cccc4c3oc3ccc(-n5c6ccccc6c6ccccc65)cc34)c12 ; c1ccc2c(c1)oc1ccc(-c3cccc4c3oc3ccc(-n5c6ccccc6c6ccccc65)cc34)cc12 ; c1ccc2c(c1)oc1cc(-c3cccc4c3oc3ccc(-n5c6ccccc6c6ccccc65)cc34)ccc12 ; c1ccc2c(c1)oc1c(-c3cccc4c3oc3ccc(-n5c6ccccc6c6ccccc65)cc34)cccc12]

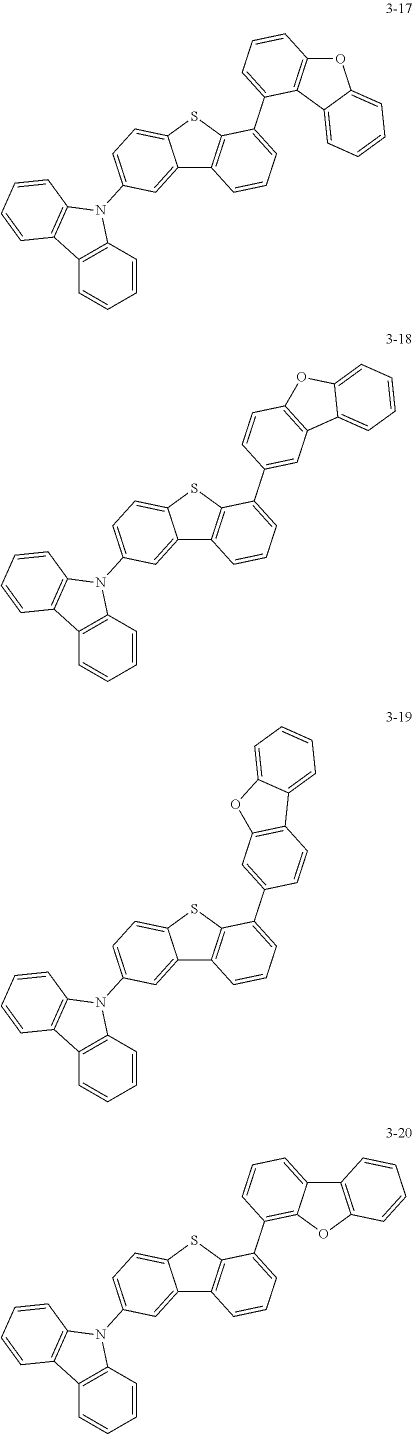 [SMILES: c1ccc2c(c1)oc1cccc(-c3cccc4c3sc3ccc(-n5c6ccccc6c6ccccc65)cc34)c12 ; c1ccc2c(c1)oc1ccc(-c3cccc4c3sc3ccc(-n5c6ccccc6c6ccccc65)cc34)cc12 ; c1ccc2c(c1)oc1cc(-c3cccc4c3sc3ccc(-n5c6ccccc6c6ccccc65)cc34)ccc12 ; c1ccc2c(c1)oc1c(-c3cccc4c3sc3ccc(-n5c6ccccc6c6ccccc65)cc34)cccc12]

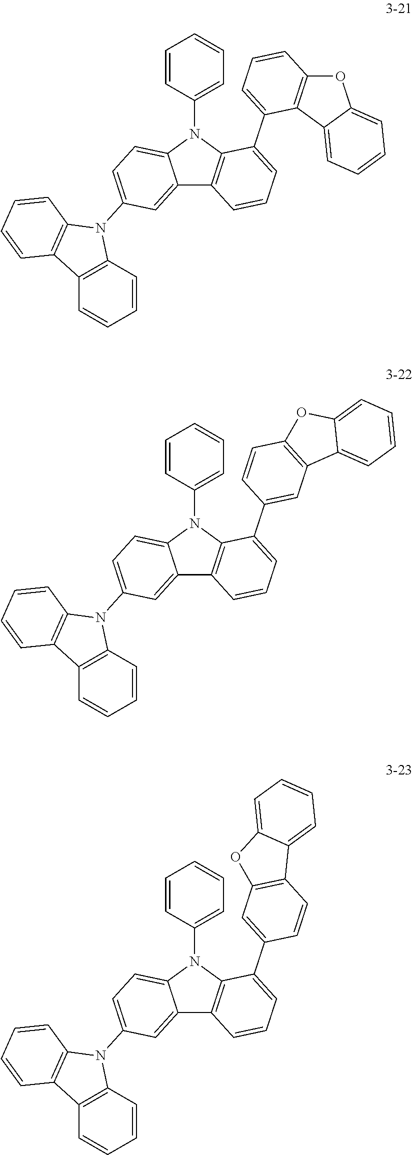 [SMILES: c1ccc(-n2c3ccc(-n4c5ccccc5c5ccccc54)cc3c3cccc(-c4cccc5oc6ccccc6c45)c32)cc1 ; c1ccc(-n2c3ccc(-n4c5ccccc5c5ccccc54)cc3c3cccc(-c4ccc5oc6ccccc6c5c4)c32)cc1 ; c1ccc(-n2c3ccc(-n4c5ccccc5c5ccccc54)cc3c3cccc(-c4ccc5c(c4)oc4ccccc45)c32)cc1]

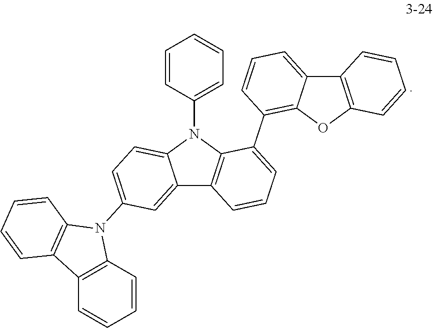 [SMILES: c1ccc(-n2c3ccc(-n4c5ccccc5c5ccccc54)cc3c3cccc(-c4cccc5c4oc4ccccc45)c32)cc1]

15. The organic light emitting diode of claim 11, wherein an excited triplet exciton energy level of the third compound is higher than an excited triplet exciton energy level of the first compound and the exited triplet exciton energy level of the first compound is higher than an excited triplet exciton energy level of the second compound, and
wherein an excited singlet exciton energy level of the third compound is higher than an excited singlet exciton energy level of the first compound and the excited singlet exciton energy level of the first compound is higher than an excited singlet exciton energy level of the second compound.

16. The organic light emitting diode of claim 1, wherein the at least one emitting material layer includes a first emitting material layer disposed between the first and second electrodes and a second emitting material layer disposed between the first electrode and the first emitting material layer or between the first emitting material layer and the second electrode, and
wherein the first emitting material layer includes the first compound and the second emitting material layer includes the second compound.

17. The organic light emitting diode of claim 16, wherein the first emitting material layer further includes a third compound and the second emitting material layer further includes a fourth compound.

18. The organic light emitting diode of claim 17, wherein the third compound has the following structure of Formula 8: 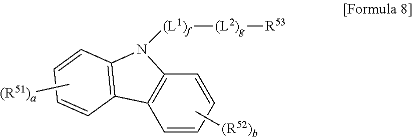
wherein, in Formula 8,
each of R⁵¹ and R⁵² is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when a is an integer of 2 or more, each R⁵¹ is identical to or different from each other, and when b is an integer of 2 or more, each R⁵² is identical to or different from each other;
R₅₃ is unsubstituted or substituted carbazolyl, unsubstituted or substituted dibenzofuranyl or unsubstituted or substituted dibenzothiophenyl;
each of L¹ and L² is independently unsubstituted or substituted C₆-C₃₀ arylene or unsubstituted or substituted C3-C30 hetero arylene; and
each of f and g is independently 0 or 1.

19. The organic light emitting diode of claim 18, wherein the third compound has the following structure of Formula 9A or Formula 9B: 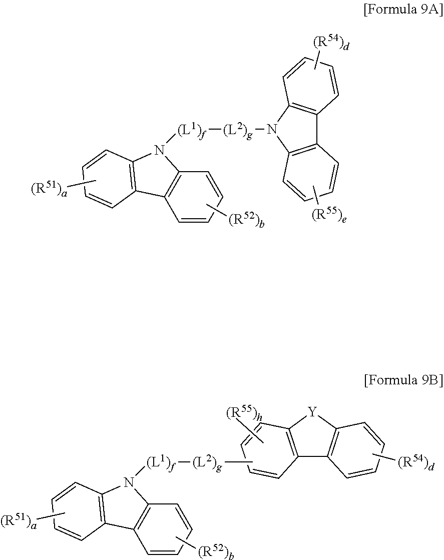
wherein, in Formulae 9A and 9B,
each of R⁵¹, R⁵², L¹, L², a, b, f and g is identical as defined in Formula 8;
each of R⁵⁴ and R⁵⁵ is independently hydrogen, protium, deuterium, tritium, unsubstituted or substituted C₁-C₁₀ alkyl, unsubstituted or substituted C₆-C₃₀ aryl or unsubstituted or substituted C₃-C₃₀ hetero aryl, when d is an integer of 2 or more, each R⁵⁴ is identical to or different from each other, and when e or h is an integer of 2 or more, each R⁵⁵ is identical to or different from each other;
Y is O, S, or NH;
each of d and e is independently an integer of 0 to 4; and
h is an integer of 0 to 3.

20. The organic light emitting diode of claim 17, wherein the third compound is selected from the following compounds:

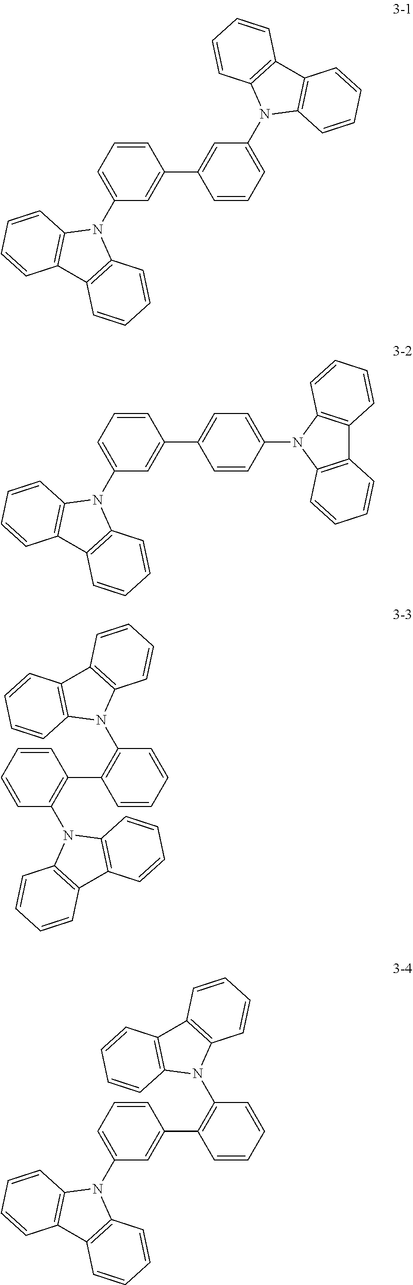

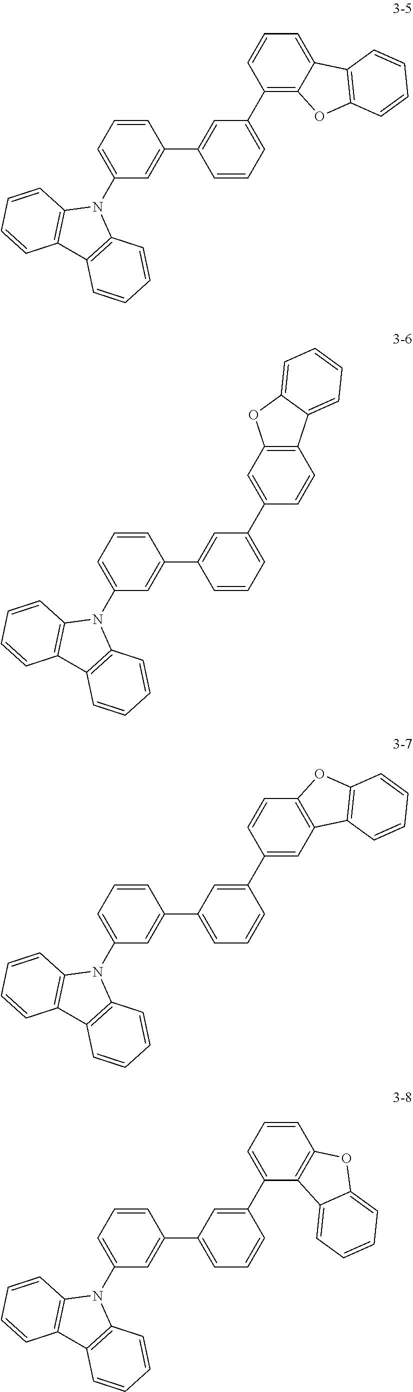 [SMILES: c1cc(-c2cccc(-n3c4ccccc4c4ccccc43)c2)cc(-c2cccc3c2oc2ccccc23)c1 ; c1cc(-c2cccc(-n3c4ccccc4c4ccccc43)c2)cc(-c2ccc3c(c2)oc2ccccc23)c1 ; c1cc(-c2cccc(-n3c4ccccc4c4ccccc43)c2)cc(-c2ccc3oc4ccccc4c3c2)c1 ; c1cc(-c2cccc(-n3c4ccccc4c4ccccc43)c2)cc(-c2cccc3oc4ccccc4c23)c1]

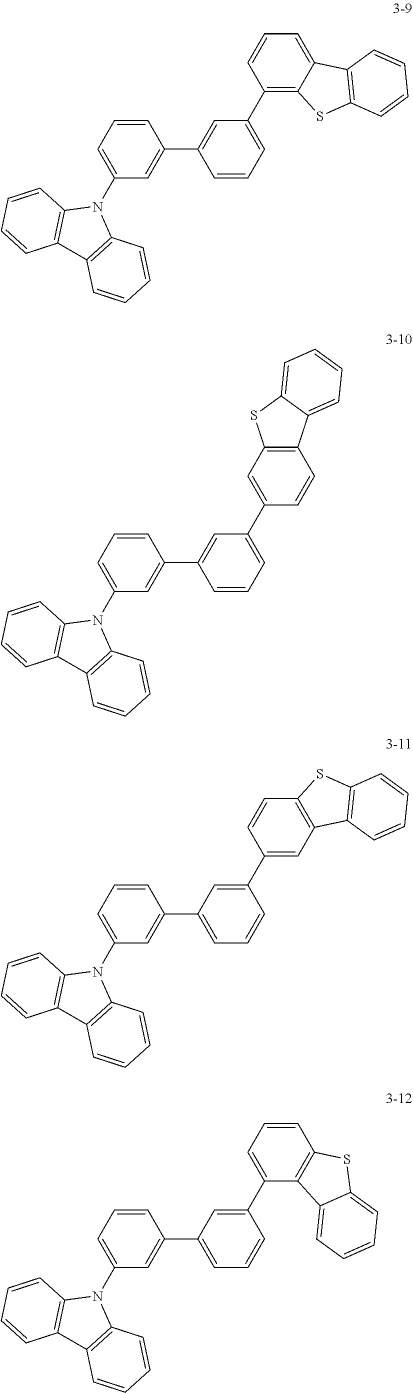 [SMILES: c1cc(-c2cccc(-n3c4ccccc4c4ccccc43)c2)cc(-c2cccc3c2sc2ccccc23)c1 ; c1cc(-c2cccc(-n3c4ccccc4c4ccccc43)c2)cc(-c2ccc3c(c2)sc2ccccc23)c1 ; c1cc(-c2cccc(-n3c4ccccc4c4ccccc43)c2)cc(-c2ccc3sc4ccccc4c3c2)c1 ; c1cc(-c2cccc(-n3c4ccccc4c4ccccc43)c2)cc(-c2cccc3sc4ccccc4c23)c1]

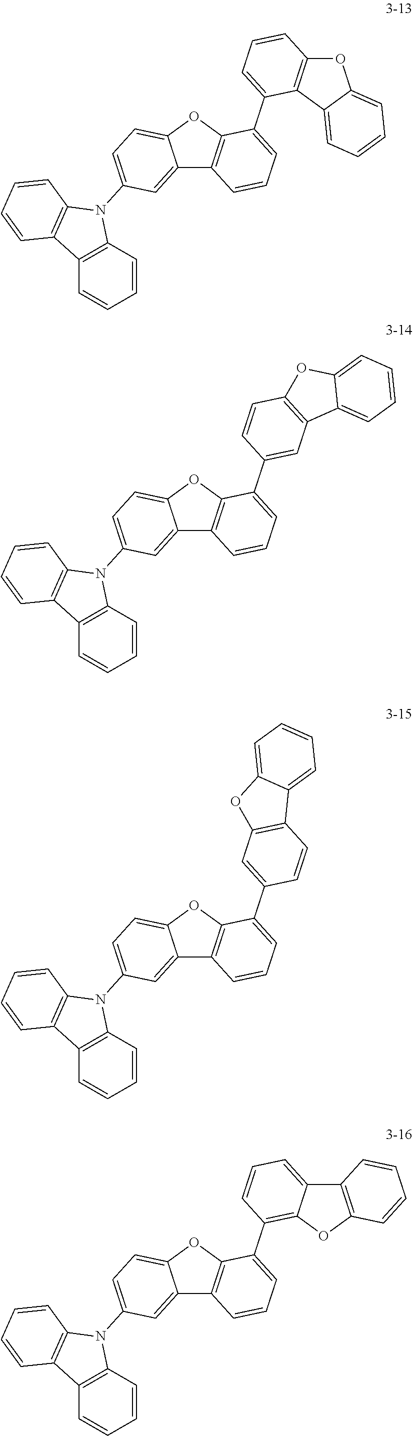 [SMILES: c1ccc2c(c1)oc1cccc(-c3cccc4c3oc3ccc(-n5c6ccccc6c6ccccc65)cc34)c12 ; c1ccc2c(c1)oc1ccc(-c3cccc4c3oc3ccc(-n5c6ccccc6c6ccccc65)cc34)cc12 ; c1ccc2c(c1)oc1cc(-c3cccc4c3oc3ccc(-n5c6ccccc6c6ccccc65)cc34)ccc12 ; c1ccc2c(c1)oc1c(-c3cccc4c3oc3ccc(-n5c6ccccc6c6ccccc65)cc34)cccc12]

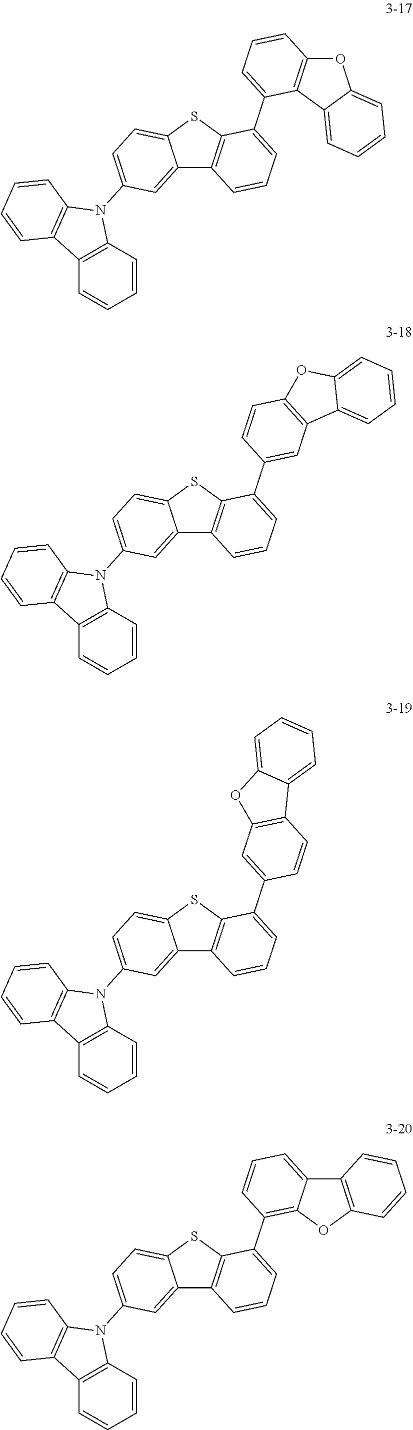 [SMILES: c1ccc2c(c1)oc1cccc(-c3cccc4c3sc3ccc(-n5c6ccccc6c6ccccc65)cc34)c12 ; c1ccc2c(c1)oc1ccc(-c3cccc4c3sc3ccc(-n5c6ccccc6c6ccccc65)cc34)cc12 ; c1ccc2c(c1)oc1cc(-c3cccc4c3sc3ccc(-n5c6ccccc6c6ccccc65)cc34)ccc12 ; c1ccc2c(c1)oc1c(-c3cccc4c3sc3ccc(-n5c6ccccc6c6ccccc65)cc34)cccc12]

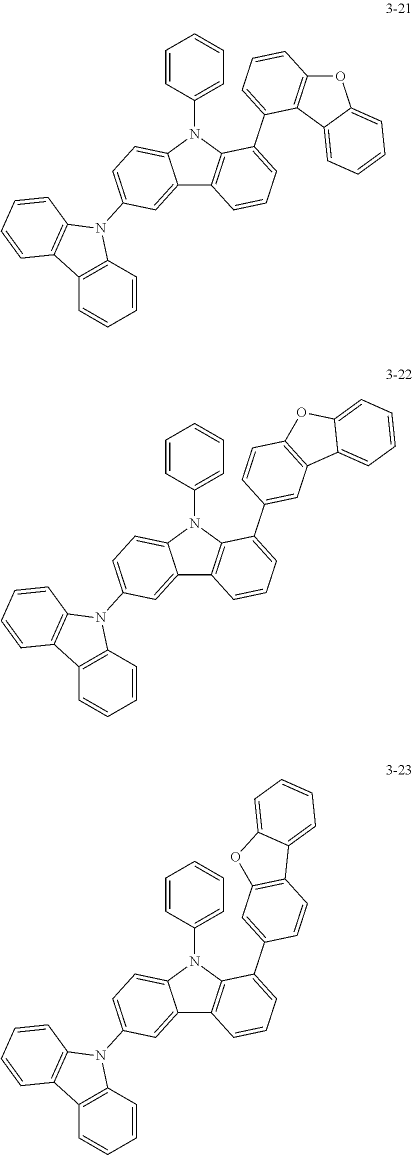 [SMILES: c1ccc(-n2c3ccc(-n4c5ccccc5c5ccccc54)cc3c3cccc(-c4cccc5oc6ccccc6c45)c32)cc1 ; c1ccc(-n2c3ccc(-n4c5ccccc5c5ccccc54)cc3c3cccc(-c4ccc5oc6ccccc6c5c4)c32)cc1 ; c1ccc(-n2c3ccc(-n4c5ccccc5c5ccccc54)cc3c3cccc(-c4ccc5c(c4)oc4ccccc45)c32)cc1]

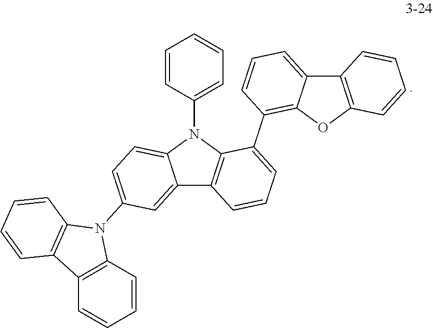 [SMILES: c1ccc(-n2c3ccc(-n4c5ccccc5c5ccccc54)cc3c3cccc(-c4cccc5c4oc4ccccc45)c32)cc1]

21. The organic light emitting diode of claim 17, wherein an excited triplet exciton energy level of the third compound is higher than an excited triplet exciton energy level of the first compound and the exited triplet exciton energy level of the first compound is higher than an excited triplet exciton energy level of the second compound, and
wherein an excited singlet exciton energy level of the third compound is higher than an excited singlet exciton energy level of the first compound and the excited singlet exciton energy level of the first compound is higher than an excited singlet exciton energy level of the second compound.

22. The organic light emitting diode of claim 16, wherein the at least one emitting material layer further includes a third emitting material layer disposed oppositely to the second emitting material layer with respect to the first emitting material layer.

23. The organic light emitting diode of claim 22, wherein the third emitting material layer includes a fifth compound and a sixth compound, and wherein the fifth compound includes the organic compound having the structure of Formula 5.

24. The organic light emitting diode of claim 23, wherein an excited singlet energy level of the sixth compound is higher than an excited singlet energy level of the fifth compound.

25. The organic light emitting diode of claim 1, wherein the emissive layer includes a first emitting part disposed between the first and second electrodes, a second emitting part disposed between the first emitting part and the second electrode and a charge generation layer disposed between the first and second emitting parts, and
wherein at least one of the first emitting part and the second emitting part includes the at least one emitting material layer.

26. An organic light emitting device, comprising:
a substrate; and
the organic light emitting diode according to claim 1 and disposed over the substrate.
